(19) United States
(12) Patent Application Publication
(10) Pub. No.: US 2011/0269814 A1
(43) Pub. Date: Nov. 3, 2011

(54) 2'-F MODIFIED RNA INTERFERENCE AGENTS

(75) Inventors: Muthiah Manoharan, Cambridge, MA (US); Kallanthottathil G. Rajeev, Cambridge, MA (US)

(73) Assignee: ALNYLAM PHARAMACEUTICALS, INC., Cambridge, MA (US)

(21) Appl. No.: 12/935,083

(22) PCT Filed: Mar. 26, 2009
(86) PCT No.: PCT/US09/38433

§ 371 Date: Mar. 31, 2011

Related U.S. Application Data
(60) Provisional application No. 61/039,574, filed on Mar. 26, 2008.
(60) Provisional application No. 61/040,414, filed on Mar. 28, 2008.
(60) Provisional application No. 61/105,307, filed on Oct. 14, 2008.

(51) Int. Cl. A61K 31/713; C07H 21/02; C07K 16/18; C07K 2/00; C07K 9/00; C07H 1/00; C12N 5/071

(52) U.S. Cl. 514/44 A; 536/25.3; 536/24.5; 435/375; 530/322; 530/358

(57) ABSTRACT

This invention relates to a method of modulating the expression of a target gene in an organism comprising administering an iRNA agent, wherein the iRNA comprises at least one 2′-deoxy-2′-fluoro (2′-F) nucleotide in the antisense strand and at least one modified nucleotide in the sense strand. The invention also relates to compositions comprising a single-stranded oligonucleotide that contains at least one 2′-deoxy-2′-fluoro (2′-F) nucleotide. siRNA molecule containing these oligonucleotides have decreased immunogenicity.

### CROSS REFERENCE TO RELATED APPLICATIONS

This application claims the benefit PCT Application No. PCT/US09/38433, filed Mar. 26, 2009, which claims priority to U.S. Provisional Patent Application No. 61/039,574, filed Mar. 26, 2008; U.S. Provisional Patent Application No. 61/040,414, filed Mar. 28, 2008; U.S. Provisional Patent Application No. 61/105,307, filed Oct. 14, 2008, all of which are hereby incorporated by reference in their entirety.

### TECHNICAL FIELD

The invention relates to modified oligonucleotide formulations, in particular oligonucleotides having 2′-deoxy-2′-fluoro modifications.

### BACKGROUND OF THE INVENTION

Many diseases (e.g., cancers, hematopoietic disorders, endocrine disorders, and immune disorders) arise from the abnormal expression or activity of a particular gene or group of genes. Similarly, disease can result through expression of a mutant form of protein, as well as from expression of viral genes that have been integrated into the genome of their host. The therapeutic benefits of being able to selectively silence these abnormal or foreign genes are obvious.

Oligonucleotide compounds have important therapeutic applications in medicine. Oligonucleotides can be used to silence genes that are responsible for a particular disease. Gene-silencing prevents formation of a protein by inhibiting translation. Importantly, gene-silencing agents are a promising alternative to traditional small, organic compounds that inhibit the function of the protein linked to the disease. siRNA, antisense oligonucleotides, and micro-RNA are oligonucleotides that prevent the formation of proteins by gene-silencing.

RNA interference (“RNAi”) is an important biological pathway that has practical applications in the fields of functional gene analysis, drug target validation, and therapeutics. The term RNA interference or “RNAi” is initially coined by Fire and co-workers to describe the observation that double-stranded RNA (dsRNA) can block gene expression when it is introduced into worms (Fire et al. (1998) Nature 391, 806-811). Short dsRNA directs gene-specific, post-transcriptional silencing in many organisms, including vertebrates, and has provided a new tool for studying gene function. RNAi is mediated by RNA-induced silencing complex (RISC), a sequence-specific, multicomponent nuclease that destroys messenger RNAs homologous to the silencing trigger. RISC is known to contain short RNAs (approximately 22 nucleotides) derived from the double-stranded RNA trigger (iRNA agent, siRNA), but the protein components of this activity remained unknown. RNAi may also involve mRNA degradation.

iRNA agents are promising agents for a variety of diagnostic and therapeutic purposes. iRNA agents can be used to identify the function of a gene. In addition, iRNA agents offer enormous potential as a new type of pharmaceutical agent which acts by silencing disease-causing genes. Research is currently underway to develop interference RNA therapeutic agents for the treatment of many diseases including central-nervous-system diseases, inflammatory diseases, metabolic disorders, oncology, infectious diseases, and ocular disease.

Current considerations impacting the use of siRNA include: (i) stability; (ii) specificity, including binding affinity; (iii) potency (iv) immune response; (v) delivery methods that impact cell internalization and subcellular localization of the delivered siRNA; and (vi) silencing longevity.

Numerous studies have revealed certain requirements for siRNA length, structure, chemical composition and sequence that are essential to mediate efficient RNAi, see for example Elbashir et al., 2001, EMBO J. 20, 6877, Tuschl et al., International PCT Publication No. WO01/75164, Nykanene et al., 2001, Cell, 107, 309, Chiu et al., 2003, RNA, 9, 1034, Li et al., International PCT Publication No. WO 00/44914, Parrish et al., 2000, Molecular Cell, 6, 1077. Modifications of the siRNAs can impart desirable properties such as resistance to degradation; alter the half life; target the siRNA to a particular target, e.g., to a particular tissue; modulate, e.g., increase or decrease, the affinity of a strand for its complement or target sequence; or hinder or promote modification of a terminal moiety, e.g., modification by a kinase or other enzymes involved in the RISC mechanism pathway. Although modification of siRNAs is desirable, previous studies revealed that modifications of siRNAs usually produce a substantial decrease in interference activity and thus such modifications may not be suitable for siRNAs.

Therefore there is a need to further study modification of siRNAs which impart a desirable property to the siRNAs without decreasing the interference activity. Moreover, there exists a need for the development of RNAi reagents suitable foe use in vivo, in particular for use in developing human therapeutics.

The inventors have discovered, inter alia, that modification of oligonucleotides, such as siRNAs, results in increased potency and silencing longevity while decreasing or eliminating the immune response.

### SUMMARY OF THE INVENTION

In one aspect the invention provides an iRNA agent comprising a sense strand and antisense strand, wherein the antisense strand comprises at least one 2′-deoxy-2′-fluoro (2′-F) nucleotide and the sense strand comprises at least one modified nucleotide with the modification chosen independently from a group consisting of 2′-deoxy-2′-fluoro, 2′-O-methyl (2′-OMe), 2′-methoxyethyl (2′-MOE) and 2′-O,4′-C-methylene (Locked Nucleic Acids, LNA).

In one aspect the invention provides a single stranded siRNA agent (ssRNA), wherein the single strand comprises at least one 2′-deoxy-2′-fluoro (2′-F) nucleotide and with or without nucleotide modification chosen independently from a group consisting of 2′-O-methyl (2′-OMe), 2′-methoxyethyl (2′-MOE) and 2′-O,4′-C-methylene (Locked Nucleic Acids, LNA). In one aspect, the present invention provides an ssRNA comprising at least one modified nucleoside selected from the group consisting of modified MOE moieties, pseudouridines, modified g-clamps and modified phenoxazines. The invention further provides oligonucleotides with 5′-phosphorothioate, 5′-phosphothoester and 5′-dithioate, dimmers with g-clamps and phenoxazine, dimers with two purines (i.e. 3′-GG, AA, AG, GA, GI, IA etc.), 5′-end position 1 nucleoside with purines which are modified at 2 and 6-positions (A, I, Purine, G), 2′-position modified with —O—CH₂—CH₂—N(CH₂—CH₂—NMe₂), C-5 alkylamine, allylamine containing pyrimidines at position of the 5′-end of the guide strand, or combinations thereof. The invention also provide single stranded siRNA containing a motif selected from the group consisting of 5′ phosphorothioate or 5′-phosphorodithioate, nucleotides 1 and 2 having cationic modifications via C-5 position of pyrimidines, 2-Position of Purines, N2-G, G-clamp, 8-position of purines, 6-position of purines, internal nucleotides having a 2′-F sugar with base modifications (Pseudouridine, G-clamp, phenoxazine, pyridopyrimidines, gem2′-Me-up/2′-F-down), 3′-end with two purines with novel 2′-substituted MOE analogs, 5′-end nucleotides with novel 2′-substituted MOE analogs, 5′-end having a 3′-F and a 2′-5′-linkage, 4′-substituted nucleoside at the nucleotide 1 at 5′-end and the substituent is cationic, alkyl, alkoxyalkyl, thioether and the like, 4′-substitution at the 3′-end of the strand, and combinations thereof.

In another aspect the invention provides a method of modulating the expression of a target gene in an organism comprising administering an iRNA agent of the present invention.

A composition, comprising a short interfering ribonucleic acid (siRNA) molecule 19 to 29 or 15 to 30 nucleotides in length, wherein at least one nucleotide comprises a 2′-deoxy-2′-fluoro modification, the siRNA molecule is at least 75% complementary to a nucleic acid molecule encoding a protein of interest, the siRNA molecule inhibits the expression of the nucleic acid molecule, and the siRNA molecule comprises at least eight consecutive nucleotides of the nucleic acid molecule.

Another aspect of the invention relates to a method of suppressing the endogenous expression of a gene, comprising contacting a cell with an effective amount of the composition or siRNA of the invention, wherein the effective amount is an amount that partially or substantially suppresses the endogenous expression of said gene.

### BRIEF DESCRIPTION OF THE DRAWINGS

FIGS. 1A and 1B show photographs of two gel electrophoresis separations demonstrating that a modified siRNA (AD-1661; FIG. 1A) has a half-life (t_{1/2}) greater than 24 hours when incubated in human serum at a temperature 37° C., as compared to an unmodified siRNA (AD-1596; FIG. 1B), which has a half-life less than 4 hours under the same incubation conditions, demonstrating that modified siRNA compositions described herein are more stable than unmodified siRNAs.

FIG. 2 is a line graph demonstrating that siRNAs containing 2′-deoxy-2′-fluoro modifications in the sense strand, antisense strand, or both strands are effective in reducing gene expression in a luciferase report assay in a dose-dependent manner.

FIG. 3A is a graph demonstrating that in HeLA SS6 cells stably transfected to express murine Factor VII (“FVII”), a 2′-deoxy-2′-fluoro modified siRNA (AD-1661) is approximately 2-fold more potent (IC₅₀ of 0.50 nM) in reducing Factor VII protein levels than an unmodified siRNA (AD-1596; IC₅₀ of 0.95 nM) having the same nucleotide sequence. FIG. 3B demonstrates the 2′-deoxy-2′-fluoro modification of antisense strand enhances the activity of siRNAs relative to unmodified siRNAs.

FIG. 4A is a bar graph demonstrating the results of an in vivo siRNA silencing time-course experiment over 25 days comparing various doses of a 2′-deoxy-2′-fluoro modified siRNA (LNP01_{—}1661) and an unmodified siRNA (LNP01_{—}1596). Mice (n=5 per group) received single intravenous doses of LNP01-1596 or LNP01-1661 at various doses. FVII protein levels are shown at different time points post administration. Duration of silencing effect with LNP01 formulation. FIG. 4B is a bar graph demonstrating the results of an in vivo siRNA silencing experiment comparing various modifications of siRNAs and an unmodified siRNA.

FIG. 5A is a bar graph demonstrating that the interferon-α (“IFNα”) immunostimulatory effect of siRNAs is reduced or eliminated in a 2′-deoxy-2′-fluoro modified siRNA (GP2_A_{—}1661) as compared to an unmodified siRNA (GP2_A_{—}1596). IFNα is measured in picograms per milliliter. FIG. 5B is a bar graph demonstrating that the tumor necrosis factor alpha (“TNFα”) immunostimulatory effect of siRNAs is reduced or eliminated in a 2′-deoxy-2′-fluoro modified siRNA (DOT_A_{—}1661) as compared to an unmodified siRNA (DOT_A_{—}1596). TNFα is measured in picograms per milliliter. DI-A-2216 and DI-A-5167 are positive controls. FIG. 5C is a bar graph demonstrating that IFNα immunostimulatory effect of siRNAs is reduced or eliminated in a 2′-deoxy-2′-fluoro modified siRNA as compared to an unmodified siRNA. FIG. 5D is a bar graph demonstrating that the TNFα immunostimulatory effect of siRNAs is reduced or eliminated in a 2′-deoxy-2′-fluoro modified siRNA as compared to an unmodified siRNA. siRNA A is AD-1596 and siRNA B is AD-1661.

FIG. 6A is sequence alignment of the sense and antisense strands of an unmodified siRNA (AD-1596). FIG. 6B is sequence alignment of the sense and antisense strands of a 2′-deoxy-2′-fluoro modified siRNA (AD-1661). Unmodified nucleotides are represented in upper case (“N”) type, while 2′-F modified nucleotides are represented in lower case (“n”) type. “dT” indicated deoxythymidine. “s” indicates a phosphorothioate internucleotide linkage.

FIG. 7 is a schematic depiction of an oligonucleotide of the present invention containing at least one 2′-deoxy-2′-deoxy-2′-fluoro ribosugar (“2′-F”) modification. R═F in at least one, and optionally more than one, occurrence. The oligonucleotide may additionally contain one or more P═S, Me-P or PS₂ modifications, either directly linked to 2′-F or located at other positions in the oligonucleotides.

FIGS. 8A and 8B are schematic illustrations of a representative gapmer oligonucleotide and a representative hemimer oligonucleotide, which are encompassed in the present invention.

FIG. 9 is a schematic illustration of a gapmer oligonucleotide with unmodified ribosugars in the gap region and one or more modified sugars in the wing regions.

FIG. 10 is a schematic illustration of a gapmer oligonucleotide with all 2′-F modified ribosugars in the gap region; the wing regions may independently have zero, one or more than one modified ribosugars.

FIG. 11 is a schematic illustration of a hemimer oligonucleotides, containing two segments (“Segment 1” and “Segment 2”), at least one of which contains a modified nucleotide, such as a 2′-F modification.

FIG. 12 is a line graph demonstrating the thermal stability of unmodified (AD1596) and 2′-F modified (AD1661) FVII siRNAs, described herein in Example 8, showing increased thermal stability of the 2′-F-modified siRNA relative to the unmodified siRNA.

FIG. 13A is a chart depicting RP-HPLC binding of unmodified (AD1596; leftmost major peak) and 2′-F modified (AD1661; rightmost major peak) FVII siRNAs, described herein in Example 9. FIG. 13B is a chart depicting RP-HPLC profile of 2′-deoxy-2′-deoxy-2′-fluoro modified siRNAs v.s. unmodified siRNAs, described herein in Example 9.

FIG. 14 depicts (a) the microRNA pathway; and (b) inhibition of the microRNA pathway by an antagomir.

FIG. 15 depicts examples of antagomir design according to the present invention.

### DETAILED DESCRIPTION OF THE INVENTION

The invention provides iRNA compositions containing modified nucleotides, as well as methods for inhibiting the expression of a target gene in a cell, tissue or mammal using these compositions. The invention also provides compositions and methods for treating diseases in a mammal caused by the aberrant expression of a target gene, or a mutant form thereof, using oligonucleotide compostions, such as siRNA compositions.

Unless stated otherwise, or implicit from context, the following terms and phrases include the meanings provided below. Unless explicitly stated otherwise, or apparent from context, the terms and phrases below do not exclude the meaning that the term or phrase has acquired in the art to which it pertains. The definitions are provided to aid in describing particular embodiments, and are not intended to limit the claimed invention, because the scope of the invention is limited only by the claims.

iRNA Agent.

An “iRNA agent” as used herein, is a modified or unmodified oligonucleotide or nucleosidic surrogate which can down regulate the expression of a target gene, preferably an endogenous or pathogen target RNA. While not wishing to be bound by theory, an iRNA agent may act by one or more of a number of mechanisms, including post-transcriptional cleavage of a target mRNA sometimes referred to in the art as RNAi, or pre-transcriptional or pre-translational mechanisms. An iRNA agent can include a single strand or can include two or more strands, e.g., it can be a double stranded iRNA agent. In one embodiment the iRNA agents are double stranded and modulate the expression of the target gene through the RNAi mechanism. In another embodiment the iRNA agents are single stranded and modulate the expression of the target gene through the RNAi mechanism.

A double stranded iRNA agent comprises an antisense strand comprising a region of complementarity which is complementary to at least a part of an mRNA formed in the expression of the target gene, and wherein the region of complementarity is less than 30 nucleotides in length, generally 19-24 nucleotides in length, and wherein said iRNA agent, upon contact with a cell expressing said target gene, inhibits the expression of said target gene. The double stranded iRNA agent comprises two oligonucleotide strands that are complementary to hybridize to form a duplex structure. Generally, the duplex structure is between 15 and 30, more generally between 18 and 25, yet more generally between 19 and 24, and most generally between 19 and 21 base pairs in length. In certain embodiments, longer double stranded iRNA agents of between 25 and 30 base pairs in length are preferred. Similarly, the region of complementarity to the target sequence is between 15 and 30, more generally between 18 and 25, yet more generally between 19 and 24, and most generally between 19 and 21 nucleotides in length. The double stranded iRNA agents of the invention may further comprise one or more single-stranded nucleotide overhang(s). In one embodiment, the antisense strand of the dsRNA has 1-10 nucleotides overhangs each at the 3′ end and/or the 5′ end over the sense strand. In one embodiment, the sense strand of the dsRNA has 1-10 nucleotides overhangs each at the 3′ end and/or the 5′ end over the antisense strand. In one embodiment, the double stranded iRNA agents of the invention may further comprise one blunt end and one end has 1-10 nucleotides overhangs.

In a preferred embodiment, the target gene is a human target gene.

The skilled person is well aware that double stranded RNAs (dsRNAs) comprising a duplex structure of between 20 and 23, but specifically 21, base pairs have been hailed as particularly effective in inducing RNA interference (Elbashir et al., EMBO 2001, 20:6877-6888). However, others have found that shorter or longer dsRNAs can be effective as well. In the embodiments described above the double stranded iRNA agents of the invention can comprise at least one strand of a length of minimally 21 nucleotides. It can be reasonably expected that shorter double stranded iRNA agents comprising a known sequence minus only a few nucleotides on one or both ends may be similarly effective as compared to the iRNA agents of the lengths described above. Hence, iRNA agents comprising a partial sequence of at least 15, 16, 17, 18, 19, 20, or more contiguous nucleotides, and differing in their ability to inhibit the expression of the target gene by not more than 5, 10, 15, 20, 25, or 30% inhibition from an iRNA agent comprising the full sequence, are contemplated by the invention. Further iRNA agents that cleave within the target sequence can readily be made using the target gene sequence and the target sequence provided.

The iRNA agents of the invention can contain one or more mismatches to the target sequence. In a preferred embodiment, the iRNA agent of the invention contains no more than 3 mismatches. If the antisense strand of the iRNA contains mismatches to a target sequence, it is preferable that the area of mismatch not be located in the center of the region of complementarity. If the antisense strand of the iRNA contains mismatches to the target sequence, it is preferable that the mismatch be restricted to 5 nucleotides from either end, for example 5, 4, 3, 2, or 1 nucleotide from either the 5′ or 3′ end of the region of complementarity. For example, for a 23 nucleotide iRNA agent antisense strand which is complementary to a region of the target gene, the antisense strand generally does not contain any mismatch within the central 13 nucleotides. The methods known in the art can be used to determine whether an iRNA agent containing a mismatch to a target sequence is effective in inhibiting the expression of the target gene. Consideration of the efficacy of iRNA agents with mismatches in inhibiting expression of the target gene is important, especially if the particular region of complementarity in the target gene is known to have polymorphic sequence variation within the population.

In one embodiment, at least one end of the double stranded iRNA agent has a single-stranded nucleotide overhang of 1 to 4, generally 1 or 2 nucleotides. Double stranded iRNA agents having at least one nucleotide overhang have unexpectedly superior inhibitory properties than their blunt-ended counterparts. Moreover, the present inventors have discovered that the presence of only one nucleotide overhang strengthens the interference activity of the iRNA agent, without affecting its overall stability. Double stranded iRNA agents having only one overhang has proven particularly stable and effective in vivo, as well as in a variety of cells, cell culture mediums, blood, and serum. Generally, the single-stranded overhang is located at the 3′-terminal end of the antisense strand or, alternatively, at the 3′-terminal end of the sense strand. The double stranded iRNA agents may also have a blunt end, generally located at the 5′-end of the antisense strand. Generally, the antisense strand of a double stranded iRNA agent has a nucleotide overhang at the 3′-end, and the 5′-end is blunt. In one embodiment, both ends of a double stranded iRNA agent have a 1-3 nucleotide overhang.

The iRNA agents of the invention may comprise any oligonucleotide modification described herein and below. In certain instances, it may be desirable to modify one or both strands of a double stranded iRNA agent. In some cases, the two strands will include different modifications. Multiple different modifications can be included on each of the strands. The modifications on a given strand may differ from each other, and may also differ from the various modifications on other strands. For example, one strand may have a modification, e.g., a modification described herein, and a different strand may have a different modification, e.g., a different modification described herein. In other cases, one strand may have two or more different modifications, and the another strand may include a modification that differs from the at least two modifications on the other strand.

In one embodiment, the iRNA agent is chemically modified to enhance stability. In one preferred embodiment, one or more backbone linkages in the overhang are replaced with phosphororthioate linkage.

The present invention also includes double stranded iRNA agents wherein the two strands are linked together, e.g., form a hairpin. The two strands can be linked together by a polynucleotide linker such as but not limited to (dT)ₙ; wherein n is 4-10, and thus forming a hairpin. The two strands can also be linked together by a non-nucleosidic linker, e.g. a linker described herein. It will be appreciated by one of skill in the art that any oligonucleotide chemical modifications or variations describe herein can be used in the polynucleotide linker.

In one embodiment the 3′-end of the antisense strand is linked to the 5′-end of the sense strand.

In one embodiment, the 5′-end of the antisense strand is linked to the 3′-end of the sense strand.

Nucleotide. The term “nucleotide” includes a ribonucleotide or a deoxyribonucleotide or modified form thereof, as well as an analog thereof. Nucleotides include species that comprise purines, e.g., adenine, hypoxanthine, guanine, and their derivatives and analogs, as well as pyrimidines, e.g., cytosine, uracil, thymine, and their derivatives and analogs.

Oligonucleotide. The term “oligonucleotide” embraces both single and double stranded polynucleotides. Oligonucleotide also embraces both RNA and DNA, for example of length less than 100, 200, 300, or 400 nucleotides.

Double-Stranded RNA. The term “double-stranded RNA” or “dsRNA” refers to a complex of ribonucleic acid molecules, having a duplex structure comprising two anti-parallel and substantially complementary, as defined above, nucleic acid strands. The two strands forming the duplex structure may be different portions of one larger RNA molecule, or they may be separate RNA molecules. Where the two strands are part of one larger molecule, and therefore are connected by an uninterrupted chain of nucleotides between the 3′-end of one strand and the 5′ end of the respective other strand forming the duplex structure, the connecting RNA chain is referred to as a “hairpin loop”. Where the two strands are connected covalently by means other than an uninterrupted chain of nucleotides between the 3′-end of one strand and the 5′ end of the respective other strand forming the duplex structure, the connecting structure is referred to as a “linker”. The RNA strands may have the same or a different number of nucleotides. The maximum number of base pairs is the number of nucleotides in the shortest strand of the dsRNA minus any overhangs that are present in the duplex. In addition to the duplex structure, a dsRNA may comprise one or more nucleotide overhangs.

Double-stranded siRNA (ds siRNA or dssiRNA) refers to siRNA, having a duplex structure comprising two anti-parallel and substantially complementary oligonulcoetides, as defined above.

Single-stranded siRNA (ss siRNA or ssiRNA or ssRNA) refers to siRNA, having single strand structure comprising substantially complementary oligonulcoetides to its biological target such as mRNA, U1 adaptor.

Nucleotide overhang. The term, a “nucleotide overhang” refers to the unpaired nucleotide or nucleotides that protrude from the duplex structure of a dsRNA when a 3′-end of one strand of the dsRNA extends beyond the 5′-end of the other strand, or vice versa. “Blunt” or “blunt end” means that there are no unpaired nucleotides at that end of the dsRNA, i.e., no nucleotide overhang. A “blunt ended” dsRNA is a dsRNA that is double-stranded over its entire length, i.e., no nucleotide overhang at either end of the molecule.

2′-deoxy-2′-fluoro modified Nucleotides. The phrases “2′-deoxy-2′-fluoro modification” and “2′-fluoro modified nucleotide” refer to a nucleotide unit having a sugar moiety, for example a ribosyl moiety, that is modified at the 2′ position such that the hydroxyl group (2′-OH) is replaced by a fluoro group (2′-F). U.S. Pat. Nos. 6,262,241, and 5,459,255 (all of which are incorporated by reference), drawn to, inter alia, methods of synthesizing 2′-fluoro modified nucleotides and oligonucleotides.

Phosphorothioate internucleotide linkage. The phrase “phosphorothioate internucleotide linkage” refers to the replacement of a P═O group with a P═S group, and includes phosphorodithioate internucleoside linkages. One, some or all of the internucleotide linkages that are present in the oligonucleotide can be phosphorothioate internucleotide linkages. U.S. Pat. Nos. 6,143,881, 5,587,361 and 5,599,797 (all of which are incorporated by reference), drawn to, inter alia, oligonucleotides having phosphorothioate linkages.

Antisense Strand. The phrase “antisense strand” as used herein, refers to a polynucleotide that is substantially or 100% complementary to a target sequence of interest. As used herein, the term “region of complementarity” refers to the region on the antisense strand that is substantially complementary to a sequence, for example a target sequence, as defined herein. Where the region of complementarity is not fully complementary to the target sequence, the mismatches are most tolerated in the terminal regions and, if present, are generally in a terminal region or regions, e.g., within 6, 5, 4, 3, or 2 nucleotides of the 5′ and/or 3′ terminus. An antisense strand may comprise a polynucleotide that is RNA, DNA or chimeric RNA/DNA. For example, an antisense strand may be complementary, in whole or in part, to a molecule of messenger RNA, an RNA sequence that is not mRNA (e.g., tRNA, rRNA and hnRNA) or a sequence of DNA that is either coding or non-coding. The phrase “antisense strand” includes the antisense region of both polynucleotides that are formed from two separate strands, as well as unimolecular polynucleotides that are capable of forming hairpin structures. The terms “antisense strand” and “guide strand” are used interchangeably herein.

Sense Strand. The phrase “sense strand” refers to a polynucleotide that has the same nucleotide sequence, in whole or in part, as a target nucleic acid such as a messenger RNA or a sequence of DNA. The sense strand is not incorporated into the functional riboprotein RISC. The terms “sense strand” and “passenger strand” are used interchangeably herein. “Sense strand” may also refer to the strand of a dsRNA that includes a region that is substantially complementary to a region of the antisense strand.

Duplex. The term “duplex” includes a region of complementarity between two regions of two or more polynucleotides that comprise separate strands, such as a sense strand and an antisense strand, or between two regions of a single contiguous polynucleotide.

Target Sequence. The term, “target sequence” refers to a contiguous portion of the nucleotide sequence of an mRNA molecule formed during the transcription of the target gene, including mRNA that is a product of RNA processing of a primary transcription product. Target sequences may further include RNA precursors, either pri or pre-microRNA, or DNA which encodes the mRNA.

Strand Comprising a Sequence. The term, “strand comprising a sequence” refers to an oligonucleotide comprising a chain of nucleotides that is described by the sequence referred to using the standard nucleotide nomenclature.

Complementary. As used herein, and unless otherwise indicated, the term “complementary,” when used to describe a first nucleotide sequence in relation to a second nucleotide sequence, refers to the ability of an oligonucleotide or polynucleotide comprising the first nucleotide sequence to hybridize and form a duplex structure under certain conditions with an oligonucleotide or polynucleotide comprising the second nucleotide sequence, as will be understood by the skilled person. Such conditions can, for example, be stringent conditions, where stringent conditions may include: 400 mM NaCl, 40 mM PIPES pH 6.4, 1 mM EDTA, 50° C. or 70° C. for 12-16 hours followed by washing. Other conditions, such as physiologically relevant conditions as may be encountered inside an organism, can apply. The skilled person will be able to determine the set of conditions most appropriate for a test of complementarity of two sequences in accordance with the ultimate application of the hybridized nucleotides.

Complementary polynucleotide strands can base pair in the Watson-Crick manner (e.g., a to t, a to u, c to g), or in any other manner that allows for the formation of stable duplexes. This includes base-pairing of the oligonucleotide or polynucleotide comprising the first nucleotide sequence to the oligonucleotide or polynucleotide comprising the second nucleotide sequence over the entire length of the first and second nucleotide sequence. Such sequences can be referred to as “fully complementary”, or “perfect or 100% complementary”, with respect to each other herein. However, where a first sequence is referred to as “substantially complementary” with respect to a second sequence herein, the two sequences can be fully complementary, or they may form one or more, but generally not more than 4, 3 or 2 mismatched base pairs upon hybridization, while retaining the ability to hybridize under the conditions most relevant to their ultimate application. However, where two oligonucleotides are designed to form, upon hybridization, one or more single stranded overhangs, such overhangs shall not be regarded as mismatches with regard to the determination of complementarity. For example, a dsRNA comprising one oligonucleotide 21 nucleotides in length and another oligonucleotide 23 nucleotides in length, wherein the longer oligonucleotide comprises a sequence of 21 nucleotides that is fully complementary to the shorter oligonucleotide, may yet be referred to as “fully complementary” for the purposes of the invention. In some embodiments, less than perfect complementarity may to used to refer to the situation in which some, but not all, nucleotide units of two strands can hydrogen bond with each other. “Substantial complementarity” refers to polynucleotide strands exhibiting 90% or greater complementarity, excluding regions of the polynucleotide strands, such as overhangs, that are selected so as to be noncomplementary. In some embodiments, “Complementary” sequences may also include, or be formed entirely from, non-Watson-Crick base pairs and/or base pairs formed from non-natural and modified nucleotides, in as far as the above requirements with respect to their ability to hybridize are fulfilled.

The terms “complementary”, “fully complementary” and “substantially complementary” herein may be used with respect to the base matching between the sense strand and the antisense strand of a dsRNA, or between the antisense strand of a dsRNA and a target sequence, as will be understood from the context of their use.

As used herein, a polynucleotide which is “substantially complementary to at least part of” a messenger RNA (mRNA) refers to a polynucleotide which is substantially complementary to a contiguous portion of the mRNA of interest (e.g., encoding target gene). For example, a polynucleotide is complementary to at least a part of a target gene mRNA if the sequence is substantially complementary to a non-interrupted portion of a mRNA encoding target gene.

First 5′ terminal nucleotide. The phrase “first 5′ terminal nucleotide” includes first 5′ terminal antisense nucleotides and first 5′ terminal antisense nucleotides. “First 5′ terminal antisense nucleotide” refers to the nucleotide of the antisense strand that is located at the 5′ most position of that strand with respect to the bases of the antisense strand that have corresponding complementary bases on the sense strand. Thus, in a double stranded polynucleotide that is made of two separate strands, it refers to the 5′ most base other than bases that are part of any 5′ overhang on the antisense strand. When the first 5′ terminal antisense nucleotide is part of a hairpin molecule, the term “terminal” refers to the 5′ most relative position within the antisense region and thus is the 5′ most nucleotide of the antisense region. The phrase “first 5′ terminal sense nucleotide” is defined in reference to the antisense nucleotide. In molecules comprising two separate strands, it refers to the nucleotide of the sense strand that is located at the 5′ most position of that strand with respect to the bases of the sense strand that have corresponding complementary bases on the antisense strand. Thus, in a double stranded polynucleotide that is made of two separate strands, it is the 5′ most base other than bases that are part of any 5′ overhang on the sense strand.

Off-Target. The term “off-target” and the phrase “off-target effects” refer to any instance in which an siRNA or shRNA directed against a given target causes an unintended affect by interacting either directly or indirectly with another mRNA sequence, a DNA sequence or a cellular protein or other moiety. For example, an “off-target effect” may occur when there is a simultaneous degradation of other transcripts due to partial homology or complementarity between that other transcript and the sense and/or antisense strand of the siRNA or shRNA

Pharmaceutically Acceptable Carrier or diluent. The phrase “pharmaceutically acceptable carrier or diluent” includes compositions that facilitate the introduction of nucleic acid therapeutics such as single stranded siRNA (ssiRNA), double stranded siRNA (dssiRNA), dsRNA, dsDNA, shRNA, microRNA, antimicroRNA, antagomir, antimir, antisense, U1 adaptor, aptamer, supermir, micro RNA (miRNA) mimic, miRNA inhibitor or dsRNA/DNA hybrids into a cell and includes but is not limited to solvents or dispersants, coatings, anti-infective agents, isotonic agents, and agents that mediate absorption time or release of the inventive polynucleotides and double stranded polynucleotides. The phrase “pharmaceutically acceptable” includes approval by a regulatory agency of a government, for example, the U.S. federal government, a non-U.S. government, or a U.S. state government, or inclusion in a listing in the U.S. Pharmacopeia or any other generally recognized pharmacopeia for use in animals, including in humans.

Introducing into a Cell. The phrase “Introducing into a cell”, when referring to an oligonucleotide, means facilitating uptake or absorption into the cell, as is understood by those skilled in the art. Absorption or uptake of oligonucleotides can occur through unaided diffusive or active cellular processes, or by auxiliary agents or devices. The meaning of this term is not limited to cells in vitro; an oligonucleotide may also be “introduced into a cell”, wherein the cell is part of a living organism. In such instance, introduction into the cell will include the delivery to the organism. For example, for in vivo delivery, oligonucleotides can be injected into a tissue site or administered systemically. In vitro introduction into a cell includes methods known in the art such as electroporation and lipofection.

Modulating the Expression of. The terms “modulating the expression of”, “silence” and “inhibit the expression of”, in as far as they refer to target gene, herein refer to the at least partial suppression of the expression of the target gene, as manifested by a reduction of the amount of mRNA, as compared to a second cell or group of cells substantially identical to the first cell or group of cells but which has or have not been so treated (control cells). The degree of inhibition is usually expressed in terms of

$$\frac{( mRNA in control cells ) - ( mRNA in treated cells )}{( mRNA in control cells )} \cdot 100 \%$$

Alternatively, the degree of inhibition may be given in terms of a reduction of a parameter that is functionally linked to the target gene transcription, e.g. the amount of protein encoded by the gene which is secreted by a cell, or the number of cells displaying a certain phenotype, e.g apoptosis. In principle, gene silencing may be determined in any cell expressing the target, either constitutively or by genomic engineering, and by any appropriate assay. However, when a reference is needed in order to determine whether a given oligonucleotide inhibits the expression of the gene by a certain degree and therefore is encompassed by the instant invention.

For example, in certain instances, expression of the gene is suppressed by at least about 20%, 25%, 35%, or 50% by administration of the compositions comprising the oligonucleotides of the invention. In some embodiments, the target gene is suppressed by at least about 60%, 70%, or 80% by administration of the compositions comprising the oligonucleotides of the invention. In some embodiments, the target gene is suppressed by at least about 85%, 90%, or 95% by of the compositions comprising the oligonucleotides of the invention.

In one aspect of the invention, the target gene is selected from the group consisting of Factor VII, Eg5, PCSK9, TPX2, apoB, SAA, TTR, RSV, PDGF beta gene, Erb-B gene, Src gene, CRK gene, GRB2 gene, RAS gene, MEKK gene, JNK gene, RAF gene, Erk1/2 gene, PCNA(p21) gene, MYB gene, JUN gene, FOS gene, BCL-2 gene, Cyclin D gene, VEGF gene, EGFR gene, Cyclin A gene, Cyclin E gene, WNT-1 gene, beta-catenin gene, c-MET gene, PKC gene, NFKB gene, STAT3 gene, survivin gene, Her2/Neu gene, topoisomerase I gene, topoisomerase II alpha gene, mutations in the p73 gene, mutations in the p21(WAF1/CIP1) gene, mutations in the p27(KIP1) gene, mutations in the PPM1D gene, mutations in the RAS gene, mutations in the caveolin I gene, mutations in the MIB I gene, mutations in the MTAI gene, mutations in the M68 gene, mutations in tumor suppressor genes, mutations in the p53 tumor suppressor gene, and combinations thereof.

In one aspect the invention provides an iRNA agent comprising a sense strand and antisense strand, wherein the antisense strand comprises at least one 2′-deoxy-2′-fluoro(2′-F) nucleotide and the sense strand comprises at least one modified nucleotide with the modification chosen independently from a group consisting of 2′-deoxy-2′-fluoro, 2′-O-methyl (2′-OMe), 2′-methoxyethyl (2′-MOE) and 2′-O,4′-C-methylene (Locked Nucleic Acids, LNA).

In one embodiment, the antisense strand comprises at least one 5′-pyrimidine-purine (5′-PyPu-3′) dinucleotide wherein the pyrimidine is 2′-deoxy-2′-fluoro.

In one embodiment the 5′-most pyrimidines in all occurrences of sequence motif 5′-pyrimidine-purine-3′ (5′-PyPu-3′) in the antisense strand are 2′-deoxy-2′-fluoro.

In one embodiment, all pyrimidines are 2′-deoxy-2′-fluoroin the antisense strand.

In one embodiment, the sense strand comprises at least one 5′-pyrimidine-purine-3′ (5′-PyPu-3′) dinucleotide wherein the pyrimidine is modified with a modification chosen independently from a group consisting of 2′-deoxy-2′-fluoro, 2′-O-methyl (2′-OMe), 2′-methoxyethyl (2′-MOE) and 2′-O,4′-C-methylene (Locked Nucleic Acids, LNA).

In one embodiment, the 5′-most pyrimidines in all occurrences of sequence motif 5′-pyrimidine-purine-3′ (5′-PyPu-3′) in the sense strand are modified with a modification chosen independently from a group consisting of 2′-deoxy-2′-fluoro, 2′-O-methyl (2′-OMe), 2′-methoxyethyl (2′-MOE) and 2′-O,4′-C-methylene (Locked Nucleic Acids, LNA).

In one embodiment, the sense strand comprises all pyrimidines that are modified with modification chosen independently from a group consisting of 2′-deoxy-2′-fluoro, 2′-O-methyl (2′-OMe), 2′-methoxyethyl (2′-MOE) and 2′-O,4′-C-methylene (Locked Nucleic Acids, LNA).

In one embodiment, the modified nucleotide in the sense strand is 2′-O-methyl.

In one embodiment, the modified nucleotide in the sense strand is 2′-O,4′-C-methylene (LNA).

In one embodiment, the modified nucleotide in the sense strand is 2′-deoxy-2′-fluoro.

In one embodiment, the antisense comprises at least one 5′-pyrimidine-purine-3′ (5′-PyPu-3′) dinucleotide wherein the pyrimidine is 2′-deoxy-2′-fluoro and the sense strand comprises at least one 5′-pyrimidine-purine-3′ (5′-PyPu-3′) dinucleotide wherein the pyrimidine is modified with a modification chosen independently from a group consisting of 2′-deoxy-2′-fluoro, 2′-O-methyl (2′-OMe), 2′-methoxyethyl (2′-MOE) and 2′-O,4′-C-methylene (Locked Nucleic Acids, LNA).

In one embodiment, the antisense comprises at least one 5′-pyrimidine-purine-3′ (5′-PyPu-3′) dinucleotide wherein the pyrimidine is 2′-deoxy-2′-fluoro and the 5′-most pyrimidines in all occurrences of sequence motif 5′-pyrimidine-purine-3′ (5′-PyPu-3′) in the sense strand are modified with modification chosen independently from a group consisting of 2′-deoxy-2′-fluoro, 2′-O-methyl (2′-OMe), 2′-methoxyethyl (2′-MOE) and 2′-O,4′-C-methylene (Locked Nucleic Acids, LNA).

In one embodiment, the antisense comprises at least one 5′-pyrimidine-purine-3′ (5′-PyPu-3′) dinucleotide wherein the pyrimidine is 2′-deoxy-2′-fluoro and all pyrimidines in the sense strand are modified with modification chosen independently from a group consisting of 2′-deoxy-2′-fluoro, 2′-O-methyl (2′-OMe), 2′-methoxyethyl (2′-MOE) and 2′-O,4′-C-methylene (Locked Nucleic Acids, LNA).

In one embodiment, the 5′-most pyrimidines in all occurrences of sequence motif 5′-pyrimidine-purine-3′ (5′-PyPu-3′) in the antisense strand are 2′-deoxy-2′-fluoro and the sense strand comprises at least one 5′-pyrimidine-purine-3′ (5′-PyPu-3′) dinucleotide wherein the pyrimidine is modified with a modification chosen independently from a group consisting of 2′-deoxy-2′-fluoro, 2′-O-methyl (2′-OMe), 2′-methoxyethyl (2′-MOE) and 2′-O,4′-C-methylene (Locked Nucleic Acids, LNA).

In one embodiment, the 5′-most pyrimidines in all occurrences of sequence motif 5′-pyrimidine-purine-3′ (5′-PyPu-3′) in the antisense strand are 2′-deoxy-2′-fluoro and the 5′-most pyrimidines in all occurrences of sequence motif 5′-pyrimidine-purine-3′ (5′-PyPu-3′) in the sense strand are modified with modification chosen independently from a group consisting of 2′-deoxy-2′-fluoro, 2′-O-methyl (2′-OMe), 2′-methoxyethyl (2′-MOE) and 2′-O,4′-C-methylene (Locked Nucleic Acids, LNA).

In one embodiment, the 5′-most pyrimidines in all occurrences of sequence motif 5′-pyrimidine-purine-3′ (5′-PyPu-3′) in the antisense strand are 2′-deoxy-2′-fluoro and all pyrimidines in the sense strand are modified with modification chosen independently from a group consisting of 2′-deoxy-2′-fluoro, 2′-O-methyl (2′-OMe), 2′-methoxyethyl (2′-MOE) and 2′-O,4′-C-methylene (Locked Nucleic Acids, LNA).

In one embodiment, all pyrimidines in the antisense strand are 2′-deoxy-2′-fluoro and the sense strand comprises at least one 5′-pyrimidine-purine-3′ (5′-PyPu-3′) dinucleotide wherein the pyrimidine is modified with a modification chosen independently from a group consisting of 2′-deoxy-2′-fluoro, 2′-O-methyl (2′-OMe), 2′-methoxyethyl (2′-MOE) and 2′-O,4′-C-methylene (Locked Nucleic Acids, LNA).

In one embodiment, all pyrimidines in the antisense strand are 2′-deoxy-2′-fluoro and the 5′-most pyrimidines in all occurrences of sequence motif 5′-pyrimidine-purine-3′ (5′-PyPu-3′) in the sense strand are modified with modification chosen independently from a group consisting of 2′-deoxy-2′-fluoro, 2′-O-methyl (2′-OMe), 2′-methoxyethyl (2′-MOE) and 2′-O,4′-C-methylene (Locked Nucleic Acids, LNA).

In one embodiment, all pyrimidines in the antisense strand are 2′-deoxy-2′-fluoro and all pyrimidines in the sense strand are modified with a modification chosen independently from a group consisting of 2′-deoxy-2′-fluoro, 2′-O-methyl (2′-OMe), 2′-methoxyethyl (2′-MOE) and 2′-O,4′-C-methylene (Locked Nucleic Acids, LNA).

In one embodiment, the sense strand and/or antisense strand comprise at least one phosphorothioate backbone linkage.

In one embodiment, the sense and the antisense strand are linked together, e.g., forms hairpin structure. In one embodiment, the 3′-end of the antisense strand is linked to the 5′-end of the sense strand.

In one aspect the invention features a method of modulating the expression of a target gene in an organism comprising administering an iRNA described herein.

In one embodiment, the target gene is an endogenous gene.

In one embodiment, the endogenous gene is the Factor VII or ApoB gene.

In one embodiment, the target gene is an exogenous gene, for example a viral gene, e.g. HCV gene.

In one embodiment, the iRNA agent is chosen from group consisting of duplex number AD-19016, AD-19017 and AD-19018.

siRNA Compositions

Provided herein are siRNA compositions containing one or more short interfering ribonucleic acid (siRNA) molecules. These siRNAs can be single stranded or double stranded. Generally, each siRNA strand will be from about 10 in length (e.g., 10, 11, 12, 13, 14, 15, 16, 17, 18, 19, 20, 21, 22, 23, 24 25 or more) to about 35 nucleotides in length (e.g., 26, 27, 28, 29, 30, 31, 32, 33, 34, 35, 36, 37, 38, 39, 40 or more). Preferably, each strand is from about 19 to about 29 nucleotides in length.

Double stranded siRNA (“dsiRNA” or “dssiRNA”) compositions contain two single strands with at least substantial complementarity. For example, the first and second strands are each about 19 to about 29 nucleotides in length, and are capable of forming a duplex of between 17 and 25 base pairs. Regions of the strands, such as overhangs, are generally selected so as to be noncomplementary, and are not included in the formed duplex. siRNA compositions may contain one or two strands that have one or more terminal deoxythymidine (dT) nucleotide bases. Generally, these dT nucleotides are included in the overhang region and do not form or contribute to a duplex structure.

As provided herein, the modified siRNAs of the invention have superior RNAi properties as compared to non-modified siRNAs. Additionally, siRNAs containing a given complement of modifications may have one or more superior RNAi properties when compared to siRNAs having fewer modifications, or different types of modifications. For example, a modified siRNA having two or more 2′-deoxy-2′-fluoro modifications, and optionally one or more phosphorothioate groups, has superior gene expression inhibitory properties to an siRNA of identical sequence that lacks 2′-deoxy-2′-fluoro modifications, or has only one 2′-deoxy-2′-fluoro modification.

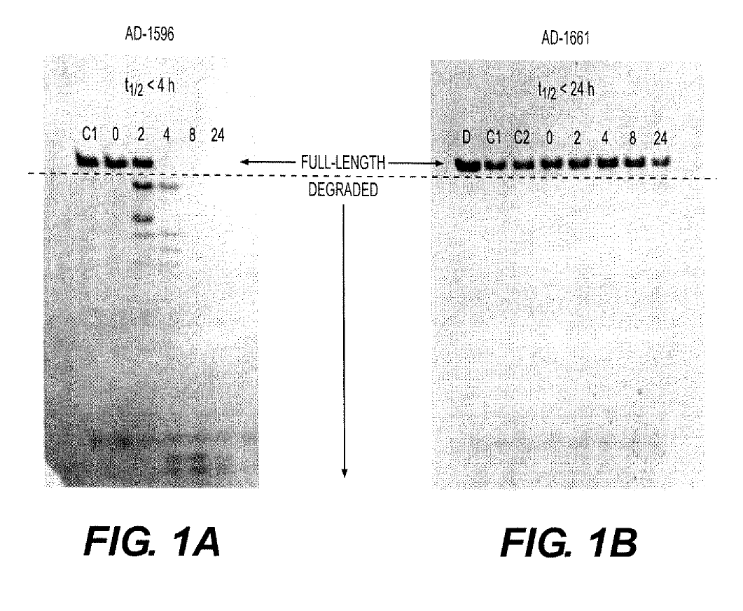

Lack of stability plagues the therapeutic uses of siRNA. For example, naked siRNA, like RNA itself, is quickly degraded by RNAses present in human serum or plasma, such that little or no intravenously injected siRNA reaches target cells or tissue. The modified siRNAs disclosed herein have superior stability as compared to unmodified siRNAs having identical sequences. In some embodiments, the modified siRNAs are 10%, 25%, 50%, 75%, 2-fold, 3-fold, 5-fold, 10-fold or more stable than unmodified siRNAs. As shown in FIG. 1, a modified siRNA (AD-1661; FIG. 1A) has a half-life (t_{1/2}) greater than 24 hours when incubated in human serum at a temperature 37° C. This siRNA is over six-fold more stable as compared to an unmodified siRNA (AD-1596; FIG. 1B), which has a half-life less than 4 hours under the same incubation conditions. Increased stability does not result in decreased efficacy.

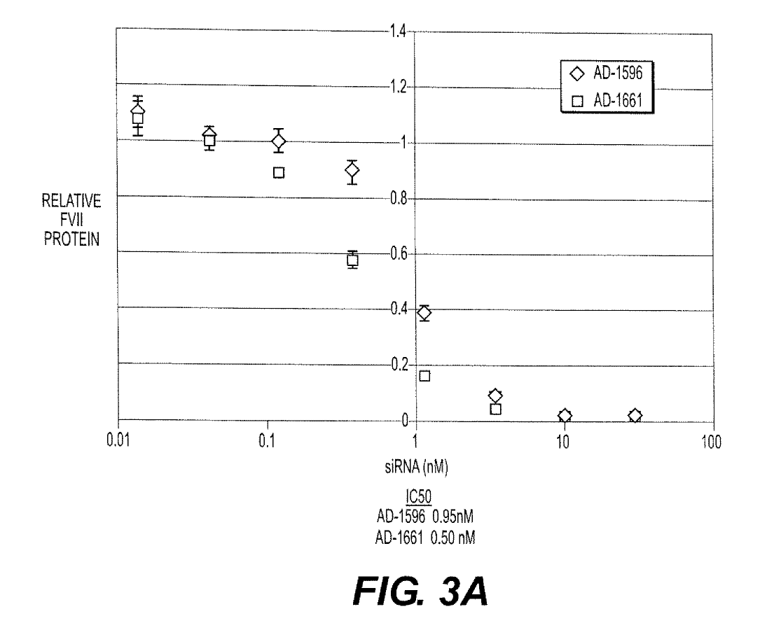

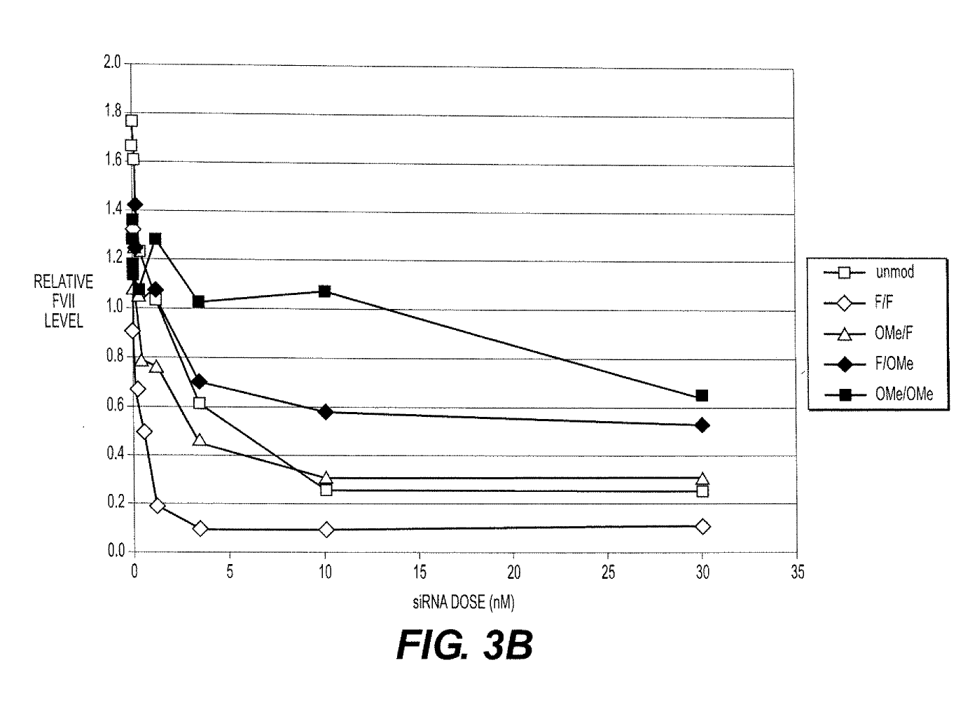

Another advantage of the modified siRNA molecules described herein is increased efficacy (or potency). The modified siRNA molecules disclosed herein have at least equivalent efficacy relative to an siRNA molecule having identical sequence comprising no or fewer modifications. Preferably, modified siRNAs such as 2′-deoxy-2′-fluoro modified siRNAs have increased efficacy relative to an siRNA molecule having identical sequence comprising no or fewer 2′-deoxy-2′-fluoro modifications. For example, as shown in FIG. 3, a 2′-deoxy-2′-fluoro modified siRNA (AD-1661) is approximately 2-fold more potent (IC₅₀ of 0.50 nM) in reducing Factor VII protein levels as compared to an unmodified siRNA (AD-1596; IC₅₀ of 0.95 nM) having the same nucleotide sequence. Here, HeLA SS6 cells were stably transfected to express murine Factor VII (“FVII”).

Induction of inflammatory cytokines and interferon responses by siRNAs, particularly single stranded siRNAs, is considered a substantial deleterious consequence, which negatively impacts their ability to function as inhibitors of gene expression. (See Sioud, J. Mol. Biol. (2005) 348: 1079-90; Sioud, Eur. J. Immunol. (2006) 36: 1222-30; and Hornung et al., Nature Med. (2005) 11: 263-70.) Many types of nucleic acid, including small interfering RNA (siRNA) duplexes, are potent activators of the mammalian innate immune system. Synthetic siRNA duplexes can induce high levels of inflammatory cytokines and type I interferons, in particular interferon-α, after systemic administration in mammals and in primary human blood cell cultures. Due to inherent differences in the nucleotide sequences of individual siRNA duplexes, their capacity to activate the immune response can vary considerably. Although the immunomodulatory effects of nucleic acids may be harnessed therapeutically, for example, in oncology and allergy applications, in many cases immune activation represents a significant undesirable side effect due to the toxicities associated with excessive cytokine release and associated inflammatory syndromes. The potential for siRNA-based drugs to be rendered immunogenic is also a cause for concern because the establishment of an antibody response may severely compromise both safety and efficacy.

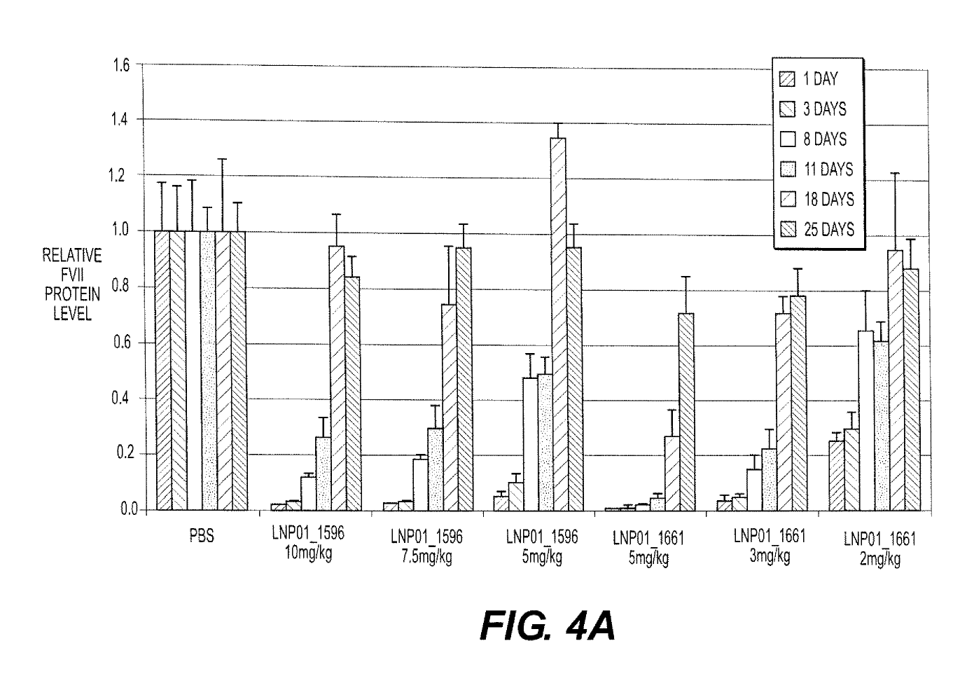

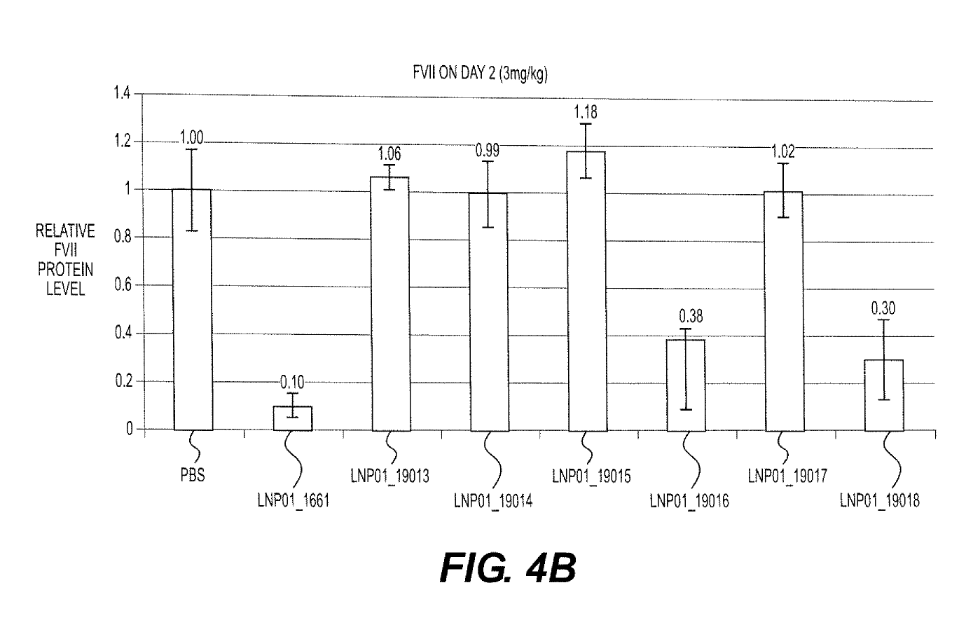

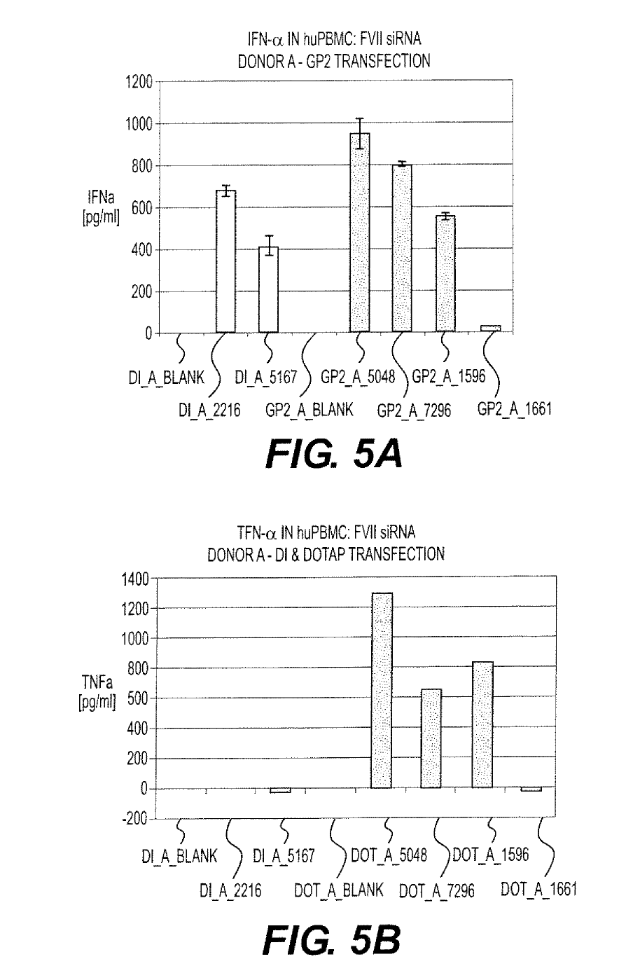

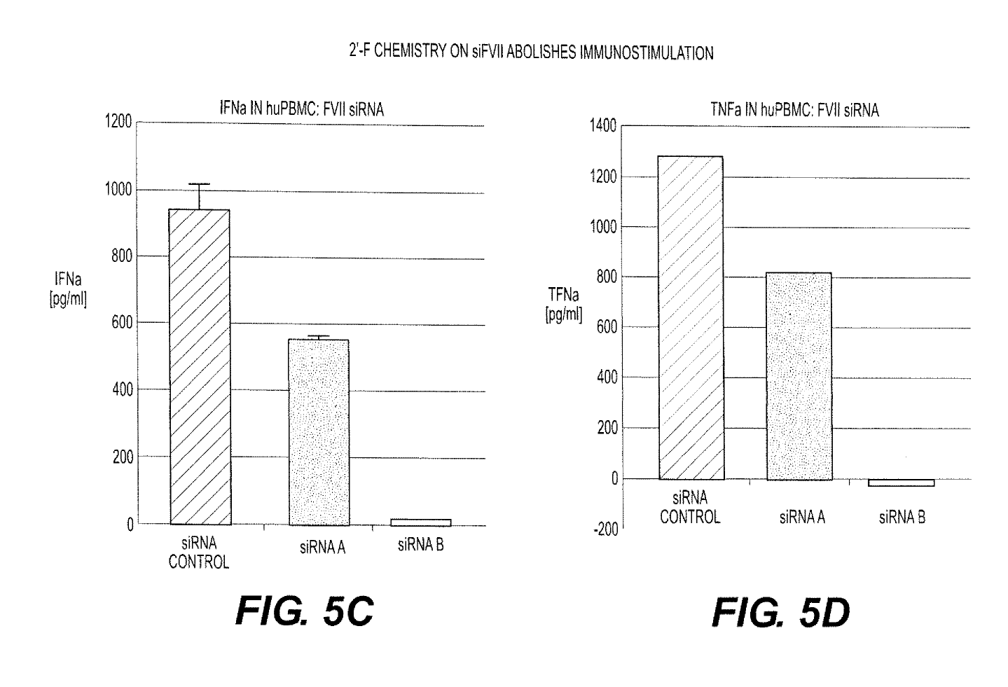

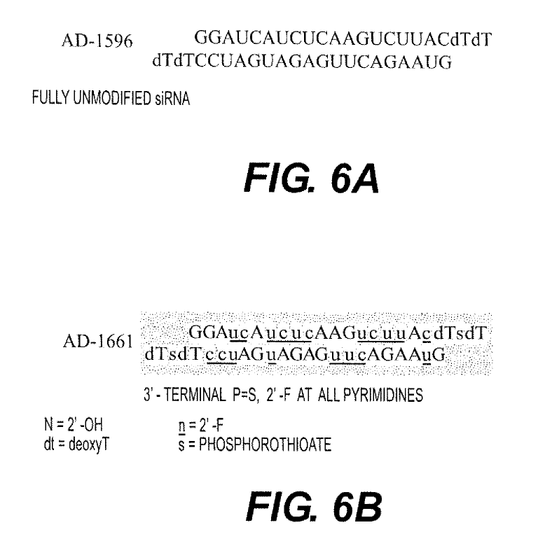

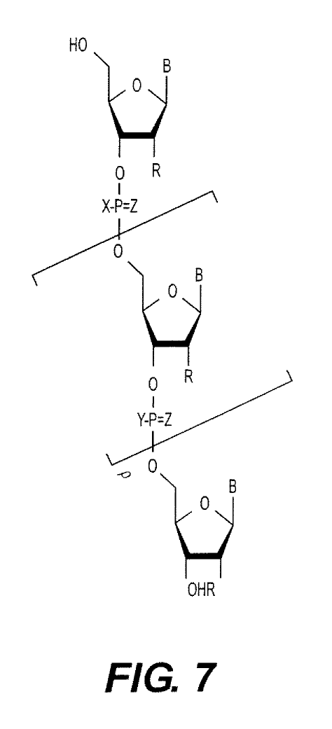

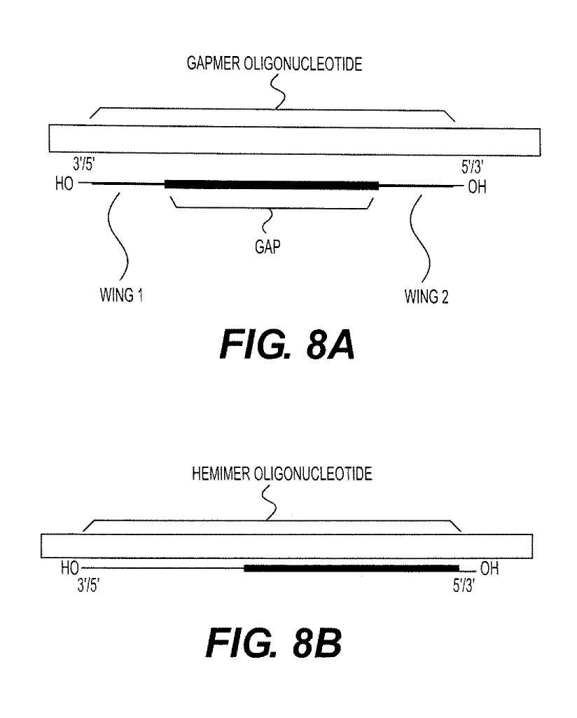

Modified siRNA molecules described herein have decreased immunogenicity relative to an siRNA molecule having identical sequence comprising fewer or no modifications. Replacement of one or more 2′-hydroxyl uridines with 2′-deoxy-2′-fluorouridine abrogates immune activation. Remarkably, the modified siRNA of the present invention elicit a decreased level of immune stimulation compared to their unmodified siRNA counterparts, while retaining the desired RISC-mediated gene silencing activity. As demonstrated herein, both interferon-α and tumor necrosis factor alpha immunostimulatory effects are reduced or eliminated when modified siRNA is introduced into human peripheral blood mononuclear cells as compared to unmodified, native siRNA. As shown in FIG. 5A, an interferon-α (“IFNα”) immunostimulatory effect (measured in picograms per milliliter) is observed when an unmodified siRNA (GP2_A_{—}1596) is introduced into human peripheral blood mononuclear cells (“huPBMC”). However, the IFNa induction when a 2′-deoxy-2′-fluoro modified siRNA (GP2_A_{—}1661) is introduced into human PBMCs is dramatically reduced comparatively. FIG. 5B is a bar graph demonstrating that the tumor necrosis factor alpha (“TNFa”) immunostimulatory effect of siRNAs is reduced or eliminated in a 2′-deoxy-2′-fluoro modified siRNA (DOT_A_{—}1661) as compared to an unmodified siRNA (DOT_A_{—}1596). TNFa is measured in picograms per milliliter. Furthermore, as compared to native siRNA, a reduction in the level of FVII protein expression was observed after administering the modified siRNA into mice and is indicative of RNAi silencing (FIG. 4).

The modified siRNA molecules described herein have increased silencing longevity relative to native siRNA molecules having identical sequence, or modified siRNA molecules containing fewer modifications such as 2′-deoxy-2′-fluoro modifications. Shown as FIG. 4 is a bar graph demonstrating the results of a time-course experiment over 25 days comparing various doses of a 2′-deoxy-2′-fluoro modified siRNA (LNP01_{—}1661) and an unmodified siRNA (LNP01_{—}1596). Mice (n=5 per group) received single intravenous doses of LNP01-1596 or LNP01-1661 at various doses. FVII protein levels are shown at different time points post administration.

2′-deoxy-2′-fluoro modified Nucleotides.

In embodiments of the invention, an siRNA contains a polynucleotide strand containing at least one nucleotide that has a 2′-deoxy-2′-fluoro modification. The siRNAs of the invention include polynucleotides with any number of 2′-deoxy-2′-fluoro modifications from a single 2′-deoxy-2′-fluoro modification to 2′-deoxy-2′-fluoro modifications to all nucleotides, and any intermediate number of nucleotides. dsiRNAs containing a sense strand and an antisense strand may contain 2′-deoxy-2′-fluoro modifications in either sense or antisense strand, or both. Preferably, a dsiRNA contains one or more (e.g., 2, 3, 4, 5, 6, 7, 8, 9 or more, up to and including all nucleotides in the strand) 2′-deoxy-2′-fluoro modified nucleotides in the antisense strand but does not contain 2′-deoxy-2′-fluoro modifications in the sense strand. 2′-deoxy-2′-fluoro modifications may be restricted to purine or pyrimidine nucleotides, or may include all or a subset of each type of nucleotide base.

Preferred embodiments of the invention provide siRNAs having combinations of modifications. For example, one such combination is one 2′-F modification and one or more phosphorothioate (P═S) modifications to the sugar backbone. It is recognized that placement of the P═S modification can be anywhere in the sequence. One, two, three, or more up to and including all the internuclear linkages present in a given siRNA can contain a phosphorothioate linkage.

Another preferred embodiment of the invention provides an siRNAs having two 2′-F modifications and one or more phosphorothioate (P═S) modifications to the sugar backbone. It is recognized that placement of the P═S modification can be anywhere in the sequence. One, two, three, or more up to and including all the internuclear linkages present in a given siRNA can contain a phosphorothioate linkage.

Another preferred embodiment of the invention provides an siRNAs having 2′-F modifications at every pyrimidine present in the oligonucleotide, and one or more phosphorothioate (P═S) modifications to the sugar backbone. It is recognized that placement of the P═S modification can be anywhere in the sequence. One, two, three, or more up to and including all the internuclear linkages present in a given siRNA can contain a phosphorothioate linkage.

Another preferred embodiment of the invention provides an siRNAs having 2′-F modifications at every purine present in the oligonucleotide, and one or more phosphorothioate (P═S) modifications to the sugar backbone. It is recognized that placement of the P═S modification can be anywhere in the sequence. One, two, three, or more up to and including all the internuclear linkages present in a given siRNA can contain a phosphorothioate linkage.

In another embodiments of the invention, an siRNA contains a polynucleotide strand containing at least one nucleotide that has a 2′-deoxy-2′-fluoro modification and a conjugated ligand or plurality of ligands and one or more phosphorothioate (P═S) modifications to the sugar backbone.

Another preferred embodiment of the invention provides therapeutically important single stranded nucleic acid such as antisense, antagomir, microRNA, antimir, microRNA mimic, supermir, U1 adaptor, aptamer having at least one 2′-F modifications and one or more phosphorothioate (P═S) modifications to the sugar backbone. The single strand of the invention include polynucleotides with any number of 2′-deoxy-2′-fluoro modifications from a single 2′-deoxy-2′-fluoro modification to 2′-deoxy-2′-fluoro modifications to all nucleotides, and any intermediate number of nucleotides. 2′-deoxy-2′-fluoro modifications may be restricted to purine or pyrimidine nucleotides, or may include all or a subset of each type of nucleotide base. One, two, three, or more up to and including all the internuclear linkages present in a given siRNA can contain a phosphorothioate linkage. The single stranded nucleic acids comprises of therapeutically.

Another preferred embodiment of the invention provides therapeutically important single stranded nucleic acid such as ssRNA, antisense, antagomir, microRNA, antimir, supermir, miRNA mimic, U1 adaptor, aptamer having at least one 2′-F modifications and one or more phosphorothioate (P═S) modifications to the sugar backbone and conjugated ligand or plurality of ligands.

Another preferred embodiment of the invention provides a hairpin nucleic acid with 2′-F replacement at single or multiple sites to the sequence or global replacement with 2′-F modification and one or more phosphorothioate (P═S) modifications to the sugar backbone. It is recognized that placement of the P═S modification can be anywhere in the sequence. One, two, three, or more up to and including all the internuclear linkages present in a given siRNA can contain a phosphorothioate linkage. The said hairpin nucleic acid can act as a substrate for Dicer, which produces siRNAs.

Another preferred embodiment of the invention provides a hairpin nucleic acid with all pyrimidines replaced with 2′-F sugar modification and one or more phosphorothioate (P═S) modifications to the sugar backbone. It is recognized that placement of the P═S modification can be anywhere in the sequence. One, two, three, or more up to and including all the internuclear linkages present in a given siRNA can contain a phosphorothioate linkage. The said hairpin nucleic acid can act as a substrate for Dicer, which produces siRNAs.

Another preferred embodiment of the invention provides a hairpin nucleic acid with all purines replaced with 2′-F sugar modification and one or more phosphorothioate (P═S) modifications to the sugar backbone. It is recognized that placement of the P═S modification can be anywhere in the sequence. One, two, three, or more up to and including all the internuclear linkages present in a given siRNA can contain a phosphorothioate linkage. The said hairpin nucleic acid can act as a substrate for Dicer, which produces siRNAs.

In another embodiment of the invention provides for double stranded such as siRNA or Dicer substrate, single stranded such as ssRNA, antisense, microRNA, antagomir, antimir, supermir, miRNA mimics, U1 adaptor, aptamer and hairpin oligonucleotides contains at least one 2′-F modification with or without phosphorothioate backbone suppress immunestimulation and makes the oligonucleotide therapeutically more relevant or viable.

In another embodiment of the invention provides for double stranded such as siRNA or Dicer substrate, single stranded such as ssRNA, antisense, microRNA, antagomir, antimir, supermir, miRNA mimic, U1 adaptor, aptamer and hairpin oligonucleotides contains at least one 2′-F modification with or without phosphorothioate backbone suppress or reduce off-target effect and makes the oligonucleotide therapeutically more relevant or viable.

### Targeting Groups.

In one embodiment, an siRNA can include an aminoglycoside ligand, which can cause the siRNA to have improved hybridization properties or improved sequence specificity. Exemplary aminoglycosides include glycosylated polylysine; galactosylated polylysine; neomycin B; tobramycin; kanamycin A; and acridine conjugates of aminoglycosides, such as Neo-N-acridine, Neo-S-acridine, Neo-C-acridine, Tobra-N-acridine, and KanaA-N-acridine. Use of an acridine analog can increase sequence specificity. For example, neomycin B has a high affinity for RNA as compared to DNA, but low sequence-specificity. In some embodiments the guanidine analog (the guanidinoglycoside) of an aminoglycoside ligand is tethered to an oligonucleotide agent. In a guanidinoglycoside, the amine group on the amino acid is exchanged for a guanidine group. Attachment of a guanidine analog can enhance cell permeability of an oligonucleotide agent.

### Cleaving Groups

In one embodiment, the ligand can include a cleaving group that contributes to target gene inhibition by cleavage of the target nucleic acid. Preferably, the cleaving group is tethered to the siRNA in a manner such that it is positioned in the bulge region, where it can access and cleave the target RNA. The cleaving group can be, for example, a bleomycin (e.g., bleomycin-A₅, bleomycin-A₂, or bleomycin-B₂), pyrene, phenanthroline (e.g., O-phenanthroline), a polyamine, a tripeptide (e.g., lys-tyr-lys tripeptide), or metal ion chelating group. The metal ion chelating group can include, e.g., an Lu(III) or EU(III) macrocyclic complex, a Zn(II) 2,9-dimethylphenanthroline derivative, a Cu(II) terpyridine, or acridine, which can promote the selective cleavage of target RNA at the site of the bulge by free metal ions, such as Lu(III). In some embodiments, a peptide ligand can be tethered to an miRNA or a pre-miRNA to promote cleavage of the target RNA, e.g., at the bulge region. For example, 1,8-dimethyl-1,3,6,8,10,13-hexaazacyclotetradecane (cyclam) can be conjugated to a peptide (e.g., by an amino acid derivative) to promote target RNA cleavage. The methods and compositions featured in the invention include siRNA oligonucleotides that inhibit target gene expression by a cleavage or non-cleavage dependent mechanism.

### Targeting Ligands

In some embodiments, the siRNAs of the present invention include a targeting ligand. In some embodiments, this targeting ligand may direct the siRNA to a particular cell. For example, the targeting ligand may specifically or non-specifically bind with a molecule on the surface of a target cell. The targeting moiety can be a molecule with a specific affinity for a target cell. Targeting moieties can include antibodies directed against a protein found on the surface of a target cell, or the ligand or a receptor-binding portion of a ligand for a molecule found on the surface of a target cell. For example, the targeting moiety can recognize a cancer-specific antigen (e.g., CA15-3, CA19-9, CEA, or HER2/neu) or a viral antigen, thus delivering the iRNA to a cancer cell or a virus-infected cell. Exemplary targeting moieties include antibodies (such as IgM, IgG, IgA, IgD, and the like, or a functional portions thereof), ligands for cell surface receptors (e.g., ectodomains thereof). Table 1 provides examples of a number of antigens which can be used to target selected cells.

TABLE 1
Antigens and the targeting cells.

| ANTIGEN | Exemplary tumor tissue |
|---|---|
| CEA (carcinoembryonic antigen) | colon, breast, lung |
| PSA (prostate specific antigen) | prostate cancer |
| CA-125 | ovarian cancer |
| CA 15-3 | breast cancer |
| CA 19-9 | breast cancer |
| HER2/neu | breast cancer |
| α-feto protein | testicular cancer, hepatic cancer |
| β-HCG (human chorionic gonadotropin) | testicular cancer, choriocarcinoma |
| MUC-1 | breast cancer |
| Estrogen receptor | breast cancer, uterine cancer |
| Progesterone receptor | breast cancer, uterine cancer |
| EGFr (epidermal growth factor receptor) | bladder cancer |

Ligand-mediated targeting to specific tissues through binding to their respective receptors on the cell surface offers an attractive approach to improve the tissue-specific delivery of drugs. Specific targeting to disease-relevant cell types and tissues may help to lower the effective dose, reduce side effects and consequently maximize the therapeutic index. Carbohydrates and carbohydrate clusters with multiple carbohydrate motifs represent an important class of targeting ligands, which allow the targeting of drugs to a wide variety of tissues and cell types. For examples, see Hashida, M., Nishikawa, M. et al. (2001) Cell-specific delivery of genes with glycosylated carriers. Adv. Drug Deliv. Rev. 52, 187-9; Monsigny, M., Roche, A.-C. et al. (1994). Glycoconjugates as carriers for specific delivery of therapeutic drugs and genes. Adv. Drug Deliv. Rev. 14, 1-24; Gabius, S., Kayser, K. et al. (1996). Endogenous lectins and neoglycoconjugates. A sweet approach to tumor diagnosis and targeted drug delivery. Eur. J. Pharm. and Biopharm. 42, 250-261; Wadhwa, M. S., and Rice, K. G. (1995) Receptor mediated glycotargeting. J. Drug Target. 3, 111-127.

One of the best characterized receptor-ligand pairs is the asialoglycoprotein receptor (ASGP-R), which is highly expressed on hepatocytes and which has a high affinity for D-galactose as well as N-acetyl-D-galactose (GalNAc). Those carbohydrate ligands have been successfully used to target a wide variety of drugs and even liposomes or polymeric carrier systems to the liver parenchyma. For examples, see Wu, G. Y., and Wu, C. H. (1987) Receptor-mediated in vitro gene transformation by a soluble DNA carrier system. J. Biol. Chem. 262, 4429-4432; Biessen, E. A. L., Vietsch, H., Rump, E. T., Flutter, K., Bijsterbosch, M. K., and Van Berkel, T. J. C. (2000) Targeted delivery of antisense oligonucleotides to parenchymal liver cells in vivo. Methods Enzymol. 313, 324-342; Zanta, M.-A., Boussif, O., Adib, A., and Behr, J.-P. (1997) In Vitro Gene Delivery to Hepatocytes with Galactosylated Polyethylenimine. Bioconjugate Chem. 8, 839-844; Managit, C., Kawakami, S. et al. (2003). Targeted and sustained drug delivery using PEGylated galactosylated liposomes. Int. J. Pharm. 266, 77-84; Sato, A., Takagi, M. et al. (2007). Small interfering RNA delivery to the liver by intravenous administration of galactosylated cationic liposomes in mice. Biomaterials 28; 1434-42.

The Mannose receptor, with its high affinity to D-mannose represents another important carbohydrate-based ligand-receptor pair. The mannose receptor is highly expressed on specific cell types such as macrophages and possibly dendritic cells Mannose conjugates as well as mannosylated drug carriers have been successfully used to target drug molecules to those cells. For examples, see Biessen, E. A. L., Noorman, F. et al. (1996). Lysine-based cluster mannosides that inhibit ligand binding to the human mannose receptor at nanomolar concentration. J. Biol. Chem. 271, 28024-28030; Kinzel, O., Fattori, D. et al. (2003). Synthesis of a functionalized high affinity mannose receptor ligand and its application in the construction of peptide-, polyamide- and PNA-conjugates. J. Peptide Sci. 9, 375-385; Barratt, G., Tenu, J. P. et al. (1986). Preparation and characterization of liposomes containing mannosylated phospholipids capable of targeting drugs to macrophages. Biochim. Biophys. Acta 862, 153-64; Diebold, S. S., Plank, C. et al. (2002). Mannose Receptor-Mediated Gene Delivery into Antigen Presenting Dendritic Cells. Somat. Cell Mol. Genetics. 27, 65-74.

Lipophilic moieties, such as cholesterol or fatty acids, when attached to highly hydrophilic molecules such as nucleic acids can substantially enhance plasma protein binding and consequently circulation half life. In addition, binding to certain plasma proteins, such as lipoproteins, has been shown to increase uptake in specific tissues expressing the corresponding lipoprotein receptors (e.g., LDL-receptor or the scavenger receptor SR-B1). For examples, see Bijsterbosch, M. K., Rump, E. T. et al. (2000). Modulation of plasma protein binding and in vivo liver cell uptake of phosphorothioate oligodeoxynucleotides by cholesterol conjugation. Nucleic Acids Res. 28, 2717-25; Wolfrum, C., Shi, S. et al. (2007). Mechanisms and optimization of in vivo delivery of lipophilic siRNAs. Nat. Biotechnol. 25, 1149-57. Lipophilic conjugates can therefore also be considered as a targeted delivery approach and their intracellular trafficking could potentially be further improved by the combination with endosomolytic agents.

Folates represent another class of ligands which has been widely used for targeted drug delivery via the folate receptor. This receptor is highly expressed on a wide variety of tumor cells, as well as other cells types, such as activated macrophages. For examples, see Matherly, L. H. and Goldman, I. D. (2003). Membrane transport of folates. Vitamins Hormones 66, 403-456; Sudimack, J. and Lee, R. J. (2000). Targeted drug delivery via the folate receptor. Adv. Drug Delivery Rev. 41, 147-162. Similar to carbohydrate-based ligands, folates have been shown to be capable of delivering a wide variety of drugs, including nucleic acids and even liposomal carriers. For examples, see Reddy, J. A., Dean, D. et al. (1999). Optimization of Folate-Conjugated Liposomal Vectors for Folate Receptor-Mediated Gene Therapy. J. Pharm. Sci. 88, 1112-1118; Lu, Y. and Low P. S. (2002). Folate-mediated delivery of macromolecular anticancer therapeutic agents. Adv. Drug Delivery Rev. 54, 675-693; Zhao, X. B. and Lee, R. J. (2004). Tumor-selective targeted delivery of genes and antisense oligodeoxyribonucleotides via the folate receptor; Leamon, C. P., Cooper, S. R. et al. (2003). Folate-Liposome-Mediated Antisense Oligodeoxynucleotide Targeting to Cancer Cells: Evaluation in Vitro and in Vivo. Bioconj. Chem. 14, 738-747.

### Endosomolytic Components

For macromolecular drugs and hydrophilic drug molecules, which cannot easily cross bilayer membranes, entrapment in endosomal/lysosomal compartments of the cell is thought to be the biggest hurdle for effective delivery to their site of action. In recent years, a number of approaches and strategies have been devised to address this problem. For liposomal formulations, the use of fusogenic lipids in the formulation have been the most common approach (Singh, R. S., Goncalves, C. et al. (2004). On the Gene Delivery Efficacies of pH-Sensitive Cationic Lipids via Endosomal Protonation. A Chemical Biology Investigation. Chem. Biol. 11, 713-723.). Other components, which exhibit pH-sensitive endosomolytic activity through protonation and/or pH-induced conformational changes, include charged polymers and peptides. Examples may be found in Hoffman, A. S., Stayton, P. S. et al. (2002). Design of “smart” polymers that can direct intracellular drug delivery. Polymers Adv. Technol. 13, 992-999; Kakudo, Chaki, T., S. et al. (2004). Transferrin-Modified Liposomes Equipped with a pH-Sensitive Fusogenic Peptide: An Artificial Viral-like Delivery System. Biochemistry 436, 5618-5628; Yessine, M. A. and Leroux, J. C. (2004). Membrane-destabilizing polyanions: interaction with lipid bilayers and endosomal escape of biomacromolecules. Adv. Drug Deliv. Rev. 56, 999-1021; Oliveira, S., van Rooy, I. et al. (2007). Fusogenic peptides enhance endosomal escape improving siRNA-induced silencing of oncogenes. Int. J. Pharm. 331, 211-4. They have generally been used in the context of drug delivery systems, such as liposomes or lipoplexes. For folate receptor-mediated delivery using liposomal formulations, for instance, a pH-sensitive fusogenic peptide has been incorporated into the liposomes and shown to enhance the activity through improving the unloading of drug during the uptake process (Turk, M. J., Reddy, J. A. et al. (2002). Characterization of a novel pH-sensitive peptide that enhances drug release from folate-targeted liposomes at endosomal pHs. Biochim. Biophys. Acta 1559, 56-68).

In certain embodiments, the endosomolytic components of the present invention may be polyanionic peptides or peptidomimetics which show pH-dependent membrane activity and/or fusogenicity. A peptidomimetic may be a small protein-like chain designed to mimic a peptide. A peptidomimetic may arise from modification of an existing peptide in order to alter the molecule's properties, or the synthesis of a peptide-like molecule using unnatural amino acids or their analogs. In certain embodiments, they have improved stability and/or biological activity when compared to a peptide. In certain embodiments, the endosomolytic component assumes its active conformation at endosomal pH (e.g., pH 5-6). The “active” conformation is that conformation in which the endosomolytic component promotes lysis of the endosome and/or transport of the siRNA of the invention from the endosome to the cytoplasm of the cell.

Libraries of compounds may be screened for their differential membrane activity at endosomal pH versus neutral pH using a hemolysis assay. Promising candidates isolated by this method may be used as components of the siRNA compositions of the invention. A method for identifying an endosomolytic component for use in the compositions and methods of the present invention may comprise: providing a library of compounds; contacting blood cells with the members of the library, wherein the pH of the medium in which the contact occurs is controlled; determining whether the compounds induce differential lysis of blood cells at a low pH (e.g., about pH 5-6) versus neutral pH (e.g., about pH 7-8).

Exemplary endosomolytic components include the GALA peptide (Subbarao et al., Biochemistry, 1987, 26: 2964-2972), the EALA peptide (Vogel et al., J. Am. Chem. Soc., 1996, 118: 1581-1586), and their derivatives (Turk et al., Biochem. Biophys. Acta, 2002, 1559: 56-68). In certain embodiments, the endosomolytic component may contain a chemical group (e.g., an amino acid) which will undergo a change in charge or protonation in response to a change in pH. The endosomolytic component may be linear or branched. Exemplary primary sequences of endosomolytic components include H₂N-(AALEALAEALEALAEALEALAEAAAAGGC)-CO₂H; H₂N-(AALAEALAEALAEALAEALAEALAAAAGGC)—CO₂H; and H₂N-(ALEALAEALEALAEA)-CONH₂.

In certain embodiments, more than one endosomolytic component may be incorporated in the siRNA of the invention. In some embodiments, this will entail incorporating more than one of the same endosomolytic component into the siRNA. In other embodiments, this will entail incorporating two or more different endosomolytic components into the siRNA.

These endosomolytic components may mediate endosomal escape by, for example, changing conformation at endosomal pH. In certain embodiments, the endosomolytic components may exist in a random coil conformation at neutral pH and rearrange to an amphipathic helix at endosomal pH. As a consequence of this conformational transition, these peptides may insert into the lipid membrane of the endosome, causing leakage of the endosomal contents into the cytoplasm. Because the conformational transition is pH-dependent, the endosomolytic components can display little or no fusogenic activity while circulating in the blood (pH ˜7.4). Fusogenic activity is defined as that activity which results in disruption of a lipid membrane by the endosomolytic component. One example of fusogenic activity is the disruption of the endosomal membrane by the endosomolytic component, leading to endosomal lysis or leakage and transport of one or more components of the siRNA of the invention (e.g., the nucleic acid) from the endosome into the cytoplasm.

In addition to the hemolysis assay described herein, suitable endosomolytic components can be tested and identified by a skilled artisan using other methods. For example, the ability of a compound to respond to, e.g., change charge depending on, the pH environment can be tested by routine methods, e.g., in a cellular assay. In certain embodiments, a test compound is combined with or contacted with a cell, and the cell is allowed to internalize the test compound, e.g., by endocytosis. An endosome preparation can then be made from the contacted cells and the endosome preparation compared to an endosome preparation from control cells. A change, e.g., a decrease, in the endosome fraction from the contacted cell vs. the control cell indicates that the test compound can function as a fusogenic agent. Alternatively, the contacted cell and control cell can be evaluated, e.g., by microscopy, e.g., by light or electron microscopy, to determine a difference in the endosome population in the cells. The test compound and/or the endosomes can labeled, e.g., to quantify endosomal leakage.

In another type of assay, a siRNA described herein is constructed using one or more test or putative fusogenic agents. The siRNA can be constructed using a labeled nucleic acid. The ability of the endosomolytic component to promote endosomal escape, once the siRNA is taken up by the cell, can be evaluated, e.g., by preparation of an endosome preparation, or by microscopy techniques, which enable visualization of the labeled nucleic acid in the cytoplasm of the cell. In certain other embodiments, the inhibition of gene expression, or any other physiological parameter, may be used as a surrogate marker for endosomal escape.

In other embodiments, circular dichroism spectroscopy can be used to identify compounds that exhibit a pH-dependent structural transition.

A two-step assay can also be performed, wherein a first assay evaluates the ability of a test compound alone to respond to changes in pH, and a second assay evaluates the ability of a siRNA that includes the test compound to respond to changes in pH.

### Linkers

In certain embodiments, the covalent linkages between the siRNA and other components of the invention may be mediated by a linker. This linker may be cleavable or non-cleavable, depending on the application. In certain embodiments, a cleavable linker may be used to release the nucleic acid after transport from the endosome to the cytoplasm. The intended nature of the conjugation or coupling interaction, or the desired biological effect, will determine the choice of linker group.

Linker groups may be connected to the oligonucleotide strand(s) at a linker group attachment point (LAP) and may include any C₁-C₁₀₀ carbon-containing moiety, (e.g., C₁-C₇₅, C₁-C₅₀, C₁-C₂₀, C₁-C₁₀; C₁, C₂, C₃, C₄, C₅, C₆, C₇, C₈, C₉, or C₁₀), in some embodiments having at least one oxygen atom, at least one phosphorous atom, and/or at least one nitrogen atom. In some embodiments, the phosphorous atom forms part of a terminal phosphate, or phosphorothioate, group on the linker group, which may serve as a connection point for the nucleic acid strand. In certain embodiments, the nitrogen atom forms part of a terminal ether, ester, amino or amido (NHC(O)—) group on the linker group, which may serve as a connection point for the endosomolytic component or targeting ligand. Preferred linker groups (underlined) include LAP-X—(CH₂)ₙNH—; LAP-X—C(O)(CH₂)ₙNH—; LAP-X—NR″″(CH₂)ₙNH—, LAP-X—C(O)—(CH₂)ₙ—C(O)—; LAP-X—C(O)—(CH₂)ₙ—C(O)O—; LAP-X—C(O)—O—; LAP-X—C(O)—(CH₂)ₙ—NH—C(O)—; LAP-X—C(O)—(CH₂)ₙ—; LAP-X—C(O)—NH—; LAP-X—C(O)—; LAP-X—(CH₂)ₙ—C(O)—; LAP-X—(CH₂)ₙ—C(O)O—; LAP-X—(CH₂)ₙ—; or LAP-X—(CH₂)ₙ—NH—C(O)—; in which —X is (—O—(R″″O)P(O)—(O)ₘ, (—O—(R″″O)P(S)—O—)ₘ, (—O—(R″″S)P(O)—(O)ₘ, (—O—(R″″S)P(S)—O)ₘ, (—O—(R″″O)P(O)—S)ₘ, (—S—(R″″O)P(O)—(O)ₘ, or nothing, n is 1-20 (e.g., 1, 2, 3, 4, 5, 6, 7, 8, 9, 10, 11, 12, 13, 14, 15, 16, 17, 18, 19, or 20), m is 1 to 3, and R″″ is H or C₁-C₆ alkyl. Preferably, n is 5, 6, or 11. In other embodiments, the nitrogen may form part of a terminal oxyamino group, e.g., —ONH₂, or hydrazino group, —NHNH₂. The linker group may optionally be substituted, e.g., with hydroxy, alkoxy, perhaloalkyl, and/or optionally inserted with one or more additional heteroatoms, e.g., N, O, or S. Certain linker groups may include, e.g., LAP-X—(CH₂)ₙNH—; LAP-X—C(O)(CH₂)ₙNH—; LAP-X—NR″″(CH₂)ₙNH—; LAP-X—(CH₂)ₙONH—; LAP-X—C(O)(CH₂)ₙONH—; LAP-X—NR″″(CH₂)ₙONH—; LAP-X—(CH₂)ₙNHNH₂—, LAP-X—C(O)(CH₂)ₙNHNH₂—; LAP-X—NR″″(CH₂)ₙNHNH₂—; LAP-X—C(O)—(CH₂)ₙ—C(O)—; LAP-X—C(O)—(CH₂)ₙ—C(O)O—; LAP-X—C(O)—O—; LAP-X—C(O)—(CH₂)ₙ—NH—C(O)—; LAP-X—C(O)—(CH₂)ₙ—; LAP-X—C(O)—NH—; LAP-X—C(O)—; LAP-X—(CH₂)ₙ—C(O)—; LAP-X—(CH₂)ₙ—C(O)O—; LAP-X—(CH₂)ₙ—; or LAP-X—(CH₂)ₙ—NH—C(O)—. In some embodiments, amino terminated linker groups (e.g., NH₂, ONH₂, NH₂NH₂) can form an imino bond (i.e., C═N) with the ligand. In some embodiments, amino terminated linker groups (e.g., NH₂, ONH₂, NH₂NH₂) can be acylated, e.g., with C(O)CF₃.

In some embodiments, the linker group can terminate with a mercapto group (i.e., SH) or an olefin (e.g., CH═CH₂). For example, the linker group can be LAP-X—X—(CH₂)ₙ—SH, LAP-X—C(O)(CH₂)ₙSH, LAP-X—(CH₂)ₙ—(CH═CH₂), or LAP-X—C(O)(CH₂)ₙ(CH═CH₂), in which X and n can be as described for the linker groups above. In certain embodiments, the olefin can be a Diels-Alder diene or dienophile. The linker group may optionally be substituted, e.g., with hydroxy, alkoxy, perhaloalkyl, and/or optionally inserted with one or more additional heteroatoms, e.g., N, O, or S. The double bond can be cis or trans or E or Z.

In other embodiments the linker group may include an electrophilic moiety, preferably at the terminal position of the linker group. Certain electrophilic moieties include, e.g., an aldehyde, alkyl halide, mesylate, tosylate, nosylate, or brosylate, or an activated carboxylic acid ester, e.g., an NHS ester, or a pentafluorophenyl ester. Other linker groups (underlined) include LAP-X—(CH₂)ₙCHO; LAP-X—C(O)(CH₂)ₙ(CH═CH₂); or LAP-X—NR″″(CH₂)ₙ—CHO, in which n is 1-6 and R″″ is C₁-C₆ alkyl; or LAP-X—(CH₂)ₙC(O)ONHS; LAP-X—C(O)(CH₂)ₙC(O)ONHS; or LAP-X—NR″″(CH₂)ₙC(O)ONHS, in which n is 1-6 and R″″ is C₁-C₆ alkyl; LAP-X—(CH₂)ₙC(O)OC₆F₅; LAP-X—C(O)(CH₂)ₙC(O)OC₆F₅; or LAP-X—NR″″(CH₂)ₙC(O)OC₆F₅, in which n is 1-11 and R″″ is C₁-C₆ alkyl; or —(CH₂)ₙCH₂LG; LAP-X—C(O)(CH₂)ₙCH₂LG; or LAP-X—NR″″(CH₂)ₙCH₂LG, in which X, R″″ and n can be as described for the linker groups above (LG can be a leaving group, e.g., halide, mesylate, tosylate, nosylate, brosylate). In some embodiments, coupling the -linker group to the endosomolytic component or targeting ligand can be carried out by coupling a nucleophilic group of the endosomolytic component or targeting ligand with an electrophilic group on the linker group.

In other embodiments, other protected amino groups can be at the terminal position of the linker group, e.g., alloc, monomethoxy trityl (MMT), trifluoroacetyl, Fmoc, or aryl sulfonyl (e.g., the aryl portion can be ortho-nitrophenyl or ortho, para-dinitrophenyl).

In any of the above linker groups, in addition, one, more than one, or all, of the n-CH₂— groups may be replaced by one or a combination of, e.g., X, as defined above, —Y—(CH₂)ₘ—, —Y—(C(CH₃)H)ₘ—, —Y—C((CH₂)ₚCH₃)H)ₘ—, —Y—(CH₂—C(CH₃)H)ₘ—, —Y—(CH₂—C((CH₂)ₚCH₃)H)ₘ—, —CH═CH—, or —C≡C—, wherein Y is O, S, Se, S—S, S(O), S(O)₂, m is 1-4 and p is 0-4.

Where more than one endosomolytic component or targeting ligand is present on the same siRNA, the more than one endosomolytic component or targeting ligand may be linked to the oligonucleotide strand or an endosomolytic component or targeting ligand in a linear fashion, or by a branched linker group.

In some embodiments, the linker group is a branched linker group, and more in ceratin cases a symmetric branched linker group. The branch point may be an at least trivalent, but may be a tetravalent, pentavalent, or hexavalent atom, or a group presenting such multiple valencies. In some embodiments, the branch point is a glycerol, or glycerol triphosphate, group.

Single Strand siRNA Compound. The phrase “single strand siRNA compound” as used herein, is an siRNA compound which is made up of a single molecule. It may include a duplexed region, formed by intra-strand pairing, e.g., it may be, or include, a hairpin or pan-handle structure. Single strand siRNA compounds may be antisense with regard to the target molecule. In certain embodiments single strand siRNA compounds are 5′ phosphorylated or include a phosphoryl analog at the 5′ prime terminus. 5′-phosphate modifications include those which are compatible with RISC mediated gene silencing. Suitable modifications include: 5′-monophosphate ((HO)₂(O)P—O-5′); 5′-diphosphate ((HO)₂(O)P—O—P(HO)(O)—O-5′); 5′-triphosphate ((HO)₂(O)P—O—(HO)(O)P—O—P(HO)(O)—O-5′); 5′-guanosine cap (7-methylated or non-methylated) (7m-G-O-5′-(HO)(O)P—O—(HO)(O)P—O—P(HO)(O)—O-5′); 5′-adenosine cap (Appp), and any modified or unmodified nucleotide cap structure (N—O-5′-(HO)(O)P—O—(HO)(O)P—O—P(HO)(O)—O-5′); 5′-monothiophosphate (phosphorothioate; (HO)₂(S)P—O-5′); 5′-monodithiophosphate (phosphorodithioate; (HO)(HS)(S)P—O-5′), 5′-phosphorothiolate ((HO)₂(O)P—S-5′); any additional combination of oxygen/sulfur replaced monophosphate, diphosphate and triphosphates (e.g., 5′-alpha-thiotriphosphate, 5′-gamma-thiotriphosphate, etc.), 5′-phosphoramidates ((HO)₂(O)P—NH-5′, (HO)(NH₂)(O)P—O-5′), 5′-alkylphosphonates (R=alkyl=methyl, ethyl, isopropyl, propyl, etc., e.g., RP(OH)(O)—O-5′-, (HO)₂(O)P-5′-CH2-), 5′-alkyletherphosphonates (R=alkylether=methoxymethyl (MeOCH₂—), ethoxymethyl, etc., e.g., RP(OH)(O)—O-5′-). (These modifications can also be used with the antisense strand of a double stranded iRNA.)

A single strand siRNA compound may be sufficiently long that it can enter the RISC and participate in RISC mediated cleavage of a target mRNA. A single strand siRNA compound is at least 14, and in other embodiments at least 15, 20, 25, 29, 35, 40, or 50 nucleotides in length. In certain embodiments, it is less than 200, 100, or 60 nucleotides in length.

Hairpin siRNA compounds will have a duplex region equal to or at least 17, 18, 19, 29, 21, 22, 23, 24, or 25 nucleotide pairs. The duplex region will may be equal to or less than 200, 100, or 50, in length. In certain embodiments, ranges for the duplex region are 15-30, 17 to 23, 19 to 23, and 19 to 21 nucleotides pairs in length. The hairpin may have a single strand overhang or terminal unpaired region, in some embodiments at the 3′, and in certain embodiments on the antisense side of the hairpin. In some embodiments, the overhangs are 2-3 nucleotides in length.

Double Stranded (ds) siRNA compound. The phrase “double stranded (ds) siRNA compound” as used herein, is an siRNA compound which includes more than one, and in some cases two, strands in which interchain hybridization can form a region of duplex structure.

The antisense strand of a double stranded siRNA compound may be equal to or at least, 14, 15, 16 17, 18, 19, 25, 29, 40, or 60 nucleotides in length. It may be equal to or less than 200, 100, or 50, nucleotides in length. Ranges may be 17 to 25, 19 to 23, and 19 to 21 nucleotides in length.

The sense strand of a double stranded siRNA compound may be equal to or at least 14, 15, 16 17, 18, 19, 25, 29, 40, or 60 nucleotides in length. It may be equal to or less than 200, 100, or 50, nucleotides in length. Ranges may be 17 to 25, 19 to 23, and 19 to 21 nucleotides in length.

The double strand portion of a double stranded siRNA compound may be equal to or at least, 14, 15, 16 17, 18, 19, 20, 21, 22, 23, 24, 25, 29, 40, or 60 nucleotide pairs in length. It may be equal to or less than 200, 100, or 50, nucleotides pairs in length. Ranges may be 15-30, 17 to 23, 19 to 23, and 19 to 21 nucleotides pairs in length.

In many embodiments, the dssiRNA compound is sufficiently large that it can be cleaved by an endogenous molecule, e.g., by Dicer, to produce smaller dssiRNA compounds, e.g., siRNAs agents

It may be desirable to modify one or both of the antisense and sense strands of a double strand siRNA compound. In some cases they will have the same modification or the same class of modification but in other cases the sense and antisense strand will have different modifications, e.g., in some cases it is desirable to modify only the sense strand. It may be desirable to modify only the sense strand, e.g., to inactivate it, e.g., the sense strand can be modified in order to inactivate the sense strand and prevent formation of an active siRNA/protein or RISC. This can be accomplished by a modification which prevents 5′-phosphorylation of the sense strand, e.g., by modification with a 5′-O-methyl ribonucleotide (see Nykänen et al., (2001) ATP requirements and small interfering RNA structure in the RNA interference pathway. Cell 107, 309-321.) Other modifications which prevent phosphorylation can also be used, e.g., simply substituting the 5′-OH by H rather than O-Me. Alternatively, a large bulky group may be added to the 5′-phosphate turning it into a phosphodiester linkage, though this may be less desirable as phosphodiesterases can cleave such a linkage and release a functional siRNA 5′-end. Antisense strand modifications include 5′ phosphorylation as well as any of the other 5′ modifications discussed herein, particularly the 5′ modifications discussed above in the section on single stranded iRNA molecules.

The sense and antisense strands may be chosen such that the dssiRNA compound includes a single strand or unpaired region at one or both ends of the molecule. Thus, a dssiRNA compound may contain sense and antisense strands, paired to contain an overhang, e.g., one or two 5′ or 3′ overhangs, or a 3′ overhang of 2-3 nucleotides. Many embodiments will have a 3′ overhang. Certain ssiRNA compounds will have single-stranded overhangs, in some embodiments 3′ overhangs, of 1 or 2 or 3 nucleotides in length at each end. The overhangs can be the result of one strand being longer than the other, or the result of two strands of the same length being staggered. 5′ ends may be phosphorylated.

In some embodiments, the length for the duplexed region is between 15 and 30, or 18, 19, 20, 21, 22, and 23 nucleotides in length, e.g., in the ssiRNA compound range discussed above. ssiRNA compounds can resemble in length and structure the natural Dicer processed products from long dsiRNAs. Embodiments in which the two strands of the ssiRNA compound are linked, e.g., covalently linked are also included. Hairpin, or other single strand structures which provide the required double stranded region, and a 3′ overhang are also within the invention.

Isolated siRNA Compounds. The isolated siRNA compounds described herein, including dssiRNA compounds and ssiRNA compounds can mediate silencing of a target RNA, e.g., mRNA, e.g., a transcript of a gene that encodes a protein. For convenience, such mRNA is also referred to herein as mRNA to be silenced. Such a gene is also referred to as a target gene. In general, the RNA to be silenced is an endogenous gene or a pathogen gene. In addition, RNAs other than mRNA, e.g., tRNAs, and viral RNAs, can also be targeted.

Mediates RNAi. As used herein, the phrase “mediates RNAi” refers to the ability to silence, in a sequence specific manner, a target RNA. While not wishing to be bound by theory, it is believed that silencing uses the RNAi machinery or process and a guide RNA, e.g., an ssiRNA compound of 21 to 23 nucleotides.

Specifically Hybridizable. As used herein, “specifically hybridizable” and “complementary” are terms which are used to indicate a sufficient degree of complementarity such that stable and specific binding occurs between a compound of the invention and a target RNA molecule. Specific binding requires a sufficient degree of complementarity to avoid non-specific binding of the oligomeric compound to non-target sequences under conditions in which specific binding is desired, i.e., under physiological conditions in the case of in vivo assays or therapeutic treatment, or in the case of in vitro assays, under conditions in which the assays are performed. The non-target sequences typically differ by at least 5 nucleotides.

In one embodiment, an siRNA compound is “sufficiently complementary” to a target RNA, e.g., a target mRNA, such that the siRNA compound silences production of protein encoded by the target mRNA. In another embodiment, the siRNA compound is “exactly complementary” to a target RNA, e.g., the target RNA and the siRNA compound anneal, for example to form a hybrid made exclusively of Watson-Crick base pairs in the region of exact complementarity. A “sufficiently complementary” target RNA can include an internal region (e.g., of at least 10 nucleotides) that is exactly complementary to a target RNA. Moreover, in some embodiments, the siRNA compound specifically discriminates a single-nucleotide difference. In this case, the siRNA compound only mediates RNAi if exact complementary is found in the region (e.g., within 7 nucleotides of) the single-nucleotide difference.

RNA agents discussed herein include unmodified RNA as well as RNA which have been modified, e.g., to improve efficacy, and polymers of nucleoside surrogates. Unmodified RNA refers to a molecule in which the components of the nucleic acid, namely sugars, bases, and phosphate moieties, are the same or essentially the same as that which occur in nature, for example as occur naturally in the human body. The art has often referred to rare or unusual, but naturally occurring, RNAs as modified RNAs, see, e.g., Limbach et al., (1994) Summary: the modified nucleosides of RNA, Nucleic Acids Res. 22: 2183-2196. Such rare or unusual RNAs, often termed modified RNAs (apparently because the are typically the result of a post transcriptionally modification) are within the term unmodified RNA, as used herein. Modified RNA refers to a molecule in which one or more of the components of the nucleic acid, namely sugars, bases, and phosphate moieties, are different from that which occur in nature, for example, different from that which occurs in the human body. While they are referred to as modified “RNAs,” they will of course, because of the modification, include molecules which are not RNAs. Nucleoside surrogates are molecules in which the ribophosphate backbone is replaced with a non-ribophosphate construct that allows the bases to the presented in the correct spatial relationship such that hybridization is substantially similar to what is seen with a ribophosphate backbone, e.g., non-charged mimics of the ribophosphate backbone. Examples of all of the above are discussed herein.

Much of the discussion below refers to single strand molecules. In many embodiments of the invention a double stranded siRNA compound, e.g., a partially double stranded siRNA compound, is envisioned. Thus, it is understood that that double stranded structures (e.g., where two separate molecules are contacted to form the double stranded region or where the double stranded region is formed by intramolecular pairing (e.g., a hairpin structure)) made of the single stranded structures described below are within the invention. Lengths are described elsewhere herein.

### Oligonucleotide Modifications

As oligonucleotide are polymers of subunits or monomers, many of the modifications described below occur at a position which is repeated within a oligonucleotide, e.g., a modification of a base, a sugar, or a phosphate moiety, or the a non-linking O of a phosphate moiety. It is not necessary for all positions in a given oligonucleotide to be uniformly modified, and in fact more than one of the aforementioned modifications may be incorporated in a single oligonucleotide or even at a single nucleoside within an oligonucleotide.

In some cases the modification will occur at all of the subject positions in the nucleic acid but in many cases it will not. By way of example, a modification may only occur at a 3′ or 5′ terminal position, may only occur in a terminal regions, e.g., at a position on a terminal nucleotide or in the last 2, 3, 4, 5, or 10 nucleotides of a strand. A modification may occur in a double strand region, a single strand region, or in both. A modification may occur only in the double strand region of an RNA or may only occur in a single strand region of an RNA. E.g., a phosphorothioate modification at a non-linking O position may only occur at one or both termini, may only occur in a terminal regions, e.g., at a position on a terminal nucleotide or in the last 2, 3, 4, 5, or 10 nucleotides of a strand, or may occur in double strand and single strand regions, particularly at termini. The 5′ end or ends can be phosphorylated.

A modification described herein may be the sole modification, or the sole type of modification included on multiple nucleotides, or a modification can be combined with one or more other modifications described herein. The modifications described herein can also be combined onto an oligonucleotide, e.g. different nucleotides of an oligonucleotide have different modifications described herein.

In some embodiments it is preferred, e.g., to enhance stability, to include particular nucleobases in overhangs, or to include modified nucleotides or nucleotide surrogates, in single strand overhangs, e.g., in a 5′ or 3′ overhang, or in both. E.g., it can be desirable to include purine nucleotides in overhangs. In some embodiments all or some of the bases in a 3′ or 5′ overhang will be modified, e.g., with a modification described herein. Modifications can include, e.g., the use of modifications at the 2′ OH group of the ribose sugar, e.g., the use of deoxyribonucleotides, e.g., deoxythymidine, instead of ribonucleotides, and modifications in the phosphate group, e.g., phosphothioate modifications. Overhangs need not be homologous with the target sequence.

Modifications and nucleotide surrogates are discussed below.

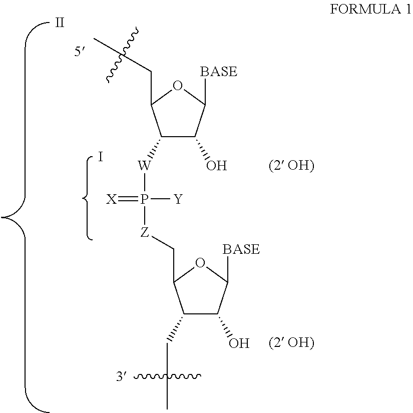

The scaffold presented above in Formula 1 represents a portion of a ribonucleic acid. The basic components are the ribose sugar, the base, the terminal phosphates, and phosphate internucleotide linkers. Where the bases are naturally occurring bases, e.g., adenine, uracil, guanine or cytosine, the sugars are the unmodified 2′ hydroxyl ribose sugar (as depicted) and W, X, Y, and Z are all 0, Formula 1 represents a naturally occurring unmodified oligoribonucleotide.

Unmodified oligoribonucleotides may be less than optimal in some applications, e.g., unmodified oligoribonucleotides can be prone to degradation by e.g., cellular nucleases. Nucleases can hydrolyze nucleic acid phosphodiester bonds. However, chemical modifications to one or more of the above RNA components can confer improved properties, and, e.g., can render oligoribonucleotides more stable to nucleases.

Modified nucleic acids and nucleotide surrogates can include one or more of:

alteration, e.g., replacement, of one or both of the non-linking (X and Y) phosphate oxygens and/or of one or more of the linking (W and Z) phosphate oxygens (When the phosphate is in the terminal position, one of the positions W or Z will not link the phosphate to an additional element in a naturally occurring ribonucleic acid. However, for simplicity of terminology, except where otherwise noted, the W position at the 5′ end of a nucleic acid and the terminal Z position at the 3′ end of a nucleic acid, are within the term “linking phosphate oxygens” as used herein);

alteration, e.g., replacement, of a constituent of the ribose sugar, e.g., of the 2′ hydroxyl on the ribose sugar;

wholesale replacement of the phosphate moiety (bracket I) with “dephospho” linkers;

modification or replacement of a naturally occurring base;

replacement or modification of the ribose-phosphate backbone (bracket II);

modification of the 3′ end or 5′ end of the RNA, e.g., removal, modification or replacement of a terminal phosphate group or conjugation of a moiety, e.g., a fluorescently labeled moiety, to either the 3′ or 5′ end of RNA.

The terms replacement, modification, alteration, and the like, as used in this context, do not imply any process limitation, e.g., modification does not mean that one must start with a reference or naturally occurring ribonucleic acid and modify it to produce a modified ribonucleic acid bur rather modified simply indicates a difference from a naturally occurring molecule.

It is understood that the actual electronic structure of some chemical entities cannot be adequately represented by only one canonical form (i.e., Lewis structure). While not wishing to be bound by theory, the actual structure can instead be some hybrid or weighted average of two or more canonical forms, known collectively as resonance forms or structures. Resonance structures are not discrete chemical entities and exist only on paper. They differ from one another only in the placement or “localization” of the bonding and nonbonding electrons for a particular chemical entity. It can be possible for one resonance structure to contribute to a greater extent to the hybrid than the others. Thus, the written and graphical descriptions of the embodiments of the present invention are made in terms of what the art recognizes as the predominant resonance form for a particular species. For example, any phosphoroamidate (replacement of a nonlinking oxygen with nitrogen) would be represented by X═O and Y═N in the above figure.

Specific modifications are discussed in more detail below.

### The Phosphate Group

The phosphate group is a negatively charged species. The charge is distributed equally over the two non-linking oxygen atoms (i.e., X and Y in Formula 1 above). However, the phosphate group can be modified by replacing one of the oxygens with a different substituent. One result of this modification to RNA phosphate backbones can be increased resistance of the oligoribonucleotide to nucleolytic breakdown. Thus while not wishing to be bound by theory, it can be desirable in some embodiments to introduce alterations which result in either an uncharged linker or a charged linker with unsymmetrical charge distribution.

Examples of modified phosphate groups include phosphorothioate, phosphoroselenates, borano phosphates, borano phosphate esters, hydrogen phosphonates, phosphoroamidates, alkyl or aryl phosphonates and phosphotriesters. In certain embodiments, one of the non-bridging phosphate oxygen atoms in the phosphate backbone moiety can be replaced by any of the following: S, Se, BR₃ (R is hydrogen, alkyl, aryl), C (i.e. an alkyl group, an aryl group, etc. . . . ), H, NR₂ (R is hydrogen, alkyl, aryl), or OR (R is alkyl or aryl). The phosphorous atom in an unmodified phosphate group is achiral. However, replacement of one of the non-bridging oxygens with one of the above atoms or groups of atoms renders the phosphorous atom chiral; in other words a phosphorous atom in a phosphate group modified in this way is a stereogenic center. The stereogenic phosphorous atom can possess either the “R” configuration (herein Rp) or the “S” configuration (herein Sp).

Phosphorodithioates may have both non-linking oxygens replaced by sulfur. Unlike the situation where only one of X or Y is altered, the phosphorus center in the phosphorodithioates is achiral which precludes the formation of oligoribonucleotides diastereomers. Diastereomer formation can result in a preparation in which the individual diastereomers exhibit varying resistance to nucleases. Further, the hybridization affinity of RNA containing chiral phosphate groups can be lower relative to the corresponding unmodified RNA species. Thus, while not wishing to be bound by theory, modifications to both X and Y which eliminate the chiral center, e.g., phosphorodithioate formation, may be desirable in that they cannot produce diastereomer mixtures. Thus, X can be any one of S, Se, B, C, H, N, or OR (R is alkyl or aryl). Thus Y can be any one of S, Se, B, C, H, N, or OR (R is alkyl or aryl). Replacement of X and/or Y with sulfur is possible.

The phosphate linker can also be modified by replacement of a linking oxygen (i.e., W or Z in Formula 1) with nitrogen (bridged phosphoroamidates), sulfur (bridged phosphorothioates) and carbon (bridged methylenephosphonates). The replacement can occur at the either linking oxygen or at both the linking oxygens. When the bridging oxygen is the 3′-oxygen of a nucleoside, replacement with carbon is preferred. When the bridging oxygen is the 5′-oxygen of a nucleoside, replacement with nitrogen is preferred. The replacement can occur at a terminal oxygen (position W (3′) or position Z (5′). Replacement of W with carbon or Z with nitrogen is possible.

Candidate agents can be evaluated for suitability as described below.

### Replacement of the Phosphate Group

The phosphate group can be replaced by non-phosphorus containing connectors (cf. Bracket I in Formula 1 above). While not wishing to be bound by theory, it is believed that since the charged phosphodiester group is the reaction center in nucleolytic degradation, its replacement with neutral structural mimics should impart enhanced nuclease stability. Again, while not wishing to be bound by theory, it can be desirable, in some embodiment, to introduce alterations in which the charged phosphate group is replaced by a neutral moiety.

Examples of moieties which can replace the phosphate group include methyl phosphonate, hydroxylamino, siloxane, carbonate, carboxymethyl, carbamate, amide, thioether, ethylene oxide linker, sulfonate, sulfonamide, thioformacetal, formacetal, oxime, methyleneimino, methylenemethylimino, methylenehydrazo, methylenedimethylhydrazo and methyleneoxymethylimino. Preferred replacements include the methylenecarbonylamino and methylenemethylimino groups.

Modified phosphate linkages where at least one of the oxygens linked to the phosphate has been replaced or the phosphate group has been replaced by a non-phosphorous group, are also referred to as “non phosphodiester backbone linkage.”

Candidate modifications can be evaluated as described below.

### Replacement of Ribophosphate Backbone

Oligonucleotide-mimicking scaffolds can also be constructed wherein the phosphate linker and ribose sugar are replaced by nuclease resistant nucleoside or nucleotide surrogates (see Bracket II of Formula 1 above). While not wishing to be bound by theory, it is believed that the absence of a repetitively charged backbone diminishes binding to proteins that recognize polyanions (e.g., nucleases). Again, while not wishing to be bound by theory, it can be desirable in some embodiment, to introduce alterations in which the bases are tethered by a neutral surrogate backbone.

Examples include the mophilino, cyclobutyl, pyrrolidine and peptide nucleic acid (PNA) nucleoside surrogates. In certain embodiments, PNA surrogates may be used.

Candidate modifications can be evaluated as described below.

### The Sugar Group

A modified RNA can include modification of all or some of the sugar groups of the ribonucleic acid. E.g., the 2′ hydroxyl group (OH) can be modified or replaced with a number of different “oxy” or “deoxy” substituents. While not being bound by theory, enhanced stability is expected since the hydroxyl can no longer be deprotonated to form a 2′ alkoxide ion. The 2′ alkoxide can catalyze degradation by intramolecular nucleophilic attack on the linker phosphorus atom. Again, while not wishing to be bound by theory, it can be desirable to some embodiments to introduce alterations in which alkoxide formation at the 2′ position is not possible.

Examples of “oxy”-2′ hydroxyl group modifications include alkoxy or aryloxy (OR, e.g., R═H, alkyl, cycloalkyl, aryl, aralkyl, heteroaryl or sugar); polyethyleneglycols (PEG), O(CH₂CH₂O)ₙCH₂CH₂OR; “locked” nucleic acids (LNA) in which the 2′ hydroxyl is connected, e.g., by a methylene bridge, to the 4′ carbon of the same ribose sugar; O-AMINE (AMINE=NH₂; alkylamino, dialkylamino, heterocyclyl, arylamino, diaryl amino, heteroaryl amino, or diheteroaryl amino, ethylene diamine, polyamino) and aminoalkoxy, 0(CH₂)ₙAMINE, (e.g., AMINE=NH₂; alkylamino, dialkylamino, heterocyclyl, arylamino, diaryl amino, heteroaryl amino, or diheteroaryl amino, ethylene diamine, polyamino). It is noteworthy that oligonucleotides containing only the methoxyethyl group (MOE), (OCH₂CH₂OCH₃, a PEG derivative), exhibit nuclease stabilities comparable to those modified with the robust phosphorothioate modification.

“Deoxy” modifications include hydrogen (i.e., deoxyribose sugars, which are of particular relevance to the overhang portions of partially ds RNA); halo (e.g., fluoro); amino (e.g., NH₂; alkylamino, dialkylamino, heterocyclyl, arylamino, diaryl amino, heteroaryl amino, diheteroaryl amino, or amino acid); NH(CH₂CH₂NH)ₙCH₂CH₂-AMINE (AMINE=NH₂; alkylamino, dialkylamino, heterocyclyl, arylamino, diaryl amino, heteroaryl amino, or diheteroaryl amino), —NHC(O)R(R=alkyl, cycloalkyl, aryl, aralkyl, heteroaryl or sugar), cyano; mercapto; alkyl-thio-alkyl; thioalkoxy; and alkyl, cycloalkyl, aryl, alkenyl and alkynyl, which may be optionally substituted with e.g., an amino functionality. Other substitutents of certain embodiments include 2′-methoxyethyl, 2′-OCH3, 2′-O-allyl, 2′-C-allyl, and 2′-fluoro.

The sugar group can also contain one or more carbons that possess the opposite stereochemical configuration than that of the corresponding carbon in ribose. Thus, a modified RNA can include nucleotides containing e.g., arabinose, as the sugar. The monomer can have an alpha linkage at the 1′ position on the sugar, e.g., alpha-nucleosides.

Modified RNA's can also include “abasic” sugars, which lack a nucleobase at C-1′. These abasic sugars can also be further contain modifications at one or more of the constituent sugar atoms. Oligonucleotides can also contain one or more sugars that are in the L form, e.g. L-nucleosides.

To maximize nuclease resistance, the 2′ modifications can be used in combination with one or more phosphate linker modifications (e.g., phosphorothioate). The so-called “chimeric” oligonucleotides are those that contain two or more different modifications.

Candidate modifications can be evaluated as described below.

### Terminal Modifications

The 3′ and 5′ ends of an oligonucleotide can be modified. Such modifications can be at the 3′ end, 5′ end or both ends of the molecule. They can include modification or replacement of an entire terminal phosphate or of one or more of the atoms of the phosphate group. E.g., the 3′ and 5′ ends of an oligonucleotide can be conjugated to other functional molecular entities such as labeling moieties, e.g., fluorophores (e.g., pyrene, TAMRA, fluorescein, Cy3 or Cy5 dyes) or protecting groups (based e.g., on sulfur, silicon, boron or ester). The functional molecular entities can be attached to the sugar through a phosphate group and/or a spacer. The terminal atom of the spacer can connect to or replace the linking atom of the phosphate group or the C-3′ or C-5′ 0, N, S or C group of the sugar. Alternatively, the spacer can connect to or replace the terminal atom of a nucleotide surrogate (e.g., PNAs). These spacers or linkers can include e.g., —(CH₂)ₙ—, —(CH₂)ₙN—, —(CH₂)ₙO—, —(CH₂)ₙS—, O(CH₂CH₂O)ₙCH₂CH₂OH (e.g., n=3 or 6), abasic sugars, amide, carboxy, amine, oxyamine, oxyimine, thioether, disulfide, thiourea, sulfonamide, or morpholino, or biotin and fluorescein reagents. When a spacer/phosphate-functional molecular entity-spacer/phosphate array is interposed between two strands of siRNA compounds, this array can substitute for a hairpin RNA loop in a hairpin-type RNA agent. The 3′ end can be an —OH group. While not wishing to be bound by theory, it is believed that conjugation of certain moieties can improve transport, hybridization, and specificity properties. Again, while not wishing to be bound by theory, it may be desirable to introduce terminal alterations that improve nuclease resistance. Other examples of terminal modifications include dyes, intercalating agents (e.g., acridines), cross-linkers (e.g., psoralene, mitomycin C), porphyrins (TPPC4, texaphyrin, Sapphyrin), polycyclic aromatic hydrocarbons (e.g., phenazine, dihydrophenazine), artificial endonucleases (e.g., EDTA), lipophilic carriers (e.g., cholesterol, cholic acid, adamantane acetic acid, 1-pyrene butyric acid, dihydrotestosterone, 1,3-Bis-O(hexadecyl)glycerol, geranyloxyhexyl group, hexadecylglycerol, borneol, menthol, 1,3-propanediol, heptadecyl group, palmitic acid, myristic acid, O3-(oleoyl)lithocholic acid, O3-(oleoyl)cholenic acid, dimethoxytrityl, or phenoxazine) and peptide conjugates (e.g., antennapedia peptide, Tat peptide), alkylating agents, phosphate, amino, mercapto, PEG (e.g., PEG-40K), MPEG, [MPEG]₂, polyamino, alkyl, substituted alkyl, radiolabeled markers, enzymes, haptens (e.g., biotin), transport/absorption facilitators (e.g., aspirin, vitamin E, folic acid), synthetic ribonucleases (e.g., imidazole, bisimidazole, histamine, imidazole clusters, acridine-imidazole conjugates, Eu3+ complexes of tetraazamacrocycles).

Terminal modifications can be added for a number of reasons, including as discussed elsewhere herein to modulate activity or to modulate resistance to degradation. Terminal modifications useful for modulating activity include modification of the 5′ end with phosphate or phosphate analogs. E.g., in certain embodiments siRNA compounds, especially antisense strands, are 5′ phosphorylated or include a phosphoryl analog at the 5′ prime terminus 5′-phosphate modifications include those which are compatible with RISC mediated gene silencing. Suitable modifications include: 5′-monophosphate ((HO)₂(O)P—O-5′); 5′-diphosphate ((HO)₂(O)P—O—P(HO)(O)—O-5′); 5′-triphosphate ((HO)₂(O)P—O—(HO)(O)P—O—P(HO)(O)—O-5′); 5′-guanosine cap (7-methylated or non-methylated) (7m-G-O-5′-(HO)(O)P—O—(HO)(O)P—O—P(HO)(O)—O-5′); 5′-adenosine cap (Appp), and any modified or unmodified nucleotide cap structure (N—O-5′-(HO)(O)P—O—(HO)(O)P—O—P(HO)(O)—O-5′); 5′-monothiophosphate (phosphorothioate; (HO)₂(S)P—O-5′); 5′-monodithiophosphate (phosphorodithioate; (HO)(HS)(S)P—O-5′), 5′-phosphorothiolate ((HO)₂(O)P—S-5′); any additional combination of oxygen/sulfur replaced monophosphate, diphosphate and triphosphates (e.g., 5′-alpha-thiotriphosphate, 5′-gamma-thiotriphosphate, etc.), 5′-phosphoramidates ((HO)₂(O)P—NH-5′, (HO)(NH2)(O)P—O-5′), 5′-alkylphosphonates (R=alkyl=methyl, ethyl, isopropyl, propyl, etc., e.g., RP(OH)(O)—O-5′-, (OH)2(O)P-5′-CH2-), 5′-alkyletherphosphonates (R=alkylether=methoxymethyl (MeOCH2-), ethoxymethyl, etc., e.g., RP(OH)(O)—O-5′-).

Terminal modifications can also be useful for monitoring distribution, and in such cases the groups to be added may include fluorophores, e.g., fluorscein or an Alexa dye, e.g., Alexa 488. Terminal modifications can also be useful for enhancing uptake, useful modifications for this include cholesterol. Terminal modifications can also be useful for cross-linking an RNA agent to another moiety; modifications useful for this include mitomycin C.

Candidate modifications can be evaluated as described below.

### The Bases

Adenine, guanine, cytosine and uracil are the most common bases found in RNA. These bases can be modified or replaced to provide RNA's having improved properties. E.g., nuclease resistant oligoribonucleotides can be prepared with these bases or with synthetic and natural nucleobases (e.g., inosine, thymine, xanthine, hypoxanthine, nubularine, isoguanisine, or tubercidine) and any one of the above modifications. Alternatively, substituted or modified analogs of any of the above bases, e.g., “unusual bases”, “modified bases”, “non-natural bases” and “universal bases” described herein can be employed. Examples include, but not limited to, 2-aminoadenine, 6-methyl and other alkyl derivatives of adenine and guanine, 2-propyl and other alkyl derivatives of adenine and guanine, 5-halouracil and cytosine, 5-propynyl uracil and cytosine, 6-azo uracil, cytosine and thymine, 5-uracil (pseudouracil), 4-thiouracil, 5-halouracil, 5-(2-aminopropyl)uracil, 5-amino allyl uracil, 8-halo, amino, thiol, thioalkyl, hydroxyl and other 8-substituted adenines and guanines, 5-trifluoromethyl and other 5-substituted uracils and cytosines, 7-methylguanine, 5-substituted pyrimidines, 6-azapyrimidines and N-2, N-6 and O-6 substituted purines, including 2-aminopropyladenine, 5-propynyluracil and 5-propynylcytosine, dihydrouracil, 3-deaza-5-azacytosine, 2-aminopurine, 5-alkyluracil, 7-alkylguanine, 5-alkyl cytosine, 7-deazaadenine, N6, N6-dimethyladenine, 2,6-diaminopurine, 5-amino-allyl-uracil, N3-methyluracil, substituted 1,2,4-triazoles, 2-pyridinone, 5-nitroindole, 3-nitropyrrole, 5-methoxyuracil, uracil-5-oxyacetic acid, 5-methoxycarbonylmethyluracil, 5-methyl-2-thiouracil, 5-methoxycarbonylmethyl-2-thiouracil, 5-methylaminomethyl-2-thiouracil, 3-(3-amino-3carboxypropyl)uracil, 3-methylcytosine, 5-methylcytosine, N⁴-acetyl cytosine, 2-thiocytosine, N6-methyladenine, N6-isopentyladenine, 2-methylthio-N6-isopentenyladenine, N-methylguanines, or O-alkylated bases. Further purines and pyrimidines include those disclosed in U.S. Pat. No. 3,687,808, those disclosed in the Concise Encyclopedia Of Polymer Science And Engineering, pages 858-859, Kroschwitz, J. I., ed. John Wiley & Sons, 1990, and those disclosed by Englisch et al., Angewandte Chemie, International Edition, 1991, 30, 613.

Generally, base changes are not used for promoting stability, but they can be useful for other reasons, e.g., some, e.g., 2,6-diaminopurine and 2 amino purine, are fluorescent. Modified bases can reduce target specificity. This may be taken into consideration in the design of siRNA compounds.

Candidate modifications can be evaluated as described below.

### Cationic Groups

Modifications to oligonucleotides can also include attachment of one or more cationic groups to the sugar, base, and/or the phosphorus atom of a phosphate or modified phosphate backbone moiety. A cationic group can be attached to any atom capable of substitution on a natural, unusual or universal base. A preferred position is one that does not interfere with hybridization, i.e., does not interfere with the hydrogen bonding interactions needed for base pairing. A cationic group can be attached e.g., through the C2′ position of a sugar or analogous position in a cyclic or acyclic sugar surrogate. Cationic groups can include e.g., protonated amino groups, derived from e.g., O-AMINE (AMINE=NH₂; alkylamino, dialkylamino, heterocyclyl, arylamino, diaryl amino, heteroaryl amino, or diheteroaryl amino, ethylene diamine, polyamino); aminoalkoxy, e.g., O(CH₂)ₙAMINE, (e.g., AMINE=NH₂; alkylamino, dialkylamino, heterocyclyl, arylamino, diaryl amino, heteroaryl amino, or diheteroaryl amino, ethylene diamine, polyamino); amino (e.g. NH₂; alkylamino, dialkylamino, heterocyclyl, arylamino, diaryl amino, heteroaryl amino, diheteroaryl amino, or amino acid); or NH(CH₂CH₂NH)ₙCH₂CH₂-AMINE (AMINE=NH₂; alkylamino, dialkylamino, heterocyclyl, arylamino, diaryl amino, heteroaryl amino, or diheteroaryl amino)

Placement within an Oligonucleotide

Some modifications may preferably be included on an oligonucleotide at a particular location, e.g., at an internal position of a strand, or on the 5′ or 3′ end of an oligonucleotide. A preferred location of a modification on an oligonucleotide, may confer preferred properties on the agent. For example, preferred locations of particular modifications may confer optimum gene silencing properties, or increased resistance to endonuclease or exonuclease activity.

One or more nucleotides of an oligonucleotide may have a 2′-5′ linkage. One or more nucleotides of an oligonucleotide may have inverted linkages, e.g. 3′-3′, 5′-5′,2′-2′ or 2′-3′ linkages.

A double-stranded oligonucleotide may include at least one 5′-uridine-adenine-3′ (5′-UA-3′) dinucleotide wherein the uridine is a 2′-modified nucleotide, or a terminal 5′-uridine-guanine-3′ (5′-UG-3′) dinucleotide, wherein the 5′-uridine is a 2′-modified nucleotide, or a terminal 5′-cytidine-adenine-3′ (5′-CA-3′) dinucleotide, wherein the 5′-cytidine is a 2′-modified nucleotide, or a terminal 5′-uridine-uridine-3′ (5′-UU-3′) dinucleotide, wherein the 5′-uridine is a 2′-modified nucleotide, or a terminal 5′-cytidine-cytidine-3′ (5′-CC-3′) dinucleotide, wherein the 5′-cytidine is a 2′-modified nucleotide, or a terminal 5′-cytidine-uridine-3′ (5′-CU-3′) dinucleotide, wherein the 5′-cytidine is a 2′-modified nucleotide, or a terminal 5′-uridine-cytidine-3′ (5′-UC-3′) dinucleotide, wherein the 5′-uridine is a 2′-modified nucleotide. Double-stranded oligonucleotides including these modifications are particularly stabilized against endonuclease activity.

### Evaluation of Candidate RNAs

One can evaluate a candidate RNA agent, e.g., a modified RNA, for a selected property by exposing the agent or modified molecule and a control molecule to the appropriate conditions and evaluating for the presence of the selected property. For example, resistance to a degradent can be evaluated as follows. A candidate modified RNA (and a control molecule, usually the unmodified form) can be exposed to degradative conditions, e.g., exposed to a milieu, which includes a degradative agent, e.g., a nuclease. E.g., one can use a biological sample, e.g., one that is similar to a milieu, which might be encountered, in therapeutic use, e.g., blood or a cellular fraction, e.g., a cell-free homogenate or disrupted cells. The candidate and control could then be evaluated for resistance to degradation by any of a number of approaches. For example, the candidate and control could be labeled prior to exposure, with, e.g., a radioactive or enzymatic label, or a fluorescent label, such as Cy3 or Cy5. Control and modified RNA's can be incubated with the degradative agent, and optionally a control, e.g., an inactivated, e.g., heat inactivated, degradative agent. A physical parameter, e.g., size, of the modified and control molecules are then determined. They can be determined by a physical method, e.g., by polyacrylamide gel electrophoresis or a sizing column, to assess whether the molecule has maintained its original length, or assessed functionally. Alternatively, Northern blot analysis can be used to assay the length of an unlabeled modified molecule.

A functional assay can also be used to evaluate the candidate agent. A functional assay can be applied initially or after an earlier non-functional assay, (e.g., assay for resistance to degradation) to determine if the modification alters the ability of the molecule to silence gene expression. For example, a cell, e.g., a mammalian cell, such as a mouse or human cell, can be co-transfected with a plasmid expressing a fluorescent protein, e.g., GFP, and a candidate RNA agent homologous to the transcript encoding the fluorescent protein (see, e.g., WO 00/44914). For example, a modified dsiRNA homologous to the GFP mRNA can be assayed for the ability to inhibit GFP expression by monitoring for a decrease in cell fluorescence, as compared to a control cell, in which the transfection did not include the candidate dsiRNA, e.g., controls with no agent added and/or controls with a non-modified RNA added. Efficacy of the candidate agent on gene expression can be assessed by comparing cell fluorescence in the presence of the modified and unmodified dssiRNA compounds.

In an alternative functional assay, a candidate dssiRNA compound homologous to an endogenous mouse gene, for example, a maternally expressed gene, such as c-mos, can be injected into an immature mouse oocyte to assess the ability of the agent to inhibit gene expression in vivo (see, e.g., WO 01/36646). A phenotype of the oocyte, e.g., the ability to maintain arrest in metaphase II, can be monitored as an indicator that the agent is inhibiting expression. For example, cleavage of c-mos mRNA by a dssiRNA compound would cause the oocyte to exit metaphase arrest and initiate parthenogenetic development (Colledge et al. Nature 370: 65-68, 1994; Hashimoto et al. Nature, 370:68-71, 1994). The effect of the modified agent on target RNA levels can be verified by Northern blot to assay for a decrease in the level of target mRNA, or by Western blot to assay for a decrease in the level of target protein, as compared to a negative control. Controls can include cells in which with no agent is added and/or cells in which a non-modified RNA is added.

### General References

The oligoribonucleotides and oligoribonucleosides used in accordance with this invention may be with solid phase synthesis, see for example “Oligonucleotide synthesis, a practical approach”, Ed. M. J. Gait, IRL Press, 1984; “Oligonucleotides and Analogues, A Practical Approach”, Ed. F. Eckstein, IRL Press, 1991 (especially Chapter 1, Modern machine-aided methods of oligodeoxyribonucleotide synthesis, Chapter 2, Oligoribonucleotide synthesis, Chapter 3,2′-O-Methyloligoribonucleotide- s: synthesis and applications, Chapter 4, Phosphorothioate oligonucleotides, Chapter 5, Synthesis of oligonucleotide phosphorodithioates, Chapter 6, Synthesis of oligo-2′-deoxyribonucleoside methylphosphonates, and. Chapter 7, Oligodeoxynucleotides containing modified bases. Other particularly useful synthetic procedures, reagents, blocking groups and reaction conditions are described in Martin, P., Helv. Chim. Acta, 1995, 78, 486-504; Beaucage, S. L. and Iyer, R. P., Tetrahedron, 1992, 48, 2223-2311 and Beaucage, S. L. and Iyer, R. P., Tetrahedron, 1993, 49, 6123-6194, or references referred to therein. Modification described in WO 00/44895, WO01/75164, or WO02/44321 can be used herein. The disclosure of all publications, patents, and published patent applications listed herein are hereby incorporated by reference.

### Phosphate Group References

The preparation of phosphinate oligoribonucleotides is described in U.S. Pat. No. 5,508,270. The preparation of alkyl phosphonate oligoribonucleotides is described in U.S. Pat. No. 4,469,863. The preparation of phosphoramidite oligoribonucleotides is described in U.S. Pat. No. 5,256,775 or U.S. Pat. No. 5,366,878. The preparation of phosphotriester oligoribonucleotides is described in U.S. Pat. No. 5,023,243. The preparation of borano phosphate oligoribonucleotide is described in U.S. Pat. Nos. 5,130,302 and 5,177,198. The preparation of 3′-Deoxy-3′-amino phosphoramidate oligoribonucleotides is described in U.S. Pat. No. 5,476,925. 3′-Deoxy-3′-methylenephosphonate oligoribonucleotides is described in An, H, et al. J. Org. Chem. 2001, 66, 2789-2801. Preparation of sulfur bridged nucleotides is described in Sproat et al. Nucleosides Nucleotides 1988, 7,651 and Crosstick et al. Tetrahedron Lett. 1989, 30, 4693.

### Sugar Group References

Modifications to the 2′ modifications can be found in Verma, S. et al. Annu. Rev. Biochem. 1998, 67, 99-134 and all references therein. Specific modifications to the ribose can be found in the following references: 2′-fluoro (Kawasaki et. al., J. Med. Chem., 1993, 36, 831-841), 2′-MOE (Martin, P. Helv. Chim. Acta 1996, 79, 1930-1938), “LNA” (Wengel, J. Acc. Chem. Res. 1999, 32, 301-310).

### Replacement of the Phosphate Group References

Methylenemethylimino linked oligoribonucleosides, also identified herein as MMI linked oligoribonucleosides, methylenedimethylhydrazo linked oligoribonucleosides, also identified herein as MDH linked oligoribonucleosides, and methylenecarbonylamino linked oligonucleosides, also identified herein as amide-3 linked oligoribonucleosides, and methyleneaminocarbonyl linked oligonucleosides, also identified herein as amide-4 linked oligoribonucleosides as well as mixed backbone compounds having, as for instance, alternating MMI and PO or PS linkages can be prepared as is described in U.S. Pat. Nos. 5,378,825, 5,386,023, 5,489,677 and in published PCT applications PCT/US92/04294 and PCT/US92/04305 (published as WO 92/20822 WO and 92/20823, respectively). Formacetal and thioformacetal linked oligoribonucleosides can be prepared as is described in U.S. Pat. Nos. 5,264,562 and 5,264,564. Ethylene oxide linked oligoribonucleosides can be prepared as is described in U.S. Pat. No. 5,223,618. Siloxane replacements are described in Cormier, J. F. et al. Nucleic Acids Res. 1988, 16, 4583. Carbonate replacements are described in Tittensor, J. R. J. Chem. Soc. C 1971, 1933. Carboxymethyl replacements are described in Edge, M. D. et al. J. Chem. Soc. Perkin Trans. 1 1972, 1991. Carbamate replacements are described in Stirchak, E. P. Nucleic Acids Res. 1989, 17, 6129.

### Replacement of the Phosphate-Ribose Backbone References

Cyclobutyl sugar surrogate compounds can be prepared as is described in U.S. Pat. No. 5,359,044. Pyrrolidine sugar surrogate can be prepared as is described in U.S. Pat. No. 5,519,134. Morpholino sugar surrogates can be prepared as is described in U.S. Pat. Nos. 5,142,047 and 5,235,033, and other related patent disclosures. Peptide Nucleic Acids (PNAs) are known per se and can be prepared in accordance with any of the various procedures referred to in Peptide Nucleic Acids (PNA): Synthesis, Properties and Potential Applications, Bioorganic & Medicinal Chemistry, 1996, 4, 5-23. They may also be prepared in accordance with U.S. Pat. No. 5,539,083.

### Terminal Modification References

Terminal modifications are described in Manoharan, M. et al. Antisense and Nucleic Acid Drug Development 12, 103-128 (2002) and references therein.

### Base References

N-2 substituted purine nucleoside amidites can be prepared as is described in U.S. Pat. No. 5,459,255. 3-Deaza purine nucleoside amidites can be prepared as is described in U.S. Pat. No. 5,457,191. 5,6-Substituted pyrimidine nucleoside amidites can be prepared as is described in U.S. Pat. No. 5,614,617. 5-Propynyl pyrimidine nucleoside amidites can be prepared as is described in U.S. Pat. No. 5,484,908. Additional references can be disclosed in the above section on base modifications.

### Additional RNA Agents

Certain RNA agents have the following structure (Formula 2):

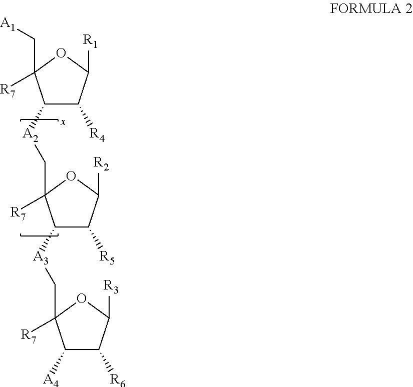

wherein:

R¹, R², and R³ are independently H, (i.e., abasic nucleotides), adenine, guanine, cytosine and uracil, inosine, thymine, xanthine, hypoxanthine, nubularine, tubercidine, isoguanisine, 2-aminoadenine, 6-methyl and other alkyl derivatives of adenine and guanine, 2-propyl and other alkyl derivatives of adenine and guanine, 5-halouracil and cytosine, 5-propynyl uracil and cytosine, 6-azo uracil, cytosine and thymine, 5-uracil (pseudouracil), 4-thiouracil, 5-halouracil, 5-(2-aminopropyl)uracil, 5-amino allyl uracil, 8-halo, amino, thiol, thioalkyl, hydroxyl and other 8-substituted adenines and guanines, 5-trifluoromethyl and other 5-substituted uracils and cytosines, 7-methylguanine, 5-substituted pyrimidines, 6-azapyrimidines and N-2, N-6 and O-6 substituted purines, including 2-aminopropyladenine, 5-propynyluracil and 5-propynylcytosine, dihydrouracil, 3-deaza-5-azacytosine, 2-aminopurine, 5-alkyluracil, 7-alkylguanine, 5-alkyl cytosine, 7-deazaadenine, 7-deazaguanine, N6, N6-dimethyladenine, 2,6-diaminopurine, 5-amino-allyl-uracil, N3-methyluracil, substituted 1,2,4-triazoles, 2-pyridinone, 5-nitroindole, 3-nitropyrrole, 5-methoxyuracil, uracil-5-oxyacetic acid, 5-methoxycarbonylmethyluracil, 5-methyl-2-thiouracil, 5-methoxycarbonylmethyl-2-thiouracil, 5-methylaminomethyl-2-thiouracil, 3-(3-amino-3carboxypropyl)uracil, 3-methylcytosine, 5-methylcytosine, N⁴-acetyl cytosine, 2-thiocytosine, N6-methyladenine, N6-isopentyladenine, 2-methylthio-N6-isopentenyladenine, N-methylguanines, or O-alkylated bases;

R⁴, R⁵, and R⁶ are independently OR⁸, O(CH₂CH₂O)ₘCH₂CH₂OR⁸; O(CH₂)ₙR⁹; O(CH₂)ₙOR⁹, H; halo; NH₂; NHR⁸; N(R⁸)₂; NH(CH₂CH₂NH)ₘCH₂CH₂NHR⁹; NHC(O)R⁸; cyano; mercapto, SR⁸; alkyl-thio-alkyl; alkyl, aralkyl, cycloalkyl, aryl, heteroaryl, alkenyl, alkynyl, each of which may be optionally substituted with halo, hydroxy, oxo, nitro, haloalkyl, alkyl, alkaryl, aryl, aralkyl, alkoxy, aryloxy, amino, alkylamino, dialkylamino, heterocyclyl, arylamino, diaryl amino, heteroaryl amino, diheteroaryl amino, acylamino, alkylcarbamoyl, arylcarbamoyl, aminoalkyl, alkoxycarbonyl, carboxy, hydroxyalkyl, alkanesulfonyl, alkanesulfonamido, arenesulfonamido, aralkylsulfonamido, alkylcarbonyl, acyloxy, cyano, or ureido; or R⁴, R⁵, or R⁶ together combine with R⁷ to form an [—O—CH₂—] covalently bound bridge between the sugar 2′ and 4′ carbons;

A¹ is:

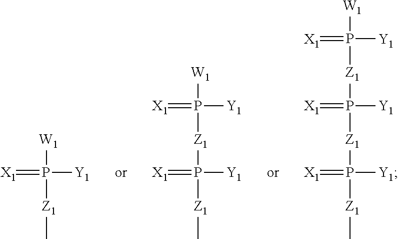

H; OH, OCH₃, W¹; an abasic nucleotide; or absent; (in some embodiments, A1, especially with regard to anti-sense strands, is chosen from 5′-monophosphate ((HO)₂(O)P—O-5′), 5′-diphosphate ((HO)₂(O)P—O—P(HO)(O)—O-5′), 5′-triphosphate ((HO)₂(O)P—O—(HO)(O)P—O—P(HO)(O)—O-5′), 5′-guanosine cap (7-methylated or non-methylated) (7m-G-O-5′-(HO)(O)P—O—(HO)(O)P—O—P(HO)(O)—O-5′), 5′-adenosine cap (Appp), and any modified or unmodified nucleotide cap structure (N—O-5′-(HO)(O)P—O—(HO)(O)P—O—P(HO)(O)—O-5′), 5′-monothiophosphate (phosphorothioate; (HO)₂(S)P—O-5′), 5′-monodithiophosphate (phosphorodithioate; (HO)(HS)(S)P—O-5′), 5′-phosphorothiolate ((HO)₂(O)P—S-5′); any additional combination of oxygen/sulfur replaced monophosphate, diphosphate and triphosphates (e.g., 5′-alpha-thiotriphosphate, 5′-gamma-thiotriphosphate, etc.), 5′-phosphoramidates ((HO)₂(O)P—NH-5′, (HO)(NH₂)(O)P—O-5′), 5′-alkylphosphonates (R=alkyl=methyl, ethyl, isopropyl, propyl, etc., e.g., RP(OH)(O)—O-5′-, (OH)₂(O)P-5′-CH₂—), 5′-alkyletherphosphonates (R=alkylether=methoxymethyl (MeOCH₂—), ethoxymethyl, etc., e.g., RP(OH)(O)—O-5′-));

A² is:

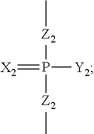

A³ is:

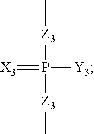

A⁴ is:

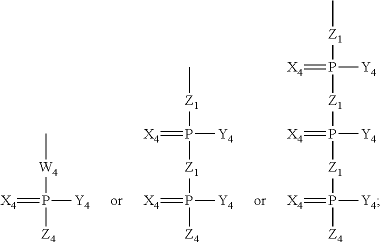

H; Z⁴; an inverted nucleotide; an abasic nucleotide; or absent;

W¹ is OH, (CH₂)ₙR¹⁰, (CH₂)ₙNHR¹⁰, (CH₂)ₙ OR¹⁰, (CH₂)ₙ SR¹⁰; O(CH₂)ₙR¹⁰; O(CH₂)ₙOR¹⁰, O(CH₂)ₙNR¹⁰, O(CH₂)ₙSR¹⁰; O(CH₂)ₙSS(CH₂)ₙOR^{SS(CH}₂)ₙOR¹⁰, O(CH₂)ₙC(O)OR¹⁰, NH(CH₂)ₙR¹⁰; NH(CH₂)ₙNR¹⁰; NH(CH₂)ₙOR¹⁰, NH(CH₂)ₙSR¹⁰; S(CH₂)ₙR¹⁰, S(CH₂)ₙNR¹⁰, S(CH₂)ₙOR¹⁰, S(CH₂)ₙSR¹⁰ O(CH₂CH₂O)ₘCH₂CH₂OR¹⁰; O(CH₂CH₂O)ₘCH₂CH₂NHR¹⁰, NH(CH₂CH₂NH)ₘCH₂CH₂NHR¹⁰; Q-R¹⁰, O-Q-R¹⁰ N-Q-R¹⁰, S-Q-R¹⁰ or —O—;

W⁴ is O, CH₂, NH, or S;

X¹, X², X³, and X⁴ are each independently O or S;

Y¹, Y², Y³, and Y⁴ are each independently OH, O⁻, OR⁸, S, Se, BH₃⁻, H, NHR⁹, N(R⁹)₂ alkyl, cycloalkyl, aralkyl, aryl, or heteroaryl, each of which may be optionally substituted;

Z¹, Z², and Z³ are each independently O, CH₂, NH, or S;

Z⁴ is OH, (CH₂)ₙR¹⁰, (CH₂)ₙNHR¹⁰, (CH₂)ₙ OR¹⁰, (CH₂)ₙ SR¹⁰: O(CH₂)ₙR¹⁰; O(CH₂)ₙOR¹⁰, O(CH₂)ₙNR¹⁰, O(CH₂)ₙSR¹⁰, O(CH₂)ₙSS(CH₂)ₙOR¹⁰, O(CH₂)ₙC(O)OR¹⁰; NH(CH₂)ₙR¹⁰; NH(CH₂)ₙNR¹⁰; NH(CH₂)ₙOR¹⁰, NH(CH₂)ₙSR¹⁰; S(CH₂)ₙR¹⁰, s(CH₂)ₙNR¹⁰, S(CH₂)ₙOR¹⁰, S(CH₂)ₙSR¹⁰ O(CH₂CH₂O)ₘCH₂CH₂OR¹⁰, O(CH₂CH₂O)ₘCH₂CH₂NHR¹⁰, NH(CH₂CH₂NH)ₘCH₂CH₂NHR¹⁰; Q-R¹⁰, O-Q-R¹⁰ N-Q-R¹⁰, S-Q-R¹⁰;

x is 5-100, chosen to comply with a length for an RNA agent described herein;

R⁷ is H; or is together combined with R⁴, R⁵, or R⁶ to form an [—O—CH₂—] covalently bound bridge between the sugar 2′ and 4′ carbons;

R⁸ is alkyl, cycloalkyl, aryl, aralkyl, heterocyclyl, heteroaryl, amino acid, or sugar;

R⁹ is NH₂, alkylamino, dialkylamino, heterocyclyl, arylamino, diaryl amino, heteroaryl amino, diheteroaryl amino, or amino acid;

R¹⁰ is H; fluorophore (pyrene, TAMRA, fluorescein, Cy3 or Cy5 dyes); sulfur, silicon, boron or ester protecting group; intercalating agents (e.g., acridines), cross-linkers (e.g., psoralene, mitomycin C), porphyrins (TPPC4, texaphyrin, Sapphyrin), polycyclic aromatic hydrocarbons (e.g., phenazine, dihydrophenazine), artificial endonucleases (e.g., EDTA), lipohilic carriers (cholesterol, cholic acid, adamantane acetic acid, 1-pyrene butyric acid, dihydrotestosterone, 1,3-Bis-O(hexadecyl)glycerol, geranyloxyhexyl group, hexadecylglycerol, borneol, menthol, 1,3-propanediol, heptadecyl group, palmitic acid, myristic acid, O3-(oleoyl)lithocholic acid, O3-(oleoyl)cholenic acid, dimethoxytrityl, or phenoxazine) and peptide conjugates (e.g., antennapedia peptide, Tat peptide), alkylating agents, phosphate, amino, mercapto, PEG (e.g., PEG-40K), MPEG, [MPEG]₂, polyamino; alkyl, cycloalkyl, aryl, aralkyl, heteroaryl; radiolabelled markers, enzymes, haptens (e.g., biotin), transport/absorption facilitators (e.g., aspirin, vitamin E, folic acid), synthetic ribonucleases (e.g., imidazole, bisimidazole, histamine, imidazole clusters, acridine-imidazole conjugates, Eu3+ complexes of tetraazamacrocycles); or an RNA agent;

m is 0-1,000,000;

n is 0-20;

Q is a spacer selected from the group consisting of abasic sugar, amide, carboxy, oxyamine, oxyimine, thioether, disulfide, thiourea, sulfonamide, or morpholino, biotin or fluorescein reagents.

Certain RNA agents in which the entire phosphate group has been replaced have the following structure (Formula 3):

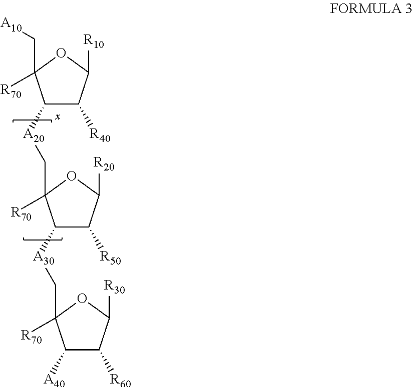

wherein:

A¹⁰-A¹⁰ is L-G-L; A¹⁰ and/or A⁴⁰ may be absent, wherein

L is a linker, wherein one or both L may be present or absent and is selected from the group consisting of CH₂(CH₂)_{g}; N(CH₂)_{g}; O(CH₂)_{g}; S(CH₂)_{g};

G is a functional group selected from the group consisting of siloxane, carbonate, carboxymethyl, carbamate, amide, thioether, ethylene oxide linker, sulfonate, sulfonamide, thioformacetal, formacetal, oxime, methyleneimino, methylenemethylimino, methylenehydrazo, methylenedimethylhydrazo and methyleneoxymethylimino;

R¹⁰, R²⁰, and R³⁰ are independently H, (i.e., abasic nucleotides), adenine, guanine, cytosine and uracil, inosine, thymine, xanthine, hypoxanthine, nubularine, tubercidine, isoguanisine, 2-aminoadenine, 6-methyl and other alkyl derivatives of adenine and guanine, 2-propyl and other alkyl derivatives of adenine and guanine, 5-halouracil and cytosine, 5-propynyl uracil and cytosine, 6-azo uracil, cytosine and thymine, 5-uracil (pseudouracil), 4-thiouracil, 5-halouracil, 5-(2-aminopropyl)uracil, 5-amino allyl uracil, 8-halo, amino, thiol, thioalkyl, hydroxyl and other 8-substituted adenines and guanines, 5-trifluoromethyl and other 5-substituted uracils and cytosines, 7-methylguanine, 5-substituted pyrimidines, 6-azapyrimidines and N-2, N-6 and O-6 substituted purines, including 2-aminopropyladenine, 5-propynyluracil and 5-propynylcytosine, dihydrouracil, 3-deaza-5-azacytosine, 2-aminopurine, 5-alkyluracil, 7-alkylguanine, 5-alkyl cytosine, 7-deazaadenine, 7-deazaguanine, N6, N6-dimethyladenine, 2,6-diaminopurine, 5-amino-allyl-uracil, N3-methyluracil substituted 1,2,4-triazoles, 2-pyridinone, 5-nitroindole, 3-nitropyrrole, 5-methoxyuracil, uracil-5-oxyacetic acid, 5-methoxycarbonylmethyluracil, 5-methyl-2-thiouracil, 5-methoxycarbonylmethyl-2-thiouracil, 5-methylaminomethyl-2-thiouracil, 3-(3-amino-3carboxypropyl)uracil, 3-methylcytosine, 5-methylcytosine, N⁴-acetyl cytosine, 2-thiocytosine, N6-methyladenine, N6-isopentyladenine, 2-methylthio-N6-isopentenyladenine, N-methylguanines, or O-alkylated bases;

R⁴⁰, R⁵⁰, and R⁶⁰ are independently OR⁸, O(CH₂CH₂O)ₘCH₂CH₂OR⁸; O(CH₂)ₙR⁹; O(CH₂)ₙOR⁹, H; halo; NH₂; NHR⁸; N(R⁸)₂; NH(CH₂CH₂NH)ₘCH₂CH₂R⁹; NHC(O)R⁸; cyano; mercapto, SR⁷; alkyl-thio-alkyl; alkyl, aralkyl, cycloalkyl, aryl, heteroaryl, alkenyl, alkynyl, each of which may be optionally substituted with halo, hydroxy, oxo, nitro, haloalkyl, alkyl, alkaryl, aryl, aralkyl, alkoxy, aryloxy, amino, alkylamino, dialkylamino, heterocyclyl, arylamino, diaryl amino, heteroaryl amino, diheteroaryl amino, acylamino, alkylcarbamoyl, arylcarbamoyl, aminoalkyl, alkoxycarbonyl, carboxy, hydroxyalkyl, alkanesulfonyl, alkanesulfonamido, arenesulfonamido, aralkylsulfonamido, alkylcarbonyl, acyloxy, cyano, and ureido groups; or R⁴⁰, R⁵⁰, or R⁶⁰ together combine with R⁷⁰ to form an [—O—CH₂—] covalently bound bridge between the sugar 2′ and 4′ carbons;

x is 5-100 or chosen to comply with a length for an RNA agent described herein;

R⁷⁰ is H; or is together combined with R⁴⁰, R⁵⁰, or R⁶⁰ to form an [—O—CH₂—] covalently bound bridge between the sugar 2′ and 4′ carbons;

R⁸ is alkyl, cycloalkyl, aryl, aralkyl, heterocyclyl, heteroaryl, amino acid, or sugar;

R⁹ is NH₂, alkylamino, dialkylamino, heterocyclyl, arylamino, diaryl amino, heteroaryl amino, diheteroaryl amino, or amino acid;

m is 0-1,000,000;

n is 0-20;

g is 0-2.

Certain nucleoside surrogates have the following structure (Formula 4):

SLR¹⁰⁰-(M—SLR²⁰⁰)ₓ-M—SLR³⁰⁰ FORMULA 4

wherein:

S is a nucleoside surrogate selected from the group consisting of mophilino, cyclobutyl, pyrrolidine and peptide nucleic acid;

L is a linker and is selected from the group consisting of CH₂(CH₂)_{g}; N(CH₂)_{g}; O(CH₂)_{g}; S(CH₂)_{g}; —C(O)(CH₂)ₙ— or may be absent;

M is an amide bond; sulfonamide; sulfinate; phosphate group; modified phosphate group as described herein; or may be absent;

R¹⁰⁰, R²⁰⁰, and R³⁰⁰ are independently H (i.e., abasic nucleotides), adenine, guanine, cytosine and uracil, inosine, thymine, xanthine, hypoxanthine, nubularine, tubercidine, isoguanisine, 2-aminoadenine, 6-methyl and other alkyl derivatives of adenine and guanine, 2-propyl and other alkyl derivatives of adenine and guanine, 5-halouracil and cytosine, 5-propynyl uracil and cytosine, 6-azo uracil, cytosine and thymine, 5-uracil (pseudouracil), 4-thiouracil, 5-halouracil, 5-(2-aminopropyl)uracil, 5-amino allyl uracil, 8-halo, amino, thiol, thioalkyl, hydroxyl and other 8-substituted adenines and guanines, 5-trifluoromethyl and other 5-substituted uracils and cytosines, 7-methylguanine, 5-substituted pyrimidines, 6-azapyrimidines and N-2, N-6 and O-6 substituted purines, including 2-aminopropyladenine, 5-propynyluracil and 5-propynylcytosine, dihydrouracil, 3-deaza-5-azacytosine, 2-aminopurine, 5-alkyluracil, 7-alkylguanine, 5-alkyl cytosine, 7-deazaadenine, 7-deazaguanine, N6, N6-dimethyladenine, 2,6-diaminopurine, 5-amino-allyl-uracil, N3-methyluracil substituted 1, 2, 4,-triazoles, 2-pyridinones, 5-nitroindole, 3-nitropyrrole, 5-methoxyuracil, uracil-5-oxyacetic acid, 5-methoxycarbonylmethyluracil, 5-methyl-2-thiouracil, 5-methoxycarbonylmethyl-2-thiouracil, 5-methylaminomethyl-2-thiouracil, 3-(3-amino-3-carboxypropyl)uracil, 3-methylcytosine, 5-methylcytosine, N⁴-acetyl cytosine, 2-thiocytosine, N6-methyladenine, N6-isopentyladenine, 2-methylthio-N6-isopentenyladenine, N-methylguanines, or O-alkylated bases.

x is 5-100, or chosen to comply with a length for an RNA agent described herein;

g is 0-2.

### DEFINITIONS

The term “halo” refers to any radical of fluorine, chlorine, bromine or iodine. The term “alkyl” refers to saturated and unsaturated non-aromatic hydrocarbon chains that may be a straight chain or branched chain, containing the indicated number of carbon atoms (these include without limitation propyl, allyl, or propargyl), which may be optionally inserted with N, O, or S. For example, C₁-C₂₀ indicates that the group may have from 1 to 20 (inclusive) carbon atoms in it. The term “alkoxy” refers to an —O-alkyl radical. The term “alkylene” refers to a divalent alkyl (i.e., —R—). The term “alkylenedioxo” refers to a divalent species of the structure —O—R—O—, in which R represents an alkylene. The term “aminoalkyl” refers to an alkyl substituted with an amino. The term “mercapto” refers to an —SH radical. The term “thioalkoxy” refers to an —S-alkyl radical.

The term “aryl” refers to a 6-carbon monocyclic or 10-carbon bicyclic aromatic ring system wherein 0, 1, 2, 3, or 4 atoms of each ring may be substituted by a substituent. Examples of aryl groups include phenyl, naphthyl and the like. The term “arylalkyl” or the term “aralkyl” refers to alkyl substituted with an aryl. The term “arylalkoxy” refers to an alkoxy substituted with aryl.

The term “cycloalkyl” as employed herein includes saturated and partially unsaturated cyclic hydrocarbon groups having 3 to 12 carbons, for example, 3 to 8 carbons, and, for example, 3 to 6 carbons, wherein the cycloalkyl group additionally may be optionally substituted. Cycloalkyl groups include, without limitation, cyclopropyl, cyclobutyl, cyclopentyl, cyclopentenyl, cyclohexyl, cyclohexenyl, cycloheptyl, and cyclooctyl.

The term “heteroaryl” refers to an aromatic 5-8 membered monocyclic, 8-12 membered bicyclic, or 11-14 membered tricyclic ring system having 1-3 heteroatoms if monocyclic, 1-6 heteroatoms if bicyclic, or 1-9 heteroatoms if tricyclic, said heteroatoms selected from O, N, or S (e.g., carbon atoms and 1-3, 1-6, or 1-9 heteroatoms of N, O, or S if monocyclic, bicyclic, or tricyclic, respectively), wherein 0, 1, 2, 3, or 4 atoms of each ring may be substituted by a substituent. Examples of heteroaryl groups include pyridyl, furyl or furanyl, imidazolyl, benzimidazolyl, pyrimidinyl, thiophenyl or thienyl, quinolinyl, indolyl, thiazolyl, and the like. The term “heteroarylalkyl” or the term “heteroaralkyl” refers to an alkyl substituted with a heteroaryl. The term “heteroarylalkoxy” refers to an alkoxy substituted with heteroaryl.

The term “heterocyclyl” refers to a nonaromatic 5-8 membered monocyclic, 8-12 membered bicyclic, or 11-14 membered tricyclic ring system having 1-3 heteroatoms if monocyclic, 1-6 heteroatoms if bicyclic, or 1-9 heteroatoms if tricyclic, said heteroatoms selected from O, N, or S (e.g., carbon atoms and 1-3, 1-6, or 1-9 heteroatoms of N, O, or S if monocyclic, bicyclic, or tricyclic, respectively), wherein 0, 1, 2 or 3 atoms of each ring may be substituted by a substituent. Examples of heterocyclyl groups include piperazinyl, pyrrolidinyl, dioxanyl, morpholinyl, tetrahydrofuranyl, and the like.

The term “oxo” refers to an oxygen atom, which forms a carbonyl when attached to carbon, an N-oxide when attached to nitrogen, and a sulfoxide or sulfone when attached to sulfur.

The term “acyl” refers to an alkylcarbonyl, cycloalkylcarbonyl, arylcarbonyl, heterocyclylcarbonyl, or heteroarylcarbonyl substituent, any of which may be further substituted by substituents.

The term “substituents” refers to a group “substituted” on an alkyl, cycloalkyl, aryl, heterocyclyl, or heteroaryl group at any atom of that group. Suitable substituents include, without limitation, halo, hydroxy, oxo, nitro, haloalkyl, alkyl, alkaryl, aryl, aralkyl, alkoxy, aryloxy, amino, acylamino, alkylcarbamoyl, arylcarbamoyl, aminoalkyl, alkoxycarbonyl, carboxy, hydroxyalkyl, alkanesulfonyl, arenesulfonyl, alkanesulfonamido, arenesulfonamido, aralkylsulfonamido, alkylcarbonyl, acyloxy, cyano, and ureido groups.

### Chimeric Oligonucleotides.

The present invention also includes compositions employing antisense compounds, including single and double stranded siRNAs, which are chimeric compounds. “Chimeric” antisense compounds or “chimeras” are antisense compounds, particularly oligonucleotides, which contain two or more chemically distinct regions, each made up of at least one monomer unit, i.e., a nucleotide in the case of an oligonucleotide compound. These oligonucleotides typically contain at least one region wherein the oligonucleotide is modified so as to confer upon the oligonucleotide increased resistance to nuclease degradation, increased cellular uptake, and/or increased binding affinity for the target nucleic acid. An additional region of the oligonucteotide may serve as a substrate for enzymes capable of cleaving RNA:DNA or RNA:RNA hybrids. By way of example, RNase H is a cellular endonuclease which cleaves the RNA strand of an RNA:DNA duplex. Activation of RNase H, therefore, results in cleavage of the RNA target, thereby greatly enhancing the efficiency of oligonucleotide inhibition of gene expression. Consequently, comparable results can often be obtained with shorter oligonucleotides when chimeric oligonucleotides are used, compared to phosphorothioate oligodeoxynucleotides hybridizing to the same target region. Cleavage of the RNA target can be routinely detected by gel electrophoresis and, if necessary, associated nucleic acid hybridization techniques known in the art. RNase H-mediated target cleavage is distinct from the use of ribozymes to cleave nucleic acids.

By way of example, such “chimeras” may be “gapmers.” Gapmers are oligonucleotides in which a central portion (the “gap” or “gap region”) of the oligonucleotide serves as a substrate for, e.g., RNase H, and the 5′ and 3′ portions (the “wings” or “wing regions”) are modified in such a fashion so as to have greater affinity for, or stability when duplexed with, the target RNA molecule but are unable to support nuclease activity (e.g., 2′-fluoro- or 2′-methoxyethoxy-substituted). Each gap region may be from about 10 to about 30 nucleotides in length. Each wing region independently may be between 0 and about 10 nucleotides in length. In one embodiment, the gapmer is a ten deoxynucleotide gap region flanked by two wings independently containing five non-deoxynucleotides. This is referred to as a 5-10-5 gapmer.

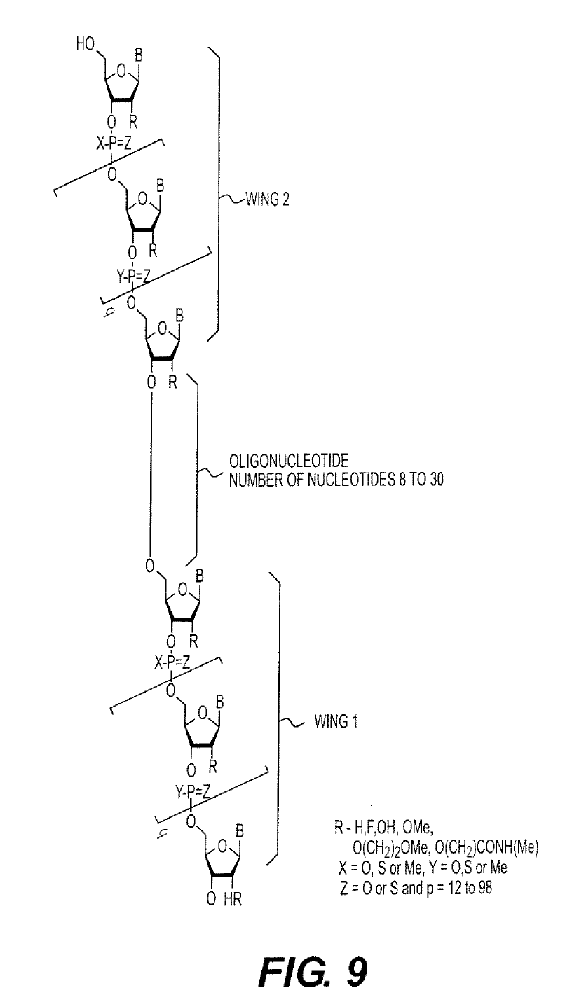

Referring to FIG. 9, shown is a gapmer oligonucleotide with unmodified ribosugar nucleotides in the gap region and modified nucleotides in the wing regions. Length of the gap region is between 8 and 30 nucleotides; preferably the length is between 14 and 21, and more preferably between 16 and 20. For example, each wing will have from 1 to about 8 2′-F modifications. Each wing may have a combination of one 2′-F and one or more 2′-OMe modifications. Alternatively, each wing may have a combination of two 2′-F modifications and one or more 2′-OH modifications. In another embodiment, each wing has a combination of one or more 2′-F modifications and one or more 2′-deoxy modifications. In another embodiment, each wing has a combination of one or more 2′-F modifications and one or more 2′-O-MOE modifications. In another embodiment, each wing has a combination of one or more 2′-F modifications and one or more 2′-O-NMA modifications. In another embodiment, each wing has a combination of one or more 2′-F modifications and one or more LNA modifications. In another embodiment, each wing has a combination of one or more 2′-F modifications and one or more ENA modifications. In another embodiment, each wing has a combination of one or more 2′-F modifications, one or more 2′-OH modifications, and one or more 2′-deoxy modifications. In another embodiment, each wing has a combination of one or more 2′-F modifications, one or more 2′-OH modifications, and one or more 2′-OMe modifications. In another embodiment, each wing has a combination of one or more 2′-F modifications, one or more 2′-OH modifications, and one or more 2′-O-MOE modifications. In another embodiment, each wing has a combination of one or more 2′-F modifications, one or more 2′-OH modifications, and one or more 2′-O-NMA modifications. In another embodiment, each wing has a combination of one or more 2′-F modifications, one or more 2′-OMe modifications, and one or more 2′-deoxy modifications. In another embodiment, each wing has a combination of one or more 2′-F modifications, one or more 2′-OMe modifications, and one or more 2′-deoxy modifications. In another embodiment, each wing has a combination of one or more 2′-F modifications, one or more 2′-OMe modifications, and one or more 2′-O-MOE modifications. In another embodiment, each wing has a combination of one or more 2′-F modifications, one or more 2′-OMe modifications, and one or more 2′-O-NMA modifications. In another embodiment, each wing has a combination of one or more 2′-F modifications, one or more 2′-OH modifications, and one or more LNA modifications. In another embodiment, each wing has a combination of one or more 2′-F modifications, one or more 2′-OMe modifications, and one or more LNA modifications. In another embodiment, each wing has a combination of one or more 2′-F modifications, one or more 2′-O-MOE modifications, and one or more LNA modifications. In another embodiment, each wing has a combination of one or more 2′-F modifications, one or more 2′-O-MOE modifications, and one or more 2′-deoxy modifications. In another embodiment, each wing has a combination of one or more 2′-F modifications, one or more LNA modifications, and one or more 2′-deoxy modifications. In another embodiment, each wing has a combination of one or more 2′-F modifications, one or more 2′-OH modifications, and one or more LNA modifications. In another embodiment, each wing has a combination of one or more 2′-F modifications, one or more 2′-O-MOE modifications, and one or more ENA modifications.

In another embodiment, each wing has a combination of one or more 2′-F modifications, one or more 2′-deoxy modifications, and one or more ENA modifications. In another embodiment, each wing has a combination of one or more 2′-F modifications, one or more 2′-OH modifications, and one or more ENA modifications.

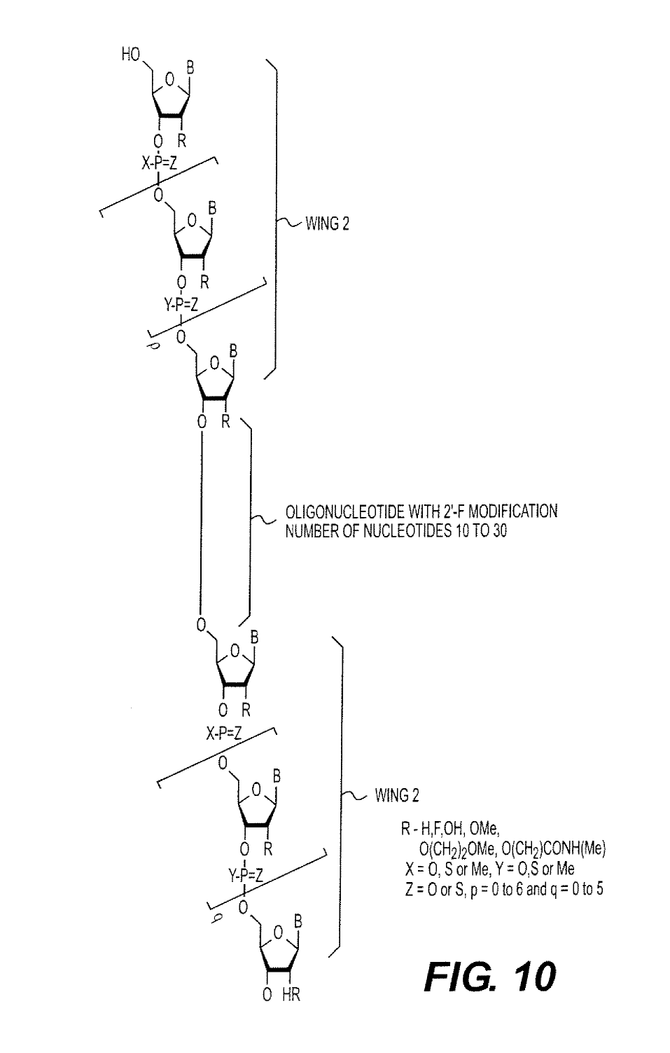

Referring to FIG. 10, showing is a gapmer oligonucleotide. In one embodiment the gap region will contain all 2′-F modified nucleotides in the gap, and the wing regions may independently have zero, one or more than modified ribosugars. Length of the gap region is between 8 and 30 nucleotides; preferably the length is between 14 and 21, and more preferably between 16 and 20. In another embodiment the gap region will contain alternating 2′-F and 2′-OH modifications, and the wing regions may independently have zero, one or more than modified ribosugars. In another embodiment the gap region will contain pyrimidines having 2′-F modifications and purines having 2′-OH modifications, and the wing regions may independently have zero, one or more than modified ribosugars. In another embodiment the gap region will contain purines having 2′-F modifications and pyrimidines having 2′-OH modifications, and the wing regions may independently have zero, one or more than modified ribosugars. In another embodiment the gap region will contain alternating 2′-F and 2′-OMe modifications, and the wing regions may independently have zero, one or more than modified ribosugars. In another embodiment the gap region will contain pyrimidines having 2′-F modifications and purines having 2′-OMe modifications, and the wing regions may independently have zero, one or more than modified ribosugars. In another embodiment the gap region will contain purines having 2′-F modifications and pyrimidines having 2′-OMe modifications, and the wing regions may independently have zero, one or more than modified ribosugars. In these embodiments, each wing will independently have zero, one, two or more than two (up to and including about eight) of the following modifications in any order: 2′-OH; 2′-deoxy; 2′-OMe; 2′-O-NMA; LNA; ENA. Included are the combination of: 2′-deoxy and 2′-OMe modifications; 2′-deoxy and 2′-OH modifications; 2′-deoxy and 2′-O-MOE modifications; 2′-deoxy and LNA modifications; 2′-OH and 2′-O-NMA modifications; 2′-OH and LNA modifications; 2′-OH and 2′-OMe modifications; 2′-OH and 2′-O-MOE modifications; 2′-OH and 2′ ara-F modifications; 2′-OH and ENA modifications; 2′-deoxy and ENA modifications; 2′-O-MOE and ENA modifications; 2′-OMe and ENA modifications; 2′-O-NMA and ENA modifications; 2′-deoxy, 2′-OH and 2′-OMe modifications; 2′-deoxy, 2′-OH and 2′-O-MOE modifications; 2′-deoxy, 2′-OH and 2′-O-NMA modifications; 2′-deoxy, 2′-OMe and 2′-O-NMA modifications; 2′-OH, 2′-OMe and 2′-O-MOE modifications; 2′-OH, 2′-OMe and 2′-O-NMA modifications; 2′-OH, 2′-O-MOE and LNA modifications; 2′-O-MOE, 2′-OMe and LNA modifications; 2′-OH, 2′-O-MOE and ENA modifications; 2′-O-MOE, 2′-OMe and ENA modifications; 2′-OH, 2′-OMe and LNA modifications; 2′-OH, 2′-OMe and ENA modifications; 2′-deoxy, 2′-OMe and ENA modifications; 2′-OH, 2′-deoxy and ENA modifications; 2′-O-NMA, 2′-OMe and ENA modifications, each of which can occur in any order. Number of each individual sugar in the wings varies between 0 and 8.

Other chimeras include “hemimers,” which are oligonucleotides in which a first segment (such as the 5′ segment) of the oligonucleotide serves as a substrate for, e.g., RNase H, whereas a second segment (such as the 3′ segment) is modified in such a fashion so as to have greater affinity for, or stability when duplexed with, the target RNA molecule but is unable to support nuclease activity (e.g., 2′-fluoro- or 2′-methoxyethoxy-substituted), or vice-versa.

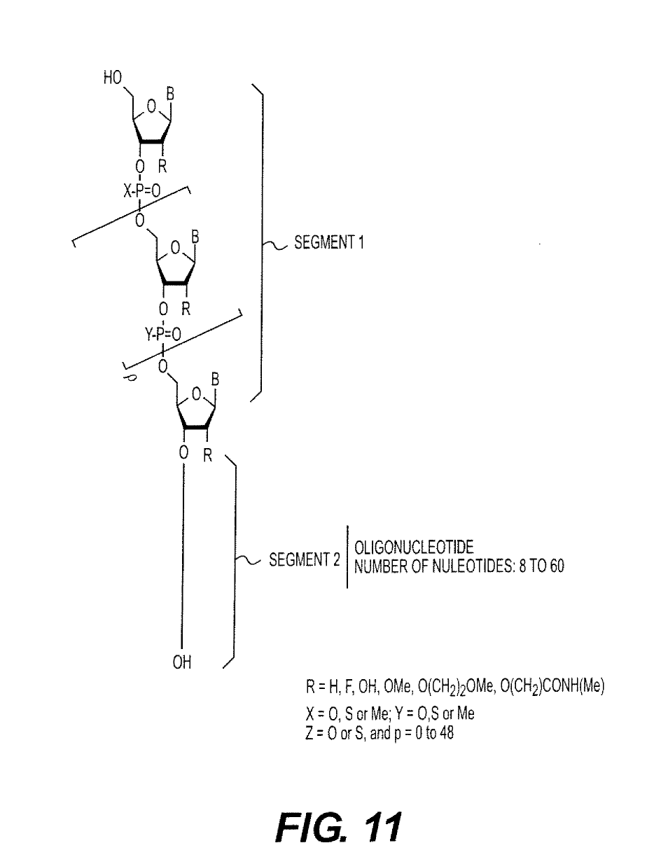

Referring to FIG. 11, shown is a hemimer oligonucleotides, where Segment 1 contains an oligonucleotide sequence that is antisense to and binds with a target mRNA, and Segment 2 contains a substrate sequence. In one embodiment, all nucleotides in Segment 1 contain 2′-F modifications, and Segment 2 may contain modified and/or unmodified sugars. In another embodiment, all nucleotides in Segment 2 contain 2′-F modifications, and Segment 1 may contain modified and/or unmodified sugars. In another embodiment, alternating nucleotides in Segment 1 contain 2′-F modifications, and Segment 2 may contain modified and/or unmodified sugars. In another embodiment, alternating nucleotides in Segment 2 contain 2′-F modifications, and Segment 1 may contain modified and/or unmodified sugars. In another embodiment, all pyrimidine nucleotides in Segment 1 contain 2′-F modifications, and Segment 2 may contain modified and/or unmodified sugars. In another embodiment, all pyrimidine nucleotides in Segment 2 contain 2′-F modifications, and Segment 1 may contain modified and/or unmodified sugars. In another embodiment, all purine nucleotides in Segment 1 contain 2′-F modifications, and Segment 2 may contain modified and/or unmodified sugars. In another embodiment, all purine nucleotides in Segment 2 contain 2′-F modifications, and Segment 1 may contain modified and/or unmodified sugars. In another embodiment, all pyrimidine nucleotides in Segment 1 contain 2′-F modifications, all purine nucleotides in Segment 1 contain 2′-OMe modifications, and Segment 2 may contain modified and/or unmodified sugars. In another embodiment, all pyrimidine nucleotides in Segment 2 contain 2′-F modifications, all purine nucleotides in Segment 2 contain 2′-OMe modifications, and Segment 1 may contain modified and/or unmodified sugars. In another embodiment, Segment 2 contains alternating 2′-F and 2′-OMe modifications, and Segment 1 may contain modified and/or unmodified sugars. In another embodiment, all pyrimidine nucleotides in Segment 1 contain 2′-F modifications, all purine nucleotides in Segment 1 contain 2′-OMe modifications, and Segment 1 may contain modified and/or unmodified sugars.

A number of chemical modifications to oligonucleotides that confer greater oligonucleotide:RNA duplex stability have been described by Freier et al. (Nucl. Acids Res., 1997, 25, 4429). Such modifications are preferred for the RNase H-refractory portions of chimeric oligonucleotides and may generally be used to enhance the affinity of an antisense compound for a target RNA.

Chimeric antisense compounds of the invention may also be formed as composite structures of two or more oligonucleotides, modified oligonucleotides, oligonucleosides and/or oligonucleotide mimetics as described herein. Such compounds have also been referred to in the art as hybrids or gapmers. Representative U.S. patents that teach the preparation of such hybrid structures include, but are not limited to, U.S. Pat. Nos. 5,013,830; 5,149,797; 5,220,007; 5,256,775; 5,366,878; 5,403,711; 5,491,133; 5,565,350; 5,623,065; 5,652,355; 5,652,356; and 5,700,922, and U.S. patent application Ser. No. 08/465,880, each of which is herein incorporated by reference.

Chimeric single and double stranded siRNAs of the invention may also be formed as composite structures oligonucleotides, modified oligonucleotides, oligonucleosides and/or oligonucleotide mimetics.

### Palindromes

The siRNA compounds of the invention can target more than one RNA region. For example, an siRNA compound can include a first and second sequence that are sufficiently complementary to each other to hybridize. The first sequence can be complementary to a first target RNA region and the second sequence can be complementary to a second target RNA region. The first and second sequences of the siRNA compound can be on different RNA strands, and the mismatch between the first and second sequences can be less than 50%, 40%, 30%, 20%, 10%, 5%, or 1%. The first and second sequences of the siRNA compound are on the same RNA strand, and in a related embodiment more than 50%, 60%, 70%, 80%, 90%, 95%, or 1% of the siRNA compound can be in bimolecular form. The first and second sequences of the siRNA compound can be fully complementary to each other.

The first target RNA region can be encoded by a first gene and the second target RNA region can encoded by a second gene, or the first and second target RNA regions can be different regions of an RNA from a single gene. The first and second sequences can differ by at least 1 nucleotide.

The first and second target RNA regions can be on transcripts encoded by first and second sequence variants, e.g., first and second alleles, of a gene. The sequence variants can be mutations, or polymorphisms, for example. The first target RNA region can include a nucleotide substitution, insertion, or deletion relative to the second target RNA region, or the second target RNA region can a mutant or variant of the first target region.

The first and second target RNA regions can comprise viral or human RNA regions. The first and second target RNA regions can also be on variant transcripts of an oncogene or include different mutations of a tumor suppressor gene transcript. In addition, the first and second target RNA regions can correspond to hot-spots for genetic variation.

The compositions of the invention can include mixtures of siRNA molecules. For example, one siRNA-containing compound can contain a first sequence and a second sequence sufficiently complementary to each other to hybridize, and in addition the first sequence is complementary to a first target RNA region and the second sequence is complementary to a second target RNA region. The mixture can also include at least one additional siRNA compound variety that includes a third sequence and a fourth sequence sufficiently complementary to each other to hybridize, and where the third sequence is complementary to a third target RNA region and the fourth sequence is complementary to a fourth target RNA region. In addition, the first or second sequence can be sufficiently complementary to the third or fourth sequence to be capable of hybridizing to each other. The first and second sequences can be on the same or different RNA strands, and the third and fourth sequences can be on the same or different RNA strands.

The target RNA regions can be variant sequences of a viral or human RNA, and in certain embodiments, at least two of the target RNA regions can be on variant transcripts of an oncogene or tumor suppressor gene. The target RNA regions can correspond to genetic hot-spots.

Methods of making an siRNA compound composition can include obtaining or providing information about a region of an RNA of a target gene (e.g., a viral or human gene, or an oncogene or tumor suppressor, e.g., p53), where the region has high variability or mutational frequency (e.g., in humans) In addition, information about a plurality of RNA targets within the region can be obtained or provided, where each RNA target corresponds to a different variant or mutant of the gene (e.g., a region including the codon encoding p53 248Q and/or p53 249S). The siRNA compound can be constructed such that a first sequence is complementary to a first of the plurality of variant RNA targets (e.g., encoding 249Q) and a second sequence is complementary to a second of the plurality of variant RNA targets (e.g., encoding 249S), and the first and second sequences can be sufficiently complementary to hybridize.

Sequence analysis, e.g., to identify common mutants in the target gene, can be used to identify a region of the target gene that has high variability or mutational frequency. A region of the target gene having high variability or mutational frequency can be identified by obtaining or providing genotype information about the target gene from a population.

Expression of a target gene can be modulated, e.g., downregulated or silenced, by providing an siRNA compound that has a first sequence and a second sequence sufficiently complementary to each other to hybridize. In addition, the first sequence can be complementary to a first target RNA region and the second sequence can be complementary to a second target RNA region.

An siRNA compound can include a first sequence complementary to a first variant RNA target region and a second sequence complementary to a second variant RNA target region. The first and second variant RNA target regions can correspond to first and second variants or mutants of a target gene, e.g., viral gene, tumor suppressor or oncogene. The first and second variant target RNA regions can include allelic variants, mutations (e.g., point mutations), or polymorphisms of the target gene. The first and second variant RNA target regions can correspond to genetic hot-spots.

A plurality of siRNA compounds (e.g., a panel or bank) can be provided.

### Other Embodiments

In yet another embodiment, siRNAs are produced in a cell in vivo, e.g., from exogenous DNA templates that are delivered into the cell. For example, the DNA templates can be inserted into vectors and used as gene therapy vectors. Gene therapy vectors can be delivered to a subject by, for example, intravenous injection, local administration (U.S. Pat. No. 5,328,470), or by stereotactic injection (see, e.g., Chen et al. (1994) Proc. Natl. Acad. Sci. USA 91:3054-3057). The pharmaceutical preparation of the gene therapy vector can include the gene therapy vector in an acceptable diluent, or can comprise a slow release matrix in which the gene delivery vehicle is imbedded. The DNA templates, for example, can include two transcription units, one that produces a transcript that includes the top strand of a siRNA compound and one that produces a transcript that includes the bottom strand of a siRNA compound. When the templates are transcribed, the siRNA compound is produced, and processed into ssiRNA compound fragments that mediate gene silencing.

### Antagomirs

Antagomirs are RNA-like oligonucleotides that harbor various modifications for RNAse protection and pharmacologic properties, such as enhanced tissue and cellular uptake. They differ from normal RNA by, for example, complete 2′-O-methylation of sugar, phosphorothioate backbone and, for example, a cholesterol-moiety at 3′-end. Antagomirs may be used to efficiently silence endogenous miRNAs by forming duplexes comprising the antagomir and endogenous miRNA, thereby preventing miRNA-induced gene silencing. An example of antagomir-mediated miRNA silencing is the silencing of miR-122, described in Krutzfeldt et al, Nature, 2005, 438: 685-689, which is expressly incorporated by reference herein in its entirety. Antagomir RNAs may be synthesized using standard solid phase oligonucleotide synthesis protocols. See U.S. patent application Ser. Nos. 11/502,158 and 11/657,341 (the disclosure of each of which are incorporated herein by reference).

An antagomir can include ligand-conjugated monomer subunits and monomers for oligonucleotide synthesis. Exemplary monomers are described in U.S. application Ser. No. 10/916,185, filed on Aug. 10, 2004. An antagomir can have a ZXY structure, such as is described in PCT Application No. PCT/US2004/07070 filed on Mar. 8, 2004. An antagomir can be complexed with an amphipathic moiety. Exemplary amphipathic moieties for use with oligonucleotide agents are described in PCT Application No. PCT/US2004/07070, filed on Mar. 8, 2004.

### Aptamers

Aptamers are nucleic acid or peptide molecules that bind to a particular molecule of interest with high affinity and specificity (Tuerk and Gold, Science 249:505 (1990); Ellington and Szostak, Nature 346:818 (1990)). DNA or RNA aptamers have been successfully produced which bind many different entities from large proteins to small organic molecules. See Eaton, Curr. Opin. Chem. Biol. 1:10-16 (1997), Famulok, Curr. Opin. Struct. Biol. 9:324-9 (1999), and Hermann and Patel, Science 287:820-5 (2000). Aptamers may be RNA or DNA based, and may include a riboswitch. A riboswitch is a part of an mRNA molecule that can directly bind a small target molecule, and whose binding of the target affects the gene's activity. Thus, an mRNA that contains a riboswitch is directly involved in regulating its own activity, depending on the presence or absence of its target molecule. Generally, aptamers are engineered through repeated rounds of in vitro selection or equivalently, SELEX (systematic evolution of ligands by exponential enrichment) to bind to various molecular targets such as small molecules, proteins, nucleic acids, and even cells, tissues and organisms. The aptamer may be prepared by any known method, including synthetic, recombinant, and purification methods, and may be used alone or in combination with other aptamers specific for the same target. Further, as described more fully herein, the term “aptamer” specifically includes “secondary aptamers” containing a consensus sequence derived from comparing two or more known aptamers to a given target.

### MicroRNAs

MicroRNAs (miRNAs) are an abundant class of short endogenous RNAs that act as post-transcriptional regulators of gene expression by base-pairing with their target mRNAs. The approximately 22 nucleotide (nt) mature miRNAs are processed sequentially from longer hairpin transcripts (primary miRNA/pri-miRNA or precursor miRNA) by the RNAse III ribonucleases Drosha (Lee et al. 2003) and Dicer (Hutvagner et al. 2001, Ketting et al. 2001). More than 3400 miRNAs have been annotated in vertebrates, invertebrates and plants according to the miRBase microRNA database release 7.1 in October 2005 (Griffith-Jones 2004, Griffith-Jones et al. 2006), and many miRNAs that correspond to putative miRNA genes have also been bioinformatically predicted. More than half of all known mammalian miRNAs are hosted within the introns of pre-mRNAs or long ncRNA transcripts (Rodriquez et al. 2004). Many miRNA genes are arranged in genomic clusters (Lagos-Quintana et al. 2001). For example, approximately 40% of human miRNA genes appear in clusters of two or more, with the largest cluster of 40 miRNA genes being located in the human imprinted 14q32 domain (Setiz et al. 2004; Altuvia et al. 2005). MicroRNAs have been associated in a variety of human diseases, including breast and lung cancer. See U.S. patent application Ser. No. 11/730,570 (the disclosure of which is incorporated herein by reference).

MicroRNAs were first discovered in C. elegans, but have now been found in plants, invertebrates, and vertebrates, including humans miRNAs regulate protein expression post-transcriptionally through a process that is biochemically indistinguishable from RNAi. The miRNAs are transcribed as long precursors, called pri-miRNAs, by pol II. The pri-miRNA is processed in the nucleus to pre-miRNA, hairpin intermediates of 60 to 70 nucleotides by the RNase III endonuclease Drosha. This enzyme activity was discovered and described as early as 2000. Following export into the cytoplasm, Dicer cleaves the pre-miRNA to produce an imperfect duplex. This duplex enters the same gene-silencing pathway described earlier for siRNAs (FIG. 1). The choice of which strand to degrade appears to be made within the RISC complex, perhaps based on the thermodynamic properties of the ends of the duplex.

Although the initial observations of miRNA regulation in C. elegans indicated that gene expression was reduced without alteration of mRNA levels, cleavage of HOXB8 was detected mRNA in mice, indicating that miRNA regulated gene expression in animals can occur through a cleavage mechanism. Thus, in mammals miRNAs can down-regulate gene expression by one of two post-transcriptional mechanisms: mRNA cleavage or translational repression. Currently, it is assumed that the choice of mechanism is driven by the extent of complementarity between the miRNA and the messenger RNA target. RISC appears to function as an RNA cleavage enzyme when miRNA is fully complementary RNA target sites. If the duplex formed between the target site and the miRNA contains mismatches, cleavage may be precluded, but RISC remains bound to the mRNA target, resulting in translational repression. The cooperative binding of multiple RISCs provides more efficient translational repression than binding of a single complex. This may explain the presence of multiple miRNA complementary sites in the UTRs of messages regulated by miRNA.

Role of miRNAs in vivo. Conservative predications suggest that up to 30% of human genes are regulated by miRNA. The relevance of these small RNAs to human health should not be underestimated. In model organisms, numerous miRNAs are involved in developmental regulation and this is presumably the case in humans Fragile X syndrome was the first human disease linked to a dysfunction in an miRNA pathway. Spinal muscular atrophy, early onset parkinsonism, and X-linked mental retardation also appear to involve loss or mutation in miRNA or components of the pathway. Evidence is mounting that miRNA dysregulation plays a role in cancer pathogenesis. Approximately half of known miRNA genes are located in cancer-associated genomic regions. For example, several studies suggest that the oncogene RAS is regulated by the let-7 miRNA family

In order to delineate the roles of miRNAs in disease processes, two approaches can be conceived in theory. The studies demonstrating the involvement of miRNAs in metabolic disease are illustrative of the two approaches to understanding the precise molecular function of mammalian miRNAs in vivo: one can treat with an agonist (to increase expression of a particular miRNA) or an antagonist (to decrease expression of an miRNA). Both of these approaches could also be used therapeutically to modulate miRNAs and hence to control gene products involved in disease processes. The islet-specific miRNA, miR-375, was over-expressed in order to study the role this miRNA in pancreatic endocrine cells. Overexpression of miR-375 suppressed glucose-induced insulin secretion miR-375 modulates glucose-stimulated insulin secretion and exocytosis by blocking the expression of myotrophin, a protein associated with neuronal secretion.

Antagomirs. The second approach to interfere with miRNAs is based on synthetic anti-miRNA oligonucleotides that can be introduced into cells or animals. Different classes of anti-miRNA oligonucleotides have been tested in cell culture and have been reviewed. The first in vivo demonstration was achieved by a cholesterol-conjugated anti-miRNA named an antagomir. The antagomir, complementary in sequence to the murine miR-122, was modified with three chemistries: uniform 2′-OMe nucleotides (for sufficient nuclease stability and binding affinity), terminal phosphorothioate linkages (for nuclease stability), and a cholesterol (for liver targeting) conjugated via a hydroxyprolinol-aminocaproic acid tether. The silencing of endogenous miRNAs using this antagomir was observed within 24 hours after administration and the silencing was specific, efficient, and long lasting.

The biological significance of silencing miR-122, an abundant liver-specific miRNA, was evaluated. Northern blot analysis revealed miR-122 was completely abolished and the effects were long lasting, at least for 23 days. The effects were sequence specific (mismatched antagomirs were not effective) and miR-122 specific (other miRs such as let-7 and miR-22 were not affected). Gene expression and bioinformatic analysis of messenger RNA from antagomir-treated animals revealed that the untranslated regions of many up-regulated genes are strongly enriched in miR-122 recognition motifs, whereas down-regulated genes are depleted in these motifs. For example, the aldolase-A gene was up-regulated nearly 600% by antagomir treatment; this was used as one of the positive readouts for this antagomir treatment. Several mRNAs in the cholesterol-biosynthesis pathway, including the cholesterol biosynthesis target HMGCR (hydroxymethylglutaryl coenzyme-A reductase, the target for many statins), MVK (mevalonate kinase), and FDPS (farnesyl diphosphate synthetase), were positively regulated by miR-122. Offering further support for the relevance of the miRNA in cholesterol biosynthesis, plasma cholesterol levels were reduced in antagomir-122-treated mice by nearly 40%.

In the same study, intravenous administration of antagomir against miR-16, which is expressed in almost all tissues, resulted in a marked reduction of miR-16 levels except brain: levels were reduced in liver, lung, kidney, heart, intestine, fat, skin, bone marrow, muscle, ovaries, and adrenals. This experiment demonstrated the biodistribution properties of the cholesterol conjugate and showed that cholesterol-conjugated, modified oligonucleotides function as an effective antagomirs

In a related study, mice were dosed with uniform 2′-O-methoxyethyl phosphorothioate oligonucleotide complementary to miR-122. Complete inhibition of miR-122 was observed after a four-week treatment. Inhibition resulted in reduced plasma cholesterol levels, increased hepatic fatty-acid oxidation, and a decrease in hepatic fatty-acid and cholesterol synthesis rates. In a diet-induced obese mouse model, miR-122 inhibition resulted in decreased plasma cholesterol levels and a significant improvement in liver steatosis; in addition, expression of several lipogenic genes was reduced. The results from both studies suggest that miR-122 is one of the regulators of cholesterol and fatty-acid metabolism in the adult liver and show that antagomirs of microRNAs are powerful tools for silencing of specific miRNAs in vivo. These two reports (99, 100) strongly suggest that antagomirs will provide a therapeutic strategy for silencing miRNAs and will allow control the miRNAs involved in the diseases described above.

miRNA mimics miRNA mimics represent a class of molecules that can be used to imitate the gene silencing ability of one or more miRNAs. Thus, the term “microRNA mimic” refers to synthetic non-coding RNAs (i.e. the miRNA is not obtained by purification from a source of the endogenous miRNA) that are capable of entering the RNAi pathway and regulating gene expression. miRNA mimics can be designed as mature molecules (e.g. single stranded) or mimic precursors (e.g., pri- or pre-miRNAs) miRNA mimics can be comprised of nucleic acid (modified or modified nucleic acids) including oligonucleotides comprising, without limitation, RNA, modified RNA, DNA, modified DNA, locked nucleic acids, or 2′-O,4′-C-ethylene-bridged nucleic acids (ENA), or any combination of the above (including DNA-RNA hybrids). In addition, miRNA mimics can comprise conjugates that can affect delivery, intracellular compartmentalization, stability, specificity, functionality, strand usage, and/or potency. In one design, miRNA mimics are double stranded molecules (e.g., with a duplex region of between about 16 and about 31 nucleotides in length) and contain one or more sequences that have identity with the mature strand of a given miRNA. Modifications can comprise 2′ modifications (including 2′-O methyl modifications and 2′ F modifications) on one or both strands of the molecule and internucleotide modifications (e.g. phorphorthioate modifications) that enhance nucleic acid stability and/or specificity. In addition, miRNA mimics can include overhangs. The overhangs can consist of 1-6 nucleotides on either the 3′ or 5′ end of either strand and can be modified to enhance stability or functionality. In one embodiment, a miRNA mimic comprises a duplex region of between 16 and 31 nucleotides and one or more of the following chemical modification patterns: the sense strand contains 2′-O-methyl modifications of nucleotides 1 and 2 (counting from the 5′ end of the sense oligonucleotide), and all of the Cs and Us; the antisense strand modifications can comprise 2′ F modification of all of the Cs and Us, phosphorylation of the 5′ end of the oligonucleotide, and stabilized internucleotide linkages associated with a 2 nucleotide 3′ overhang.

Supermir. A supermir refers to a single stranded, double stranded or partially double stranded oligomer or polymer of ribonucleic acid (RNA) or deoxyribonucleic acid (DNA) or both or modifications thereof, which has a nucleotide sequence that is substantially identical to an miRNA and that is antisense with respect to its target. This term includes oligonucleotides composed of naturally-occurring nucleobases, sugars and covalent internucleoside (backbone) linkages and which contain at least one non-naturally-occurring portion which functions similarly. Such modified or substituted oligonucleotides are preferred over native forms because of desirable properties such as, for example, enhanced cellular uptake, enhanced affinity for nucleic acid target and increased stability in the presence of nucleases. In a preferred embodiment, the supermir does not include a sense strand, and in another preferred embodiment, the supermir does not self-hybridize to a significant extent. An supermir featured in the invention can have secondary structure, but it is substantially single-stranded under physiological conditions. An supermir that is substantially single-stranded is single-stranded to the extent that less than about 50% (e.g., less than about 40%, 30%, 20%, 10%, or 5%) of the supermir is duplexed with itself. The supermir can include a hairpin segment, e.g., sequence, preferably at the 3′ end can self hybridize and form a duplex region, e.g., a duplex region of at least 1, 2, 3, or 4 and preferably less than 8, 7, 6, or n nucleotides, e.g., 5 nucleotides. The duplexed region can be connected by a linker, e.g., a nucleotide linker, e.g., 3, 4, 5, or 6 dTs, e.g., modified dTs. In another embodiment the supermir is duplexed with a shorter oligo, e.g., of 5, 6, 7, 8, 9, or 10 nucleotides in length, e.g., at one or both of the 3′ and 5′ end or at one end and in the non-terminal or middle of the supermir

Antimir or miRNA inhibitor. The terms “antimir” “microRNA inhibitor”, “miR inhibitor”, or “inhibitor” are synonymous and refer to oligonucleotides or modified oligonucleotides that interfere with the ability of specific miRNAs. In general, the inhibitors are nucleic acid or modified nucleic acids in nature including oligonucleotides comprising RNA, modified RNA, DNA, modified DNA, locked nucleic acids (LNAs), or any combination of the above. Modifications include 2′ modifications (including 2′-0 alkyl modifications and 2′ F modifications) and internucleotide modifications (e.g. phosphorothioate modifications) that can affect delivery, stability, specificity, intracellular compartmentalization, or potency. In addition, miRNA inhibitors can comprise conjugates that can affect delivery, intracellular compartmentalization, stability, and/or potency. Inhibitors can adopt a variety of configurations including single stranded, double stranded (RNA/RNA or RNA/DNA duplexes), and hairpin designs, in general, microRNA inhibitors comprise contain one or more sequences or portions of sequences that are complementary or partially complementary with the mature strand (or strands) of the miRNA to be targeted, in addition, the miRNA inhibitor may also comprise additional sequences located 5′ and 3′ to the sequence that is the reverse complement of the mature miRNA. The additional sequences may be the reverse complements of the sequences that are adjacent to the mature miRNA in the pri-miRNA from which the mature miRNA is derived, or the additional sequences may be arbitrary sequences (having a mixture of A, G, C, or U). In some embodiments, one or both of the additional sequences are arbitrary sequences capable of forming hairpins. Thus, in some embodiments, the sequence that is the reverse complement of the miRNA is flanked on the 5′ side and on the 3′ side by hairpin structures. Micro-RNA inhibitors, when double stranded, may include mismatches between nucleotides on opposite strands. Furthermore, micro-RNA inhibitors may be linked to conjugate moieties in order to facilitate uptake of the inhibitor into a cell. For example, a micro-RNA inhibitor may be linked to cholesteryl 5-(bis(4-methoxyphenyl)(phenyl)methoxy)-3 hydroxypentylcarbamate) which allows passive uptake of a micro-RNA inhibitor into a cell. Micro-RNA inhibitors, including hairpin miRNA inhibitors, are described in detail in Vermeulen et al., “Double-Stranded Regions Are Essential Design Components Of Potent Inhibitors of RISC Function,” RNA 13: 723-730 (2007) and in WO2007/095387 and WO 2008/036825 each of which is incorporated herein by reference in its entirety. A person of ordinary skill in the art can select a sequence from the database for a desired miRNA and design an inhibitor useful for the methods disclosed herein.

Viral miRNAs. In small-sized viral genomes, miRNAs offer an efficient strategy for specific inactivation of host cell defense factors miRNAs have been cloned from herpes viruses, Epstein-Barr virus, human cytomegalovirus, Kaposi's sarcoma-associated virus and are predicted in the genomes of double-stranded DNA (dsDNA) viruses such as herpes simplex virus 1 and 2, variola and vaccinia virus, molluscum contagiosum virus, and human adenoviruses and in the genomes of the single-stranded RNA viruses, measles virus and yellow fever virus. Clear evidence of the importance of miRNA in the viral life cycle has been shown for the simian virus 40 (SV40). The miRNAs from the circular dsDNA SV40 are perfectly complementary to the early viral mRNAs coding for T antigen. The miRNAs accumulate late in infection and reduce the expression of viral T antigens. The cells with miRNAs are less sensitive than cells without miRNA to lysis by cytotoxic T cells and trigger less cytokine production by such cells.

Viral suppression of silencing. Given the huge number of host miRNAs and the potential for complementarity with viral genomes, viruses have an incentive to interfere with the silencing pathway. There is evidence that some viruses inhibit the RNAi pathway. Primate foamy virus type 1 (PFV-1) expresses a protein that sequesters siRNAs. Adenovirus-infected cells accumulate polymerase III transcripts known as virus-associated RNAs (VA RNAs). The VA RNAs appear to inhibit the RNAi pathway through binding of Dicer as well as through competition for the nuclear export factor.

In contrast, hepatitis C virus (HCV) exploits a cellular miRNA to maintain viral abundance. The liver-specific miRNA, miR-122, discussed above in the section on antagomirs is required for high levels of HCV replication. In Huh7 cells containing replicating HCV genomes, the sequestration of miR-122 with antagomirs resulted in a reduction in the amount of HCV RNA. There are two potential binding sites for miR-122 in the HCV RNA: one in the 3′ UTR and the other in the 5′ UTR. Surprisingly, experiments showed that a direct interaction occurs between miR-122 and the 5′ UTR site of HCV RNA. The regulation is likely to occur during replication, rather than during translation or by interference with RNA stability. The binding of miR-122 might allow a conformational rearrangement in the 5′ UTR of the HCV RNA that allows replication to proceed or components of the miRISC that are recruited by miR-122 might be required for viral replication. Current treatments for HCV are often ineffective and a compound directed against conserved sequences of a cellular target such as miR-122 could be attractive. The work described above that dissected the role of miR-122 in cellular metabolism is a first step toward development of an miR-directed therapeutic.

A recent study with HSV-1 showed that the latency-associated transcript (LAT) gene is responsible for survival of HSV-1 in infected neurons. The microRNA generated from the exon 1 region of the HSV-1 LAT gene (miR-LAT) down-regulates two important genes: transforming-growth factor-β (TGF-β) and SMAD3. Both genes are involved in the TGF-β pathway and can either inhibit cell proliferation or induce cell death. Antagomir approaches to inhibition of miR-LAT could be a viable therapeutic approach for abolishing HSV-1 in neurons.

### Decoy Oligonucleotides

Because transcription factors recognize their relatively short binding sequences, even in the absence of surrounding genomic DNA, short oligonucleotides bearing the consensus binding sequence of a specific transcription factor can be used as tools for manipulating gene expression in living cells. This strategy involves the intracellular delivery of such “decoy oligonucleotides”, which are then recognized and bound by the target factor. Occupation of the transcription factor's DNA-binding site by the decoy renders the transcription factor incapable of subsequently binding to the promoter regions of target genes. Decoys can be used as therapeutic agents, either to inhibit the expression of genes that are activated by a transcription factor, or to upregulate genes that are suppressed by the binding of a transcription factor. Examples of the utilization of decoy oligonucleotides may be found in Mann et al., J. Clin. Invest., 2000, 106: 1071-1075, which is expressly incorporated by reference herein, in its entirety.

### U1 Adaptor

U1 adaptor inhibit polyA sites and are bifunctional oligonucleotides with a target domain complementary to a site in the target gene's terminal exon and a ‘U1 domain’ that binds to the U1 smaller nuclear RNA component of the U1 snRNP (Goraczniak, et al., 2008, Nature Biotechnology, 27(3), 257-263, which is expressly incorporated by reference herein, in its entirety). U1 snRNP is a ribonucleoprotein complex that functions primarily to direct early steps in spliceosome formation by binding to the pre-mRNA exon-intron boundary (Brown and Simpson, 1998, Annu Rev Plant Physiol Plant MoI Biol 49:77-95). Nucleotides 2-11 of the 5′ end of U1 snRNA base pair bind with the 5′ ss of the pre mRNA. In one embodiment, oligonucleotides of the invention are U1 adaptors. In one embodiment, the U1 adaptor can be administered in combination with at least one other iRNA agent.

### Physiological Effects

The siRNA compounds described herein can be designed such that determining therapeutic toxicity is made easier by the complementarity of the siRNA with both a human and a non-human animal sequence. By these methods, an siRNA can consist of a sequence that is fully complementary to a nucleic acid sequence from a human and a nucleic acid sequence from at least one non-human animal, e.g., a non-human mammal, such as a rodent, ruminant or primate. For example, the non-human mammal can be a mouse, rat, dog, pig, goat, sheep, cow, monkey, Pan paniscus, Pan troglodytes, Macaca mulatto, or Cynomolgus monkey. The sequence of the siRNA compound could be complementary to sequences within homologous genes, e.g., oncogenes or tumor suppressor genes, of the non-human mammal and the human. By determining the toxicity of the siRNA compound in the non-human mammal, one can extrapolate the toxicity of the siRNA compound in a human. For a more strenuous toxicity test, the siRNA can be complementary to a human and more than one, e.g., two or three or more, non-human animals.

The methods described herein can be used to correlate any physiological effect of an siRNA compound on a human, e.g., any unwanted effect, such as a toxic effect, or any positive, or desired effect.

Increasing Cellular Uptake of siRNAs

Described herein are various siRNA compositions that contain covalently attached conjugates that increase cellular uptake and/or intracellular targeting of the siRNAs.

Additionally provided are methods of the invention that include administering an siRNA compound and a drug that affects the uptake of the siRNA into the cell. The drug can be administered before, after, or at the same time that the siRNA compound is administered. The drug can be covalently or non-covalently linked to the siRNA compound. The drug can be, for example, a lipopolysaccharide, an activator of p38 MAP kinase, or an activator of NF-κB. The drug can have a transient effect on the cell. The drug can increase the uptake of the siRNA compound into the cell, for example, by disrupting the cell's cytoskeleton, e.g., by disrupting the cell's microtubules, microfilaments, and/or intermediate filaments. The drug can be, for example, taxon, vincristine, vinblastine, cytochalasin, nocodazole, japlakinolide, latrunculin A, phalloidin, swinholide A, indanocine, or myoservin. The drug can also increase the uptake of the siRNA compound into a given cell or tissue by activating an inflammatory response, for example. Exemplary drugs that would have such an effect include tumor necrosis factor alpha (TNFalpha), interleukin-1 beta, a CpG motif, gamma interferon or more generally an agent that activates a toll-like receptor.

### Cationic Lipid Compounds and Lipid Preparations

### Polyamine Lipid Preparations

Applicants have discovered that certain polyamine lipid moieties provide desirable properties for administration of nucleic acids, such as siRNA. For example, in some embodiments, a lipid moiety is complexed with a Factor VII-targeting siRNA and administered to an animal such as a mouse. The level of secreted serum Factor VII is then quantified (24 h post administration), where the degree of Factor VII silencing indicates the degree of in vivo siRNA delivery. Accordingly, lipids providing enhanced in vivo delivery of a nucleic acid such as siRNA are preferred. In particular, Applicants have discovered polyamines having substitutions described herein can have desirable properties for delivering siRNA, such as bioavailability, biodegradability, and tolerability.

In one embodiment, a lipid preparation includes a polyamine moiety having a plurality of substituents, such as acrylamide or acrylate substituents attached thereto. For example, a lipid moiety can include a polyamine moiety as provided below,

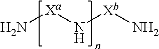

where one or more of the hydrogen atoms are substituted, for example with a substituent including a long chain alkyl, alkenyl, or alkynyl moiety, which in some embodiments is further substituted. X^{a} and X^{b} are alkylene moieties. In some embodiments, X^{a} and X^{b} have the same chain length, for example X^{a} and X^{b} are both ethylene moieties. In other embodiments X^{a} and X^{b} are of differing chain lengths. In some embodiments, where the polyamine includes a plurality of X^{a} moieties, X^{a} can vary with one or more occurrences. For example, where the polyamine is spermine, X^{a} in one occurrence is propylene, X^{a} in another occurrence is butylenes, and X^{b} is propylene.

Applicants have discovered that in some instances it is desirable to have a relatively high degree of substitution on the polyamine. For example, in some embodiments, Applicants have discovered that polyamine preparations where at least 80% (e.g., at least about 85%, at least about 90%, at least about 95%, at least about 97%, at least about 98%, at least about 99%, or substantially all) of the polyamines in the preparation have at least n+2 of the hydrogens substituted with a substituent provide desirable properties, for example for use in administering a nucleic acid such as siRNA.

In some instances it is desirable (preferably) to have one or more of hetero atoms present on the substituent on the nitrogen of polyamine

In some embodiments, a preparation comprises a compound of formula 5 or a pharmaceutically acceptable salt thereof,

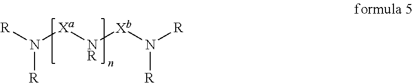

each X^{a} and X^{b}, for each occurrence, is independently C₁₋₆ alkylene; n is 0, 1, 2, 3, 4, or 5; each R is independently H,

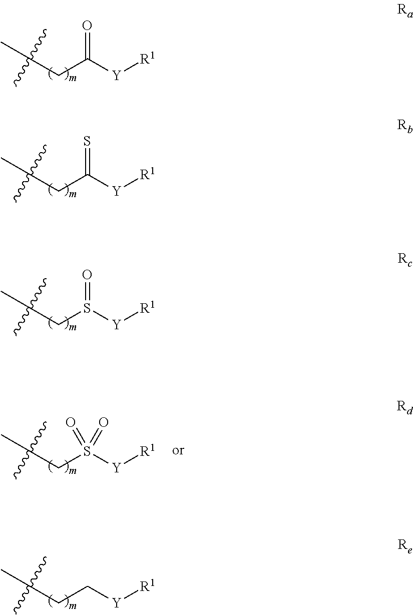

wherein at least n+2 of the R moieties in at least about 80% of the molecules of the compound of formula 5 in the preparation are not H; m is 1, 2, 3 or 4; Y is O, NR², or S; R¹ is alkyl alkenyl or alkynyl; each of which is optionally substituted; and R² is H, alkyl alkenyl or alkynyl; each of which is optionally substituted; provided that, if n=0, than at least n+3 of the R moieties are not H.

As noted above, the preparation includes molecules containing symmetrical as well as asymmetrical polyamine derivatives. Accordingly, X^{a} is independent for each occurrence and X^{b} is independent of X^{a}. For example, where n is 2, X^{a} can either be the same for each occurrence or can be different for each occurrence or can be the same for some occurrences and different for one or more other occurrences. X^{b} is independent of X^{a} regardless of the number of occurrences of X^{a} in each polyamine derivative. X^{a}, for each occurrence and independent of X^{b}, can be methylene, ethylene, propylene, butylene, pentylene, or hexylene. Exemplary polyamine derivatives include those polyamines derived from N¹,N^{1′}-(ethane-1,2-diyl)diethane-1,2-diamine, ethane-1,2-diamine, propane-1,3-diamine, spermine, spermidine, putrecine, and N¹-(2-Aminoethyl)-propane-1,3-diamine. Preferred polyamine derivatives include propane-1,3-diamine and N¹,N^{1′}-(ethane-1,2-diyl)diethane-1,2-diamine.

The polyamine of formula 5 is substituted with at least n+2 R moieties that are not H. In general, each non-hydrogen R moiety includes an alkyl, alkenyl, or alkynyl moiety, which is optionally substituted with one or more substituents, attached to a nitrogen of the polyamine derivative via a linker. Suitable linkers include amides, esters, thioesters, sulfones, sulfoxides, ethers, amines, and thioethers. In many instances, the linker moiety is bound to the nitrogen of the polyamine via an alkylene moiety (e.g., methylene, ethylene, propylene, or butylene). For example, an amide or ester linker is attached to the nitrogen of the polyamine through a methylene or ethylene moiety.

Examples of preferred amine substituents are provided below:

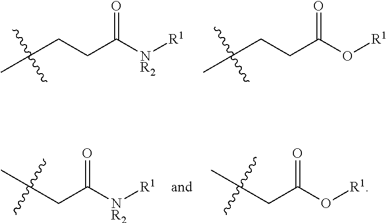

In instances where the amine is bound to the linker-R¹ portion via an ethylene group, a 1,4 conjugated precursor acrylate or acrylamide can be reacted with the polyamine to provide the substituted polyamine. In instances where the amine is bound to the linker-R¹ portion via a methylene group, an amide or ester including an alpha-halo substituent, such as an alpha-chloro moiety, can be reacted with the polyamine to provide the substituted polyamine. In preferred embodiments, R² is H.

R moieties that are not H, all require an R¹ moiety as provided above. In general, the R¹ moiety is a long chain moiety, such as C₆-C₃₂ alkyl, C₆-C₃₂ alkenyl, or C₆-C₃₂ alkynyl.

In some preferred embodiments, R¹ is an alkyl moiety. For example R¹ is C₁₀-C₁₈ alkyl, such as C₁₂ alkyl. Examples of especially preferred R moieties are provided below.

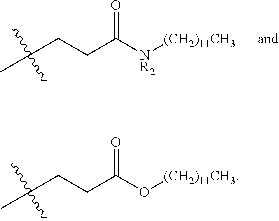

The preparations including a compound of formula 5 can be mixtures of a plurality of compounds of formula 5. For example, the preparation can include a mixture of compounds of formula 5 having varying degrees of substitution on the polyamine moiety. However, the preparations described herein are selected such that at least n+2 of the R moieties in at least about 80% (e.g., at least about 85%, at least about 90%, at least about 95%, at least about 97%, at least about 98%, at least about 99%, or substantially all) of the molecules of the compound of formula 5 in the preparation are not H.

In some embodiments, a preparation includes a polyamine moiety having two amino groups wherein in at least 80% (e.g., at least about 85%, at least about 90%, at least about 95%, at least about 97%, at least about 98%, at least about 99%, or substantially all) of the molecules of formula 5 in the mixture are substituted with three R moieties that are not H. Exemplary compounds of formula 5 are provided below.

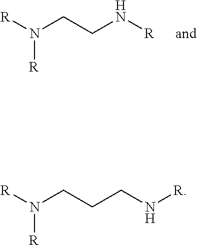

In some preferred embodiments R is

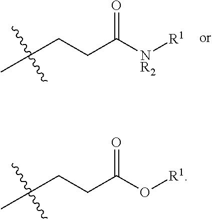

In some preferred embodiments, R¹ is C₁₀-C₁₈ alkyl, or C₁₀-C₃₀ alkenyl.

In some embodiments, a preparation includes a polyamine moiety having three or four (e.g., four) amino groups wherein at least n+2 of the R moieties in at least about 80% (e.g., at least about 85%, at least about 90%, at least about 95%, at least about 97%, at least about 98%, at least about 99%, or substantially all) of the molecules of formula 5 are not H. Exemplary compounds of formula 5 having 4 amino moieties are provided below.

Examples of polyamine moiety where all (i.e., n+4) R moieties are not H are below:

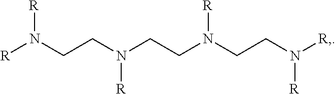

In some preferred embodiments R is

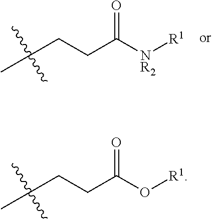

In some preferred embodiments, R¹ is C₁₀-C₁₈ alkyl (e.g., C₁₂ alkyl), or C₁₀-C₃₀ alkenyl.

Examples of polyamine moieties where five (i.e., n+3) R moieties are not H are provided below:

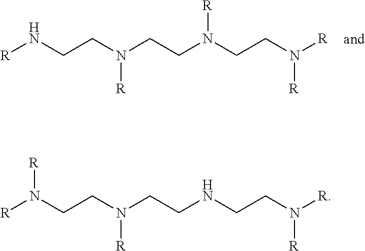

In some preferred embodiments R is

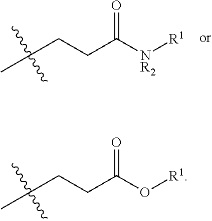

In some preferred embodiments, R¹ is C₁₀-C₁₈ alkyl (e.g., C₁₂ alkyl), or C₁₀-C₃₀ alkenyl.

Examples of polyamine moieties where four (i.e, n+2) R moieties are not H are provided below:

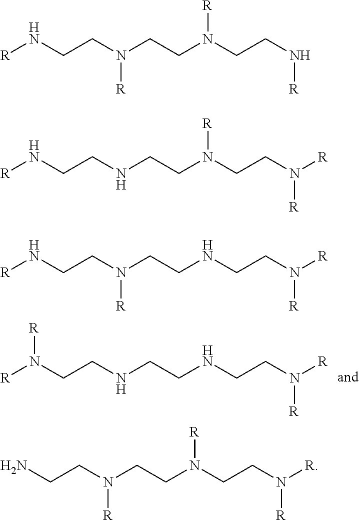

In some preferred embodiments R is

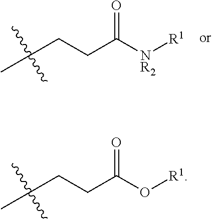

In some preferred embodiments, R¹ is C₁₀-C₁₈ alkyl (e.g., C₁₂ alkyl), or C₁₀-C₃₀ alkenyl.

In some preferred embodiments, the polyamine is a compound of isomer (1) or (2) below, preferably a compound of isomer (1)

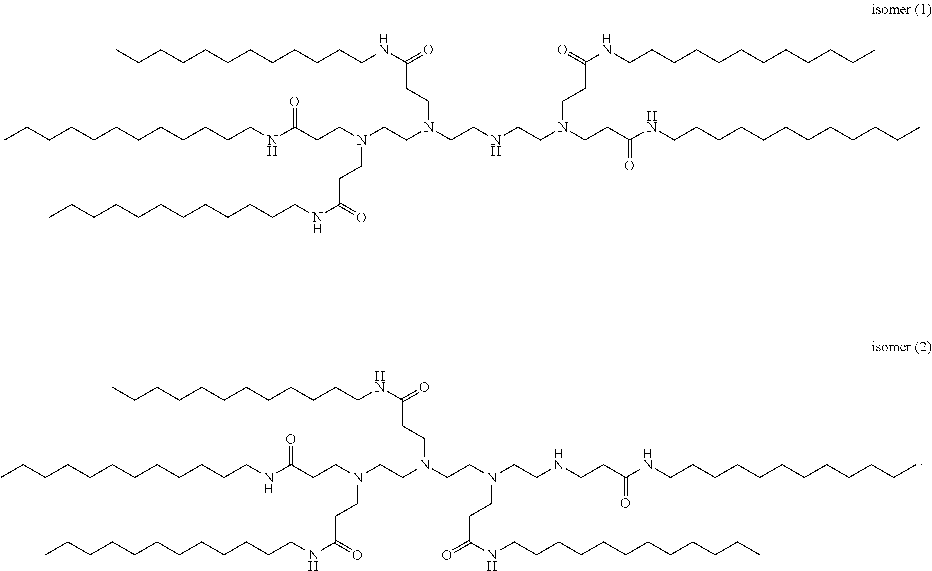

In some embodiments, the preparation including a compound of formula 5 includes a mixture of molecules having formula 5. For example, the mixture can include molecules having the same polyamine core but differing R substituents, such as differing degrees of R substituents that are not H.

In some embodiments, a preparation described herein includes a compound of formula 5 having a single polyamine core wherein each R of the polyamine core is either R or a single moiety such as

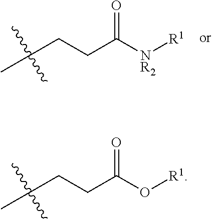

The preparation, therefore includes a mixture of molecules having formula 5, wherein the mixture is comprised of either polyamine compounds of formula 5 having a varied number of R moieties that are H and/or a polyamine compounds of formula 5 having a single determined number of R moieties that are not H where the compounds of formula 5 are structural isomers of the polyamine, such as the structural isomers provided above.

In some preferred embodiments the preparation includes molecules of formula 5 such that at least 80% (e.g., at least about 85%, at least about 90%, at least about 95%, at least about 97%, at least about 98%, at least about 99%, or substantially all) of the molecules are a single structural isomer.

In some embodiments, the preparation includes a mixture of two or more compounds of formula 5. In some embodiments, the preparation is a mixture of structural isomers of the same chemical formula. In some embodiments, the preparation is a mixture of compounds of formula 5 where the compounds vary in the chemical nature of the R substituents. For example, the preparation can include a mixture of the following compounds:

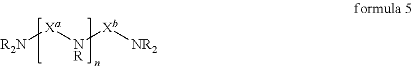

wherein n is 0 and each R is independently H or

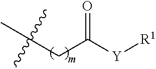

and

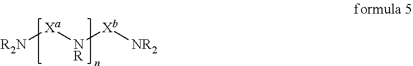

wherein n is 2 and each R is independently H or

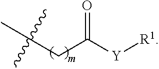

In some embodiments, the compound of formula 5 is in the form of a salt, such as a pharmaceutically acceptable salt. A salt, for example, can be formed between an anion and a positively charged substituent (e.g., amino) on a compound described herein. Suitable anions include fluoride, chloride, bromide, iodide, sulfate, bisulfate, nitrate, phosphate, citrate, methanesulfonate, trifluoroacetate, acetate, fumarate, oleate, valerate, maleate, oxalate, isonicotinate, lactate, salicylate, tartrate, tannate, pantothenate, bitartrate, ascorbate, succinate, gentisinate, gluconate, glucaronate, saccharate, formate, benzoate, glutamate, ethanesulfonate, benzenesulfonate, p-toluensulfonate, and pamoate. In some preferred embodiments, the compound of formula 5 is a hydrohalide salt, such as a hydrochloride salt.

Compounds of formula 5 can also be present in the form of hydrates (e.g., (H₂O)ₙ) and solvates, which are included herewith in the disclosure.

### Biocleavable Cationic Lipids

Applicants have discovered that certain cationic lipids that include one or more biocleavable moieties can be used as a component in an association complex, such as a liposome, for the delivery of nucleic acid therapies (e.g., dsRNA). For example, disclosed herein are cationic lipids that are subject to cleavage in vivo, for example, via an enzyme such as an esterase, an amidase, or a disulfide cleaving enzyme. In some instances, the lipid is cleaved chemically, for example by hydrolysis of an acid labile moiety such as an acetal or ketal. In some embodiments, the lipid includes a moiety that is hydrolyzed in vitro and then subject to enzymatic cleavage by one or more of an esterase, amidase, or a disulfide cleaving enzyme. This can happen in vesicular compartments of the cell such as endosomes. Another acid sensitive cleavable linkage is β-thiopropionate linkage which is cleaved in the acidic environment of endosomes (Jeong et al. Bioconjugate chem. 2003, 4, 1426).

In some embodiments, the invention features a compound of formula 6 or a pharmaceutically acceptable salt thereof, wherein

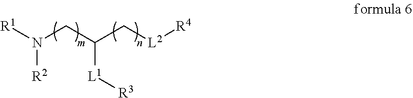

wherein

R¹ and R² are each independently H, C₁-C₆ alkyl, optionally substituted with 1-4 R⁵, C₂-C₆ alkenyl, optionally substituted with 1-4 R⁵, or C(NR⁶)(NR⁶)₂;

R³ and R⁴ are each independently alkyl, alkenyl, alkynly, each of which is optionally substituted with fluoro, chloro, bromo, or iodo;

L¹ and L² are each independently —NR⁶C(O)—, —C(O)NR⁶—, —OC(O)—, —C(O)O—, —S—S—, —N(R⁶)C(O)N(R⁶)—, —OC(O)N(R⁶)—, —N(R⁶)C(O)O—, —O—N═O—, OR—OC(O)NH; or

L¹-R³ and L²-R⁴ can be taken together to form an acetal or a ketal;

R⁵ is fluoro, chloro, bromo, iodo, —OR⁷, —N(R⁸)(R⁹), —CN, Se, S(O)R¹⁰, S(O)₂R¹⁰

R⁶ is H, C₁-C₆ alkyl,

R⁷ is H or C₁-C₆ alkyl;

each R⁸ and R⁹ are independently H or C₁-C₆ alkyl;

R¹⁰ is H or C₁-C₆ alkyl;

m is 1, 2, 3, 4, 5, or 6;

n is 0, 1, 2, 3, 4, 5, or 6;

and pharmaceutically acceptable salts thereof.

In some embodiments, R¹ is H, a lower alkyl, such as methyl, ethyl, propyl, or isopropyl, or a substituted alkyl, such as 2-hydroxyethyl.

In some embodiments, R² is H or a lower alkyl, such as methyl, ethyl, propyl, or isopropyl.

In some embodiments, R¹ or R² form a quanadine moiety with the nitrogen of formula (6).

L¹-R³ and L²-R⁴ or the combination thereof provide at least one moiety that is cleaved in vivo. In some embodiments, both L¹-R³ and L²-R⁴ are biocleavable. For example, both L¹-R³ and L²-R⁴ are independently subject to enzymatic cleavage (e.g., by an esterase, amidase, or a disulfide cleaving enzyme). In some embodiments, both L¹ and L² are the same chemical moiety such as an ester, amide or disulfide. In other instances, L¹ and L² are different, for example, one of L¹ or L² is an ester an the other of L¹ or L² is a disulfide.

In some embodiments, L¹-R³ and L²-R⁴ together form an acetal or ketal moiety, which is hydrolyzed in vivo.

In some embodiments, one of L¹-R³ or L²-R⁴ is subject to enzymatic cleavage. For example, one of L¹-R³ or L²-R⁴ is cleaved in vivo, providing a free hydroxyl moiety or free amine on the lipid, which becomes available to chemically react with the remaining L¹-R³ or L²-R⁴ moiety. Exemplary embodiments are provided below:

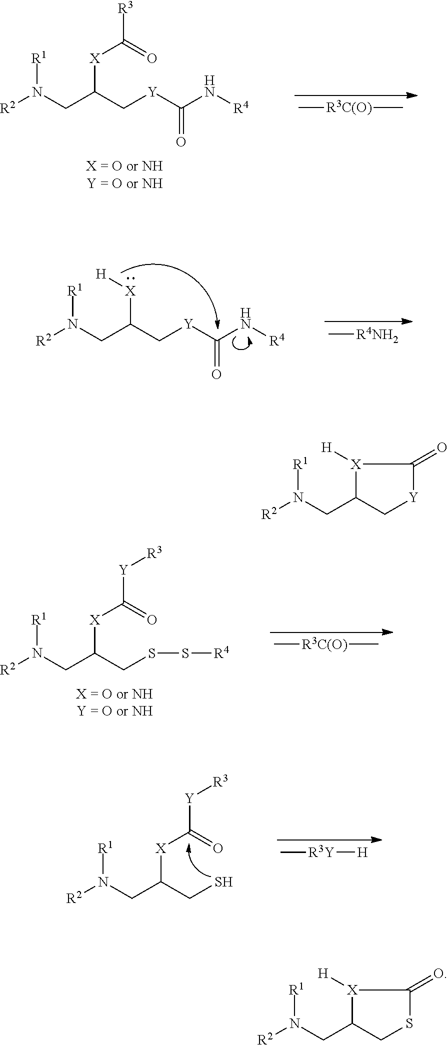

In some preferred embodiments, a carbamate or urea moiety is included in combination with an amide, ester or disulfide moiety. For example, the lipid includes an ester moiety, which upon cleavage (e.g., enzymatic cleavage) becomes available to chemically react with the carbamate or urea moiety. Some preferred combinations of L¹ and L² include two amides, two esters, an amide and an ester, two disulfides, an amide and a disulfide, an ester and a disulfide, a carbamate and a disulfide, and a urea and a disulfide. Exemplary compounds are provided below:

Amide and ester linkages with Z configuration (two double bonds)

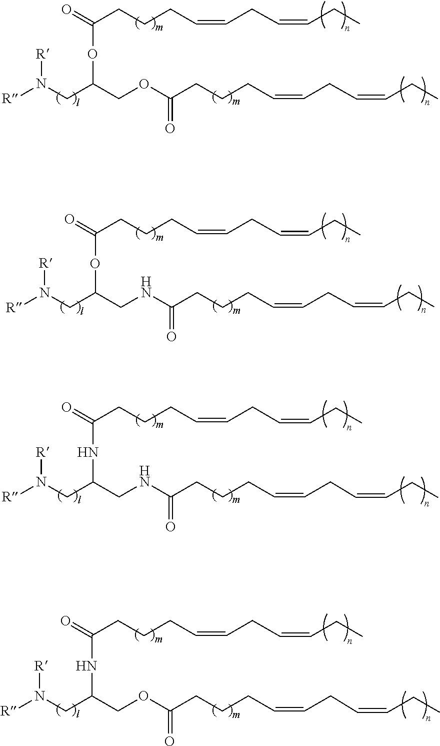

R′═H, Me, Et, propyl, isopropyl or 2-hydroxyethyl and R″═H; I=1 to 6, m=1-8, n=1−10

R′═H, Me, Et, propyl, isopropyl or 2-hydroxyethyl and R″=Me; I=1 to 6, m=1-8, n=1−10

R′═H, Me, Et, propyl, isopropyl or 2-hydroxyethyl and R″=Et; I=1 to 6, m=1-8, n=1−10

R′═H, Me, Et, propyl, isopropyl or 2-hydroxyethyl and R″=propyl; I=1 to 6, m=1-8, n=1−10

R′═H, Me, Et, propyl, isopropyl or 2-hydroxyethyl and R″=isopropyl; I=1 to 6, m=1-8, n=1-10

Amide Ester linkage with Z configuration (three double bonds)

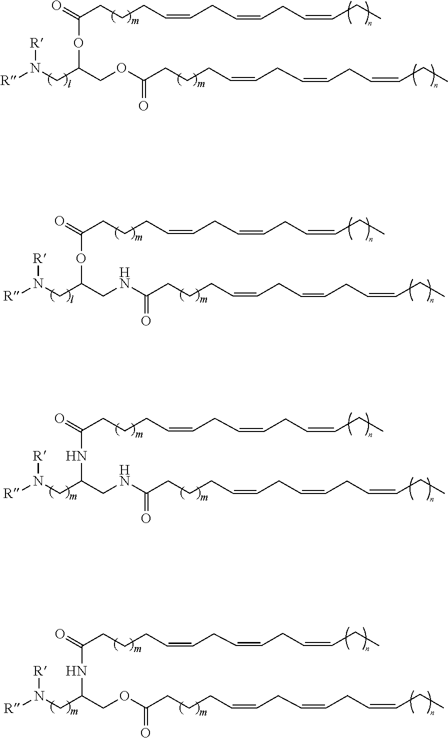

R′═H, Me, Et, propyl, isopropyl or 2-hydroxyethyl and R″═H; I=1 to 6, m=1-8, n=1−10

R′═H, Me, Et, propyl, isopropyl or 2-hydroxyethyl and R″=Me; I=1 to 6, m=1-8, n=1−10

R′═H, Me, Et, propyl, isopropyl or 2-hydroxyethyl and R″=Et; I=1 to 6, m=1-8, n=1−10

R′═H, Me, Et, propyl, isopropyl or 2-hydroxyethyl and R″=propyl; I=1 to 6, m=1-8, n=1−10

R′═H, Me, Et, propyl, isopropyl or 2-hydroxyethyl and R″=isopropyl; I=1 to 6, m=1-8, n=1-10

Amides and ester linkages with E configuration (two double bonds)

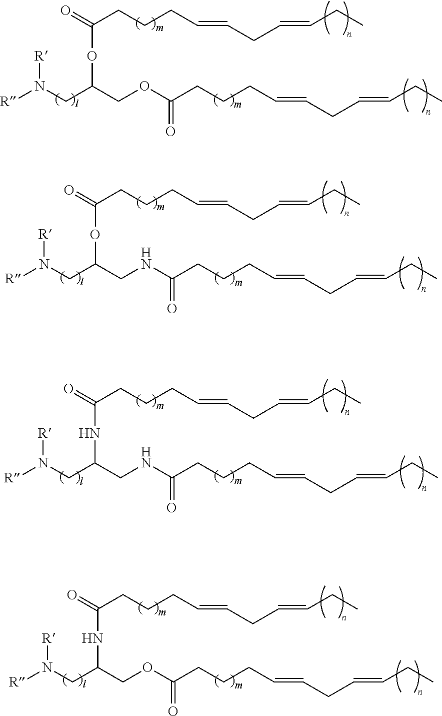

R′═H, Me, Et, propyl, isopropyl or 2-hydroxyethyl and R″═H; I=1 to 6, m=1-8, n=1−10

R′═H, Me, Et, propyl, isopropyl or 2-hydroxyethyl and R″=Me; I=1 to 6, m=1-8, n=1−10

R′═H, Me, Et, propyl, isopropyl or 2-hydroxyethyl and R″=Et; I=1 to 6, m=1-8, n=1−10

R′═H, Me, Et, propyl, isopropyl or 2-hydroxyethyl and R″=propyl; I=1 to 6, m=1-8, n=1−10

R′═H, Me, Et, propyl, isopropyl or 2-hydroxyethyl and R″=isopropyl; I=1 to 6, m=1-8, n=1-10

Amides and ester linkages with E configuration (three double bonds)

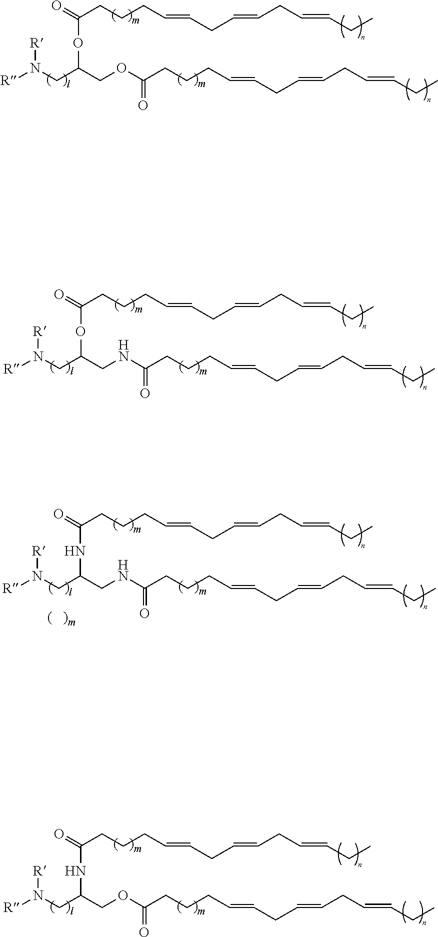

R′═H, Me, Et, propyl, isopropyl or 2-hydroxyethyl and R″═H; I=1 to 6, m=1-8, n=1−10

R′═H, Me, Et, propyl, isopropyl or 2-hydroxyethyl and R″=Me; I=1 to 6, m=1-8, n=1−10

R′═H, Me, Et, propyl, isopropyl or 2-hydroxyethyl and R″=Et; I=1 to 6, m=1-8, n=1−10

R′═H, Me, Et, propyl, isopropyl or 2-hydroxyethyl and R″=propyl; I=1 to 6, m=1-8, n=1−10

R′═H, Me, Et, propyl, isopropyl or 2-hydroxyethyl and R″=isopropyl; I=1 to 6, m=1-8, n=1-10

Disulfide linkages

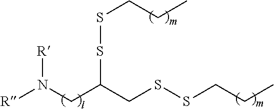

R′═H, Me, Et, propyl, isopropyl or 2-hydroxyethyl and R″═H; I=1 to 6, m=6-28

R′═H, Me, Et, propyl, isopropyl or 2-hydroxyethyl and R″=Me; I=1 to 6, m=6-28

R′═H, Me, Et, propyl, isopropyl or 2-hydroxyethyl and R″=Et; I=1 to 6, m=6-28

R′═H, Me, Et, propyl, isopropyl or 2-hydroxyethyl and R″=propyl; I=1 to 6, m=6-28

R′═H, Me, Et, propyl, isopropyl or 2-hydroxyethyl and R″=isopropyl; I=1 to 6, m=6-28

Disulfide linkages with unsaturated alkyl chains, E and Z configuration

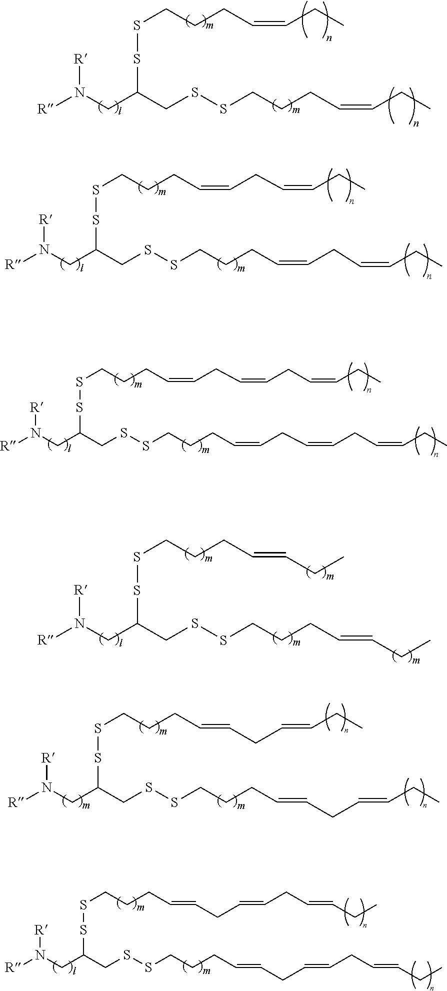

R′═H, Me, Et, propyl, isopropyl or 2-hydroxyethyl and R″═H; I=1 to 6, m=1-8, n=1−10

R′═H, Me, Et, propyl, isopropyl or 2-hydroxyethyl and R″=Me; I=1 to 6, m=1-8, n=1−10

R′═H, Me, Et, propyl, isopropyl or 2-hydroxyethyl and R″=Et; I=1 to 6, m=1-8, n=1−10

R′═H, Me, Et, propyl, isopropyl or 2-hydroxyethyl and R″=propyl; I=1 to 6, m=1-8, n=1−10

R′═H, Me, Et, propyl, isopropyl or 2-hydroxyethyl and R″=isopropyl; I=1 to 6, m=1-8, n=1-10

Amide and disulfide linkages with saturated and unsaturated alkyl chains

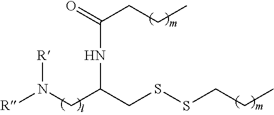

R′═H, Me, Et, propyl, isopropyl or 2-hydroxyethyl and R″═H; I=1 to 6, m=6-28

R′═H, Me, Et, propyl, isopropyl or 2-hydroxyethyl and R″=Me; I=1 to 6, m=6-28

R′═H, Me, Et, propyl, isopropyl or 2-hydroxyethyl and R″=Et; I=1 to 6, m=6-28

R′═H, Me, Et, propyl, isopropyl or 2-hydroxyethyl and R″=propyl; I=1 to 6, m=6-28

R′═H, Me, Et, propyl, isopropyl or 2-hydroxyethyl and R″=isopropyl; I=1 to 6, m=6-28

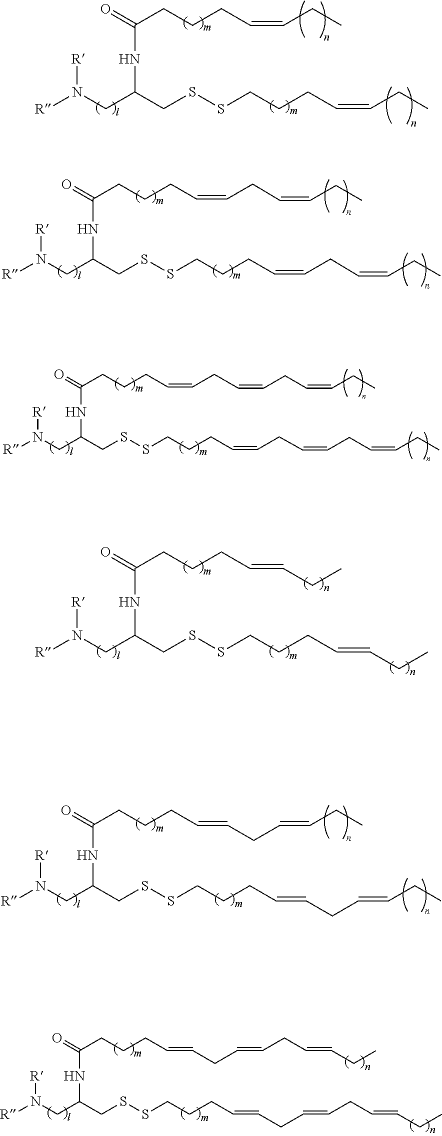

R′═H, Me, Et, propyl, isopropyl or 2-hydroxyethyl and R″═H; I=1 to 6, m=1-8, n=1−10

R′═H, Me, Et, propyl, isopropyl or 2-hydroxyethyl and R″=Me; I=1 to 6, m=1-8, n=1−10

R′═H, Me, Et, propyl, isopropyl or 2-hydroxyethyl and R″=Et; I=1 to 6, m=1-8, n=1−10

R′═H, Me, Et, propyl, isopropyl or 2-hydroxyethyl and R″=propyl; I=1 to 6, m=1-8, n=1−10

R′═H, Me, Et, propyl, isopropyl or 2-hydroxyethyl and R″=isopropyl; I=1 to 6, m=1-8, n=1-10

Ester and disulfide linkages with saturated and unsaturated alkyl chains

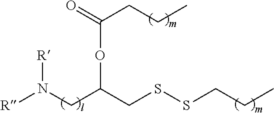

R′═H, Me, Et, propyl, isopropyl or 2-hydroxyethyl and R″═H; I=1 to 6, m=6-28

R′═H, Me, Et, propyl, isopropyl or 2-hydroxyethyl and R″=Me; I=1 to 6, m=6-28

R′═H, Me, Et, propyl, isopropyl or 2-hydroxyethyl and R″=Et; I=1 to 6, m=6-28

R′═H, Me, Et, propyl, isopropyl or 2-hydroxyethyl and R″=propyl; I=1 to 6, m=6-28

R′═H, Me, Et, propyl, isopropyl or 2-hydroxyethyl and R″=isopropyl; I=1 to 6, m=6-28

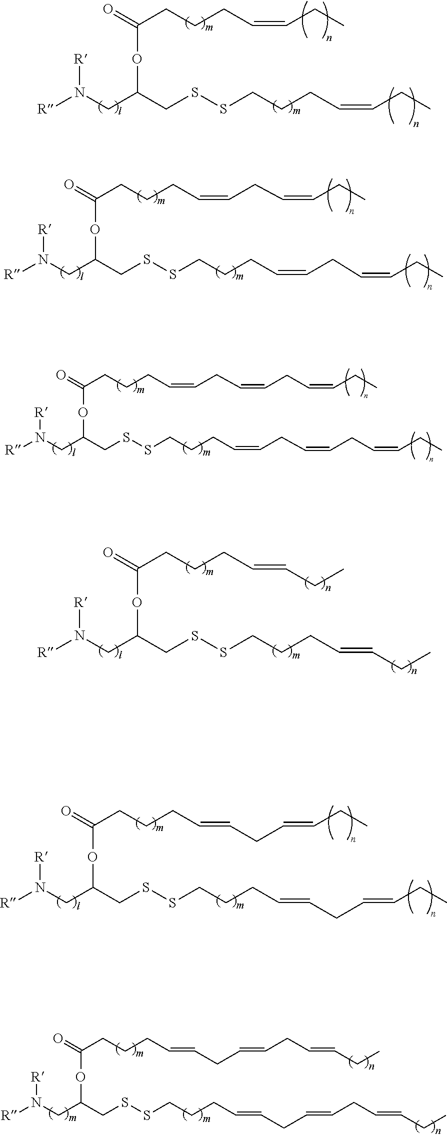

R′═H, Me, Et, propyl, isopropyl or 2-hydroxyethyl and R″═H; I=1 to 6, m=1-8, n=1−10

R′═H, Me, Et, propyl, isopropyl or 2-hydroxyethyl and R″=Me; I=1 to 6, m=1-8, n=1−10

R′═H, Me, Et, propyl, isopropyl or 2-hydroxyethyl and R″=Et; I=1 to 6, m=1-8, n=1−10

R′═H, Me, Et, propyl, isopropyl or 2-hydroxyethyl and R″=propyl; I=1 to 6, m=1-8, n=1−10

R′═H, Me, Et, propyl, isopropyl or 2-hydroxyethyl and R″=isopropyl; I=1 to 6, m=1-8, n=1-10

Carbamate or urea and disulfide linkages with alkyl chains

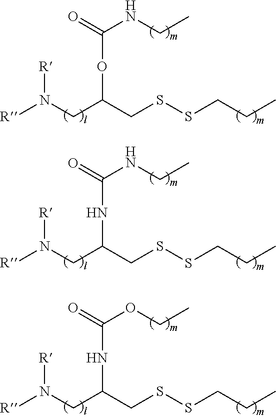

R′═H, Me, Et, propyl, isopropyl or 2-hydroxyethyl and R″═H; I=1 to 6, m=6-28

R′═H, Me, Et, propyl, isopropyl or 2-hydroxyethyl and R″=Me; I=1 to 6, m=6-28

R′═H, Me, Et, propyl, isopropyl or 2-hydroxyethyl and R″=Et; I=1 to 6, m=6-28

R′═H, Me, Et, propyl, isopropyl or 2-hydroxyethyl and R″=propyl; I=1 to 6, m=6-28

R′═H, Me, Et, propyl, isopropyl or 2-hydroxyethyl and R″=isopropyl; I=1 to 6, m=6-28

Carbamate or urea and disulfide linkages with unsaturated alkyl chains

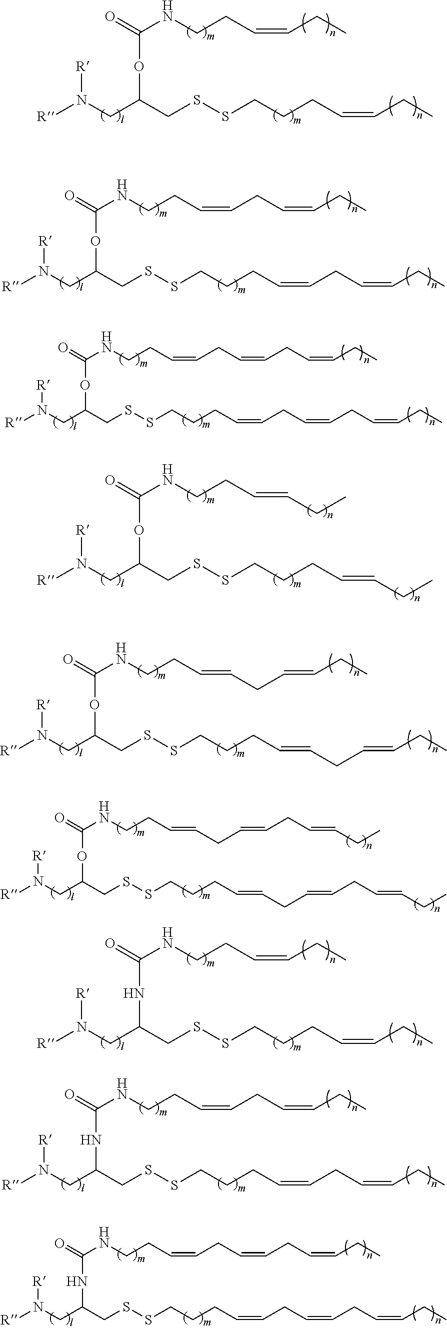

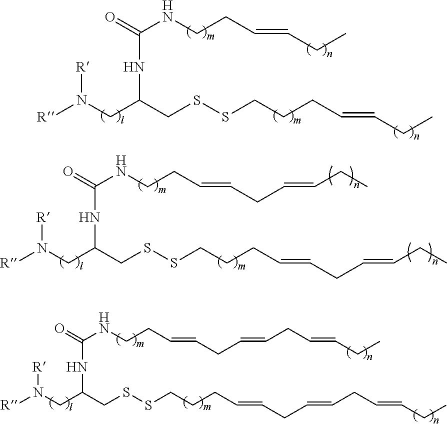

R′═H, Me, Et, propyl, isopropyl or 2-hydroxyethyl and R″═H; I=1 to 6, m=6-28

R′═H, Me, Et, propyl, isopropyl or 2-hydroxyethyl and R″=Me; I=1 to 6, m=6-28

R′═H, Me, Et, propyl, isopropyl or 2-hydroxyethyl and R″=Et; I=1 to 6, m=6-28

R′═H, Me, Et, propyl, isopropyl or 2-hydroxyethyl and R″=propyl; I=1 to 6, m=6-28

R′═H, Me, Et, propyl, isopropyl or 2-hydroxyethyl and R″=isopropyl; I=1 to 6, m=6-28

Carbamate or urea and disulfide linkages with unsaturated alkyl chains

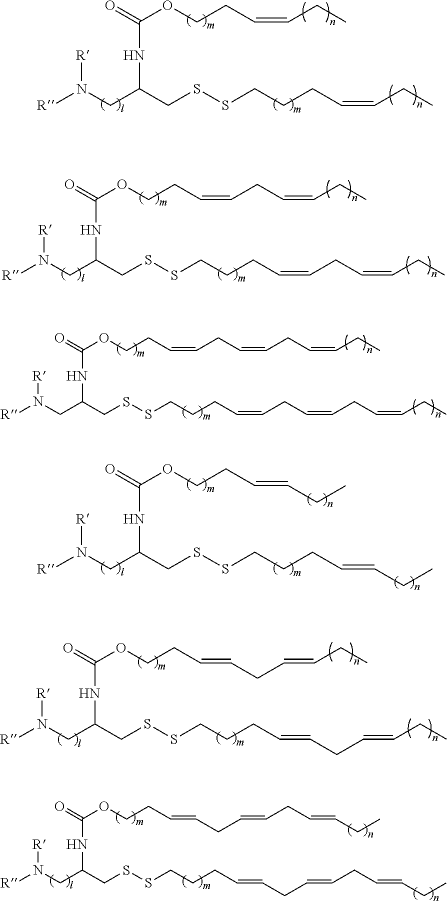

R′═H, Me, Et, propyl, isopropyl or 2-hydroxyethyl and R″═H; I=1 to 6, m=6-28

R′═H, Me, Et, propyl, isopropyl or 2-hydroxyethyl and R″=Me; I=1 to 6, m=6-28

R′═H, Me, Et, propyl, isopropyl or 2-hydroxyethyl and R″=Et; I=1 to 6, m=6-28

R′═H, Me, Et, propyl, isopropyl or 2-hydroxyethyl and R″=propyl; I=1 to 6, m=6-28

R′═H, Me, Et, propyl, isopropyl or 2-hydroxyethyl and R″=isopropyl; I=1 to 6, m=6-28

Carbamate and urea linkages with unsaturated alkyl chains

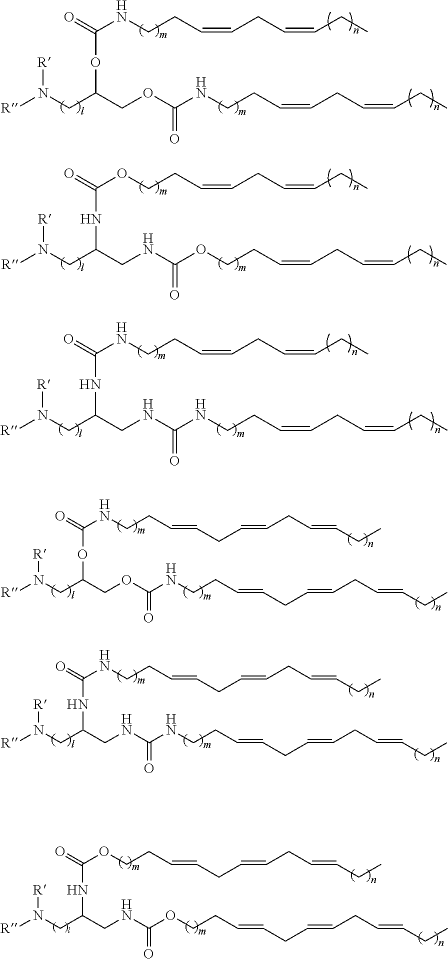

R′═H, Me, Et, propyl, isopropyl or 2-hydroxyethyl and R″═H; I=1 to 6, m=1-10, n=1−10

R′═H, Me, Et, propyl, isopropyl or 2-hydroxyethyl and R″=Me; I=1 to 6, m=1-10, n=1−10

R′═H, Me, Et, propyl, isopropyl or 2-hydroxyethyl and R″=Et; I=1 to 6, m=1-10, n=1−10

R′═H, Me, Et, propyl, isopropyl or 2-hydroxyethyl and R″=propyl; I=1 to 6, m=1-10, n=1−10

R′═H, Me, Et, propyl, isopropyl or 2-hydroxyethyl and R″=isopropyl; I=1 to 6, m=1-10, n=1-10

In some embodiments, the lipid includes an oxime or hydrazone, which can undergo acidic cleavage.

R³ and R⁴ are generally long chain hydrophobic moieties, such as alkyl, alkenyl, or alkynyl. In some embodiments, R³ or R⁴ are substituted with a halo moiety, for example, to provide a perfluoroalkyl or perfluoroalkenyl moiety. Each of R³ and R⁴ are independent of each other. In some embodiments, both of R³ and R⁴ are the same. In some embodiments, R³ and R⁴ are different.

In some embodiments R³ and/or R⁴ are alkyl. For example one or both of R³ and/or

R⁴ are C₆ to C₃₀ alkyl, e.g., C₁₀ to C₂₆ alkyl, C₁₂ to C₂₀ alkyl, or C₁₂ alkyl.

In some embodiments, R³ and/or R⁴ are alkenyl. In some preferred embodiments, R³ and/or R⁴ include 2 or 3 double bonds. For example R³ and/or R⁴ includes 2 double bonds or R³ and/or R⁴ includes 3 double bonds. The double bonds can each independently have a Z or E configuration. Exemplary alkenyl moieties are provided below:

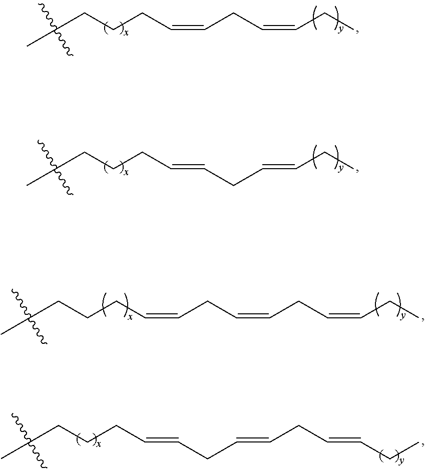

wherein x is an integer from 1 to 8; and y is an integer from 1-10. In some preferred embodiments, R³ and/or R⁴ are C₆ to C₃₀ alkenyl, e.g., C₁₀ to C₂₆ alkenyl, C₁₂ to C₂₀ alkenyl, or C₁₇ alkenyl, for example having two double bonds, such as two double bonds with Z configuration. R³ and/or R⁴ can be the same or different. In some preferred embodiments, R³ and R⁴ are the same.

In some embodiments, R³ and/or R⁴ are alkynyl. For example C₆ to C₃₀ alkynyl, e.g., C₁₀ to C₂₆ alkynyl, C₁₂ to C₂₀ alkynyl. R³ and/or R⁴ can have from 1 to 3 triple bonds, for example, one, two, or three triple bonds.

In some embodiments, the compound of formula 6 is in the form of a salt, such as a pharmaceutically acceptable salt. A salt, for example, can be formed between an anion and a positively charged substituent (e.g., amino) on a compound described herein. Suitable anions include fluoride, chloride, bromide, iodide, sulfate, bisulfate, nitrate, phosphate, citrate, methanesulfonate, trifluoroacetate, acetate, fumarate, oleate, valerate, maleate, oxalate, isonicotinate, lactate, salicylate, tartrate, tannate, pantothenate, bitartrate, ascorbate, succinate, gentisinate, gluconate, glucaronate, saccharate, formate, benzoate, glutamate, ethanesulfonate, benzenesulfonate, p-toluensulfonate, and pamoate. In some preferred embodiments, the compound of formula 6 is a hydrohalide salt, such as a hydrochloride salt.

Compounds of formula 6 can also be present in the form of hydrates (e.g., (H₂O)ₙ) and solvates, which are included herewith in the disclosure.

### PEG-Lipid Compounds

Applicants have discovered that certain PEG containing lipid moieties provide desirable properties for administration of a nucleic acid agent such as single stranded or double stranded nucleic acid, for example siRNA. For example, when a PEG containing lipid, such as a lipid described herein, is formulated into an association complex with a nucleic acid moiety, such as siRNA and administered to a subject, the lipid provides enhanced delivery of the nucleic acid moiety. This enhanced delivery can be determined, for example, by evaluation in a gene silencing assay such as silencing of FVII. Applicants have discovered certain PEG-lipids can have desirable properties for the delivery of siRNA, including improved bioavailability, diodegradability, and tolerability.

In some embodiment, the PEG is attached via a linker moiety to a structure including two hydrophobic moieties, such as a long chanin alkyl moiety. In some preferred embodiments, the PEG-lipid has the structure below:

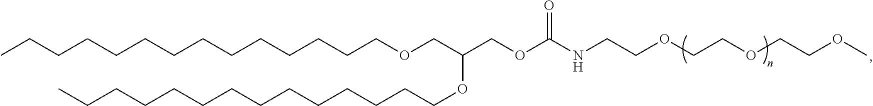

wherein the preferred stereochemistry of the chiral center is ‘R’ and the repeating PEG moiety has a total average molecular weight of about 2000 daltons.

In some embodiments, a PEG lipid described herein is conjugated to a targeting moiety, e.g., a glycosyl moiety such as a

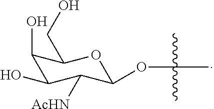

In some embodiments, the targeting moiety is attached to the PEG lipid through a linker, for example a linker described herein.

### Methods of Making Cationic Lipid Compounds and Cationic Lipid Containing Preparations

The compounds described herein can be obtained from commercial sources (e.g., Asinex, Moscow, Russia; Bionet, Camelford, England; ChemDiv, SanDiego, Calif.; Comgenex, Budapest, Hungary; Enamine, Kiev, Ukraine; IF Lab, Ukraine; Interbioscreen, Moscow, Russia; Maybridge, Tintagel, UK; Specs, The Netherlands; Timtec, Newark, Del.; Vitas-M Lab, Moscow, Russia) or synthesized by conventional methods as shown below using commercially available starting materials and reagents.

### Methods of Making Polyamine Lipids

In some embodiments, a compound of formula 5 can be made by reacting a polyamine of formula 7 as provided below:

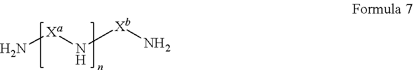

wherein X^{a}, X^{b}, and n are defined as above with a 1,4 conjugated system of formula 8:

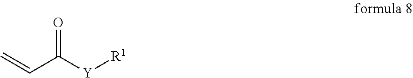

wherein Y and R₁ are defined as above to provide a compound of formula 5.

In some embodiments, the compounds of formula 7 and 8 are reacted together neat (i.e., free of solvent). For example, the compounds of formula 7 and 8 are reacted together neat at elevated temperature (e.g., at least about 60° C., at least about 65° C., at least about 70° C., at least about 75° C., at least about 80° C., at least about 85° C., or at least about 90° C.), preferably at about 90° C.

In some embodiments, the compounds of formula 7 and 8 are reacted together with a solvent (e.g., a polar aprotic solvent such as acetonitrile or DMF). For example, the compounds of formula 7 and 8 are reacted together in solvent at an elevated temperature from about 50° C. to about 120° C.

In some embodiments, the compounds of formula 7 and 8 are reacted together in the presence of a radical quencher or scavenger (e.g., hydroquinone). The reaction conditions including a radical quencher can be neat or in a solvent e.g., a polar aprotic solvent such as acetonitrile or DMF. The reaction can be at an elevated temperature (e.g., neat at an elevated temperature such as 90° C. or with solvent at an elevated temperature such as from about 50° C. to about 120° C.). The term “radical quencher” or “radical scavenger” as used herein refers to a chemical moiety that can absorb free radicals in a reaction mixture. Examples of radical quenchers/scavengers include hydroquinone, ascorbic acid, cresols, thiamine, 3,5-Di-tert-butyl-4-hydroxytoluene, tert-Butyl-4-hydroxyanisole and thiol containing moieties.

In some embodiments, the compounds of formula 7 and 8 are reacted together in the presence of a reaction promoter (e.g., water or a Michael addition promoter such as acetic acid, boric acid, citric acid, benzoic acid, tosic acid, pentafluorophenol, picric acid aromatic acids, salts such as bicarbonate, bisulphate, mono and di-hydrogen phophates, phenols, perhalophenols, nitrophenols, sulphonic acids, PTTS, etc.), preferably boric acid such as a saturated aqueous boric acid. The reaction conditions including a reaction promoter can be neat or in a solvent e.g., a polar aprotic solvent such as acetonitrile or DMF. The reaction can be at an elevated temperature (e.g., neat at an elevated temperature such as 90° C. or with solvent at an elevated temperature such as from about 50° C. to about 120° C.). The term “reaction promoter” as used herein refers to a chemical moiety that, when used in a reaction mixture, accelerates/enhances the rate of reaction.

The ratio of compounds of formula 7 to formula 8 can be varied, providing variability in the substitution on the polyamine of formula 7. In general, polyamines having at least about 50% of the hydrogen moieties substituted with a non-hydrogen moiety are preferred. Accordingly, ratios of compounds of formula 7/formula 8 are selected to provide for products having a relatively high degree of substitution of the free amine (e.g., at least about 50%, at least about 55%, at least about 60%, at least about 65%, at least about 70%, at least about 75%, at least about 80%, at least about 85%, at least about 90%, at least about 95%, at least about 97%, at least about 99%, or substantially all). In some preferred embodiments n is 0 in the polyamine of formula 7, and the ratio of compounds of formula 7 to compounds of formula 8 is from about 1:3 to about 1:5, preferable about 1:4. In some preferred embodiments, n is 2 in the polyamine of formula 7, and the ratio of compound of formula 7 to compounds of formula 8 is from about 1:3 to about 1:6, preferably about 1:5.

In some embodiments, the compounds of formula 7 and formula 8 are reacted in a two step process. For example, the first step process includes a reaction mixture having from about 0.8 about 1.2 molar equivalents of a compound of formula 7, with from about 3.8 to about 4.2 molar equivalents of a compound of formula 8 and the second step process includes addition of about 0.8 to 1.2 molar equivalent of compound of formula 8 to the reaction mixture.

Upon completion of the reaction, one or more products having formula 5 can be isolated from the reaction mixture. For example, a compound of formula 5 can be isolated as a single product (e.g., a single structural isomer) or as a mixture of product (e.g., a plurality of structural isomers and/or a plurality of compounds of formula 5). In some embodiments, one or more reaction products can be isolated and/or purified using chromatography, such as flash chromatography, gravity chromatography (e.g., gravity separation of isomers using silica gel), column chromatography (e.g., normal phase HPLC or RPHPLC), or moving bed chromatography. In some embodiments, a reaction product is purified to provide a preparation containing at least about 80% of a single compound, such as a single structural isomer (e.g., at least about 85%, at least about 90%, at least about 95%, at least about 97%, at least about 99%).

In some embodiments, a free amine product is treated with an acid such as HCl to prove an amine salt of the product (e.g., a hydrochloride salt). In some embodiments a salt product provides improved properties, e.g., for handling and/or storage, relative to the corresponding free amine product. In some embodiments, a salt product can prevent or reduce the rate of formation of breakdown product such as N-oxide or N-carbonate formation relative to the corresponding free amine. In some embodiments, a salt product can have improved properties for use in a therapeutic formulation relative to the corresponding free amine

In some embodiments, the reaction mixture is further treated, for example, to purify one or more products or to remove impurities such as unreacted starting materials. In some embodiments the reaction mixture is treated with an immobilized (e.g., polymer bound) thiol moiety, which can trap unreacted acrylamide. In some embodiments, an isolated product can be treated to further remove impurities, e.g., an isolated product can be treated with an immobilized thiol moiety, trapping unreacted acrylamide compounds.

In some embodiments a reaction product can be treated with an immobilized (e.g., polymer bound) isothiocyanate. For example, a reaction product including tertiary amines can be treated with an immobilized isothiocyanate to remove primary and/or secondary amines from the product.

In some embodiments, a compound of formula 5 can be made by reacting a polyamine of formula 7 as provided below

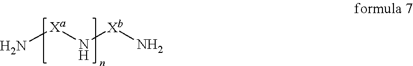

wherein X^{a}, X^{b}, and n are defined as above
with a compound of formula 9,

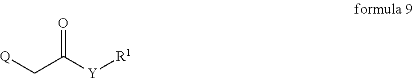

wherein Q is Cl, Br, or I, and Y and R¹ are as defined above.

In some embodiments, the compound of formula 7 and formula 9 are reacted together neat. In some embodiments, the compound of formula 7 and formula 9 are reacted together in the presence of one or more solvents, for example a polar aprotic solvent such as acetonitrile or DMF. In some embodiments, the reactants (formula 7 and formula 9) are reacted together at elevated temperature (e.g., at least about 50° C., at least about 60° C., at least about 70° C., at least about 80° C., at least about 90° C., at least about 100° C.).

In some embodiments, the reaction mixture also includes a base, for example a carbonate such as K₂CO₃.

In some embodiments, the reaction mixture also includes a catalyst.

In some embodiments, the compound of formula 9 is prepared by reacting an amine moiety with an activated acid such as an acid anhydrate or acid halide (e.g., acid chloride) to provide a compound of formula 9.

The ratio of compounds of formula 7 and formula 9 can be varied, providing variability in the substitution on the polyamine of formula 7. In general, polyamines having at least about 50% of the hydrogen moieties substituted with a non-hydrogen moiety are preferred. Accordingly, ratios of compounds of formula 7/formula 9 are selected to provide for products having a relatively high degree of substitution of the free amine (e.g., at least about 50%, at least about 55%, at least about 60%, at least about 65%, at least about 70%, at least about 75%, at least about 80%, at least about 85%, at least about 90%, at least about 95%, at least about 97%, at least about 99%, or substantially all). In some preferred embodiments n is 0 in the polyamine of formula 7, and the ratio of compounds of formula 7 to compounds of formula 9 is from about 1:3 to about 1:5, preferable about 1:4. In some preferred embodiments, n is 2 in the polyamine of formula 7, and the ratio of compound of formula 7 to compounds of formula 9 is from about 1:3 to about 1:6, preferably about 1:5.

In some embodiments, the compounds of formula 7 and formula 9 are reacted in a two step process. For example, the first step process includes a reaction mixture having from about 0.8 about 1.2 molar equivalents of a compound of formula 7, with from about 3.8 to about 4.2 molar equivalents of a compound of formula 9 and the second step process includes addition of about 0.8 to 1.2 molar equivalent of compound of formula 9 to the reaction mixture.

In some embodiments, one or more amine moieties of formula 7 are selectively protected using a protecting group prior to reacting the polyamine of formula 7 with a compound of formula 8 or 9, thereby providing improved selectivity in the synthesis of the final product. For example, one or more primary amines of the polyamine of formula 7 can be protected prior to reaction with a compound of formula 8 or 9, providing selectivity for the compound of formula 8 or 9 to react with secondary amines. Other protecting group strategies can be employed to provide for selectivity towards primary amines, for example, use of orthogonal protecting groups that can be selectively removed.

Upon completion of the reaction, one or more products having formula 5 can be isolated from the reaction mixture. For example, a compound of formula 5 can be isolated as a single product (e.g., a single structural isomer) or as a mixture of product (e.g., a plurality of structural isomers and/or a plurality of compounds of formula 5). In some embodiments, on or more reaction products can be isolated and/or purified using chromatography, such as flash chromatography, gravity chromatography (e.g., gravity separation of isomers using silica gel), column chromatography (e.g., normal phase HPLC or RPHPLC), or moving bed chromatography. In some embodiments, a reaction product is purified to provide a preparation containing at least about 80% of a single compound, such as a single structural isomer (e.g., at least about 85%, at least about 90%, at least about 95%, at least about 97%, at least about 99%).

In some embodiments, a free amine product is treated with an acid such as HCl to prove an amine salt of the product (e.g., a hydrochloride salt). In some embodiments a salt product provides improved properties, e.g., for handling and/or storage, relative to the corresponding free amine product. In some embodiments, a salt product can prevent or reduce the rate of formation of breakdown product such as N-oxide or N-carbonate formation relative to the corresponding free amine. In some embodiments, a salt product can have improved properties for use in a therapeutic formulation relative to the corresponding free amine

In some embodiments, a polyamine cationic lipid can be made in using a regioselective synthesis approach. The regioselective synthetic approach provides a convenient way to make site specific alkylation on nitrogen(s) of the polyamine backbone that leads to synthesis of specific alkylated derivatives of interest. In general a compound of formula 5 is initially reacted with a reagent that selectively reacts with primary amines or terminal amines to block them from reacting or interfering with further reactions and these blockages could be selectively removed at appropriate stages during the synthesis of a target compound. After blocking terminal amines of a compound of formula 5, one or more of the secondary amines could be selectively blocked with an orthogonal amine protecting groups by using appropriate molar ratios of the reagent and reaction conditions. Selective alkylations, followed by selective deprotection of the blocked amines and further alkylation of regenerated amines and appropriate repetition of the sequence of reactions described provides specific compound of interest. For example, terminal amines of triethylenetetramine (A) is selectively blocked with primary amine specific protecting groups (e.g., trifluoroacetamide) under appropriate reaction conditions and subsequently reacted with excess of orthogonal amine protecting reagent [(Boc)₂O, for e.g.)] in the presence of a base (for e.g., diisopropylethylamine) to block all internal amines (e.g., Boc). Selective removal of the terminal protecting group and subsequent alkylation of the terminal amines, for instance with an acrylamide provides a fully terminal amine alkylated derivative of compound A. Deblocking of the internal amine protection and subsequent alkylation with calculated amount of an acrylamide for instance yields a partially alkylated product B. Another approach to make compound B is to react terminally protected compound A with calculated amount of an orthogonal amine protecting reagent [(Boc)₂O, for e.g.)] to obtain a partially protected derivatives of compound A. Removal of the terminal amine protecting groups of partially and selectively protected A and subsequent alkylation of all unprotected amines with an acrylamide, for instance, yields compound B of interest.

Methods of Making Lipids Having a Biocleavable Moiety

In some embodiments, a compound of formula 6 can be made by reacting a compound of formula 10

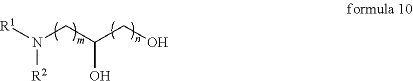

with a compound of formula 11

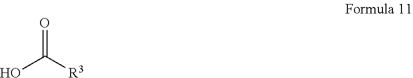

wherein R¹, R², and R³ are as defined above.

In some embodiments, the compounds of formulas 10 and 11 are reacted in the presence of a coupling agent such as a carbodiimide (e.g., a water soluble carbodiimide such as EDCI).

Other chemical reactions and starting materials can be employed to provide a compound of formula 6 having two linking groups L¹ and L². For example, the hydroxyl moieties of formula 10 could be replaced with amine moieties to provide a precursor to amide or urea linking groups.

Upon completion of the reaction, one or more products having formula 6 can be isolated from the reaction mixture. For example, a compound of formula 6 can be isolated as a single product (e.g., a single structural isomer) or as a mixture of product (e.g., a plurality of structural isomers and/or a plurality of compounds of formula 6). In some embodiments, on or more reaction products can be isolated and/or purified using chromatography, such as flash chromatography, gravity chromatography (e.g., gravity separation of isomers using silica gel), column chromatography (e.g., normal phase HPLC or RPHPLC), or moving bed chromatography. In some embodiments, a reaction product is purified to provide a preparation containing at least about 80% of a single compound, such as a single structural isomer (e.g., at least about 85%, at least about 90%, at least about 95%, at least about 97%, at least about 99%).

In some embodiments, a free amine product is treated with an acid such as HCl to prove an amine salt of the product (e.g., a hydrochloride salt). In some embodiments a salt product provides improved properties, e.g., for handling and/or storage, relative to the corresponding free amine product. In some embodiments, a salt product can prevent or reduce the rate of formation of breakdown product such as N-oxide or N-carbonate formation relative to the corresponding free amine. In some embodiments, a salt product can have improved properties for use in a therapeutic formulation relative to the corresponding free amine

### Methods of Making PEG-Lipids

The PEG-lipid compounds can be made, for example, by reacting a glyceride moiety (e.g., a dimyristyl glyceride, dipalmityl glyceride, or distearyl glyceride) with an activating moiety under appropriate conditions, for example, to provide an activated intermediate that could be subsequently reacted with a PEG component having a reactive moiety such as an amine or a hydroxyl group to obtain a PEG-lipid. For example, a dalkylglyceride (e.g., dimyristyl glyceride) is initially reacted with N,N′-disuccinimidyl carbonate in the presence of a base (for e.g., triethylamine) and subsequent reaction of the intermediate formed with a PEG-amine (e.g., mPEG2000-NH₂) in the presence of base such as pyridine affords a PEG-lipid of interest. Under these conditions the PEG component is attached to the lipid moiety via a carbamate linkage. In another instance a PEG-lipid can be made, for example, by reacting a glyceride moiety (e.g., dimyristyl glyceride, dipalmityl glyceride, distearyl glyceride, dimyristoyl glyceride, dipalmitoyl glyceride or distearoyl glyceride) with succinic anhydride and subsequent activation of the carboxyl generated followed by reaction of the activated intermediate with a PEG component with an amine or a hydroxyl group, for instance, to obtain a PEG-lipid. In one example, dimyristyl glyceride is reacted with succinic anhydride in the presence of a base such as DMAP to obtain a hemi-succinate. The free carboxyl moiety of the hemi-succinate thus obtained is activated using standard carboxyl activating agents such as HBTU and diisopropylethylamine and subsequent reaction of the activated carboxyl with mPEH2000-NH₂, for instance, yields a PEG-lipid. In this approach the PEG component is linked to the lipid component via a succinate bridge.

### Association Complexes

The lipid compounds and lipid preparations described herein can be used as a component in an association complex, for example a liposome or a lipoplex. Such association complexes can be used to administer a nucleic acid based therapy such as an RNA, for example a single stranded or double stranded RNA such as dsRNA.

The association complexes disclosed herein can be useful for packaging an oligonucleotide agent capable of modifying gene expression by targeting and binding to a nucleic acid. An oligonucleotide agent can be single-stranded or double-stranded, and can include, e.g., a dsRNA, aa pre-mRNA, an mRNA, a microRNA (miRNA), a mi-RNA precursor (pre-miRNA), plasmid or DNA, or to a protein. An oligonucleotide agent featured in the invention can be, e.g., a dsRNA, a microRNA, antisense RNA, antagomir, supermir, miRNA mimic, antimir, decoy RNA, DNA, U1 adaptor, plasmid and aptamer.

Association complexes can include a plurality of components. In some embodiments, an association complex such as a liposome can include an active ingredient such as a nucleic acid therapeutic (such as an oligonucleotide agent, e.g., dsRNA), a cationic lipid such as a lipid described herein. In some embodiments, the association complex can include a plurality of therapeutic agents, for example two or three single or double stranded nucleic acid moieties targeting more than one gene or different regions of the same gene. Other components can also be included in an association complex, including a PEG-lipid such as a PEG-lipid described herein, or a structural component, such as cholesterol. In some embodiments the association complex also includes a fusogenic lipid or component and/or a targeting molecule. In some preferred embodiments, the association complex is a liposome including an oligonucleotide agent such as dsRNA, a lipid described herein such as a compound of formula 5 or 6, a PEG-lipid such as a PEG-lipid described herein, and a structural component such as cholesterol.

### Formulated Association Complexes

ND98 is generated by reacting ND, the structure of which is provided below:

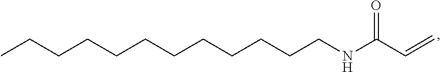

with amine 98, the structure of which is provided below:

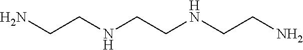

in the ratios provided above (i.e., ND:98=1:1, 2:1, 3:1, 4:1, 5:1, and 6:1).
Liposomes were formulated at ND98:cholesterol:FED2000-CerC16:siRNA=15:0.8:7:1 (wt ratios).

Association complexes having two different nucleic acid moieties were prepared as follows. Stock solutions of ND98, cholesterol, and PEG-C14 in ethanol were prepared at the following concentrations: 133 mg/mL, 25 mg/mL, and 100 mg/mL for ND98, cholesterol, and PEG-C14, respectively. The lipid stocks were then mixed to yield ND98:cholesterol:PEG-C14 molar ratios of 42:48:10. This mixture was then added to aqueous buffer resulting in the spontaneous formulation of lipid nanoparticles in 35% ethanol, 100 mM sodium acetate, pH 5. The unloaded lipid nanoparticles were then passed twice through a 0.08 μm membrane (Whatman, Nucleopore) using an extruder (Lipex, Northern Lipids) to yield unimodal vesicles 20-100 nm in size. The appropriate amount of siRNA in 35% ethanol was then added to the pre-sized, unloaded vesicles at a total excipient:siRNA ratio of 7.5:1 (wt:wt). The resulting mixture was then incubated at 37° C. for 30 min to allow for loading of siRNA into the lipid nanoparticles. After incubation, ethanol removal and buffer exchange was performed by either dialysis or tangential flow filtration against PBS. The final formulation was then sterile filtered through a 0.2 μm filter.

A 1:1 mixture of siRNAs targeting ApoB and Factor VII were formulated as follows. Empty liposomes with composition ND98:cholesterol:PEG-C14=42:48:10 (molar ratio) were prepared as described herein. Different amounts of siRNA were then added to the pre-formed, extruded empty liposomes to yield formulations with initial total excipient:siRNA ratios of 30:1, 20:1, 15:1, 10:1, and 5:1 (wt:wt). Preparation of a formulation at a total excipient:siRNA ratio of 5:1 results in an excess of siRNA in the formulation, saturating the lipid loading capacity. Excess siRNA was then removed by tangential flow filtration using a 100,000 MWCO membrane against 5 volumes of PBS. The resulting formulations were then administered to C57BL/6 mice via tail vein injection at 10 mg/kg siRNA dose. Tolerability of the formulations was assessed by measuring the body weight gain of the animals 24 h and 48 h post administration of the formulation.

Separately, the same ApoB- and Factor VII-targeting siRNAs were individually formulated. The three formulations were then administered at varying doses in an injection volume of 10 μL/g animal body weight. Forty-eight hours after administration, serum samples were collected by retroorbital bleed, animals were sacrificed, and livers were harvested. Serum Factor VII concentrations were determined using a chromogenic diagnostic kit (Coaset Factor VII Assay Kit, DiaPharma) according to manufacturer protocols. Liver mRNA levels of ApoB and Factor VII were determined using a branched-DNA (bDNA) assay (Quantigene, Panomics). No evidence of inhibition between the two therapeutic agents was observed. Rather, both of the therapeutic agents demonstrated effectiveness when administered.

### Oligonucleotide Production

The oligonucleotide compounds of the invention can be prepared using solution-phase or solid-phase organic synthesis. Organic synthesis offers the advantage that the oligonucleotide strands comprising non-natural or modified nucleotides can be easily prepared. Any other means for such synthesis known in the art may additionally or alternatively be employed. It is also known to use similar techniques to prepare other oligonucleotides, such as the phosphorothioates, phosphorodithioates and alkylated derivatives. The double-stranded oligonucleotide compounds of the invention may be prepared using a two-step procedure. First, the individual strands of the double-stranded molecule are prepared separately. Then, the component strands are annealed.

Regardless of the method of synthesis, the oligonucleotide can be prepared in a solution (e.g., an aqueous and/or organic solution) that is appropriate for formulation. For example, the iRNA preparation can be precipitated and redissolved in pure double-distilled water, and lyophilized. The dried iRNA can then be resuspended in a solution appropriate for the intended formulation process.

Teachings regarding the synthesis of particular modified oligonucleotides may be found in the following U.S. patents or pending patent applications: U.S. Pat. Nos. 5,138,045 and 5,218,105, drawn to polyamine conjugated oligonucleotides; U.S. Pat. No. 5,212,295, drawn to monomers for the preparation of oligonucleotides having chiral phosphorus linkages; U.S. Pat. Nos. 5,378,825 and 5,541,307, drawn to oligonucleotides having modified backbones; U.S. Pat. No. 5,386,023, drawn to backbone-modified oligonucleotides and the preparation thereof through reductive coupling; U.S. Pat. No. 5,457,191, drawn to modified nucleobases based on the 3-deazapurine ring system and methods of synthesis thereof; U.S. Pat. No. 5,459,255, drawn to modified nucleobases based on N-2 substituted purines; U.S. Pat. No. 5,521,302, drawn to processes for preparing oligonucleotides having chiral phosphorus linkages; U.S. Pat. No. 5,539,082, drawn to peptide nucleic acids; U.S. Pat. No. 5,554,746, drawn to oligonucleotides having .beta.-lactam backbones; U.S. Pat. No. 5,571,902, drawn to methods and materials for the synthesis of oligonucleotides; U.S. Pat. No. 5,578,718, drawn to nucleosides having alkylthio groups, wherein such groups may be used as linkers to other moieties attached at any of a variety of positions of the nucleoside; U.S. Pat. Nos. 5,587,361 and 5,599,797, drawn to oligonucleotides having phosphorothioate linkages of high chiral purity; U.S. Pat. No. 5,506,351, drawn to processes for the preparation of 2′-O-alkyl guanosine and related compounds, including 2,6-diaminopurine compounds; U.S. Pat. No. 5,587,469, drawn to oligonucleotides having N-2 substituted purines; U.S. Pat. No. 5,587,470, drawn to oligonucleotides having 3-deazapurines; U.S. Pat. No. 5,223,168, and U.S. Pat. No. 5,608,046, both drawn to conjugated 4′-desmethyl nucleoside analogs; U.S. Pat. Nos. 5,602,240, and 5,610,289, drawn to backbone-modified oligonucleotide analogs; and U.S. Pat. Nos. 6,262,241, and 5,459,255, drawn to, inter alia, methods of synthesizing 2′-fluoro-oligonucleotides.

It is not necessary for all positions in a given compound to be uniformly modified, and in fact more than one type of modification may be incorporated in a single oligonucleotide compound or even in a single nucleotide thereof.

siRNA Production

An siRNA can be produced, e.g., in bulk, by a variety of methods. Exemplary methods include: organic synthesis and RNA cleavage, e.g., in vitro cleavage.

### Organic Synthesis

An siRNA can be made by separately synthesizing a single stranded RNA molecule, or each respective strand of a double-stranded RNA molecule, after which the component strands can then be annealed.

A large bioreactor, e.g., the OligoPilot II from Pharmacia Biotec AB (Uppsala Sweden), can be used to produce a large amount of a particular RNA strand for a given siRNA. The OligoPilotII reactor can efficiently couple a nucleotide using only a 1.5 molar excess of a phosphoramidite nucleotide. To make an RNA strand, ribonucleotides amidites are used. Standard cycles of monomer addition can be used to synthesize the 21 to 23 nucleotide strand for the siRNA. Typically, the two complementary strands are produced separately and then annealed, e.g., after release from the solid support and deprotection.

Organic synthesis can be used to produce a discrete siRNA species. The complementary of the species to a particular target gene can be precisely specified. For example, the species may be complementary to a region that includes a polymorphism, e.g., a single nucleotide polymorphism. Further the location of the polymorphism can be precisely defined. In some embodiments, the polymorphism is located in an internal region, e.g., at least 4, 5, 7, or 9 nucleotides from one or both of the termini

dsiRNA Cleavage

siRNAs can also be made by cleaving a larger siRNA. The cleavage can be mediated in vitro or in vivo. For example, to produce iRNAs by cleavage in vitro, the following method can be used:

In vitro transcription. dsiRNA is produced by transcribing a nucleic acid (DNA) segment in both directions. For example, the HiScribe™ RNAi transcription kit (New England Biolabs) provides a vector and a method for producing a dsiRNA for a nucleic acid segment that is cloned into the vector at a position flanked on either side by a T7 promoter. Separate templates are generated for T7 transcription of the two complementary strands for the dsiRNA. The templates are transcribed in vitro by addition of T7 RNA polymerase and dsiRNA is produced. Similar methods using PCR and/or other RNA polymerases (e.g., T3 or SP6 polymerase) can also be used. In one embodiment, RNA generated by this method is carefully purified to remove endotoxins that may contaminate preparations of the recombinant enzymes.

In vitro cleavage. dsiRNA is cleaved in vitro into siRNAs, for example, using a Dicer or comparable RNAse III-based activity. For example, the dsiRNA can be incubated in an in vitro extract from Drosophila or using purified components, e.g., a purified RNAse or RISC complex (RNA-induced silencing complex). See, e.g., Ketting et al. Genes Dev 2001 Oct. 15; 15(20):2654-9. and Hammond Science 2001 Aug. 10; 293(5532):1146-50.

dsiRNA cleavage generally produces a plurality of siRNA species, each being a particular 21 to 23 nt fragment of a source dsiRNA molecule. For example, siRNAs that include sequences complementary to overlapping regions and adjacent regions of a source dsiRNA molecule may be present.

Regardless of the method of synthesis, the siRNA preparation can be prepared in a solution (e.g., an aqueous and/or organic solution) that is appropriate for formulation. For example, the siRNA preparation can be precipitated and redissolved in pure double-distilled water, and lyophilized. The dried siRNA can then be resuspended in a solution appropriate for the intended formulation process.

### Ligands

A wide variety of entities (ligands) can be conjugated to the iRNA agents of the invention. In some embodiments, ligands can be conjugated to nucleobases, sugar moieties, or internucleosidic linkages of nucleic acid molecules. Conjugation to purine nucleobases or derivatives thereof can occur at any position including, endocyclic and exocyclic atoms. In some embodiments, the 2-, 6-, 7-, or 8-positions of a purine nucleobase are attached to a conjugate moiety. Conjugation to pyrimidine nucleobases or derivatives thereof can also occur at any position. In some embodiments, the 2-, 5-, and 6-positions of a pyrimidine nucleobase can be substituted with a conjugate moiety. Conjugation to sugar moieties of nucleosides can occur at any carbon atom. Example carbon atoms of a sugar moiety that can be attached to a conjugate moiety include the 2′, 3′, and 5′ carbon atoms. The 1′ position can also be attached to a conjugate moiety, such as in an abasic residue. Internucleosidic linkages can also bear conjugate moieties. For phosphorus-containing linkages (e.g., phosphodiester, phosphorothioate, phosphorodithiotate, phosphoroamidate, and the like), the conjugate moiety can be attached directly to the phosphorus atom or to an O, N, or S atom bound to the phosphorus atom. For amine- or amide-containing internucleosidic linkages (e.g., PNA), the conjugate moiety can be attached to the nitrogen atom of the amine or amide or to an adjacent carbon atom. In some embodiments the ligands can be conjugated to a non-nucleosidic monomer that can be incorporated into the iRNA agent.

The ligand may be present on a monomer when said monomer is incorporated into the growing strand. In some embodiments, the ligand may be incorporated via coupling to a “precursor” monomer after said “precursor” monomer has been incorporated into the growing strand. In one embodiment, the conjugation of the ligand to the precursor monomer takes place while the oligonucleotide is still attached to the solid support. In one embodiment, the precursor carrying oligonucleotide is first deprotected but not purified before the ligand conjugation takes place. In one embodiment, the precursor monomer carrying oligonucleotide is first deprotected and purified before the ligand conjugation takes place. In certain embodiments, the ligand is conjugated to the monomer under microwave.

In preferred embodiments, a ligand alters the distribution, targeting or lifetime of an oligonucleotide into which it is incorporated. In preferred embodiments a ligand provides an enhanced affinity for a selected target, e.g., molecule, cell or cell type, compartment, e.g., a cellular or organ compartment, tissue, organ or region of the body, as, e.g., compared to a species absent such a ligand.

Ligands can have endosomolytic properties. The endosomolytic ligands promote the lysis of the endosome and/or transport of the composition of the invention, or its components, from the endosome to the cytoplasm of the cell. The endosomolytic ligand may be a polyanionic peptide or peptidomimetic which shows pH-dependent membrane activity and fusogenicity. Exemplary endosomolytic ligands include the GALA peptide (Subbarao et al., Biochemistry, 1987, 26: 2964-2972), the EALA peptide (Vogel et al., J. Am. Chem. Soc., 1996, 118: 1581-1586), and their derivatives (Turk et al., Biochem. Biophys. Acta, 2002, 1559: 56-68). In certain embodiments, the endosomolytic component may contain a chemical group (e.g., an amino acid) which will undergo a change in charge or protonation in response to a change in pH. The endosomolytic component may be linear or branched. Exemplary primary sequences of peptide based endosomolytic ligands are shown in Table 2.

TABLE 2
List of peptides with endosomolytic activity.

| Name | Sequence (N to C) | Ref. |
|---|---|---|
| GALA | AALEALAEALEALAEALEALAEAAAAGGC | 1 |
| EALA | AALAEALAEALAEALAEALAEALAAAAGGC | 2 |
| | ALEALAEALEALAEA | 3 |
| INF-7 | GLFEAIEGFIENGWEGMIWDYG | 4 |
| Inf HA-2 | GLFGAIAGFIENGWEGMIDGWYG | 5 |
| diINF-7 | GLF EAI EGFI ENGW EGMI DGWYGC | 5 |
| | GLF EAI EGFI ENGW EGMI DGWYGC | |
| diINF3 | GLF EAI EGFI ENGW EGMI DGGC | 6 |
| | GLF EAI EGFI ENGW EGMI DGGC | |
| GLF | GLFGALAEALAEALAEHLAEALAEALEALAAGGSC | 6 |
| GALA-INF3 | GLFEAIEGFIENGWEGLAEALAEALEALAAGGSC | 6 |
| INF-5 | GLF EAI EGFI ENGW EGnI DG K | 4 |
| | GLF EAI EGFI ENGW EGnI DG | |

n, norleucine
References
1 Subbarao et al. (1987) Biochemistry 26: 2964-2972.
2 Vogel, et al. (1996) J. Am. Chem. Soc. 118: 1581-1586
3 Turk, et al. (2002) Biochim. Biophys. Acta 1559: 56-68.
4 Plank, et al. (1994) J. Biol. Chem. 269: 12918-12924.
5 Mastrobattista, et al. (2002) J. Biol. Chem. 277: 27135-43.
6 Oberhauser, et al. (1995) Deliv. Strategies Antisense Oligonucleotide Ther. 247-66.

Preferred ligands can improve transport, hybridization, and specificity properties and may also improve nuclease resistance of the resultant natural or modified oligoribonucleotide, or a polymeric molecule comprising any combination of monomers described herein and/or natural or modified ribonucleotides.

Ligands in general can include therapeutic modifiers, e.g., for enhancing uptake; diagnostic compounds or reporter groups e.g., for monitoring distribution; cross-linking agents; and nuclease-resistance conferring moieties. General examples include lipids, steroids, vitamins, sugars, proteins, peptides, polyamines, and peptide mimics

Ligands can include a naturally occurring substance, such as a protein (e.g., human serum albumin (HSA), low-density lipoprotein (LDL), high-density lipoprotein (HDL), or globulin); an carbohydrate (e.g., a dextran, pullulan, chitin, chitosan, inulin, cyclodextrin or hyaluronic acid); or a lipid. The ligand may also be a recombinant or synthetic molecule, such as a synthetic polymer, e.g., a synthetic polyamino acid, an oligonucleotide (e.g. an aptamer). Examples of polyamino acids include polyamino acid is a polylysine (PLL), poly L-aspartic acid, poly L-glutamic acid, styrene-maleic acid anhydride copolymer, poly(L-lactide-co-glycolied) copolymer, divinyl ether-maleic anhydride copolymer, N-(2-hydroxypropyl)methacrylamide copolymer (HMPA), polyethylene glycol (PEG), polyvinyl alcohol (PVA), polyurethane, poly(2-ethylacryllic acid), N-isopropylacrylamide polymers, or polyphosphazine. Example of polyamines include: polyethylenimine, polylysine (PLL), spermine, spermidine, polyamine, pseudopeptide-polyamine, peptidomimetic polyamine, dendrimer polyamine, arginine, amidine, protamine, cationic lipid, cationic porphyrin, quaternary salt of a polyamine, or an alpha helical peptide.

Ligands can also include targeting groups, e.g., a cell or tissue targeting agent, e.g., a lectin, glycoprotein, lipid or protein, e.g., an antibody, that binds to a specified cell type such as a kidney cell. A targeting group can be a thyrotropin, melanotropin, lectin, glycoprotein, surfactant protein A, Mucin carbohydrate, multivalent lactose, multivalent galactose, N-acetyl-galactosamine, N-acetyl-gulucosamine multivalent mannose, multivalent fucose, glycosylated polyaminoacids, multivalent galactose, transferrin, bisphosphonate, polyglutamate, polyaspartate, a lipid, cholesterol, a steroid, bile acid, folate, vitamin B12, biotin, an RGD peptide, an RGD peptide mimetic or an aptamer. Table 3 shows some examples of targeting ligands and their associated receptors.

Other examples of ligands include dyes, intercalating agents (e.g. acridines), cross-linkers (e.g. psoralene, mitomycin C), porphyrins (TPPC4, texaphyrin, Sapphyrin), polycyclic aromatic hydrocarbons (e.g., phenazine, dihydrophenazine), artificial endonucleases (e.g. EDTA), lipophilic molecules, e.g, cholesterol, cholic acid, adamantane acetic acid, 1-pyrene butyric acid, dihydrotestosterone, 1,3-Bis-O(hexadecyl)glycerol, geranyloxyhexyl group, hexadecylglycerol, borneol, menthol, 1,3-propanediol, heptadecyl group, palmitic acid, myristic acid, O3-(oleoyl)lithocholic acid, O3-(oleoyl)cholenic acid, dimethoxytrityl, or phenoxazine) and peptide conjugates (e.g., antennapedia peptide, Tat peptide), alkylating agents, phosphate, amino, mercapto, PEG (e.g., PEG-40K), MPEG, [MPEG]₂, polyamino, alkyl, substituted alkyl, radiolabeled markers, enzymes, haptens (e.g. biotin), transport/absorption facilitators (e.g., aspirin, vitamin E, folic acid), synthetic ribonucleases (e.g., imidazole, bisimidazole, histamine, imidazole clusters, acridine-imidazole conjugates, Eu3+ complexes of tetraazamacrocycles), dinitrophenyl, HRP, or AP.

TABLE 3
Liver targeting Ligands and their associated receptors.

| Liver Cells | Ligand | Receptor |
|---|---|---|
| 1) Parenchymal | Galactose | ASGP-R (Asiologlyco- |
| Cell (PC) | | protein receptor) |
| (Hepatocytes) | Gal NAc (n-acetyl- | ASPG-R (GalNAc |
| | galactosamine) | Receptor) |
| | Lactose | |
| | Asialofetuin | ASPG-r |
| 2) Sinusoidal | Hyaluronan | Hyaluronan receptor |
| Endothelial | Procollagen | Procollagen receptor |
| Cell (SEC) | Negatively charged | Scavenger receptors |
| | molecules | |
| | Mannose | Mannose receptors |
| | N-acetyl Glucosamine | Scavenger receptors |
| | Immunoglobulins | Fc Receptor |
| | LPS | CD14 Receptor |
| | Insulin | Receptor mediated transcytosis |
| | Transferrin | Receptor mediated transcytosis |
| | Albumins | Non-specific |
| | Sugar-Albumin | |
| | conjugates | |
| | Mannose-6-phosphate | Mannose-6-phosphate receptor |
| 3) Kupffer Cell | Mannose | Mannose receptors |
| (KC) | Fucose | Fucose receptors |
| | Albumins | Non-specific |
| | Mannose-albumin | |
| | conjugates | |

Ligands can be proteins, e.g., glycoproteins, or peptides, e.g., molecules having a specific affinity for a co-ligand, or antibodies e.g., an antibody, that binds to a specified cell type such as a cancer cell, endothelial cell, or bone cell. Ligands may also include hormones and hormone receptors. They can also include non-peptidic species, such as lipids, lectins, carbohydrates, vitamins, cofactors, multivalent lactose, multivalent galactose, N-acetyl-galactosamine, N-acetyl-glucosamine, multivalent mannose, multivalent fucose, or aptamers. The ligand can be, for example, a lipopolysaccharide, an activator of p38 MAP kinase, or an activator of NF-κB.

The ligand can be a substance, e.g, a drug, which can increase the uptake of the iRNA agent into the cell, for example, by disrupting the cell's cytoskeleton, e.g., by disrupting the cell's microtubules, microfilaments, and/or intermediate filaments. The drug can be, for example, taxon, vincristine, vinblastine, cytochalasin, nocodazole, japlakinolide, latrunculin A, phalloidin, swinholide A, indanocine, or myoservin.

The ligand can increase the uptake of the iRNA agent into the cell by activating an inflammatory response, for example. Exemplary ligands that would have such an effect include tumor necrosis factor alpha (TNFalpha), interleukin-1 beta, or gamma interferon.

In one aspect, the ligand is a lipid or lipid-based molecule. Such a lipid or lipid-based molecule preferably binds a serum protein, e.g., human serum albumin (HSA). An HSA binding ligand allows for distribution of the conjugate to a target tissue, e.g., a non-kidney target tissue of the body. For example, the target tissue can be the liver, including parenchymal cells of the liver. Other molecules that can bind HSA can also be used as ligands. For example, neproxin or aspirin can be used. A lipid or lipid-based ligand can (a) increase resistance to degradation of the conjugate, (b) increase targeting or transport into a target cell or cell membrane, and/or (c) can be used to adjust binding to a serum protein, e.g., HSA.

A lipid based ligand can be used to modulate, e.g., control the binding of the conjugate to a target tissue. For example, a lipid or lipid-based ligand that binds to HSA more strongly will be less likely to be targeted to the kidney and therefore less likely to be cleared from the body. A lipid or lipid-based ligand that binds to HSA less strongly can be used to target the conjugate to the kidney.

In a preferred embodiment, the lipid based ligand binds HSA. Preferably, it binds HSA with a sufficient affinity such that the conjugate will be preferably distributed to a non-kidney tissue. However, it is preferred that the affinity not be so strong that the HSA-ligand binding cannot be reversed.

In another preferred embodiment, the lipid based ligand binds HSA weakly or not at all, such that the conjugate will be preferably distributed to the kidney. Other moieties that target to kidney cells can also be used in place of or in addition to the lipid based ligand.

In another aspect, the ligand is a moiety, e.g., a vitamin, which is taken up by a target cell, e.g., a proliferating cell. These are particularly useful for treating disorders characterized by unwanted cell proliferation, e.g., of the malignant or non-malignant type, e.g., cancer cells. Exemplary vitamins include vitamin A, E, and K. Other exemplary vitamins include are B vitamin, e.g., folic acid, B12, riboflavin, biotin, pyridoxal or other vitamins or nutrients taken up by cancer cells. Also included are HAS, low density lipoprotein (LDL) and high-density lipoprotein (HDL).

In another aspect, the ligand is a cell-permeation agent, preferably a helical cell-permeation agent. Preferably, the agent is amphipathic. An exemplary agent is a peptide such as tat or antennopedia. If the agent is a peptide, it can be modified, including a peptidylmimetic, invertomers, non-peptide or pseudo-peptide linkages, and use of D-amino acids. The helical agent is preferably an alpha-helical agent, which preferably has a lipophilic and a lipophobic phase.

The ligand can be a peptide or peptidomimetic. A peptidomimetic (also referred to herein as an oligopeptidomimetic) is a molecule capable of folding into a defined three-dimensional structure similar to a natural peptide. The attachment of peptide and peptidomimetics to iRNA agents can affect pharmacokinetic distribution of the iRNA, such as by enhancing cellular recognition and absorption. The peptide or peptidomimetic moiety can be about 5-50 amino acids long, e.g., about 5, 10, 15, 20, 25, 30, 35, 40, 45, or 50 amino acids long (see Table 4, for example).

TABLE 4
Exemplary Cell Permeation Peptides

| Cell Permeation | | |
|---|---|---|
| Peptide | Amino acid Sequence | Reference |
| Penetratin | RQIKIWFQNRRMKWKK | Derossi et al., J. Biol. Chem. |
| | | 269: 10444, 1994 |
| Tat fragment (48- | GRKKRRQRRRPPQC | Vives et al., J. Biol. Chem., |
| 60) | | 272: 16010, 1997 |
| Signal Sequence- | GALFLGWLGAAGSTMGAWSQPKKKRKV | Chaloin et al., Biochem. |
| based peptide | | Biophys. Res. Commun., |
| | | 243: 601, 1998 |
| PVEC | LLIILRRRIRKQAHAHSK | Elmquist et al., Exp. Cell |
| | | Res., 269: 237, 2001 |
| Transportan | GWTLNSAGYLLKINLKALAALAKKIL | Pooga et al., FASEB J., |
| | | 12: 67, 1998 |
| Amphiphilic | KLALKLALKALKAALKLA | Oehlke et al., Mol. Ther., |
| model peptide | | 2: 339, 2000 |
| Arg₉ | RRRRRRRRR | Mitchell et al., J. Pept. Res., |
| | | 56: 318, 2000 |
| Bacterial cell wall | KFFKFFKFFK | |
| permeating | | |
| LL-37 | LLGDFFRKSKEKIGKEFKRIVQRIKDFLRNLVPR | |
| | TES | |
| Cecropin P1 | SWLSKTAKKLENSAKKRISEGIAIAIQGGPR | |
| α-defensin | ACYCRIPACIAGERRYGTCIYQGRLWAFCC | |
| b-defensin | DHYNCVSSGGQCLYSACPIFTKIQGTCYRGKAK | |
| | CCK | |
| Bactenecin | RKCRIVVIRVCR | |
| PR-39 | RRRPRPPYLPRPRPPPFFPPRLPPRIPPGFPPRFPP | |
| | RFPGKR-NH2 | |
| Indolicidin | ILPWKWPWWPWRR-NH2 | |

A peptide or peptidomimetic can be, for example, a cell permeation peptide, cationic peptide, amphipathic peptide, or hydrophobic peptide (e.g., consisting primarily of Tyr, Trp or Phe). The peptide moiety can be a dendrimer peptide, constrained peptide or crosslinked peptide. In another alternative, the peptide moiety can include a hydrophobic membrane translocation sequence (MTS). An exemplary hydrophobic MTS-containing peptide is RFGF having the amino acid sequence AAVALLPAVLLALLAP. An RFGF analogue (e.g., amino acid sequence AALLPVLLAAP) containing a hydrophobic MTS can also be a targeting moiety. The peptide moiety can be a “delivery” peptide, which can carry large polar molecules including peptides, oligonucleotides, and protein across cell membranes. For example, sequences from the HIV Tat protein (GRKKRRQRRRPPQ) and the Drosophila Antennapedia protein (RQIKIWFQNRRMKWKK) have been found to be capable of functioning as delivery peptides. A peptide or peptidomimetic can be encoded by a random sequence of DNA, such as a peptide identified from a phage-display library, or one-bead-one-compound (OBOC) combinatorial library (Lam et al., Nature, 354:82-84, 1991). Preferably the peptide or peptidomimetic tethered to an iRNA agent via an incorporated monomer unit is a cell targeting peptide such as an arginine-glycine-aspartic acid (RGD)-peptide, or RGD mimic A peptide moiety can range in length from about 5 amino acids to about 40 amino acids. The peptide moieties can have a structural modification, such as to increase stability or direct conformational properties. Any of the structural modifications described below can be utilized.

An RGD peptide moiety can be used to target a tumor cell, such as an endothelial tumor cell or a breast cancer tumor cell (Zitzmann et al., Cancer Res., 62:5139-43, 2002). An RGD peptide can facilitate targeting of an iRNA agent to tumors of a variety of other tissues, including the lung, kidney, spleen, or liver (Aoki et al., Cancer Gene Therapy 8:783-787, 2001). Preferably, the RGD peptide will facilitate targeting of an iRNA agent to the kidney. The RGD peptide can be linear or cyclic, and can be modified, e.g., glycosylated or methylated to facilitate targeting to specific tissues. For example, a glycosylated RGD peptide can deliver an iRNA agent to a tumor cell expressing αᵥβ₃ (Haubner et al., Jour. Nucl. Med., 42:326-336, 2001).

Peptides that target markers enriched in proliferating cells can be used. E.g., RGD containing peptides and peptidomimetics can target cancer cells, in particular cells that exhibit an Iᵥθ₃ integrin. Thus, one could use RGD peptides, cyclic peptides containing RGD, RGD peptides that include D-amino acids, as well as synthetic RGD mimics. In addition to RGD, one can use other moieties that target the Iᵥθ₃ integrin ligand. Generally, such ligands can be used to control proliferating cells and angiogeneis. Preferred conjugates of this type ligands that targets PECAM-1, VEGF, or other cancer gene, e.g., a cancer gene described herein.

A “cell permeation peptide” is capable of permeating a cell, e.g., a microbial cell, such as a bacterial or fungal cell, or a mammalian cell, such as a human cell. A microbial cell-permeating peptide can be, for example, an α-helical linear peptide (e.g., LL-37 or Ceropin P1), a disulfide bond-containing peptide (e.g., α-defensin, β-defensin or bactenecin), or a peptide containing only one or two dominating amino acids (e.g., PR-39 or indolicidin). A cell permeation peptide can also include a nuclear localization signal (NLS). For example, a cell permeation peptide can be a bipartite amphipathic peptide, such as MPG, which is derived from the fusion peptide domain of HIV-1 gp41 and the NLS of SV40 large T antigen (Simeoni et al., Nucl. Acids Res. 31:2717-2724, 2003).

In one embodiment, a targeting peptide tethered to an iRNA agent and/or the carrier oligomer can be an amphipathic α-helical peptide. Exemplary amphipathic α-helical peptides include, but are not limited to, cecropins, lycotoxins, paradaxins, buforin, CPF, bombinin-like peptide (BLP), cathelicidins, ceratotoxins, S. clava peptides, hagfish intestinal antimicrobial peptides (HFIAPs), magainines, brevinins-2, dermaseptins, melittins, pleurocidin, H₂A peptides, Xenopus peptides, esculentinis-1, and caerins. A number of factors will preferably be considered to maintain the integrity of helix stability. For example, a maximum number of helix stabilization residues will be utilized (e.g., leu, ala, or lys), and a minimum number helix destabilization residues will be utilized (e.g., proline, or cyclic monomeric units. The capping residue will be considered (for example Gly is an exemplary N-capping residue and/or C-terminal amidation can be used to provide an extra H-bond to stabilize the helix. Formation of salt bridges between residues with opposite charges, separated by i±3, or i±4 positions can provide stability. For example, cationic residues such as lysine, arginine, homo-arginine, ornithine or histidine can form salt bridges with the anionic residues glutamate or aspartate.

Peptide and peptidomimetic ligands include those having naturally occurring or modified peptides, e.g., D or L peptides; N-methyl peptides; azapeptides; peptides having one or more amide, i.e., peptide, linkages replaced with one or more urea, thiourea, carbamate, or sulfonyl urea linkages; or cyclic peptides.

The targeting ligand can be any ligand that is capable of targeting a specific receptor. Examples are: folate, GalNAc, galactose, mannose, mannose-6P, clusters of sugars such as GalNAc cluster, mannose cluster, galactose cluster, or an apatamer. A cluster is a combination of two or more sugar units. The targeting ligands also include integrin receptor ligands, Chemokine receptor ligands, transferrin, biotin, serotonin receptor ligands, PSMA, endothelin, GCPII, somatostatin, LDL and HDL ligands. The ligands can also be based on nucleic acid, e.g., an aptamer. The aptamer can be unmodified or have any combination of modifications disclosed herein.

Endosomal release agents include imidazoles, poly or oligoimidazoles, PEIs, peptides, fusogenic peptides, polycarboxylates, polyacations, masked oligo or poly cations or anions, acetals, polyacetals, ketals/polyketyals, orthoesters, polymers with masked or unmasked cationic or anionic charges, dendrimers with masked or unmasked cationic or anionic charges.

PK modulator stands for pharmacokinetic modulator. PK modulator include lipophiles, bile acids, steroids, phospholipid analogues, peptides, protein binding agents, PEG, vitamins etc. Examplary PK modulator include, but are not limited to, cholesterol, fatty acids, cholic acid, lithocholic acid, dialkylglycerides, diacylglyceride, phospholipids, sphingolipids, naproxen, ibuprofen, vitamin E, biotin etc. Oligonucleotides that comprise multiple phosphorothioate linkages are also known to bind to serum protein, thus short oligonucleotides, e.g. oligonucleotides of about 5 bases, 10 bases, 15 bases or 20 bases, comprising multiple of phosphorothioate linkages in the backbone are also amenable to the present invention as ligands (e.g. as PK modulating ligands). In addition, aptamers that bind serum components (e.g. serum proteins) are also amenable to the present invention as ligands.

Other ligands amenable to the invention are described in copending applications U.S. Ser. No. 10/916,185, filed Aug. 10, 2004; U.S. Ser. No. 10/946,873, filed Sep. 21, 2004; U.S. Ser. No.: 10/833,934, filed Aug. 3, 2007; U.S. Ser. No. 11/115,989 filed Apr. 27, 2005 and U.S. Ser. No.: 11/944,227 filed Nov. 21, 2007, which are incorporated by reference in their entireties for all purposes.

When two or more ligands are present, the ligands can all have same properties, all have different properties or some ligands have the same properties while others have different properties. For example, a ligand can have targeting properties or have PK modulating properties. In a preferred embodiment, all the ligands have different properties.

There are numerous methods for preparing conjugates of oligomeric compounds. Generally, an oligomeric compound is attached to a conjugate moiety by contacting a reactive group (e.g., OH, SH, amine, carboxyl, aldehyde, and the like) on the oligomeric compound with a reactive group on the conjugate moiety. In some embodiments, one reactive group is electrophilic and the other is nucleophilic. For example, an electrophilic group can be a carbonyl-containing functionality and a nucleophilic group can be an amine or thiol. Methods for conjugation of nucleic acids and related oligomeric compounds with and without linking groups are well described in the literature such as, for example, in Manoharan in Antisense Research and Applications, Crooke and LeBleu, eds., CRC Press, Boca Raton, Fla., 1993, Chapter 17, which is incorporated herein by reference in its entirety.

Representative United States patents that teach the preparation of oligonucleotide conjugates include, but are not limited to, U.S. Pat. Nos. 4,828,979; 4,948,882; 5,218,105; 5,525,465; 5,541,313; 5,545,730; 5,552,538; 5,578,717, 5,580,731; 5,580,731; 5,591,584; 5,109,124; 5,118,802; 5,138,045; 5,414,077; 5,486,603; 5,512,439; 5,578,718; 5,608,046; 4,587,044; 4,605,735; 4,667,025; 4,762,779; 4,789,737; 4,824,941; 4,835,263; 4,876,335; 4,904, 582; 4,958,013; 5,082,830; 5,112,963; 5,214,136; 5,082,830; 5,112,963; 5,149,782; 5,214,136; 5,245,022; 5,254,469; 5,258,506; 5,262,536; 5,272,250; 5,292,873; 5,317,098; 5,371,241, 5,391,723; 5,416,203, 5,451,463; 5,510,475; 5,512,667; 5,514,785; 5,565,552; 5,567,810; 5,574, 142; 5,585,481; 5,587,371; 5,595,726; 5,597,696; 5,599,923; 5,599,928; 5,672,662; 5,688,941; 5,714,166; 6,153,737; 6,172,208; 6,300,319; 6,335,434; 6,335,437; 6,395,437; 6,444,806; 6,486,308; 6,525,031; 6,528,631; 6,559,279; each of which is herein incorporated by reference.

### Formulations

The oligonucleotide compounds described herein can be formulated for administration to a subject It is understood that these formulations, compositions and methods can be practiced with modified siRNA compounds, and such practice is within the invention.

A formulated siRNA composition can assume a variety of states. In some examples, the composition is at least partially crystalline, uniformly crystalline, and/or anhydrous (e.g., less than 80, 50, 30, 20, or 10% water). In another example, the siRNA is in an aqueous phase, e.g., in a solution that includes water.

The aqueous phase or the crystalline compositions can, e.g., be incorporated into a delivery vehicle, e.g., a liposome (particularly for the aqueous phase) or a particle (e.g., a microparticle as can be appropriate for a crystalline composition). Generally, the oligonucleotide composition is formulated in a manner that is compatible with the intended method of administration, as described herein. For example, in particular embodiments the composition is prepared by at least one of the following methods: spray drying, lyophilization, vacuum drying, evaporation, fluid bed drying, or a combination of these techniques; or sonication with a lipid, freeze-drying, condensation and other self-assembly.

A oligonucleotide preparation can be formulated in combination with another agent, e.g., another therapeutic agent or an agent that stabilizes an oligonucleotide, e.g., a protein that complexes with oligonucleotide to form an iRNP. Still other agents include chelators, e.g., EDTA (e.g., to remove divalent cations such as Mg²⁺), salts, RNAse inhibitors (e.g., a broad specificity RNAse inhibitor such as RNAsin) and so forth.

In one embodiment, the oligonucleotide preparation includes another siRNA compound, e.g., a second oligonucleotide that can mediate RNAi that targets a second gene, or that targets the same gene. Still other preparation can include at least 3, 5, ten, twenty, fifty, or a hundred or more different oligonucleotide species. Such oligonucleotides can mediate RNAi that targets a similar number of different genes.

In one embodiment, the oligonucleotide preparation includes at least a second therapeutic agent (e.g., an agent other than an RNA or a DNA). For example, an oligonucleotide composition for the treatment of a viral disease, e.g., HIV, might include a known antiviral agent (e.g., a protease inhibitor or reverse transcriptase inhibitor). In another example, an oligonucleotide composition for the treatment of a cancer might further comprise a chemotherapeutic agent.

Exemplary formulations are discussed below:

### Liposomes

The oligonucleotides of the invention, e.g. single-stranded oligonucleotide and double-stranded oligonucleotide, can be formulated in liposomes. As used herein, a liposome is a structure having lipid-containing membranes enclosing an aqueous interior. Liposomes may have one or more lipid membranes. Liposomes may be characterized by membrane type and by size. Small unilamellar vesicles (SUVs) have a single membrane and typically range between 0.02 and 0.05 μM in diameter; large unilamellar vesicles (LUVS) are typically larger than 0.05 μM. Oligolamellar large vesicles and multilamellar vesicles have multiple, usually concentric, membrane layers and are typically larger than 0.1 μM. Liposomes with several nonconcentric membranes, i.e., several smaller vesicles contained within a larger vesicle, are termed multivesicular vesicles.

For ease of exposition the formulations, compositions and methods in this section are discussed largely with regard to unmodified oligonucleotide compounds. It may be understood, however, that these formulations, compositions and methods can be practiced with other oligonucleotide compounds, e.g., modified siRNAs, and such practice is within the invention. An siRNA compound, e.g., a double-stranded siRNA compound, or ssiRNA compound, (e.g., a precursor, e.g., a larger siRNA compound which can be processed into a ssiRNA compound, or a DNA which encodes an siRNA compound, e.g., a double-stranded siRNA compound, or ssiRNA compound, or precursor thereof) preparation can be formulated for delivery in a membranous molecular assembly, e.g., a liposome or a micelle. As used herein, the term “liposome” refers to a vesicle composed of amphiphilic lipids arranged in at least one bilayer, e.g., one bilayer or a plurality of bilayers. Liposomes include unilamellar and multilamellar vesicles that have a membrane formed from a lipophilic material and an aqueous interior. The aqueous portion contains the siRNA composition. The lipophilic material isolates the aqueous interior from an aqueous exterior, which typically does not include the siRNA composition, although in some examples, it may. Liposomes are useful for the transfer and delivery of active ingredients to the site of action. Because the liposomal membrane is structurally similar to biological membranes, when liposomes are applied to a tissue, the liposomal bilayer fuses with bilayer of the cellular membranes. As the merging of the liposome and cell progresses, the internal aqueous contents that include the siRNA are delivered into the cell where the siRNA can specifically bind to a target RNA and can mediate RNAi. In some cases the liposomes are also specifically targeted, e.g., to direct the siRNA to particular cell types.

A liposome containing an siRNA can be prepared by a variety of methods. In one example, the lipid component of a liposome is dissolved in a detergent so that micelles are formed with the lipid component. For example, the lipid component can be an amphipathic cationic lipid or lipid conjugate. The detergent can have a high critical micelle concentration and may be nonionic. Exemplary detergents include cholate, CHAPS, octylglucoside, deoxycholate, and lauroyl sarcosine. The siRNA preparation is then added to the micelles that include the lipid component. The cationic groups on the lipid interact with the siRNA and condense around the siRNA to form a liposome. After condensation, the detergent is removed, e.g., by dialysis, to yield a liposomal preparation of siRNA.

If necessary a carrier compound that assists in condensation can be added during the condensation reaction, e.g., by controlled addition. For example, the carrier compound can be a polymer other than a nucleic acid (e.g., spermine or spermidine). pH can also adjusted to favor condensation.

Further description of methods for producing stable polynucleotide delivery vehicles, which incorporate a polynucleotide/cationic lipid complex as structural components of the delivery vehicle, are described in, e.g., WO 96/37194. Liposome formation can also include one or more aspects of exemplary methods described in Felgner, P. L. et al., Proc. Natl. Acad. Sci., USA 8:7413-7417, 1987; U.S. Pat. No. 4,897,355; U.S. Pat. No. 5,171,678; Bangham, et al. M. Mol. Biol. 23:238, 1965; Olson, et al. Biochim. Biophys. Acta 557:9, 1979; Szoka, et al. Proc. Natl. Acad. Sci. 75: 4194, 1978; Mayhew, et al. Biochim. Biophys. Acta 775:169, 1984; Kim, et al. Biochim. Biophys. Acta 728:339, 1983; and Fukunaga, et al. Endocrinol. 115:757, 1984. Commonly used techniques for preparing lipid aggregates of appropriate size for use as delivery vehicles include sonication and freeze-thaw plus extrusion (see, e.g., Mayer, et al. Biochim. Biophys. Acta 858:161, 1986). Microfluidization can be used when consistently small (50 to 200 nm) and relatively uniform aggregates are desired (Mayhew, et al. Biochim. Biophys. Acta 775:169, 1984). These methods are readily adapted to packaging siRNA preparations into liposomes.

Liposomes may further include one or more additional lipids and/or other components such as cholesterol. Other lipids may be included in the liposome compositions for a variety of purposes, such as to prevent lipid oxidation, to stabilize the bilayer, to reduce aggregation during formation or to attach ligands onto the liposome surface. Any of a number of lipids may be present, including amphipathic, neutral, cationic, and anionic lipids. Such lipids can be used alone or in combination.

Additional components that may be present in a liposomes include bilayer stabilizing components such as polyamide oligomers (see, e.g., U.S. Pat. No. 6,320,017), peptides, proteins, detergents, lipid-derivatives, such as PEG conjugated to phosphatidylethanolamine, PEG conjugated to phosphatidic acid, PEG conjugated to ceramides (see, U.S. Pat. No. 5,885,613), PEG conjugated dialkylamines and PEG conjugated 1,2-diacyloxypropan-3-amines

Liposome can include components selected to reduce aggregation of lipid particles during formation, which may result from steric stabilization of particles which prevents charge-induced aggregation during formation. Suitable components that reduce aggregation include, but are not limited to, polyethylene glycol (PEG)-modified lipids, monosialoganglioside Gm1, and polyamide oligomers (“PAO”) such as (described in U.S. Pat. No. 6,320,017). Exemplary suitable PEG-modified lipids include, but are not limited to, PEG-modified phosphatidylethanolamine and phosphatidic acid, PEG-ceramide conjugates (e.g., PEG-CerC14 or PEG-CerC20), PEG-modified dialkylamines and PEG-modified 1,2-diacyloxypropan-3-amines Particularly preferred are PEG-modified diacylglycerols and dialkylglycerols. Other compounds with uncharged, hydrophilic, steric-barrier moieties, which prevent aggregation during formation, like PEG, Gm1, or ATTA, can also be coupled to lipids to reduce aggregation during formation. ATTA-lipids are described, e.g., in U.S. Pat. No. 6,320,017, and PEG-lipid conjugates are described, e.g., in U.S. Pat. Nos. 5,820,873, 5,534,499 and 5,885,613. Typically, the concentration of the lipid component selected to reduce aggregation is about 1 to 15% (by mole percent of lipids). It should be noted that aggregation preventing compounds do not necessarily require lipid conjugation to function properly. Free PEG or free ATTA in solution may be sufficient to prevent aggregation. If the liposomes are stable after formulation, the PEG or ATTA can be dialyzed away before administration to a subject.

Neutral lipids, when present in the liposome composition, can be any of a number of lipid species which exist either in an uncharged or neutral zwitterionic form at physiological pH. Such lipids include, for example diacylphosphatidylcholine, diacylphosphatidylethanolamine, ceramide, sphingomyelin, dihydrosphingomyelin, cephalin, and cerebrosides. The selection of neutral lipids for use in liposomes described herein is generally guided by consideration of, e.g., liposome size and stability of the liposomes in the bloodstream. Preferably, the neutral lipid component is a lipid having two acyl groups, (i.e., diacylphosphatidylcholine and diacylphosphatidylethanolamine). Lipids having a variety of acyl chain groups of varying chain length and degree of saturation are available or may be isolated or synthesized by well-known techniques. In one group of embodiments, lipids containing saturated fatty acids with carbon chain lengths in the range of C₁₄ to C₂₂ are preferred. In another group of embodiments, lipids with mono or diunsaturated fatty acids with carbon chain lengths in the range of C₁₄ to C₂₂ are used. Additionally, lipids having mixtures of saturated and unsaturated fatty acid chains can be used. Preferably, the neutral lipids used in the present invention are DOPE, DSPC, POPC, DMPC, DPPC or any related phosphatidylcholine. The neutral lipids useful in the present invention may also be composed of sphingomyelin, dihydrosphingomyeline, or phospholipids with other head groups, such as serine and inositol.

The sterol component of the lipid mixture, when present, can be any of those sterols conventionally used in the field of liposome, lipid vesicle or lipid particle preparation. A preferred sterol is cholesterol.

Cationic lipids, when present in the liposome composition, can be any of a number of lipid species which carry a net positive charge at about physiological pH. Such lipids include, but are not limited to, N,N-dioleyl-N,N-dimethylammonium chloride (“DODAC”); N-(2,3-dioleyloxy)propyl-N,N—N-triethylammonium chloride (“DOTMA”); N,N-distearyl-N,N-dimethylammonium bromide (“DDAB”); N-(2,3-dioleoyloxy)propyl)-N,N,N-trimethylammonium chloride (“DOTAP”); 1,2-Dioleyloxy-3-trimethylaminopropane chloride salt (“DOTAP.C1”); 3β-(N—(N′,N′-dimethylaminoethane)-carbamoyl)cholesterol (“DC-Chol”), N-(1-(2,3-dioleyloxy)propyl)-N-2-(sperminecarboxamido)ethyl)-N,N-dimethyl ammonium trifluoracetate (“DOSPA”), dioctadecylamidoglycyl carboxyspermine (“DOGS”), 1,2-dileoyl-sn-3-phosphoethanolamine (“DOPE”), 1,2-dioleoyl-3-dimethylammonium propane (“DODAP”), N, N-dimethyl-2,3-dioleyloxy)propylamine (“DODMA”), N-(1,2-dimyristyloxyprop-3-yl)-N,N-dimethyl-N-hydroxyethyl ammonium bromide (“DMRIE”), 5-carboxyspermylglycine diocaoleyamide (“DOGS”), and dipalmitoylphosphatidylethanolamine 5-carboxyspermyl-amide (“DPPES”). Additionally, a number of commercial preparations of cationic lipids can be used, such as, e.g., LIPOFECTIN (including DOTMA and DOPE, available from GIBCO/BRL), and LIPOFECTAMINE (comprising DOSPA and DOPE, available from GIBCO/BRL). Other cationic lipids suitable for lipid particle formation are described in WO98/39359, WO96/37194. Other cationic lipids suitable for liposome formation are described in U.S. Provisional applications No. 61/018,616 (filed Jan. 2, 2008), No. 61/039,748 (filed Mar. 26, 2008), No. 61/047,087 (filed Apr. 22, 2008) and No. 61/051,528 (filed May 21-2008), all of which are incorporated by reference in their entireties for all purposes.

Anionic lipids, when present in the liposome composition, can be any of a number of lipid species which carry a net negative charge at about physiological pH. Such lipids include, but are not limited to, phosphatidylglycerol, cardiolipin, diacylphosphatidylserine, diacylphosphatidic acid, N-dodecanoyl phosphatidylethanoloamine, N-succinyl phosphatidylethanolamine, N-glutaryl phosphatidylethanolamine, lysylphosphatidylglycerol, and other anionic modifying groups joined to neutral lipids.

“Amphipathic lipids” refer to any suitable material, wherein the hydrophobic portion of the lipid material orients into a hydrophobic phase, while the hydrophilic portion orients toward the aqueous phase. Such compounds include, but are not limited to, phospholipids, aminolipids, and sphingolipids. Representative phospholipids include sphingomyelin, phosphatidylcholine, phosphatidylethanolamine, phosphatidylserine, phosphatidylinositol, phosphatidic acid, palmitoyloleoyl phosphatdylcholine, lysophosphatidylcholine, lysophosphatidylethanolamine, dipalmitoylphosphatidylcholine, dioleoylphosphatidylcholine, distearoylphosphatidylcholine, or dilinoleoylphosphatidylcholine. Other phosphorus-lacking compounds, such as sphingolipids, glycosphingolipid families, diacylglycerols, and β-acyloxyacids, can also be used. Additionally, such amphipathic lipids can be readily mixed with other lipids, such as triglycerides and sterols.

Liposomes that are pH-sensitive or negatively-charged entrap nucleic acid molecules rather than complex with them. Since both the nucleic acid molecules and the lipid are similarly charged, repulsion rather than complex formation occurs. Nevertheless, some nucleic acid molecules are entrapped within the aqueous interior of these liposomes. pH-sensitive liposomes have been used to deliver DNA encoding the thymidine kinase gene to cell monolayers in culture. Expression of the exogenous gene was detected in the target cells (Zhou et al., Journal of Controlled Release, 19, (1992) 269-274).

One major type of liposomal composition includes phospholipids other than naturally-derived phosphatidylcholine. Neutral liposome compositions, for example, can be formed from dimyristoyl phosphatidylcholine (DMPC) or dipalmitoyl phosphatidylcholine (DPPC). Anionic liposome compositions generally are formed from dimyristoyl phosphatidylglycerol, while anionic fusogenic liposomes are formed primarily from dioleoyl phosphatidylethanolamine (DOPE). Another type of liposomal composition is formed from phosphatidylcholine (PC) such as, for example, soybean PC, and egg PC. Another type is formed from mixtures of phospholipid and/or phosphatidylcholine and/or cholesterol.

Examples of other methods to introduce liposomes into cells in vitro and in vivo include U.S. Pat. No. 5,283,185; U.S. Pat. No. 5,171,678; WO 94/00569; WO 93/24640; WO 91/16024; Felgner, J. Biol. Chem. 269:2550, 1994; Nabel, Proc. Natl. Acad. Sci. 90:11307, 1993; Nabel, Human Gene Ther. 3:649, 1992; Gershon, Biochem. 32:7143, 1993; and Strauss EMBO J. 11:417, 1992.

Also suitable for inclusion in the liposome compostions of the present invention are programmable fusion lipids. Liposomes containing programmable fusion lipids have little tendency to fuse with cell membranes and deliver their payload until a given signal event occurs. This allows the liposome to distribute more evenly after injection into an organism or disease site before it starts fusing with cells. The signal event can be, for example, a change in pH, temperature, ionic environment, or time. In the latter case, a fusion delaying or “cloaking” component, such as an ATTA-lipid conjugate or a PEG-lipid conjugate, can simply exchange out of the liposome membrane over time. By the time the liposome is suitably distributed in the body, it has lost sufficient cloaking agent so as to be fusogenic. With other signal events, it is desirable to choose a signal that is associated with the disease site or target cell, such as increased temperature at a site of inflammation.

A liposome can also include a targeting moiety, e.g., a targeting moiety that is specific to a cell type or tissue. Targeting of liposomes with a surface coating of hydrophilic polymer chains, such as polyethylene glycol (PEG) chains, for targeting has been proposed (Allen, et al., Biochimica et Biophysica Acta 1237: 99-108 (1995); DeFrees, et al., Journal of the American Chemistry Society 118: 6101-6104 (1996); Blume, et al., Biochimica et Biophysica Acta 1149: 180-184 (1993); Klibanov, et al., Journal of Liposome Research 2: 321-334 (1992); U.S. Pat. No. 5,013,556; Zalipsky, Bioconjugate Chemistry 4: 296-299 (1993); Zalipsky, FEBS Letters 353: 71-74 (1994); Zalipsky, in Stealth Liposomes Chapter 9 (Lasic and Martin, Eds) CRC Press, Boca Raton Fla. (1995). Other targeting moieties, such as ligands, cell surface receptors, glycoproteins, vitamins (e.g., riboflavin), aptamers and monoclonal antibodies, can also be used. The targeting moieties can include the entire protein or fragments thereof. Targeting mechanisms generally require that the targeting agents be positioned on the surface of the liposome in such a manner that the targeting moiety is available for interaction with the target, for example, a cell surface receptor.

In one approach, a targeting moiety, such as receptor binding ligand, for targeting the liposome is linked to the lipids forming the liposome. In another approach, the targeting moiety is attached to the distal ends of the PEG chains forming the hydrophilic polymer coating (Klibanov, et al., Journal of Liposome Research 2: 321-334 (1992); Kirpotin et al., FEBS Letters 388: 115-118 (1996)). A variety of different targeting agents and methods are known and available in the art, including those described, e.g., in Sapra, P. and Allen, T M, Prog. Lipid Res. 42(5):439-62 (2003); and Abra, R M et al., J. Liposome Res. 12:1-3, (2002).

In one embodiment, cationic liposomes are used. Cationic liposomes possess the advantage of being able to fuse to the cell membrane. Non-cationic liposomes, although not able to fuse as efficiently with the plasma membrane, are taken up by macrophages in vivo and can be used to deliver siRNAs to macrophages.

Further advantages of liposomes include: liposomes obtained from natural phospholipids are biocompatible and biodegradable; liposomes can incorporate a wide range of water and lipid soluble drugs; liposomes can protect encapsulated siRNAs in their internal compartments from metabolism and degradation (Rosoff, in “Pharmaceutical Dosage Forms,” Lieberman, Rieger and Banker (Eds.), 1988, volume 1, p. 245) Important considerations in the preparation of liposome formulations are the lipid surface charge, vesicle size and the aqueous volume of the liposomes.

A positively charged synthetic cationic lipid, N-[1-(2,3-dioleyloxy)propyl]-N,N,N-trimethylammonium chloride (DOTMA) can be used to form small liposomes that interact spontaneously with nucleic acid to form lipid-nucleic acid complexes which are capable of fusing with the negatively charged lipids of the cell membranes of tissue culture cells, resulting in delivery of siRNA (see, e.g., Felgner, P. L. et al., Proc. Natl. Acad. Sci., USA 8:7413-7417, 1987 and U.S. Pat. No. 4,897,355 for a description of DOTMA and its use with DNA).

A DOTMA analogue, 1,2-bis(oleoyloxy)-3-(trimethylammonia)propane (DOTAP) can be used in combination with a phospholipid to form DNA-complexing vesicles. Lipofectin™ Bethesda Research Laboratories, Gaithersburg, Md.) is an effective agent for the delivery of highly anionic nucleic acids into living tissue culture cells that comprise positively charged DOTMA liposomes which interact spontaneously with negatively charged polynucleotides to form complexes. When enough positively charged liposomes are used, the net charge on the resulting complexes is also positive. Positively charged complexes prepared in this way spontaneously attach to negatively charged cell surfaces, fuse with the plasma membrane, and efficiently deliver functional nucleic acids into, for example, tissue culture cells. Another commercially available cationic lipid, 1,2-bis(oleoyloxy)-3,3-(trimethylammonia)propane (“DOTAP”) (Boehringer Mannheim, Indianapolis, Ind.) differs from DOTMA in that the oleoyl moieties are linked by ester, rather than ether linkages.

Other reported cationic lipid compounds include those that have been conjugated to a variety of moieties including, for example, carboxyspermine which has been conjugated to one of two types of lipids and includes compounds such as 5-carboxyspermylglycine dioctaoleoylamide (“DOGS”) (Transfectam™, Promega, Madison, Wis.) and dipalmitoylphosphatidylethanolamine 5-carboxyspermyl-amide (“DPPES”) (see, e.g., U.S. Pat. No. 5,171,678).

Another cationic lipid conjugate includes derivatization of the lipid with cholesterol (“DC-Chol”) which has been formulated into liposomes in combination with DOPE (See, Gao, X. and Huang, L., Biochim. Biophys. Res. Commun. 179:280, 1991). Lipopolylysine, made by conjugating polylysine to DOPE, has been reported to be effective for transfection in the presence of serum (Zhou, X. et al., Biochim. Biophys. Acta 1065:8, 1991). For certain cell lines, these liposomes containing conjugated cationic lipids, are said to exhibit lower toxicity and provide more efficient transfection than the DOTMA-containing compositions. Other commercially available cationic lipid products include DMRIE and DMRIE-HP (Vical, La Jolla, Calif.) and Lipofectamine (DOSPA) (Life Technology, Inc., Gaithersburg, Md.). Other cationic lipids suitable for the delivery of oligonucleotides are described in WO 98/39359 and WO 96/37194.

Liposomal formulations are particularly suited for topical administration, liposomes present several advantages over other formulations. Such advantages include reduced side effects related to high systemic absorption of the administered drug, increased accumulation of the administered drug at the desired target, and the ability to administer siRNA, into the skin. In some implementations, liposomes are used for delivering siRNA to epidermal cells and also to enhance the penetration of siRNA into dermal tissues, e.g., into skin. For example, the liposomes can be applied topically. Topical delivery of drugs formulated as liposomes to the skin has been documented (see, e.g., Weiner et al., Journal of Drug Targeting, 1992, vol. 2,405-410 and du Plessis et al., Antiviral Research, 18, 1992, 259-265; Mannino, R. J. and Fould-Fogerite, S., Biotechniques 6:682-690, 1988; Itani, T. et al. Gene 56:267-276. 1987; Nicolau, C. et al. Meth. Enz. 149:157-176, 1987; Straubinger, R. M. and Papahadjopoulos, D. Meth. Enz. 101:512-527, 1983; Wang, C. Y. and Huang, L., Proc. Natl. Acad. Sci. USA 84:7851-7855, 1987).

Non-ionic liposomal systems have also been examined to determine their utility in the delivery of drugs to the skin, in particular systems comprising non-ionic surfactant and cholesterol. Non-ionic liposomal formulations comprising Novasome I (glyceryl dilaurate/cholesterol/polyoxyethylene-10-stearyl ether) and Novasome II (glyceryl distearate/cholesterol/polyoxyethylene-10-stearyl ether) were used to deliver a drug into the dermis of mouse skin. Such formulations with siRNA are useful for treating a dermatological disorder.

Liposomes that include siRNA can be made highly deformable. Such deformability can enable the liposomes to penetrate through pore that are smaller than the average radius of the liposome. For example, transfersomes are a type of deformable liposomes. Transferosomes can be made by adding surface edge activators, usually surfactants, to a standard liposomal composition. Transfersomes that include siRNA can be delivered, for example, subcutaneously by infection in order to deliver siRNA to keratinocytes in the skin. In order to cross intact mammalian skin, lipid vesicles must pass through a series of fine pores, each with a diameter less than 50 nm, under the influence of a suitable transdermal gradient. In addition, due to the lipid properties, these transferosomes can be self-optimizing (adaptive to the shape of pores, e.g., in the skin), self-repairing, and can frequently reach their targets without fragmenting, and often self-loading.

A liposome composition of the invention can be prepared by a variety of methods that are known in the art. See e.g., U.S. Pat. No. 4,235,871, No. 4,897,355 and No. 5,171,678; published PCT applications WO 96/14057 and WO 96/37194; Felgner, P. L. et al., Proc. Natl. Acad. Sci., USA (1987) 8:7413-7417, Bangham, et al. M. Mol. Biol. (1965) 23:238, Olson, et al. Biochim. Biophys. Acta (1979) 557:9, Szoka, et al. Proc. Natl. Acad. Sci. (1978) 75: 4194, Mayhew, et al. Biochim. Biophys. Acta (1984) 775:169, Kim, et al. Biochim. Biophys. Acta (1983) 728:339, and Fukunaga, et al. Endocrinol. (1984) 115:757.

For example, a liposome composition of the invention can be prepared by first dissolving the lipid components of a liposome in a detergent so that micelles are formed with the lipid component. The detergent can have a high critical micelle concentration and may be nonionic. Exemplary detergents include, but are not limited to, cholate, CHAPS, octylglucoside, deoxycholate and lauroyl sarcosine. The oligonucleotide preparation e.g., an emulsion, is then added to the micelles that include the lipid components. After condensation, the detergent is removed, e.g., by dialysis, to yield a liposome containing the oligonucleotide. If necessary a carrier compound that assists in condensation can be added during the condensation reaction, e.g., by controlled addition. For example, the carrier compound can be a polymer other than a nucleic acid (e.g., spermine or spermidine). To favor condensation, pH of the mixture can also be adjusted.

In another example, liposomes of the present invention may be prepared by diffusing a lipid derivatized with a hydrophilic polymer into preformed liposome, such as by exposing preformed liposomes to micelles composed of lipid-grafted polymers, at lipid concentrations corresponding to the final mole percent of derivatized lipid which is desired in the liposome. Liposomes containing a hydrophilic polymer can also be formed by homogenization, lipid-field hydration, or extrusion techniques, as are known in the art.

In another exemplary formulation procedure, the iRNA agent is first dispersed by sonication in a lysophosphatidylcholine or other low CMC surfactant (including polymer grafted lipids). The resulting micellar suspension of oligonucleotide is then used to rehydrate a dried lipid sample that contains a suitable mole percent of polymer-grafted lipid, or cholesterol. The lipid and active agent suspension is then formed into liposomes using extrusion techniques as are known in the art, and the resulting liposomes separated from the unencapsulated solution by standard column separation.

In one aspect of the present invention, the liposomes are prepared to have substantially homogeneous sizes in a selected size range. One effective sizing method involves extruding an aqueous suspension of the liposomes through a series of polycarbonate membranes having a selected uniform pore size; the pore size of the membrane will correspond roughly with the largest sizes of liposomes produced by extrusion through that membrane. See e.g., U.S. Pat. No. 4,737,323.

Micelles and other Membranous Formulations

Recently, the pharmaceutical industry introduced microemulsification technology to improve bioavailability of some lipophilic (water insoluble) pharmaceutical agents. Examples include Trimetrine (Dordunoo, S. K., et al., Drug Development and Industrial Pharmacy, 17(12), 1685-1713, 1991 and REV 5901 (Sheen, P. C., et al., J Pharm Sci 80(7), 712-714, 1991). Among other things, microemulsification provides enhanced bioavailability by preferentially directing absorption to the lymphatic system instead of the circulatory system, which thereby bypasses the liver, and prevents destruction of the compounds in the hepatobiliary circulation.

For ease of exposition the micelles and other formulations, compositions and methods in this section are discussed largely with regard to unmodified oligonucleotide compounds. It may be understood, however, that these micelles and other formulations, compositions and methods can be practiced with other oligonucleotide compounds, e.g., modified siRNA compounds, and such practice is within the invention. The siRNA compound, e.g., a double-stranded siRNA compound, or ssiRNA compound, (e.g., a precursor, e.g., a larger siRNA compound which can be processed into a ssiRNA compound, or a DNA which encodes an siRNA compound, e.g., a double-stranded siRNA compound, or ssiRNA compound, or precursor thereof)) composition can be provided as a micellar formulation. As defined herein, “micelles” are a particular type of molecular assembly in which amphipathic molecules are arranged in a spherical structure such that all hydrophobic portions on the molecules are directed inward, leaving the hydrophilic portions in contact with the surrounding aqueous phase. The converse arrangement exists if the environment is hydrophobic.

In one aspect of invention, the formulations contain micelles formed from a compound of the present invention and at least one amphiphilic carrier, in which the micelles have an average diameter of less than about 100 nm. More preferred embodiments provide micelles having an average diameter less than about 50 nm, and even more preferred embodiments provide micelles having an average diameter less than about 30 nm, or even less than about 20 nm.

While all suitable amphiphilic carriers are contemplated, the presently preferred carriers are generally those that have Generally-Recognized-as-Safe (GRAS) status, and that can both solubilize the compound of the present invention and microemulsify it at a later stage when the solution comes into a contact with a complex water phase (such as one found in human gastro-intestinal tract). Usually, amphiphilic ingredients that satisfy these requirements have HLB (hydrophilic to lipophilic balance) values of 2-20, and their structures contain straight chain aliphatic radicals in the range of C-6 to C-20. Examples are polyethylene-glycolized fatty glycerides and polyethylene glycols.

Exemplary amphiphilic carriers include, but are not limited to, lecithin, hyaluronic acid, pharmaceutically acceptable salts of hyaluronic acid, glycolic acid, lactic acid, chamomile extract, cucumber extract, oleic acid, linoleic acid, linolenic acid, monoolein, monooleates, monolaurates, borage oil, evening of primrose oil, menthol, trihydroxy oxo cholanyl glycine and pharmaceutically acceptable salts thereof, glycerin, polyglycerin, lysine, polylysine, triolein, polyoxyethylene ethers and analogues thereof, polidocanol alkyl ethers and analogues thereof, chenodeoxycholate, deoxycholate, and mixtures thereof.

Particularly preferred amphiphilic carriers are saturated and monounsaturated polyethyleneglycolyzed fatty acid glycerides, such as those obtained from fully or partially hydrogenated various vegetable oils. Such oils may advantageously consist of tri-. di- and mono-fatty acid glycerides and di- and mono-polyethyleneglycol esters of the corresponding fatty acids, with a particularly preferred fatty acid composition including capric acid 4-10, capric acid 3-9, lauric acid 40-50, myristic acid 14-24, palmitic acid 4-14 and stearic acid 5-15%. Another useful class of amphiphilic carriers includes partially esterified sorbitan and/or sorbitol, with saturated or mono-unsaturated fatty acids (SPAN-series) or corresponding ethoxylated analogs (TWEEN-series).

Commercially available amphiphilic carriers are particularly contemplated, including Gelucire-series, Labrafil, Labrasol, or Lauroglycol (all manufactured and distributed by Gattefos se Corporation, Saint Priest, France), PEG-mono-oleate, PEG-di-oleate, PEG-mono-laurate and di-laurate, Lecithin, Polysorbate 80, etc (produced and distributed by a number of companies in USA and worldwide).

A mixed micellar formulation suitable for delivery through transdermal membranes may be prepared by mixing an aqueous solution of the oligonucleotide composition, an alkali metal C₈ to C₂₂ alkyl sulphate, and a micelle forming compounds. Exemplary micelle forming compounds include lecithin, hyaluronic acid, pharmaceutically acceptable salts of hyaluronic acid, glycolic acid, lactic acid, chamomile extract, cucumber extract, oleic acid, linoleic acid, linolenic acid, monoolein, monooleates, monolaurates, borage oil, evening of primrose oil, menthol, trihydroxy oxo cholanyl glycine and pharmaceutically acceptable salts thereof, glycerin, polyglycerin, lysine, polylysine, triolein, polyoxyethylene ethers and analogues thereof, polidocanol alkyl ethers and analogues thereof, chenodeoxycholate, deoxycholate, and mixtures thereof. The micelle forming compounds may be added at the same time or after addition of the alkali metal alkyl sulphate. Mixed micelles will form with substantially any kind of mixing of the ingredients but vigorous mixing in order to provide smaller size micelles.

In one method a first micellar composition is prepared which contains the oligonucleotide composition and at least the alkali metal alkyl sulphate. The first micellar composition is then mixed with at least three micelle forming compounds to form a mixed micellar composition. In another method, the micellar composition is prepared by mixing the oligonucleotide composition, the alkali metal alkyl sulphate and at least one of the micelle forming compounds (e.g., the amphiphilic carrier), followed by addition of the remaining micelle forming compounds, with vigorous mixing.

Phenol and/or m-cresol may be added to the mixed micellar composition to stabilize the formulation and protect against bacterial growth. Alternatively, phenol and/or m-cresol may be added with the micelle forming ingredients. An isotonic agent such as glycerin may also be added after formation of the mixed micellar composition.

For delivery of the micellar formulation as a spray, the formulation can be put into an aerosol dispenser and the dispenser is charged with a propellant. The propellant, which is under pressure, is in liquid form in the dispenser. The ratios of the ingredients are adjusted so that the aqueous and propellant phases become one, i.e., there is one phase. If there are two phases, it is necessary to shake the dispenser prior to dispensing a portion of the contents, e.g., through a metered valve. The dispensed dose of pharmaceutical agent is propelled from the metered valve in a fine spray.

Propellants may include hydrogen-containing chlorofluorocarbons, hydrogen-containing fluorocarbons, dimethyl ether and diethyl ether. In certain embodiments, HFA 134a (1,1,1,2 tetrafluoroethane) may be used.

The specific concentrations of the essential ingredients can be determined by relatively straightforward experimentation. For absorption through the oral cavities, it is often desirable to increase, e.g., at least double or triple, the dosage for through injection or administration through the gastrointestinal tract.

### Emulsions

The oligonucleotides of the present invention may be prepared and formulated as emulsions. Emulsions are typically heterogenous systems of one liquid dispersed in another in the form of droplets (Idson, in Pharmaceutical Dosage Forms, Lieberman, Rieger and Banker (Eds.), 1988, Marcel Dekker, Inc., New York, N.Y., volume 1, p. 199; Rosoff, in Pharmaceutical Dosage Forms, Lieberman, Rieger and Banker (Eds.), 1988, Marcel Dekker, Inc., New York, N.Y., Volume 1, p. 245; Block in Pharmaceutical Dosage Forms, Lieberman, Rieger and Banker (Eds.), 1988, Marcel Dekker, Inc., New York, N.Y., volume 2, p. 335; Higuchi et al., in Remington's Pharmaceutical Sciences, Mack Publishing Co., Easton, Pa., 1985, p. 301). Emulsions are often biphasic systems comprising two immiscible liquid phases intimately mixed and dispersed with each other. In general, emulsions may be of either the water-in-oil (w/o) or the oil-in-water (o/w) variety. When an aqueous phase is finely divided into and dispersed as minute droplets into a bulk oily phase, the resulting composition is called a water-in-oil (w/o) emulsion. Alternatively, when an oily phase is finely divided into and dispersed as minute droplets into a bulk aqueous phase, the resulting composition is called an oil-in-water (o/w) emulsion. Emulsions may contain additional components in addition to the dispersed phases, and the active drug which may be present as a solution in either the aqueous phase, oily phase or itself as a separate phase. Pharmaceutical excipients such as emulsifiers, stabilizers, dyes, and anti-oxidants may also be present in emulsions as needed. Pharmaceutical emulsions may also be multiple emulsions that are comprised of more than two phases such as, for example, in the case of oil-in-water-in-oil (o/w/o) and water-in-oil-in-water (w/o/w) emulsions. Such complex formulations often provide certain advantages that simple binary emulsions do not. Multiple emulsions in which individual oil droplets of an o/w emulsion enclose small water droplets constitute a w/o/w emulsion Likewise a system of oil droplets enclosed in globules of water stabilized in an oily continuous phase provides an o/w/o emulsion.

Emulsions are characterized by little or no thermodynamic stability. Often, the dispersed or discontinuous phase of the emulsion is well dispersed into the external or continuous phase and maintained in this form through the means of emulsifiers or the viscosity of the formulation. Either of the phases of the emulsion may be a semisolid or a solid, as is the case of emulsion-style ointment bases and creams. Other means of stabilizing emulsions entail the use of emulsifiers that may be incorporated into either phase of the emulsion. Emulsifiers may broadly be classified into four categories: synthetic surfactants, naturally occurring emulsifiers, absorption bases, and finely dispersed solids (Idson, in Pharmaceutical Dosage Forms, Lieberman, Rieger and Banker (Eds.), 1988, Marcel Dekker, Inc., New York, N.Y., volume 1, p. 199).

Synthetic surfactants, also known as surface active agents, have found wide applicability in the formulation of emulsions and have been reviewed in the literature (Rieger, in Pharmaceutical Dosage Forms, Lieberman, Rieger and Banker (Eds.), 1988, Marcel Dekker, Inc., New York, N.Y., volume 1, p. 285; Idson, in Pharmaceutical Dosage Forms, Lieberman, Rieger and Banker (Eds.), Marcel Dekker, Inc., New York, N.Y., 1988, volume 1, p. 199). Surfactants are typically amphiphilic and comprise a hydrophilic and a hydrophobic portion. The ratio of the hydrophilic to the hydrophobic nature of the surfactant has been termed the hydrophile/lipophile balance (HLB) and is a valuable tool in categorizing and selecting surfactants in the preparation of formulations. Surfactants may be classified into different classes based on the nature of the hydrophilic group: nonionic, anionic, cationic and amphoteric (Rieger, in Pharmaceutical Dosage Forms, Lieberman, Rieger and Banker (Eds.), 1988, Marcel Dekker, Inc., New York, N.Y., volume 1, p. 285).

Naturally occurring emulsifiers used in emulsion formulations include lanolin, beeswax, phosphatides, lecithin and acacia. Absorption bases possess hydrophilic properties such that they can soak up water to form w/o emulsions yet retain their semisolid consistencies, such as anhydrous lanolin and hydrophilic petrolatum. Finely divided solids have also been used as good emulsifiers especially in combination with surfactants and in viscous preparations. These include polar inorganic solids, such as heavy metal hydroxides, nonswelling clays such as bentonite, attapulgite, hectorite, kaolin, montmorillonite, colloidal aluminum silicate and colloidal magnesium aluminum silicate, pigments and nonpolar solids such as carbon or glyceryl tristearate.

A large variety of non-emulsifying materials is also included in emulsion formulations and contributes to the properties of emulsions. These include fats, oils, waxes, fatty acids, fatty alcohols, fatty esters, humectants, hydrophilic colloids, preservatives and antioxidants (Block, in Pharmaceutical Dosage Forms, Lieberman, Rieger and Banker (Eds.), 1988, Marcel Dekker, Inc., New York, N.Y., volume 1, p. 335; Idson, in Pharmaceutical Dosage Forms, Lieberman, Rieger and Banker (Eds.), 1988, Marcel Dekker, Inc., New York, N.Y., volume 1, p. 199).

Hydrophilic colloids or hydrocolloids include naturally occurring gums and synthetic polymers such as polysaccharides (for example, acacia, agar, alginic acid, carrageenan, guar gum, karaya gum, and tragacanth), cellulose derivatives (for example, carboxymethylcellulose and carboxypropylcellulose), and synthetic polymers (for example, carbomers, cellulose ethers, and carboxyvinyl polymers). These disperse or swell in water to form colloidal solutions that stabilize emulsions by forming strong interfacial films around the dispersed-phase droplets and by increasing the viscosity of the external phase.

Since emulsions often contain a number of ingredients such as carbohydrates, proteins, sterols and phosphatides that may readily support the growth of microbes, these formulations often incorporate preservatives. Commonly used preservatives included in emulsion formulations include methyl paraben, propyl paraben, quaternary ammonium salts, benzalkonium chloride, esters of p-hydroxybenzoic acid, and boric acid. Antioxidants are also commonly added to emulsion formulations to prevent deterioration of the formulation. Antioxidants used may be free radical scavengers such as tocopherols, alkyl gallates, butylated hydroxyanisole, butylated hydroxytoluene, or reducing agents such as ascorbic acid and sodium metabisulfite, and antioxidant synergists such as citric acid, tartaric acid, and lecithin.

The application of emulsion formulations via dermatological, oral and parenteral routes and methods for their manufacture has been reviewed in the literature (Idson, in Pharmaceutical Dosage Forms, Lieberman, Rieger and Banker (Eds.), 1988, Marcel Dekker, Inc., New York, N.Y., volume 1, p. 199). Emulsion formulations for oral delivery have been very widely used because of ease of formulation, as well as efficacy from an absorption and bioavailability standpoint (Rosoff, in Pharmaceutical Dosage Forms, Lieberman, Rieger and Banker (Eds.), 1988, Marcel Dekker, Inc., New York, N.Y., volume 1, p. 245; Idson, in Pharmaceutical Dosage Forms, Lieberman, Rieger and Banker (Eds.), 1988, Marcel Dekker, Inc., New York, N.Y., volume 1, p. 199). Mineral-oil base laxatives, oil-soluble vitamins and high fat nutritive preparations are among the materials that have commonly been administered orally as o/w emulsions.

In one embodiment of the present invention, the compositions are formulated as microemulsions. A microemulsion may be defined as a system of water, oil and amphiphile which is a single optically isotropic and thermodynamically stable liquid solution (Rosoff, in Pharmaceutical Dosage Forms, Lieberman, Rieger and Banker (Eds.), 1988, Marcel Dekker, Inc., New York, N.Y., volume 1, p. 245). Typically microemulsions are systems that are prepared by first dispersing an oil in an aqueous surfactant solution and then adding a sufficient amount of a fourth component, generally an intermediate chain-length alcohol to form a transparent system. Therefore, microemulsions have also been described as thermodynamically stable, isotropically clear dispersions of two immiscible liquids that are stabilized by interfacial films of surface-active molecules (Leung and Shah, in: Controlled Release of Drugs: Polymers and Aggregate Systems, Rosoff, M., Ed., 1989, VCH Publishers, New York, pages 185-215). Microemulsions commonly are prepared via a combination of three to five components that include oil, water, surfactant, cosurfactant and electrolyte. Whether the microemulsion is of the water-in-oil (w/o) or an oil-in-water (o/w) type is dependent on the properties of the oil and surfactant used and on the structure and geometric packing of the polar heads and hydrocarbon tails of the surfactant molecules (Schott, in Remington's Pharmaceutical Sciences, Mack Publishing Co., Easton, Pa., 1985, p. 271).

The phenomenological approach utilizing phase diagrams has been extensively studied and has yielded a comprehensive knowledge, to one skilled in the art, of how to formulate microemulsions (Rosoff, in Pharmaceutical Dosage Forms, Lieberman, Rieger and Banker (Eds.), 1988, Marcel Dekker, Inc., New York, N.Y., volume 1, p. 245; Block, in Pharmaceutical Dosage Forms, Lieberman, Rieger and Banker (Eds.), 1988, Marcel Dekker, Inc., New York, N.Y., volume 1, p. 335). Compared to conventional emulsions, microemulsions offer the advantage of solubilizing water-insoluble drugs in a formulation of thermodynamically stable droplets that are formed spontaneously.

Surfactants used in the preparation of microemulsions include, but are not limited to, ionic surfactants, non-ionic surfactants, Brij 96, polyoxyethylene oleyl ethers, polyglycerol fatty acid esters, tetraglycerol monolaurate (ML310), tetraglycerol monooleate (M0310), hexaglycerol monooleate (PO310), hexaglycerol pentaoleate (PO500), decaglycerol monocaprate (MCA750), decaglycerol monooleate (M0750), decaglycerol sequioleate (SO750), decaglycerol decaoleate (DAO750), alone or in combination with cosurfactants. The cosurfactant, usually a short-chain alcohol such as ethanol, 1-propanol, and 1-butanol, serves to increase the interfacial fluidity by penetrating into the surfactant film and consequently creating a disordered film because of the void space generated among surfactant molecules. Microemulsions may, however, be prepared without the use of cosurfactants and alcohol-free self-emulsifying microemulsion systems are known in the art. The aqueous phase may typically be, but is not limited to, water, an aqueous solution of the drug, glycerol, PEG300, PEG400, polyglycerols, propylene glycols, and derivatives of ethylene glycol. The oil phase may include, but is not limited to, materials such as Captex 300, Captex 355, Capmul MCM, fatty acid esters, medium chain (C8-C12) mono, di, and tri-glycerides, polyoxyethylated glyceryl fatty acid esters, fatty alcohols, polyglycolized glycerides, saturated polyglycolized C8-C10 glycerides, vegetable oils and silicone oil.

Microemulsions are particularly of interest from the standpoint of drug solubilization and the enhanced absorption of drugs. Lipid based microemulsions (both o/w and w/o) have been proposed to enhance the oral bioavailability of drugs, including peptides (Constantinides et al., Pharmaceutical Research, 1994, 11, 1385-1390; Ritschel, Meth. Find. Exp. Clin. Pharmacol., 1993, 13, 205). Microemulsions afford advantages of improved drug solubilization, protection of drug from enzymatic hydrolysis, possible enhancement of drug absorption due to surfactant-induced alterations in membrane fluidity and permeability, ease of preparation, ease of oral administration over solid dosage forms, improved clinical potency, and decreased toxicity (Constantinides et al., Pharmaceutical Research, 1994, 11, 1385; Ho et al., J. Pharm. Sci., 1996, 85, 138-143). Often microemulsions may form spontaneously when their components are brought together at ambient temperature. This may be particularly advantageous when formulating thermolabile drugs, peptides or dsRNAs. Microemulsions have also been effective in the transdermal delivery of active components in both cosmetic and pharmaceutical applications. It is expected that the microemulsion compositions and formulations of the present invention will facilitate the increased systemic absorption of dsRNAs and nucleic acids from the gastrointestinal tract, as well as improve the local cellular uptake of dsRNAs and nucleic acids.

Microemulsions of the present invention may also contain additional components and additives such as sorbitan monostearate (Grill 3), Labrasol, and penetration enhancers to improve the properties of the formulation and to enhance the absorption of the dsRNAs and nucleic acids of the present invention. Penetration enhancers used in the microemulsions of the present invention may be classified as belonging to one of five broad categories—surfactants, fatty acids, bile salts, chelating agents, and non-chelating non-surfactants (Lee et al., Critical Reviews in Therapeutic Drug Carrier Systems, 1991, p. 92). Each of these classes has been discussed above.

### Particles

For ease of exposition the particles, formulations, compositions and methods in this section are discussed largely with regard to modified oligonucleotide compounds. It may be understood, however, that these particles, formulations, compositions and methods can be practiced with other oligonucleotide compounds, e.g., unmodified siRNA compounds, and such practice is within the invention. In another embodiment, an siRNA compound, e.g., a double-stranded siRNA compound, or ssiRNA compound, (e.g., a precursor, e.g., a larger siRNA compound which can be processed into a ssiRNA compound, or a DNA which encodes an siRNA compound, e.g., a double-stranded siRNA compound, or ssiRNA compound, or precursor thereof) preparations may be incorporated into a particle, e.g., a microparticle. Microparticles can be produced by spray-drying, but may also be produced by other methods including lyophilization, evaporation, fluid bed drying, vacuum drying, or a combination of these techniques. See below for further description.

Sustained-Release Formulations. An siRNA compound, e.g., a double-stranded siRNA compound, or ssiRNA compound, (e.g., a precursor, e.g., a larger siRNA compound which can be processed into a ssiRNA compound, or a DNA which encodes an siRNA compound, e.g., a double-stranded siRNA compound, or ssiRNA compound, or precursor thereof) described herein can be formulated for controlled, e.g., slow release. Controlled release can be achieved by disposing the siRNA within a structure or substance which impedes its release. E.g., siRNA can be disposed within a porous matrix or in an erodable matrix, either of which allow release of the siRNA over a period of time.

Polymeric particles, e.g., polymeric in microparticles can be used as a sustained-release reservoir of siRNA that is taken up by cells only released from the microparticle through biodegradation. The polymeric particles in this embodiment should therefore be large enough to preclude phagocytosis (e.g., larger than 10 μm or larger than 20 μm). Such particles can be produced by the same methods to make smaller particles, but with less vigorous mixing of the first and second emulsions. That is to say, a lower homogenization speed, vortex mixing speed, or sonication setting can be used to obtain particles having a diameter around 100 μm rather than 10 μm. The time of mixing also can be altered.

Larger microparticles can be formulated as a suspension, a powder, or an implantable solid, to be delivered by intramuscular, subcutaneous, intradermal, intravenous, or intraperitoneal injection; via inhalation (intranasal or intrapulmonary); orally; or by implantation. These particles are useful for delivery of any siRNA when slow release over a relatively long term is desired. The rate of degradation, and consequently of release, varies with the polymeric formulation.

Microparticles may include pores, voids, hollows, defects or other interstitial spaces that allow the fluid suspension medium to freely permeate or perfuse the particulate boundary. For example, the perforated microstructures can be used to form hollow, porous spray dried microspheres.

Polymeric particles containing siRNA (e.g., a siRNA) can be made using a double emulsion technique, for instance. First, the polymer is dissolved in an organic solvent. A polymer may be polylactic-co-glycolic acid (PLGA), with a lactic/glycolic acid weight ratio of 65:35, 50:50, or 75:25. Next, a sample of nucleic acid suspended in aqueous solution is added to the polymer solution and the two solutions are mixed to form a first emulsion. The solutions can be mixed by vortexing or shaking, and in the mixture can be sonicated. Any method by which the nucleic acid receives the least amount of damage in the form of nicking, shearing, or degradation, while still allowing the formation of an appropriate emulsion is possible. For example, acceptable results can be obtained with a Vibra-cell model VC-250 sonicator with a ⅛″ microtip probe, at setting No. 3.

### Lipid Particles

It has been shown that cholesterol-conjugated siRNAs bind to HDL and LDL lipoprotein particles which mediate cellular uptake upon binding to their respective receptors. Both high-density lipoproteins (HDL) and low density lipoproteins (LDL) play a critical role in cholesterol transport. HDL directs siRNA delivery into liver, gut, kidney and steroidogenic organs, whereas LDL targets siRNA primarily to liver (Wolfrum et al. Nature Biotechnology Vol. 25 (2007)). Thus in one aspect the invention provides formulated lipid particles (FLiPs) comprising (a) an oligonucleotide of the invention, e.g., antisense, antagomir, supermir, antimir, miRNA mimic, U1 adaptor, aptamer, ribozyme and an iRNA agent, where said oligonucleotide has been conjugated to a lipophile and (b) at least one lipid component, for example an emulsion, liposome, isolated lipoprotein, reconstituted lipoprotein or phospholipid, to which the conjugated oligonucleotide has been aggregated, admixed or associated.

The stoichiometry of oligonucleotide to the lipid component may be 1:1. Alternatively the stoichiometry may be 1:many, many:1 or many:many, where many is greater than 2.

The FLiP may comprise triacylglycerol, phospholipids, glycerol and one or several lipid-binding proteins aggregated, admixed or associated via a lipophilic linker molecule with a single- or double-stranded oligonucleotide, wherein said FLiP has an affinity to heart, lung and/or muscle tissue. Surprisingly, it has been found that due to said one or several lipid-binding proteins in combination with the above mentioned lipids, the affinity to heart, lung and/or muscle tissue is very specific. These FLiPs may therefore serve as carrier for oligonucleotides. Due to their affinity to heart, lung and muscle cells, they may specifically transport the oligonucleotides to these tissues. Therefore, the FLiPs according to the present invention may be used for many severe heart, lung and muscle diseases, for example myocarditis, ischemic heart disease, myopathies, cardiomyopathies, metabolic diseases, rhabdomyosarcomas.

One suitable lipid component for FLiP is Intralipid. Intralipid® is a brand name for the first safe fat emulsion for human use. Intralipid® 20% (a 20% intravenous fat emulsion) is a sterile, non-pyrogenic fat emulsion prepared for intravenous administration as a source of calories and essential fatty acids. It is made up of 20% soybean oil, 1.2% egg yolk phospholipids, 2.25% glycerin, and water for injection. Intralipid® 10% is made up of 10% soybean oil, 1.2% egg yolk phospholipids, 2.25% glycerin, and water for injection. It is further within the present invention that other suitable oils, such as safflower oil, may serve to produce the lipid component of the FLiP.

In one embodiment of the invention is a FLiP comprising a lipid particle comprising 15-25% triacylglycerol, about 1-2% phospholipids and 2-3% glycerol, and one or several lipid-binding proteins.

In another embodiment of the invention the lipid particle comprises about 20% triacylglycerol, about 1.2% phospholipids and about 2.25% glycerol, which corresponds to the total composition of Intralipid, and one or several lipid-binding proteins.

Another suitable lipid component for FLiPs is lipoproteins, for example isolated lipoproteins or more preferably reconstituted lipoprotieins. Liporoteins are particles that contain both proteins and lipids. The lipids or their derivatives may be covalently or non-covalently bound to the proteins. Exemplary lipoproteins include chylomicrons, VLDL (Very Low Density Lipoproteins), IDL (Intermediate Density Lipoproteins), LDL (Low Density Lipoproteins) and HDL (High Density Lipoproteins).

Methods of producing reconstituted lipoproteins have been described in scientific literature, for example see A. Jones, Experimental Lung Res. 6, 255-270 (1984), U.S. Pat. No. 4,643,988 and No. 5128318, PCT publication WO87/02062, Canadian patent No. 2,138,925. Other methods of producing reconstituted lipoproteins, especially for apolipoproteins A-I, A-II, A-IV, apoC and apoE have been described in A. Jonas, Methods in Enzymology 128, 553-582 (1986) and G. Franceschini et al. J. Biol. Chem., 260(30), 16321-25 (1985).

The most frequently used lipid for reconstitution is phosphatidyl choline, extracted either from eggs or soybeans. Other phospholipids are also used, also lipids such as triglycerides or cholesterol. For reconstitution the lipids are first dissolved in an organic solvent, which is subsequently evaporated under nitrogen. In this method the lipid is bound in a thin film to a glass wall. Afterwards the apolipoproteins and a detergent, normally sodium cholate, are added and mixed. The added sodium cholate causes a dispersion of the lipid. After a suitable incubation period, the mixture is dialyzed against large quantities of buffer for a longer period of time; the sodium cholate is thereby removed for the most part, and at the same time lipids and apolipoproteins spontaneously form themselves into lipoproteins or so-called reconstituted lipoproteins. As alternatives to dialysis, hydrophobic adsorbents are available which can adsorb detergents (Bio-Beads SM-2, Bio Rad; Amberlite XAD-2, Rohm & Haas) (E. A. Bonomo, J. B. Swaney, J. Lipid Res., 29, 380-384 (1988)), or the detergent can be removed by means of gel chromatography (Sephadex G-25, Pharmacia). Lipoproteins can also be produced without detergents, for example through incubation of an aqueous suspension of a suitable lipid with apolipoproteins, the addition of lipid which was dissolved in an organic solvent, to apolipoproteins, with or without additional heating of this mixture, or through treatment of an apoA-I-lipid-mixture with ultrasound. With these methods, starting, for example, with apoA-I and phosphatidyl choline, disk-shaped particles can be obtained which correspond to lipoproteins in their nascent state. Normally, following the incubation, unbound apolipoproteins and free lipid are separated by means of centrifugation or gel chromatography in order to isolate the homogeneous, reconstituted lipoproteins particles.

Phospholipids used for reconstituted lipoproteins can be of natural origin, such as egg yolk or soybean phospholipids, or synthetic or semisynthetic origin. The phospholipids can be partially purified or fractionated to comprise pure fractions or mixtures of phosphatidyl cholines, phosphatidyl ethanolamines, phosphatidyl inositols, phosphatidic acids, phosphatidyl serines, sphingomyelin or phosphatidyl glycerols. According to specific embodiments of the present invention it is preferred to select phospholipids with defined fatty acid radicals, such as dimyristoyl phosphatidyl choline (DMPC), dioleoylphosphatidylethanolamine (DOPE), palmitoyloleoylphosphatidylcholine (POPC), egg phosphatidylcholine (EPC), distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), -phosphatidylethanolamine (POPE), dioleoyl-phosphatidylethanolamine 4-(N-maleimidomethyl)-cyclohexane-1-carboxylate (DOPE-mal), and combinations thereof, and the like phosphatidyl cholines with defined acyl groups selected from naturally occurring fatty acids, generally having 8 to 22 carbon atoms. According to a specific embodiment of the present invention phosphatidyl cholines having only saturated fatty acid residues between 14 and 18 carbon atoms are preferred, and of those dipalmitoyl phosphatidyl choline is especially preferred.

Other phospholipids suitable for reconstitution with lipoproteins include, e.g., phosphatidylcholine, phosphatidylglycerol, lecithin, b, g-dipalmitoyl-a-lecithin, sphingomyelin, phosphatidylserine, phosphatidic acid, N-(2,3-di(9-(Z)-octadecenyloxy))-prop-1-yl-N,N,N-trimethylammonium chloride, phosphatidylethanolamine, lysolecithin, lysophosphatidylethanolamine, phosphatidylinositol, cephalin, cardiolipin, cerebrosides, dicetylphosphate, dioleoylphosphatidylcholine, dipalmitoylphosphatidylcholine, dipalmitoylphosphatidylglycerol, dioleoylphosphatidylglycerol, palmitoyl-oleoyl-phosphatidylcholine, di-stearoyl-phosphatidylcholine, stearoyl-palmitoyl-phosphatidylcholine, di-palmitoyl-phosphatidylethanolamine, di-stearoyl-phosphatidylethanol amine, di-myrstoyl-phosphatidylserine, di-oleyl-phosphatidylcholine, and the like. Non-phosphorus containing lipids may also be used in the liposomes of the compositions of the present invention. These include, e.g., stearylamine, docecylamine, acetyl palmitate, fatty acid amides, and the like.

Besides the phospholipids, the lipoprotein may comprise, in various amounts at least one nonpolar component which can be selected among pharmaceutical acceptable oils (triglycerides) exemplified by the commonly employed vegetabilic oils such as soybean oil, safflower oil, olive oil, sesame oil, borage oil, castor oil and cottonseed oil or oils from other sources like mineral oils or marine oils including hydrogenated and/or fractionated triglycerides from such sources. Also medium chain triglycerides (MCT-oils, e.g. Miglyol®), and various synthetic or semisynthetic mono-, di- or triglycerides, such as the defined nonpolar lipids disclosed in WO 92/05571 may be used in the present invention as well as acetylated monoglycerides, or alkyl esters of fatty acids, such isopropyl myristate, ethyl oleate (see EP 0 353 267) or fatty acid alcohols, such as oleyl alcohol, cetyl alcohol or various nonpolar derivatives of cholesterol, such as cholesterol esters.

One or more complementary surface active agent can be added to the reconstituted lipoproteins, for example as complements to the characteristics of amphiphilic agent or to improve its lipid particle stabilizing capacity or enable an improved solubilization of the protein. Such complementary agents can be pharmaceutically acceptable non-ionic surfactants which preferably are alkylene oxide derivatives of an organic compound which contains one or more hydroxylic groups. For example ethoxylated and/or propoxylated alcohol or ester compounds or mixtures thereof are commonly available and are well known as such complements to those skilled in the art. Examples of such compounds are esters of sorbitol and fatty acids, such as sorbitan monopalmitate or sorbitan monopalmitate, oily sucrose esters, polyoxyethylene sorbitane fatty acid esters, polyoxyethylene sorbitol fatty acid esters, polyoxyethylene fatty acid esters, polyoxyethylene alkyl ethers, polyoxyethylene sterol ethers, polyoxyethylene-polypropoxy alkyl ethers, block polymers and cethyl ether, as well as polyoxyethylene castor oil or hydrogenated castor oil derivatives and polyglycerine fatty acid esters. Suitable non-ionic surfactants, include, but are not limited to various grades of Pluronic®, Poloxamer®, Span®, Tween®, Polysorbate®, Tyloxapol®, Emulphor® or Cremophor® and the like. The complementary surface active agents may also be of an ionic nature, such as bile duct agents, cholic acid or deoxycholic their salts and derivatives or free fatty acids, such as oleic acid, linoleic acid and others. Other ionic surface active agents are found among cationic lipids like C10-C24: alkylamines or alkanolamine and cationic cholesterol esters.

In the final FLiP, the oligonucleotide component is aggregated, associated or admixed with the lipid components via a lipophilic moiety. This aggregation, association or admixture may be at the surface of the final FLiP formulation. Alternatively, some integration of any of a portion or all of the lipophilic moiety may occur, extending into the lipid particle. Any lipophilic linker molecule that is able to bind oligonucleotides to lipids can be chosen. Examples include pyrrolidine and hydroxyprolinol.

The process for making the lipid particles comprises the steps of:

mixing a lipid components with one or several lipophile (e.g. cholesterol) conjugated oligonucleotides that may be chemically modified;

fractionating this mixture; and

selecting the fraction with particles of 30-50 nm, preferably of about 40 nm in size.

Alternatively, the FLiP can be made by first isolating the lipid particles comprising triacylglycerol, phospholipids, glycerol and one or several lipid-binding proteins and then mixing the isolated particles with >2-fold molar excess of lipophile (e.g. cholesterol) conjugated oligonucleotide. The steps of fractionating and selecting the particles are deleted by this alternative process for making the FLiPs.

Other pharmacologically acceptable components can be added to the FLiPs when desired, such as antioxidants (exemplified by alpha-tocopherol) and solubilization adjuvants (exemplified by benzylalcohol).

### Release Modifiers

The release characteristics of a formulation of the present invention depend on the encapsulating material, the concentration of encapsulated drug, and the presence of release modifiers. For example, release can be manipulated to be pH dependent, for example, using a pH sensitive coating that releases only at a low pH, as in the stomach, or a higher pH, as in the intestine. An enteric coating can be used to prevent release from occurring until after passage through the stomach. Multiple coatings or mixtures of cyanamide encapsulated in different materials can be used to obtain an initial release in the stomach, followed by later release in the intestine. Release can also be manipulated by inclusion of salts or pore forming agents, which can increase water uptake or release of drug by diffusion from the capsule. Excipients which modify the solubility of the drug can also be used to control the release rate. Agents which enhance degradation of the matrix or release from the matrix can also be incorporated. They can be added to the drug, added as a separate phase (i.e., as particulates), or can be co-dissolved in the polymer phase depending on the compound. In all cases the amount should be between 0.1 and thirty percent (w/w polymer). Types of degradation enhancers include inorganic salts such as ammonium sulfate and ammonium chloride, organic acids such as citric acid, benzoic acid, and ascorbic acid, inorganic bases such as sodium carbonate, potassium carbonate, calcium carbonate, zinc carbonate, and zinc hydroxide, and organic bases such as protamine sulfate, spermine, choline, ethanolamine, diethanolamine, and triethanolamine and surfactants such as Tween® and Pluronic®. Pore forming agents which add microstructure to the matrices (i.e., water soluble compounds such as inorganic salts and sugars) are added as particulates. The range should be between one and thirty percent (w/w polymer).

Uptake can also be manipulated by altering residence time of the particles in the gut. This can be achieved, for example, by coating the particle with, or selecting as the encapsulating material, a mucosal adhesive polymer. Examples include most polymers with free carboxyl groups, such as chitosan, celluloses, and especially polyacrylates (as used herein, polyacrylates refers to polymers including acrylate groups and modified acrylate groups such as cyanoacrylates and methacrylates).

### Polymers

Hydrophilic polymers suitable for use in the formulations of the present invention are those which are readily water-soluble, can be covalently attached to a vesicle-forming lipid, and which are tolerated in vivo without toxic effects (i.e., are biocompatible). Suitable polymers include polyethylene glycol (PEG), polylactic (also termed polylactide), polyglycolic acid (also termed polyglycolide), a polylactic-polyglycolic acid copolymer, and polyvinyl alcohol. Preferred polymers are those having a molecular weight of from about 100 or 120 daltons up to about 5,000 or 10,000 daltons, and more preferably from about 300 daltons to about 5,000 daltons. In a particularly preferred embodiment, the polymer is polyethyleneglycol having a molecular weight of from about 100 to about 5,000 daltons, and more preferably having a molecular weight of from about 300 to about 5,000 daltons. In a particularly preferred embodiment, the polymer is polyethyleneglycol of 750 daltons (PEG(750)). Polymers may also be defined by the number of monomers therein; a preferred embodiment of the present invention utilizes polymers of at least about three monomers, such PEG polymers consisting of three monomers (approximately 150 daltons).

Other hydrophilic polymers which may be suitable for use in the present invention include polyvinylpyrrolidone, polymethoxazoline, polyethyloxazoline, polyhydroxypropyl methacrylamide, polymethacrylamide, polydimethylacrylamide, and derivatized celluloses such as hydroxymethylcellulose or hydroxyethylcellulose.

In certain embodiments, a formulation of the present invention comprises a biocompatible polymer selected from the group consisting of polyamides, polycarbonates, polyalkylenes, polymers of acrylic and methacrylic esters, polyvinyl polymers, polyglycolides, polysiloxanes, polyurethanes and co-polymers thereof, celluloses, polypropylene, polyethylenes, polystyrene, polymers of lactic acid and glycolic acid, polyanhydrides, poly(ortho)esters, poly(butic acid), poly(valeric acid), poly(lactide-co-caprolactone), polysaccharides, proteins, polyhyaluronic acids, polycyanoacrylates, and blends, mixtures, or copolymers thereof.

### Surfactants

The above discussed formulation may also include one or more surfactants. Surfactants find wide application in formulations such as emulsions (including microemulsions) and liposomes. The use of surfactants in drug products, formulations and in emulsions has been reviewed (Rieger, in “Pharmaceutical Dosage Forms,” Marcel Dekker, Inc., New York, N.Y., 1988, p. 285). Surfactants may be classified into different classes based on the nature of the hydrophilic group: nonionic, anionic, cationic and amphoteric (Rieger, in Pharmaceutical Dosage Forms, Lieberman, Rieger and Banker (Eds.), 1988, Marcel Dekker, Inc., New York, N.Y., volume 1, p. 285).

For ease of exposition the formulations, compositions and methods in this section are discussed largely with regard to unmodified oligonucleotide compounds. It may be understood, however, that these formulations, compositions and methods can be practiced with other oligonucleotide compounds, e.g., modified siRNA compounds, and such practice is within the invention. Surfactants find wide application in formulations such as emulsions (including microemulsions) and liposomes (see above). siRNA (or a precursor, e.g., a larger dsiRNA which can be processed into a siRNA, or a DNA which encodes a siRNA or precursor) compositions can include a surfactant. In one embodiment, the siRNA is formulated as an emulsion that includes a surfactant. The most common way of classifying and ranking the properties of the many different types of surfactants, both natural and synthetic, is by the use of the hydrophile/lipophile balance (HLB). The nature of the hydrophilic group provides the most useful means for categorizing the different surfactants used in formulations (Rieger, in “Pharmaceutical Dosage Forms,” Marcel Dekker, Inc., New York, N.Y., 1988, p. 285).

If the surfactant molecule is not ionized, it is classified as a nonionic surfactant. Nonionic surfactants find wide application in pharmaceutical products and are usable over a wide range of pH values. In general their HLB values range from 2 to about 18 depending on their structure. Nonionic surfactants include, but not limited to, nonionic esters such as ethylene glycol esters, propylene glycol esters, glyceryl esters, polyglyceryl esters, sorbitan esters, sucrose esters, and ethoxylated esters. Nonionic alkanolamides and ethers such as fatty alcohol ethoxylates, propoxylated alcohols, and ethoxylated/propoxylated block polymers are also included in this class. The polyoxyethylene surfactants are the most popular members of the nonionic surfactant class.

If the surfactant molecule carries a negative charge when it is dissolved or dispersed in water, the surfactant is classified as anionic. Anionic surfactants include, but not limited to, carboxylates such as soaps, acyl lactylates, acyl amides of amino acids, esters of sulfuric acid such as alkyl sulfates and ethoxylated alkyl sulfates, sulfonates such as alkyl benzene sulfonates, acyl isethionates, acyl taurates and sulfosuccinates, and phosphates. The most important members of the anionic surfactant class are the alkyl sulfates and the soaps.

If the surfactant molecule carries a positive charge when it is dissolved or dispersed in water, the surfactant is classified as cationic. Cationic surfactants include, but not limited to, quaternary ammonium salts and ethoxylated amines. The quaternary ammonium salts are the most used members of this class.

If the surfactant molecule has the ability to carry either a positive or negative charge, the surfactant is classified as amphoteric. Amphoteric surfactants include, but not limited to, acrylic acid derivatives, substituted alkylamides, N-alkylbetaines and phosphatides.

A surfactant may also be selected from any suitable aliphatic, cycloaliphatic or aromatic surfactant, including but not limited to biocompatible lysophosphatidylcholines (LPCs) of varying chain lengths (for example, from about C14 to about C20). Polymer-derivatized lipids such as PEG-lipids may also be utilized for micelle formation as they will act to inhibit micelle/membrane fusion, and as the addition of a polymer to surfactant molecules decreases the CMC of the surfactant and aids in micelle formation. Preferred are surfactants with CMCs in the micromolar range; higher CMC surfactants may be utilized to prepare micelles entrapped within liposomes of the present invention, however, micelle surfactant monomers could affect liposome bilayer stability and would be a factor in designing a liposome of a desired stability.

### Penetration Enhancers

In one embodiment, the formulations of the present invention employ various penetration enhancers to affect the efficient delivery of iRNA agents to the skin of animals. Most drugs are present in solution in both ionized and nonionized forms. However, usually only lipid soluble or lipophilic drugs readily cross cell membranes. It has been discovered that even non-lipophilic drugs may cross cell membranes if the membrane to be crossed is treated with a penetration enhancer. In addition to aiding the diffusion of non-lipophilic drugs across cell membranes, penetration enhancers also enhance the permeability of lipophilic drugs.

### Pharmaceutical Compositions

In another aspect, the present invention provides pharmaceutically acceptable compositions which comprise a therapeutically-effective amount of one or more of the compounds described above, e.g. an iRNA agent, formulated together with one or more pharmaceutically acceptable carriers (additives) and/or diluents. As described in detail below, the pharmaceutical compositions of the present invention may be specially formulated for administration in solid or liquid form, including those adapted for the following: (1) oral administration, for example, drenches (aqueous or non-aqueous solutions or suspensions), tablets, e.g., those targeted for buccal, sublingual, and systemic absorption, boluses, powders, granules, pastes for application to the tongue; (2) parenteral administration, for example, by subcutaneous, intramuscular, intravenous or epidural injection as, for example, a sterile solution or suspension, or sustained-release formulation; (3) topical application, for example, as a cream, ointment, or a controlled-release patch or spray applied to the skin; (4) intravaginally or intrarectally, for example, as a pessary, cream or foam; (5) sublingually; (6) ocularly; (7) transdermally; or (8) nasally.

The phrase “therapeutically-effective amount” as used herein means that amount of a compound, material, or composition comprising a compound of the present invention which is effective for producing some desired therapeutic effect in at least a sub-population of cells in an animal at a reasonable benefit/risk ratio applicable to any medical treatment.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically-acceptable carrier” as used herein means a pharmaceutically-acceptable material, composition or vehicle, such as a liquid or solid filler, diluent, excipient, manufacturing aid (e.g., lubricant, talc magnesium, calcium or zinc stearate, or steric acid), or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) lubricating agents, such as magnesium state, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids (23) serum component, such as serum albumin, HDL and LDL; and (22) other non-toxic compatible substances employed in pharmaceutical formulations.

As set out above, certain embodiments of the present compounds may contain a basic functional group, such as amino or alkylamino, and are, thus, capable of forming pharmaceutically-acceptable salts with pharmaceutically-acceptable acids. The term “pharmaceutically-acceptable salts” in this respect, refers to the relatively non-toxic, inorganic and organic acid addition salts of compounds of the present invention. These salts can be prepared in situ in the administration vehicle or the dosage form manufacturing process, or by separately reacting a purified compound of the invention in its free base form with a suitable organic or inorganic acid, and isolating the salt thus formed during subsequent purification. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, napthylate, mesylate, glucoheptonate, lactobionate, and laurylsulphonate salts and the like. (See, for example, Berge et al. (1977) “Pharmaceutical Salts”, J. Pharm. Sci. 66:1-19)

The pharmaceutically acceptable salts of the subject compounds include the conventional nontoxic salts or quaternary ammonium salts of the compounds, e.g., from non-toxic organic or inorganic acids. For example, such conventional nontoxic salts include those derived from inorganic acids such as hydrochloride, hydrobromic, sulfuric, sulfamic, phosphoric, nitric, and the like; and the salts prepared from organic acids such as acetic, propionic, succinic, glycolic, stearic, lactic, malic, tartaric, citric, ascorbic, palmitic, maleic, hydroxymaleic, phenylacetic, glutamic, benzoic, salicyclic, sulfanilic, 2-acetoxybenzoic, fumaric, toluenesulfonic, methanesulfonic, ethane disulfonic, oxalic, isothionic, and the like.

In other cases, the compounds of the present invention may contain one or more acidic functional groups and, thus, are capable of forming pharmaceutically-acceptable salts with pharmaceutically-acceptable bases. The term “pharmaceutically-acceptable salts” in these instances refers to the relatively non-toxic, inorganic and organic base addition salts of compounds of the present invention. These salts can likewise be prepared in situ in the administration vehicle or the dosage form manufacturing process, or by separately reacting the purified compound in its free acid form with a suitable base, such as the hydroxide, carbonate or bicarbonate of a pharmaceutically-acceptable metal cation, with ammonia, or with a pharmaceutically-acceptable organic primary, secondary or tertiary amine Representative alkali or alkaline earth salts include the lithium, sodium, potassium, calcium, magnesium, and aluminum salts and the like. Representative organic amines useful for the formation of base addition salts include ethylamine, diethylamine, ethylenediamine, ethanolamine, diethanolamine, piperazine and the like. (See, for example, Berge et al., supra)

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically-acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Formulations of the present invention include those suitable for oral, nasal, topical (including buccal and sublingual), rectal, vaginal and/or parenteral administration. The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient which can be combined with a carrier material to produce a single dosage form will generally be that amount of the compound which produces a therapeutic effect. Generally, out of one hundred percent, this amount will range from about 0.1 percent to about ninety-nine percent of active ingredient, preferably from about 5 percent to about 70 percent, most preferably from about 10 percent to about 30 percent.

In certain embodiments, a formulation of the present invention comprises an excipient selected from the group consisting of cyclodextrins, celluloses, liposomes, micelle forming agents, e.g., bile acids, and polymeric carriers, e.g., polyesters and polyanhydrides; and a compound of the present invention. In certain embodiments, an aforementioned formulation renders orally bioavailable a compound of the present invention.

Methods of preparing these formulations or compositions include the step of bringing into association a compound of the present invention with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Formulations of the invention suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient. A compound of the present invention may also be administered as a bolus, electuary or paste.

In solid dosage forms of the invention for oral administration (capsules, tablets, pills, dragees, powders, granules, trouches and the like), the active ingredient is mixed with one or more pharmaceutically-acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds and surfactants, such as poloxamer and sodium lauryl sulfate; (7) wetting agents, such as, for example, cetyl alcohol, glycerol monostearate, and non-ionic surfactants; (8) absorbents, such as kaolin and bentonite clay; (9) lubricants, such as talc, calcium stearate, magnesium stearate, solid polyethylene glycols, sodium lauryl sulfate, zinc stearate, sodium stearate, stearic acid, and mixtures thereof; (10) coloring agents; and (11) controlled release agents such as crospovidone or ethyl cellulose. In the case of capsules, tablets and pills, the pharmaceutical compositions may also comprise buffering agents. Solid compositions of a similar type may also be employed as fillers in soft and hard-shelled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions of the present invention, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be formulated for rapid release, e.g., freeze-dried. They may be sterilized by, for example, filtration through a bacteria-retaining filter, or by incorporating sterilizing agents in the form of sterile solid compositions which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner Examples of embedding compositions which can be used include polymeric substances and waxes. The active ingredient can also be in micro-encapsulated form, if appropriate, with one or more of the above-described excipients.

Liquid dosage forms for oral administration of the compounds of the invention include pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations of the pharmaceutical compositions of the invention for rectal or vaginal administration may be presented as a suppository, which may be prepared by mixing one or more compounds of the invention with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active compound.

Formulations of the present invention which are suitable for vaginal administration also include pessaries, tampons, creams, gels, pastes, foams or spray formulations containing such carriers as are known in the art to be appropriate.

Dosage forms for the topical or transdermal administration of a compound of this invention include powders, sprays, ointments, pastes, creams, lotions, gels, solutions, patches and inhalants. The active compound may be mixed under sterile conditions with a pharmaceutically-acceptable carrier, and with any preservatives, buffers, or propellants which may be required.

The ointments, pastes, creams and gels may contain, in addition to an active compound of this invention, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to a compound of this invention, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a compound of the present invention to the body. Such dosage forms can be made by dissolving or dispersing the compound in the proper medium. Absorption enhancers can also be used to increase the flux of the compound across the skin. The rate of such flux can be controlled by either providing a rate controlling membrane or dispersing the compound in a polymer matrix or gel.

Ophthalmic formulations, eye ointments, powders, solutions and the like, are also contemplated as being within the scope of this invention. Formulations for ocular administration can include mucomimetics such as hyaluronic acid, chondroitin sulfate, hydroxypropyl methylcellulose or poly(vinyl alcohol), preservatives such as sorbic acid, EDTA or benzylchronium chloride, and the usual quantities of diluents and/or carriers.

Pharmaceutical compositions of this invention suitable for parenteral administration comprise one or more compounds of the invention in combination with one or more pharmaceutically-acceptable sterile isotonic aqueous or nonaqueous solutions, dispersions, suspensions or emulsions, or sterile powders which may be reconstituted into sterile injectable solutions or dispersions just prior to use, which may contain sugars, alcohols, antioxidants, buffers, bacteriostats, solutes which render the formulation isotonic with the blood of the intended recipient or suspending or thickening agents.

Examples of suitable aqueous and nonaqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms upon the subject compounds may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution which, in turn, may depend upon crystal size and crystalline form. Alternatively, delayed absorption of a parenterally-administered drug form is accomplished by dissolving or suspending the drug in an oil vehicle.

Injectable depot forms are made by forming microencapsule matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissue.

When the compounds of the present invention are administered as pharmaceuticals, to humans and animals, they can be given per se or as a pharmaceutical composition containing, for example, 0.1 to 99% (more preferably, 10 to 30%) of active ingredient in combination with a pharmaceutically acceptable carrier.

The preparations of the present invention may be given orally, parenterally, topically, or rectally. They are of course given in forms suitable for each administration route. For example, they are administered in tablets or capsule form, by injection, inhalation, eye lotion, ointment, suppository, etc. administration by injection, infusion or inhalation; topical by lotion or ointment; and rectal by suppositories. Oral administrations are preferred.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticulare, subcapsular, subarachnoid, intraspinal and intrasternal injection and infusion.

The phrases “systemic administration,” “administered systemically,” “peripheral administration” and “administered peripherally” as used herein mean the administration of a compound, drug or other material other than directly into the central nervous system, such that it enters the patient's system and, thus, is subject to metabolism and other like processes, for example, subcutaneous administration.

These compounds may be administered to humans and other animals for therapy by any suitable route of administration, including orally, nasally, as by, for example, a spray, rectally, intravaginally, parenterally, intracisternally and topically, as by powders, ointments or drops, including buccally and sublingually.

Regardless of the route of administration selected, the compounds of the present invention, which may be used in a suitable hydrated form, and/or the pharmaceutical compositions of the present invention, are formulated into pharmaceutically-acceptable dosage forms by conventional methods known to those of skill in the art.

Actual dosage levels of the active ingredients in the pharmaceutical compositions of this invention may be varied so as to obtain an amount of the active ingredient which is effective to achieve the desired therapeutic response for a particular patient, composition, and mode of administration, without being toxic to the patient.

The selected dosage level will depend upon a variety of factors including the activity of the particular compound of the present invention employed, or the ester, salt or amide thereof, the route of administration, the time of administration, the rate of excretion or metabolism of the particular compound being employed, the rate and extent of absorption, the duration of the treatment, other drugs, compounds and/or materials used in combination with the particular compound employed, the age, sex, weight, condition, general health and prior medical history of the patient being treated, and like factors well known in the medical arts.

A physician or veterinarian having ordinary skill in the art can readily determine and prescribe the effective amount of the pharmaceutical composition required. For example, the physician or veterinarian could start doses of the compounds of the invention employed in the pharmaceutical composition at levels lower than that required in order to achieve the desired therapeutic effect and gradually increase the dosage until the desired effect is achieved.

In general, a suitable daily dose of a compound of the invention will be that amount of the compound which is the lowest dose effective to produce a therapeutic effect. Such an effective dose will generally depend upon the factors described above. Generally, oral, intravenous, intracerebroventricular and subcutaneous doses of the compounds of this invention for a patient, when used for the indicated analgesic effects, will range from about 0.0001 to about 100 mg per kilogram of body weight per day.

If desired, the effective daily dose of the active compound may be administered as two, three, four, five, six or more sub-doses administered separately at appropriate intervals throughout the day, optionally, in unit dosage forms. Preferred dosing is one administration per day.

While it is possible for a compound of the present invention to be administered alone, it is preferable to administer the compound as a pharmaceutical formulation (composition).

The compounds according to the invention may be formulated for administration in any convenient way for use in human or veterinary medicine, by analogy with other pharmaceuticals.

In another aspect, the present invention provides pharmaceutically acceptable compositions which comprise a therapeutically-effective amount of one or more of the subject compounds, as described above, formulated together with one or more pharmaceutically acceptable carriers (additives) and/or diluents. As described in detail below, the pharmaceutical compositions of the present invention may be specially formulated for administration in solid or liquid form, including those adapted for the following: (1) oral administration, for example, drenches (aqueous or non-aqueous solutions or suspensions), tablets, boluses, powders, granules, pastes for application to the tongue; (2) parenteral administration, for example, by subcutaneous, intramuscular or intravenous injection as, for example, a sterile solution or suspension; (3) topical application, for example, as a cream, ointment or spray applied to the skin, lungs, or mucous membranes; or (4) intravaginally or intrarectally, for example, as a pessary, cream or foam; (5) sublingually or buccally; (6) ocularly; (7) transdermally; or (8) nasally.

The term “treatment” is intended to encompass also prophylaxis, therapy and cure.

The patient receiving this treatment is any animal in need, including primates, in particular humans, and other mammals such as equines, cattle, swine and sheep; and poultry and pets in general.

The compound of the invention can be administered as such or in admixtures with pharmaceutically acceptable carriers and can also be administered in conjunction with antimicrobial agents such as penicillins, cephalosporins, aminoglycosides and glycopeptides. Conjunctive therapy, thus includes sequential, simultaneous and separate administration of the active compound in a way that the therapeutical effects of the first administered one is not entirely disappeared when the subsequent is administered.

The addition of the active compound of the invention to animal feed is preferably accomplished by preparing an appropriate feed premix containing the active compound in an effective amount and incorporating the premix into the complete ration.

Alternatively, an intermediate concentrate or feed supplement containing the active ingredient can be blended into the feed. The way in which such feed premixes and complete rations can be prepared and administered are described in reference books (such as “Applied Animal Nutrition”, W.H. Freedman and CO., San Francisco, U.S.A., 1969 or “Livestock Feeds and Feeding” O and B books, Corvallis, Ore., U.S.A., 1977).

### Spray Drying

An siRNA compound, e.g., a double-stranded siRNA compound, or ssiRNA compound, (e.g., a precursor, e.g., a larger siRNA compound which can be processed into a ssiRNA compound, or a DNA which encodes an siRNA compound, e.g., a double-stranded siRNA compound, or ssiRNA compound, or precursor thereof)) can be prepared by spray drying. Spray dried siRNA can be administered to a subject or be subjected to further formulation. A pharmaceutical composition of siRNA can be prepared by spray drying a homogeneous aqueous mixture that includes a siRNA under conditions sufficient to provide a dispersible powdered composition, e.g., a pharmaceutical composition. The material for spray drying can also include one or more of: a pharmaceutically acceptable excipient, or a dispersibility-enhancing amount of a physiologically acceptable, water-soluble protein. The spray-dried product can be a dispersible powder that includes the siRNA.

Spray drying is a process that converts a liquid or slurry material to a dried particulate form. Spray drying can be used to provide powdered material for various administrative routes including inhalation. See, for example, M. Sacchetti and M. M. Van Oort in: Inhalation Aerosols: Physical and Biological Basis for Therapy, A. J. Hickey, ed. Marcel Dekkar, New York, 1996.

Spray drying can include atomizing a solution, emulsion, or suspension to form a fine mist of droplets and drying the droplets. The mist can be projected into a drying chamber (e.g., a vessel, tank, tubing, or coil) where it contacts a drying gas. The mist can include solid or liquid pore forming agents. The solvent and pore forming agents evaporate from the droplets into the drying gas to solidify the droplets, simultaneously forming pores throughout the solid. The solid (typically in a powder, particulate form) then is separated from the drying gas and collected.

Spray drying includes bringing together a highly dispersed liquid, and a sufficient volume of air (e.g., hot air) to produce evaporation and drying of the liquid droplets. The preparation to be spray dried can be any solution, course suspension, slurry, colloidal dispersion, or paste that may be atomized using the selected spray drying apparatus. Typically, the feed is sprayed into a current of warm filtered air that evaporates the solvent and conveys the dried product to a collector. The spent air is then exhausted with the solvent.

Several different types of apparatus may be used to provide the desired product. For example, commercial spray dryers manufactured by Buchi Ltd. or Niro Corp. can effectively produce particles of desired size.

Spray-dried powdered particles can be approximately spherical in shape, nearly uniform in size and frequently hollow. There may be some degree of irregularity in shape depending upon the incorporated medicament and the spray drying conditions. In many instances the dispersion stability of spray-dried microspheres appears to be more effective if an inflating agent (or blowing agent) is used in their production. Certain embodiments may comprise an emulsion with an inflating agent as the disperse or continuous phase (the other phase being aqueous in nature). An inflating agent may be dispersed with a surfactant solution, using, for instance, a commercially available microfluidizer at a pressure of about 5000 to 15,000 psi. This process forms an emulsion, which may be stabilized by an incorporated surfactant, typically comprising submicron droplets of water immiscible blowing agent dispersed in an aqueous continuous phase. The formation of such dispersions using this and other techniques are common and well known to those in the art. The blowing agent may be a fluorinated compound (e.g., perfluorohexane, perfluorooctyl bromide, perfluorodecalin, perfluorobutyl ethane) which vaporizes during the spray-drying process, leaving behind generally hollow, porous aerodynamically light microspheres. As will be discussed in more detail below, other suitable blowing agents include chloroform, freons, and hydrocarbons. Nitrogen gas and carbon dioxide are also contemplated as a suitable blowing agent.

Although the perforated microstructures may be formed using a blowing agent as described above, it will be appreciated that, in some instances, no blowing agent is required and an aqueous dispersion of the medicament and surfactant(s) are spray dried directly. In such cases, the formulation may be amenable to process conditions (e.g., elevated temperatures) that generally lead to the formation of hollow, relatively porous microparticles. Moreover, the medicament may possess special physicochemical properties (e.g., high crystallinity, elevated melting temperature, surface activity, etc.) that make it particularly suitable for use in such techniques.

The perforated microstructures may optionally be associated with, or comprise, one or more surfactants. Moreover, miscible surfactants may optionally be combined with the suspension medium liquid phase. It will be appreciated by those skilled in the art that the use of surfactants may further increase dispersion stability, simplify formulation procedures or increase bioavailability upon administration. Of course combinations of surfactants, including the use of one or more in the liquid phase and one or more associated with the perforated microstructures are contemplated as being within the scope of the invention. By “associated with or comprise” it is meant that the structural matrix or perforated microstructure may incorporate, adsorb, absorb, be coated with or be formed by the surfactant.

Surfactants suitable for use include any compound or composition that aids in the formation and maintenance of the stabilized respiratory dispersions by forming a layer at the interface between the structural matrix and the suspension medium. The surfactant may comprise a single compound or any combination of compounds, such as in the case of co-surfactants. Particularly certain surfactants are substantially insoluble in the propellant, nonfluorinated, and selected from the group consisting of saturated and unsaturated lipids, nonionic detergents, nonionic block copolymers, ionic surfactants, and combinations of such agents. It may be emphasized that, in addition to the aforementioned surfactants, suitable (i.e., biocompatible) fluorinated surfactants are compatible with the teachings herein and may be used to provide the desired stabilized preparations.

Lipids, including phospholipids, from both natural and synthetic sources may be used in varying concentrations to form a structural matrix. Generally, compatible lipids comprise those that have a gel to liquid crystal phase transition greater than about 40° C. In certain embodiments, the incorporated lipids are relatively long chain (i.e., C₆-C₂₂) saturated lipids and may comprise phospholipids. Exemplary phospholipids useful in the disclosed stabilized preparations comprise egg phosphatidylcholine, dilauroylphosphatidylcholine, dioleylphosphatidylcholine, dipalmitoylphosphatidyl-choline, disteroylphosphatidylcholine, short-chain phosphatidylcholines, phosphatidylethanolamine, dioleylphosphatidylethanolamine, phosphatidylserine, phosphatidylglycerol, phosphatidylinositol, glycolipids, ganglioside GM1, sphingomyelin, phosphatidic acid, cardiolipin; lipids bearing polymer chains such as, polyethylene glycol, chitin, hyaluronic acid, or polyvinylpyrrolidone; lipids bearing sulfonated mono-, di-, and polysaccharides; fatty acids such as palmitic acid, stearic acid, and oleic acid; cholesterol, cholesterol esters, and cholesterol hemisuccinate. Due to their excellent biocompatibility characteristics, phospholipids and combinations of phospholipids and poloxamers are particularly suitable for use in the stabilized dispersions disclosed herein.

Compatible nonionic detergents comprise: sorbitan esters including sorbitan trioleate (Spans™ 85), sorbitan sesquioleate, sorbitan monooleate, sorbitan monolaurate, polyoxyethylene (20) sorbitan monolaurate, and polyoxyethylene (20) sorbitan monooleate, oleyl polyoxyethylene (2) ether, stearyl polyoxyethylene (2) ether, lauryl polyoxyethylene (4) ether, glycerol esters, and sucrose esters. Other suitable nonionic detergents can be easily identified using McCutcheon's Emulsifiers and Detergents (McPublishing Co., Glen Rock, N.J.). Certain block copolymers include diblock and triblock copolymers of polyoxyethylene and polyoxypropylene, including poloxamer 188 (Pluronic F68), poloxamer 407 (Pluronic F-127), and poloxamer 338. Ionic surfactants such as sodium sulfosuccinate, and fatty acid soaps may also be utilized. In certain embodiments, the microstructures may comprise oleic acid or its alkali salt.

In addition to the aforementioned surfactants, cationic surfactants or lipids may be used, especially in the case of delivery of an siRNA compound, e.g., a double-stranded siRNA compound, or ssiRNA compound, (e.g., a precursor, e.g., a larger siRNA compound which can be processed into a ssiRNA compound, or a DNA which encodes an siRNA compound, e.g., a double-stranded siRNA compound, or ssiRNA compound, or precursor thereof). Examples of suitable cationic lipids include: DOTMA, N-[-(2,3-dioleyloxy)propyl]-N,N,N-trimethylammonium-chloride;DOTAP,1,2-dioleyloxy-3-(trimethylammonio)propane; and DOTB, 1,2-dioleyl-3-(4′-trimethylammonio)butanoyl-sn-glycerol. Polycationic amino acids such as polylysine, and polyarginine are also contemplated.

For the spraying process, such spraying methods as rotary atomization, pressure atomization and two-fluid atomization can be used. Examples of the devices used in these processes include “Parubisu [phonetic rendering] Mini-Spray GA-32” and “Parubisu Spray Drier DL-41”, manufactured by Yamato Chemical Co., or “Spray Drier CL-8,” “Spray Drier L-8,” “Spray Drier FL-12,” “Spray Drier FL-16” or “Spray Drier FL-20,” manufactured by Okawara Kakoki Co., can be used for the method of spraying using rotary-disk atomizer.

While no particular restrictions are placed on the gas used to dry the sprayed material, it is recommended to use air, nitrogen gas or an inert gas. The temperature of the inlet of the gas used to dry the sprayed materials such that it does not cause heat deactivation of the sprayed material. The range of temperatures may vary between about 50° C. to about 200° C., for example, between about 50° C. and 100° C. The temperature of the outlet gas used to dry the sprayed material, may vary between about 0° C. and about 150° C., for example, between 0° C. and 90° C., and for example between 0° C. and 60° C.

The spray drying is done under conditions that result in substantially amorphous powder of homogeneous constitution having a particle size that is respirable, a low moisture content and flow characteristics that allow for ready aerosolization. In some cases, the particle size of the resulting powder is such that more than about 98% of the mass is in particles having a diameter of about 10 μm or less with about 90% of the mass being in particles having a diameter less than 5 μm. Alternatively, about 95% of the mass will have particles with a diameter of less than 10 μm with about 80% of the mass of the particles having a diameter of less than 5 μm.

The dispersible pharmaceutical-based dry powders that include the siRNA preparation may optionally be combined with pharmaceutical carriers or excipients which are suitable for respiratory and pulmonary administration. Such carriers may serve simply as bulking agents when it is desired to reduce the siRNA concentration in the powder which is being delivered to a patient, but may also serve to enhance the stability of the siRNA compositions and to improve the dispersibility of the powder within a powder dispersion device in order to provide more efficient and reproducible delivery of the siRNA and to improve handling characteristics of the siRNA such as flowability and consistency to facilitate manufacturing and powder filling.

Such carrier materials may be combined with the drug prior to spray drying, i.e., by adding the carrier material to the purified bulk solution. In that way, the carrier particles will be formed simultaneously with the drug particles to produce a homogeneous powder. Alternatively, the carriers may be separately prepared in a dry powder form and combined with the dry powder drug by blending. The powder carriers will usually be crystalline (to avoid water absorption), but might in some cases be amorphous or mixtures of crystalline and amorphous. The size of the carrier particles may be selected to improve the flowability of the drug powder, typically being in the range from 25 μm to 100 μm. A carrier material may be crystalline lactose having a size in the above-stated range.

Powders prepared by any of the above methods will be collected from the spray dryer in a conventional manner for subsequent use. For use as pharmaceuticals and other purposes, it will frequently be desirable to disrupt any agglomerates which may have formed by screening or other conventional techniques. For pharmaceutical uses, the dry powder formulations will usually be measured into a single dose, and the single dose sealed into a package. Such packages are particularly useful for dispersion in dry powder inhalers, as described in detail below. Alternatively, the powders may be packaged in multiple-dose containers.

Methods for spray drying hydrophobic and other drugs and components are described in U.S. Pat. Nos. 5,000,888; 5,026,550; 4,670,419, 4,540,602; and 4,486,435 (all of which are incorporated by reference). Bloch and Speison (1983) Pharm. Acta Helv 58:14-22 teaches spray drying of hydrochlorothiazide and chlorthalidone (lipophilic drugs) and a hydrophilic adjuvant (pentaerythritol) in azeotropic solvents of dioxane-water and 2-ethoxyethanol-water. A number of Japanese Patent application Abstracts relate to spray drying of hydrophilic-hydrophobic product combinations, including JP 806766; JP 7242568; JP 7101884; JP 7101883; JP 71018982; JP 7101881; and JP 4036233. Other foreign patent publications relevant to spray drying hydrophilic-hydrophobic product combinations include FR 2594693; DE 2209477; and WO 88/07870.

### Lyophilization

An siRNA compound, e.g., a double-stranded siRNA compound, or ssiRNA compound, (e.g., a precursor, e.g., a larger siRNA compound which can be processed into a ssiRNA compound, or a DNA which encodes an siRNA compound, e.g., a double-stranded siRNA compound, or ssiRNA compound, or precursor thereof) preparation can be made by lyophilization. Lyophilization is a freeze-drying process in which water is sublimed from the composition after it is frozen. The particular advantage associated with the lyophilization process is that biologicals and pharmaceuticals that are relatively unstable in an aqueous solution can be dried without elevated temperatures (thereby eliminating the adverse thermal effects), and then stored in a dry state where there are few stability problems. With respect to the instant invention such techniques are particularly compatible with the incorporation of nucleic acids in perforated microstructures without compromising physiological activity. Methods for providing lyophilized particulates are known to those of skill in the art and it would clearly not require undue experimentation to provide dispersion compatible microstructures in accordance with the teachings herein. Accordingly, to the extent that lyophilization processes may be used to provide microstructures having the desired porosity and size, they are conformance with the teachings herein and are expressly contemplated as being within the scope of the instant invention.

### Genes

In one aspect, the invention provides a method of treating a subject at risk for or afflicted with a disease that may benefit from the administration of the siRNA of the invention. The method comprises administering the siRNA of the invention to a subject in need thereof, thereby treating the subject. The nucleic acid that is administered will depend on the disease being treated.

The transcriptional complex hypoxia inducible factor (HIF) is a key regulator of oxygen homeostasis. Hypoxia induces the expression of genes participating in many cellular and physiological processes, including oxygen transport and iron metabolism, erythropoiesis, angiogenesis, glycolysis and glucose uptake, transcription, metabolism, pH regulation, growth-factor signaling, response to stress and cell adhesion. These gene products participate in either increasing oxygen delivery to hypoxic tissues or activating an alternative metabolic pathway (glycolysis) which does not require oxygen. Hypoxia-induced pathways, in addition to being required for normal cellular processes, can also aid tumor growth by allowing or aiding angiogenesis, immortalization, genetic instability, tissue invasion and metastasis (Harris, Nat. Rev. Cancer, 2002, 2, 38-47; Maxwell et al., Curr. Opin. Genet. Dev., 2001, 11, 293-299). The transcription factor hypoxia-inducible factor 1 (HIF-1) plays an essential role in homeostatic responses to hypoxia by binding to the DNA sequence 5′-TACGTGCT-3′ and activating the transcription of dozens of genes in vivo under hypoxic conditions (Wang and Semenza, J. Biol. Chem., 1995, 270, 1230-1237). Hypoxia-inducible factor-1 alpha is a heterodimer composed of a 120 kDa alpha subunit complexed with a 91 to 94 kDa beta subunit, both of which contain a basic helix-loop-helix. The gene encoding hypoxia-inducible factor-1 alpha (HIF1α also called HIF-1 alpha, HIF1A, HIF-1A, HIF1-A, and MOP1) was cloned in 1995 (Wang et al., Proc. Natl. Acad. Sci. U.S.A., 1995, 92, 5510-5514). A nucleic acid sequence encoding HIF1α is disclosed and claimed in U.S. Pat. No. 5,882,914, as are expression vectors expressing the recombinant DNA, and host cells containing said vectors (Semenza, 1999). U.S. Pat. No. 7,217,572 (the disclosure of which is incorporated herein by reference) discloses at SEQ ID NO: 189 the antisense oligonucleotides sequence: GTGCAGTATT GTAGCCAGGC, and discloses at SEQ ID NO: 446 the antisense oligonucleotide sequence: CCTCATGGTC ACATGGATGA.

Aberrant expression of or constitutive expression of STAT3 is associated with a number of disease processes. STAT3 has been shown to be involved in cell transformation. Constitutive activation and/or overexpression of STAT3 appears to be involved in several forms of cancer, including myeloma, breast carcinomas, prostate cancer, brain tumors, head and neck carcinomas, melanoma, leukemias and lymphomas, particularly chronic myelogenous leukemia and multiple myeloma. Niu et al., Cancer Res., 1999, 59, 5059-5063. Breast cancer cell lines that overexpress EGFR constitutively express phosphorylated STAT3 (Sartor, C. I., et al., Cancer Res., 1997, 57, 978-987; Garcia, R., et al., Cell Growth and Differentiation, 1997, 8, 1267-1276). Activated STAT3 levels were also found to be elevated in low grade glioblastomas and medulloblastomas (Cattaneo, E., et al., Anticancer Res., 1998, 18, 2381-2387). U.S. Pat. No. 7,307,069 (the disclosure of which is incorporated herein by reference) discloses at SEQ ID NO: 184 the antisense oligonucleotide sequence: TTGGCTTCTC AAGATACCTG, and discloses at SEQ ID NO: 342 the antisense oligonucleotides sequence: GACTCTTGCA GGAAGCGGCT.

Huntington's disease is a progressive neurodegenerative disorder characterized by motor disturbance, cognitive loss and psychiatric manifestations (Martin and Gusella, N. Engl. J. Med. 315:1267-1276 (1986). Although an actual mechanism for Huntington's disease remains elusive, Huntington's disease has been shown to be an autosomal dominant neurodegenerative disorder caused by an expanding glutamine repeat in a gene termed IT15 or Huntingtin (HD). Although this gene is widely expressed and is required for normal development, the pathology of Huntington's disease is restricted to the brain, for reasons that remain poorly understood. The Huntingtin gene product is expressed at similar levels in patients and controls, and the genetics of the disorder suggest that the expansion of the polyglutamine repeat induces a toxic gain of function, perhaps through interactions with other cellular proteins. U.S. Pat. No. 7,320,965 (the disclosure of which is incorporated herein by reference) discloses an antisense strand for inhibiting the expression of a human Huntingtin gene at SEQ ID NO: 793: CUGCACGGUU CUUUGUGACT T.

The intracellular transport of proteins, lipids, and mRNA to specific locations within the cell, as well as the proper alignment and separation of chromosomes in dividing cells, is essential to the functioning of the cell. The superfamily of proteins called kinesins (KIF), along with the myosins and dyneins, function as molecular engines to bind and transport vesicles and organelles along microtubules with energy supplied by ATP. KIFs have been identified in many species ranging from yeast to humans. The amino acid sequences which comprise the motor domain are highly conserved among eukaryotic phyla, while the region outside of the motor domain serves to bind to the cargo and varies in amino acid sequence among KIFs. The movement of a kinesin along a microtubule can occur in either the plus or minus direction, but any given kinesin can only travel in one direction, an action that is mediated by the polarity of the motor and the microtubule. The KIFs have been grouped into three major types depending on the position of the motor domain: the amino-terminal domain, the middle motor domain, and the carboxyl-terminal domain, referred to respectively as N-kinesin, M-kinesin, and C-kinesins. These are further classified into 14 classes based on a phylogenetic analysis of the 45 known human and mouse kinesin genes (Miki et al., Proc. Natl. Acad. Sci. U.S.A., 2001, 98, 7004-7011). One such kinesin, kinesin-like 1, a member of the N-2 (also called bimC) family of kinesins and is involved in separating the chromosomes by directing their movement along microtubules in the bipolar spindle. During mitosis, the microtubule bipolar spindle functions to distribute the duplicated chromosomes equally to daughter cells. Kinesin-like 1 is first phosphorylated by the kinase p34^{cdc2} and is essential for centrosome separation and assembly of bipolar spindles at prophase (Blangy et al., Cell, 1995, 83, 1159-1169). In rodent neurons, kinesin-like 1 is expressed well past their terminal mitotic division, and has been implicated in regulating microtubule behaviors within the developing axons and dendrites (Ferhat et al., J. Neurosci., 1998, 18, 7822-7835). The gene encoding human kinesin-like 1 (also called KNSL1, Eg5, HsEg5, HKSP, KIF11, thyroid interacting protein 5, and TRIPS) was cloned in 1995 (Blangy et al., Cell, 1995, 83, 1159-1169). Inhibition of kinesin-like 1 has been suggested as a target for arresting cellular proliferation in cancer because of the central role kinesin-like 1 holds in mitosis. Expression of kinesin-like 1 may also contribute to other disease states. A contribution of kinesin-like 1 to B-cell leukemia has been demonstrated in mice as a result of upregulated expression of kinesin-like 1 following a retroviral insertion mutation in the proximity of the kinesin-like 1 gene (Hansen and Justice, Oncogene, 1999, 18, 6531-6539). Autoantibodies to a set of proteins in the mitotic spindle assembly have been detected in human sera and these autoantibodies have been associated with autoimmune diseases including carpal tunnel syndrome, Raynaud's phenomenon, systemic sclerosis, Sjorgren's syndrome, rheumatoid arthritis, polymyositis, and polyarteritis. One of these autoantigens is kinesin-like 1 and has been identified in systemic lupus erythematosus (Whitehead et al., Arthritis Rheum., 1996, 39, 1635-1642). U.S. Pat. No. 7,199,107 (the disclosure of which is incorporated herein by reference) discloses an antisense strand for inhibiting the expression of a human kinesin-1 gene at SEQ ID NO: 122: ACGTGGAATT ATACCAGCCA.

A number of therapeutic strategies exist for inhibiting aberrant angiogenesis, which attempt to reduce the production or effect of VEGF. For example, anti-VEGF or anti-VEGF receptor antibodies (Kim E S et al. (2002), PNAS USA 99: 11399-11404), and soluble VEGF “traps” which compete with endothelial cell receptors for VEGF binding (Holash J et al. (2002), PNAS USA 99: 11393-11398) have been developed. Classical VEGF “antisense” or aptamer therapies directed against VEGF gene expression have also been proposed (U.S. published application 2001/0021772 of Uhlmann et al., the disclosure of which is incorporated herein by reference). However, the anti-angiogenic agents used in these therapies can produce only a stoichiometric reduction in VEGF or VEGF receptor, and the agents are typically overwhelmed by the abnormally high production of VEGF by the diseased tissue. The results achieved with available anti-angiogenic therapies have therefore been unsatisfactory. U.S. Pat. No. 7,345,027 (the disclosure of which is incorporated herein by reference) discloses an antisense strand for inhibiting the expression of a human VEGF gene at SEQ ID NO: 78: GUGCUGGCCUUGGUGAGGUTT (The terminal two Ts are overhangs).

The NF-κB or nuclear factor κB is a transcription factor that plays a critical role in inflammatory diseases by inducing the expression of a large number of proinflammatory and anti-apoptotic genes. These include cytokines such as IL-1, IL-2, IL-11, TNF-α and IL-6, chemokines including IL-8, GRO1 and RANTES, as well as other proinflammatory molecules including COX-2 and cell adhesion molecules such as ICAM-1, VCAM-1, and E-selectin. Pahl H L, (1999) Oncogene 18, 6853-6866; Jobin et al, (2000) Am. J. Physiol. Cell. Physiol. 278: 451-462. Under resting conditions, NF-κB is present in the cytosol of cells as a complex with IκB. The IκB family of proteins serve as inhibitors of NF-κB, interfering with the function of its nuclear localization signal (see for example U. Siebenlist et al, (1994) Ann. Rev. Cell Bio., 10: 405). Upon disruption of the IκB-NF-κB complex following cell activation, NF-κB translocates to the nucleus and activates gene transcription. Disruption of the IκB-NF-κB complex and subsequent activation of NF-κB is initiated by degradation of IκB. Activators of NF-κB mediate the site-specific phosphorylation of two amino terminal serines in each IκB which makes nearby lysines targets for ubiquitination, thereby resulting in IκB proteasomal destruction. NF-κB is then free to translocate to the nucleus and bind DNA leading to the activation of a host of inflammatory response target genes. (Baldwin, A., Jr., (1996) Annu Rev Immunol 14: 649-683, Ghosh, S. et al, (1998) Annu Rev Immunol 16, 225-260.) Recent evidence has shown that NF-κB subunits dynamically shuttle between the cytoplasm and the nucleus but a dominant acting nuclear export signal in IκBα ensures their transport back to the cytoplasm. Even though NF-κB is largely considered to be a transcriptional activator, under certain circumstances it can also be involved in directly repressing gene expression (reviewed in Ghosh, S. et al. (1998) Annu. Rev. Immunol., 16: 225-260). U.S. Pat. No. 7,235,654 (the disclosure of which is incorporated herein by reference) discloses an siRNA at SEQ ID NO: 3: GUCUGUGUAU CACGUGACGN N (wherein N is a 2′-deoxy-thymidine).

Control of the risk factors involved in hypercholesterolemia and cardiovascular disease has been the focus of much research in academia and industry. Because an elevated level of circulating plasma low-density lipoprotein cholesterol has been identified as an independent risk factor in the development of hypercholesterolemia and cardiovascular disease, many strategies have been directed at lowering the levels of cholesterol carried in this atherogenic lipoprotein. AcylCoA cholesterol acyltransferase (ACAT) enzymes catalyze the synthesis of cholesterol esters from free cholesterol and fatty acyl-CoA. These enzymes are also involved in regulation of the concentration of cellular free sterols (Buhman et al., Biochim. Biophys. Acta, 2000, 1529, 142-154; Burnett et al., Clin. Chim. Acta, 1999, 286, 231-242; Chang et al., Annu. Rev. Biochem., 1997, 66, 613-638; Rudel et al., Curr. Opin. Lipidol., 2001, 12, 121-127; Rudel and Shelness, Nat. Med., 2000, 6, 1313-1314). Chang et al. cloned the first example of a human ACAT gene in 1993 (Chang et al., J. Biol. Chem., 1993, 268, 20747-20755). This original ACAT enzyme is now known as ACAT-1. Subsequently, the work of Meiner et al. suggested the presence of more than one ACAT gene in mammals (Meiner et al., J. Lipid Res., 1997, 38, 1928-1933). The cloning and expression of a second human ACAT isoform now known as acyl CoA cholesterol acyltransferase-2, was accomplished recently (Oelkers et al., J. Biol. Chem., 1998, 273, 26765-26771). Murine acyl CoA cholesterol acyltransferase-2 has also been identified and cloned (Cases et al., J. Biol. Chem., 1998, 273, 26755-26764). U.S. Pat. No. 7,335,764 (the disclosure of which is incorporated herein by reference) discloses siRNAs targeted to a nucleic acid molecule encoding acyl CoA cholesterol acyltransferase-2 at SEQ ID NOs: 25 (GCACGAAGGA TCCCAGGCAC), 26 (GGATCCCCTC ACCTCGTCTG) and 27 (GTTCTTGGCC ACATAATTCC).

Lp(a) contains two disulfide-linked distinct proteins, apolipoprotein(a) (or ApoA) and apolipoprotein B (or ApoB) (Rainwater and Kammerer, J. Exp. Zool., 1998, 282, 54-61). Apolipoprotein(a) is a unique apolipoprotein encoded by the LPA gene which has been shown to exclusively control the physiological concentrations of Lp(a) (Rainwater and Kammerer, J. Exp. Zool., 1998, 282, 54-61). It varies in size due to interallelic differences in the number of tandemly repeated Kringle 4-encoding 5.5 kb sequences in the LPA gene (Rainwater and Kammerer, J. Exp. Zool., 1998, 282, 54-61). Elevated plasma levels of Lp(a), caused by increased expression of apolipoprotein(a), are associated with increased risk for atherosclerosis and its manifestations, which include hypercholesterolemia (Seed et al., N. Engl. J. Med., 1990, 322, 1494-1499), myocardial infarction (Sandkamp et al., Clin. Chem., 1990, 36, 20-23), and thrombosis (Nowak-Gottl et al., Pediatrics, 1997, 99, E11). Moreover, the plasma concentration of Lp(a) is strongly influenced by heritable factors and is refractory to most drug and dietary manipulation (Katan and Beynen, Am. J. Epidemiol., 1987, 125, 387-399; Vessby et al., Atherosclerosis, 1982, 44, 61-71.). Pharmacologic therapy of elevated Lp(a) levels has been only modestly successful and apheresis remains the most effective therapeutic modality (Hajjar and Nachman, Annu. Rev. Med., 1996, 47, 423-442). U.S. Pat. No. 7,259,150 (the disclosure of which is incorporated herein by reference) discloses an siRNA for inhibiting the expression of apolipoprotein(a) at SEQ ID NO: 23: ACCTGACACC GGGATCCCTC.

In certain embodiments, the siRNA compound (e.g., the siRNA in a composition described herein) silences a growth factor or growth factor receptor gene, a kinase, e.g., a protein tyrosine, serine or threonine kinase gene, an adaptor protein gene, a gene encoding a G protein superfamily molecule, or a gene encoding a transcription factor.

In some embodiments the siRNA compound (e.g., the siRNA in a composition described herein) silences the PDGF beta gene, and thus can be used to treat a subject having or at risk for a disorder characterized by unwanted PDGF beta expression, e.g., testicular and lung cancers.

In some embodiments the siRNA compound (e.g., the siRNA in a composition described herein) silences the Erb-B gene, and thus can be used to treat a subject having or at risk for a disorder characterized by unwanted Erb-B expression, e.g., breast cancer.

In some embodiments the siRNA compound (e.g., the siRNA in a composition described herein) silences the Src gene, and thus can be used to treat a subject having or at risk for a disorder characterized by unwanted Src expression, e.g., colon cancers.

In some embodiments the siRNA compound (e.g., the siRNA in a composition described herein) silences the CRK gene, and thus can be used to treat a subject having or at risk for a disorder characterized by unwanted CRK expression, e.g., colon and lung cancers.

In some embodiments the siRNA compound (e.g., the siRNA in a composition described herein) silences the GRB2 gene, and thus can be used to treat a subject having or at risk for a disorder characterized by unwanted GRB2 expression, e.g., squamous cell carcinoma.

In another embodiment the siRNA compound (e.g., the siRNA in a composition described herein) silences the RAS gene, and thus can be used to treat a subject having or at risk for a disorder characterized by unwanted RAS expression, e.g., pancreatic, colon and lung cancers, and chronic leukemia.

In another embodiment the siRNA compound (e.g., the siRNA in a composition described herein) silences the MEKK gene, and thus can be used to treat a subject having or at risk for a disorder characterized by unwanted MEKK expression, e.g., squamous cell carcinoma, melanoma or leukemia.

In another embodiment the siRNA compound (e.g., the siRNA in a composition described herein) silences the JNK gene, and thus can be used to treat a subject having or at risk for a disorder characterized by unwanted JNK expression, e.g., pancreatic or breast cancers.

In some embodiments the siRNA compound (e.g., the siRNA in a composition described herein) silences the RAF gene, and thus can be used to treat a subject having or at risk for a disorder characterized by unwanted RAF expression, e.g., lung cancer or leukemia.

In some embodiments the siRNA compound (e.g., the siRNA in a composition described herein) silences the Erk1/2 gene, and thus can be used to treat a subject having or at risk for a disorder characterized by unwanted Erk1/2 expression, e.g., lung cancer.

In another embodiment the siRNA compound (e.g., the siRNA in a composition described herein) silences the PCNA(p21) gene, and thus can be used to treat a subject having or at risk for a disorder characterized by unwanted PCNA expression, e.g., lung cancer.

In some embodiments the siRNA compound (e.g., the siRNA in a composition described herein) silences the MYB gene, and thus can be used to treat a subject having or at risk for a disorder characterized by unwanted MYB expression, e.g., colon cancer or chronic myelogenous leukemia.

In some embodiments the siRNA compound (e.g., the siRNA in a composition described herein) silences the c-MYC gene, and thus can be used to treat a subject having or at risk for a disorder characterized by unwanted c-MYC expression, e.g., Burkitt's lymphoma or neuroblastoma.

In another embodiment the siRNA compound (e.g., the siRNA in a composition described herein) silences the JUN gene, and thus can be used to treat a subject having or at risk for a disorder characterized by unwanted JUN expression, e.g., ovarian, prostate or breast cancers.

In another embodiment the siRNA compound (e.g., the siRNA in a composition described herein) silences the FOS gene, and thus can be used to treat a subject having or at risk for a disorder characterized by unwanted FOS expression, e.g., skin or prostate cancers.

In some embodiments the siRNA compound (e.g., the siRNA in a composition described herein) silences the BCL-2 gene, and thus can be used to treat a subject having or at risk for a disorder characterized by unwanted BCL-2 expression, e.g., lung or prostate cancers or Non-Hodgkin lymphoma.

In some embodiments the siRNA compound (e.g., the siRNA in a composition described herein) silences the Cyclin D gene, and thus can be used to treat a subject having or at risk for a disorder characterized by unwanted Cyclin D expression, e.g., esophageal and colon cancers.

In some embodiments the siRNA compound (e.g., the siRNA in a composition described herein) silences the VEGF gene, and thus can be used to treat a subject having or at risk for a disorder characterized by unwanted VEGF expression, e.g., esophageal and colon cancers.

In some embodiments the siRNA compound (e.g., the siRNA in a composition described herein) silences the EGFR gene, and thus can be used to treat a subject having or at risk for a disorder characterized by unwanted EGFR expression, e.g., breast cancer.

In another embodiment the siRNA compound (e.g., the siRNA in a composition described herein) silences the Cyclin A gene, and thus can be used to treat a subject having or at risk for a disorder characterized by unwanted Cyclin A expression, e.g., lung and cervical cancers.

In another embodiment the siRNA compound (e.g., the siRNA in a composition described herein) silences the Cyclin E gene, and thus can be used to treat a subject having or at risk for a disorder characterized by unwanted Cyclin E expression, e.g., lung and breast cancers.

In another embodiment the siRNA compound (e.g., the siRNA in a composition described herein) silences the WNT-1 gene, and thus can be used to treat a subject having or at risk for a disorder characterized by unwanted WNT-1 expression, e.g., basal cell carcinoma.

In another embodiment the siRNA compound (e.g., the siRNA in a composition described herein) silences the beta-catenin gene, and thus can be used to treat a subject having or at risk for a disorder characterized by unwanted beta-catenin expression, e.g., adenocarcinoma or hepatocellular carcinoma.

In another embodiment the siRNA compound (e.g., the siRNA in a composition described herein) silences the c-MET gene, and thus can be used to treat a subject having or at risk for a disorder characterized by unwanted c-MET expression, e.g., hepatocellular carcinoma.

In another embodiment the siRNA compound (e.g., the siRNA in a composition described herein) silences the PKC gene, and thus can be used to treat a subject having or at risk for a disorder characterized by unwanted PKC expression, e.g., breast cancer.

In some embodiments the siRNA compound (e.g., the siRNA in a composition described herein) silences the NFκB gene, and thus can be used to treat a subject having or at risk for a disorder characterized by unwanted NFκB expression, e.g., breast cancer.

In some embodiments the siRNA compound (e.g., the siRNA in a composition described herein) silences the STAT3 gene, and thus can be used to treat a subject having or at risk for a disorder characterized by unwanted STAT3 expression, e.g., prostate cancer.

In another embodiment the siRNA compound (e.g., the siRNA in a composition described herein) silences the survivin gene, and thus can be used to treat a subject having or at risk for a disorder characterized by unwanted survivin expression, e.g., cervical or pancreatic cancers.

In another embodiment the siRNA compound (e.g., the siRNA in a composition described herein) silences the Her2/Neu gene, and thus can be used to treat a subject having or at risk for a disorder characterized by unwanted Her2/Neu expression, e.g., breast cancer.

In another embodiment the siRNA compound (e.g., the siRNA in a composition described herein) silences the topoisomerase I gene, and thus can be used to treat a subject having or at risk for a disorder characterized by unwanted topoisomerase I expression, e.g., ovarian and colon cancers.

In some embodiments the siRNA compound (e.g., the siRNA in a composition described herein) silences the topoisomerase II alpha gene, and thus can be used to treat a subject having or at risk for a disorder characterized by unwanted topoisomerase II expression, e.g., breast and colon cancers.

In some embodiments the siRNA compound (e.g., the siRNA in a composition described herein) silences mutations in the p73 gene, and thus can be used to treat a subject having or at risk for a disorder characterized by unwanted p73 expression, e.g., colorectal adenocarcinoma.

In some embodiments the siRNA compound (e.g., the siRNA in a composition described herein) silences mutations in the p21(WAF1/CIP1) gene, and thus can be used to treat a subject having or at risk for a disorder characterized by unwanted p21(WAF1/CIP1) expression, e.g., liver cancer.

In some embodiments the siRNA compound (e.g., the siRNA in a composition described herein) silences mutations in the p27(KIP1) gene, and thus can be used to treat a subject having or at risk for a disorder characterized by unwanted p27(KIP1) expression, e.g., liver cancer.

In some embodiments the siRNA compound (e.g., the siRNA in a composition described herein) silences mutations in the PPM1D gene, and thus can be used to treat a subject having or at risk for a disorder characterized by unwanted PPM1D expression, e.g., breast cancer.

In some embodiments the siRNA compound (e.g., the siRNA in a composition described herein) silences mutations in the RAS gene, and thus can be used to treat a subject having or at risk for a disorder characterized by unwanted RAS expression, e.g., breast cancer.

In another embodiment the siRNA compound (e.g., the siRNA in a composition described herein) silences mutations in the caveolin I gene, and thus can be used to treat a subject having or at risk for a disorder characterized by unwanted caveolin I expression, e.g., esophageal squamous cell carcinoma.

In another embodiment the siRNA compound (e.g., the siRNA in a composition described herein) silences mutations in the MIB I gene, and thus can be used to treat a subject having or at risk for a disorder characterized by unwanted MIB I expression, e.g., male breast carcinoma (MBC).

In another embodiment the siRNA compound (e.g., the siRNA in a composition described herein) silences mutations in the MTAI gene, and thus can be used to treat a subject having or at risk for a disorder characterized by unwanted MTAI expression, e.g., ovarian carcinoma.

In another embodiment the siRNA compound (e.g., the siRNA in a composition described herein) silences mutations in the M68 gene, and thus can be used to treat a subject having or at risk for a disorder characterized by unwanted M68 expression, e.g., human adenocarcinomas of the esophagus, stomach, colon, and rectum.

In certain embodiments the siRNA compound (e.g., the siRNA in a composition described herein) silences mutations in tumor suppressor genes, and thus can be used as a method to promote apoptotic activity in combination with chemotherapeutics.

In some embodiments the siRNA compound (e.g., the siRNA in a composition described herein) silences mutations in the p53 tumor suppressor gene, and thus can be used to treat a subject having or at risk for a disorder characterized by unwanted p53 expression, e.g., gall bladder, pancreatic and lung cancers.

In some embodiments the siRNA compound (e.g., the siRNA in a composition described herein) silences mutations in the p53 family member DN-p63, and thus can be used to treat a subject having or at risk for a disorder characterized by unwanted DN-p63 expression, e.g., squamous cell carcinoma.

In some embodiments the siRNA compound (e.g., the siRNA in a composition described herein) silences mutations in the pRb tumor suppressor gene, and thus can be used to treat a subject having or at risk for a disorder characterized by unwanted pRb expression, e.g., oral squamous cell carcinoma.

In some embodiments the siRNA compound (e.g., the siRNA in a composition described herein) silences mutations in the APC1 tumor suppressor gene, and thus can be used to treat a subject having or at risk for a disorder characterized by unwanted APC1 expression, e.g., colon cancer.

In some embodiments the siRNA compound (e.g., the siRNA in a composition described herein) silences mutations in the BRCA1 tumor suppressor gene, and thus can be used to treat a subject having or at risk for a disorder characterized by unwanted BRCA1 expression, e.g., breast cancer.

In some embodiments the siRNA compound (e.g., the siRNA in a composition described herein) silences mutations in the PTEN tumor suppressor gene, and thus can be used to treat a subject having or at risk for a disorder characterized by unwanted PTEN expression, e.g., hamartomas, gliomas, and prostate and endometrial cancers.

In some embodiments the siRNA compound (e.g., the siRNA in a composition described herein) silences MLL fusion genes, e.g., MLL-AF9, and thus can be used to treat a subject having or at risk for a disorder characterized by unwanted MLL fusion gene expression, e.g., acute leukemias.

In another embodiment the siRNA compound (e.g., the siRNA in a composition described herein) silences the BCR/ABL fusion gene, and thus can be used to treat a subject having or at risk for a disorder characterized by unwanted BCR/ABL fusion gene expression, e.g., acute and chronic leukemias.

In another embodiment the siRNA compound (e.g., the siRNA in a composition described herein) silences the TEL/AML1 fusion gene, and thus can be used to treat a subject having or at risk for a disorder characterized by unwanted TEL/AML1 fusion gene expression, e.g., childhood acute leukemia.

In another embodiment the siRNA compound (e.g., the siRNA in a composition described herein) silences the EWS/FLI1 fusion gene, and thus can be used to treat a subject having or at risk for a disorder characterized by unwanted EWS/FLI1 fusion gene expression, e.g., Ewing Sarcoma.

In another embodiment the siRNA compound (e.g., the siRNA in a composition described herein) silences the TLS/FUS1 fusion gene, and thus can be used to treat a subject having or at risk for a disorder characterized by unwanted TLS/FUS1 fusion gene expression, e.g., Myxoid liposarcoma.

In another embodiment the siRNA compound (e.g., the siRNA in a composition described herein) silences the PAX3/FKHR fusion gene, and thus can be used to treat a subject having or at risk for a disorder characterized by unwanted PAX3/FKHR fusion gene expression, e.g., Myxoid liposarcoma.

In another embodiment the siRNA compound (e.g., the siRNA in a composition described herein) silences the AML1/ETO fusion gene, and thus can be used to treat a subject having or at risk for a disorder characterized by unwanted AML1/ETO fusion gene expression, e.g., acute leukemia.

### Diseases and Disorders

### Angiogenesis

In another aspect, the invention provides a method of treating a subject, e.g., a human, at risk for or afflicted with a disease or disorder that may benefit by angiogenesis inhibition, e.g., cancer. The method comprises administering the siRNA of the invention to a subject in need thereof, thereby treating the subject. The nucleic acid that is administered will depend on the type of angiogenesis-related gene being treated.

In some embodiments the siRNA compound (e.g., the siRNA in a composition described herein) silences the alpha v-integrin gene, and thus can be used to treat a subject having or at risk for a disorder characterized by unwanted alpha V integrin, e.g., brain tumors or tumors of epithelial origin.

In some embodiments the siRNA compound (e.g., the siRNA in a composition described herein) silences the Flt-1 receptor gene, and thus can be used to treat a subject having or at risk for a disorder characterized by unwanted Flt-1 receptors, eg. cancer and rheumatoid arthritis.

In some embodiments the siRNA compound (e.g., the siRNA in a composition described herein) silences the tubulin gene, and thus can be used to treat a subject having or at risk for a disorder characterized by unwanted tubulin, eg. cancer and retinal neovascularization.

In some embodiments the siRNA compound (e.g., the siRNA in a composition described herein) silences the tubulin gene, and thus can be used to treat a subject having or at risk for a disorder characterized by unwanted tubulin, eg. cancer and retinal neovascularization.

### Viral Diseases

In yet another aspect, the invention features a method of treating a subject infected with a virus or at risk for or afflicted with a disorder or disease associated with a viral infection. The method comprises administering the siRNA of the invention to a subject in need thereof, thereby treating the subject. The nucleic acid that is administered will depend on the type of viral disease being treated. In some embodiments, the nucleic acid may target a viral gene. In other embodiments, the nucleic acid may target a host gene.

Thus, the invention provides for a method of treating patients infected by the Human Papilloma Virus (HPV) or at risk for or afflicted with a disorder mediated by HPV, e.g, cervical cancer. HPV is linked to 95% of cervical carcinomas and thus an antiviral therapy is an attractive method to treat these cancers and other symptoms of viral infection. In some embodiments, the expression of a HPV gene is reduced. In another embodiment, the HPV gene is one of the group of E2, E6, or E7. In some embodiments the expression of a human gene that is required for HPV replication is reduced.

The invention also includes a method of treating patients infected by the Human Immunodeficiency Virus (HIV) or at risk for or afflicted with a disorder mediated by HIV, e.g., Acquired Immune Deficiency Syndrome (AIDS). In some embodiments, the expression of a HIV gene is reduced. In another embodiment, the HIV gene is CCR5, Gag, or Rev. In some embodiments the expression of a human gene that is required for HIV replication is reduced. In another embodiment, the gene is CD4 or Tsg101.

The invention also includes a method for treating patients infected by the Hepatitis B Virus (HBV) or at risk for or afflicted with a disorder mediated by HBV, e.g., cirrhosis and heptocellular carcinoma. In some embodiments, the expression of a HBV gene is reduced. In another embodiment, the targeted HBV gene encodes one of the group of the tail region of the HBV core protein, the pre-cregious (pre-c) region, or the cregious (c) region. In another embodiment, a targeted HBV-RNA sequence is comprised of the poly(A) tail. In certain embodiment the expression of a human gene that is required for HBV replication is reduced.

The invention also provides for a method of treating patients infected by the Hepatitis A Virus (HAV), or at risk for or afflicted with a disorder mediated by HAV. In some embodiments the expression of a human gene that is required for HAV replication is reduced.

The present invention provides for a method of treating patients infected by the Hepatitis C Virus (HCV), or at risk for or afflicted with a disorder mediated by HCV, e.g., cirrhosis. In some embodiments, the expression of a HCV gene is reduced. In another embodiment the expression of a human gene that is required for HCV replication is reduced.

The present invention also provides for a method of treating patients infected by the any of the group of Hepatitis Viral strains comprising hepatitis D, E, F, G, or H, or patients at risk for or afflicted with a disorder mediated by any of these strains of hepatitis. In some embodiments, the expression of a Hepatitis, D, E, F, G, or H gene is reduced. In another embodiment the expression of a human gene that is required for hepatitis D, E, F, G or H replication is reduced.

Methods of the invention also provide for treating patients infected by the Respiratory Syncytial Virus (RSV) or at risk for or afflicted with a disorder mediated by RSV, e.g, lower respiratory tract infection in infants and childhood asthma, pneumonia and other complications, e.g., in the elderly. In some embodiments, the expression of a RSV gene is reduced. In another embodiment, the targeted HBV gene encodes one of the group of genes N, L, or P. In some embodiments the expression of a human gene that is required for RSV replication is reduced.

Methods of the invention provide for treating patients infected by the Herpes Simplex Virus (HSV) or at risk for or afflicted with a disorder mediated by HSV, e.g, genital herpes and cold sores as well as life-threatening or sight-impairing disease mainly in immunocompromised patients. In some embodiments, the expression of a HSV gene is reduced. In another embodiment, the targeted HSV gene encodes DNA polymerase or the helicase-primase. In some embodiments the expression of a human gene that is required for HSV replication is reduced.

The invention also provides a method for treating patients infected by the herpes Cytomegalovirus (CMV) or at risk for or afflicted with a disorder mediated by CMV, e.g., congenital virus infections and morbidity in immunocompromised patients. In some embodiments, the expression of a CMV gene is reduced. In some embodiments the expression of a human gene that is required for CMV replication is reduced.

Methods of the invention also provide for a method of treating patients infected by the herpes Epstein Barr Virus (EBV) or at risk for or afflicted with a disorder mediated by EBV, e.g., NK/T-cell lymphoma, non-Hodgkin lymphoma, and Hodgkin disease. In some embodiments, the expression of a EBV gene is reduced. In some embodiments the expression of a human gene that is required for EBV replication is reduced.

Methods of the invention also provide for treating patients infected by Kaposi's Sarcoma-associated Herpes Virus (KSHV), also called human herpesvirus 8, or patients at risk for or afflicted with a disorder mediated by KSHV, e.g., Kaposi's sarcoma, multicentric Castleman's disease and AIDS-associated primary effusion lymphoma. In some embodiments, the expression of a KSHV gene is reduced. In some embodiments the expression of a human gene that is required for KSHV replication is reduced.

The invention also includes a method for treating patients infected by the JC Virus (JCV) or a disease or disorder associated with this virus, e.g., progressive multifocal leukoencephalopathy (PML). In some embodiments, the expression of a JCV gene is reduced. In certain embodiments the expression of a human gene that is required for JCV replication is reduced.

Methods of the invention also provide for treating patients infected by the myxovirus or at risk for or afflicted with a disorder mediated by myxovirus, e.g., influenza. In some embodiments, the expression of a myxovirus gene is reduced. In some embodiments the expression of a human gene that is required for myxovirus replication is reduced.

Methods of the invention also provide for treating patients infected by the rhinovirus or at risk for of afflicted with a disorder mediated by rhinovirus, e.g., the common cold. In some embodiments, the expression of a rhinovirus gene is reduced. In certain embodiments the expression of a human gene that is required for rhinovirus replication is reduced.

Methods of the invention also provide for treating patients infected by the coronavirus or at risk for of afflicted with a disorder mediated by coronavirus, e.g., the common cold. In some embodiments, the expression of a coronavirus gene is reduced. In certain embodiments the expression of a human gene that is required for coronavirus replication is reduced.

Methods of the invention also provide for treating patients infected by the flavivirus West Nile or at risk for or afflicted with a disorder mediated by West Nile Virus. In some embodiments, the expression of a West Nile Virus gene is reduced. In another embodiment, the West Nile Virus gene is E, NS3, or NS5. In some embodiments the expression of a human gene that is required for West Nile Virus replication is reduced.

Methods of the invention also provide for treating patients infected by the St. Louis Encephalitis flavivirus, or at risk for or afflicted with a disease or disorder associated with this virus, e.g., viral haemorrhagic fever or neurological disease. In some embodiments, the expression of a St. Louis Encephalitis gene is reduced. In some embodiments the expression of a human gene that is required for St. Louis Encephalitis virus replication is reduced.

Methods of the invention also provide for treating patients infected by the Tick-borne encephalitis flavivirus, or at risk for or afflicted with a disorder mediated by Tick-borne encephalitis virus, e.g., viral haemorrhagic fever and neurological disease. In some embodiments, the expression of a Tick-borne encephalitis virus gene is reduced. In some embodiments the expression of a human gene that is required for Tick-borne encephalitis virus replication is reduced.

Methods of the invention also provide for methods of treating patients infected by the Murray Valley encephalitis flavivirus, which commonly results in viral haemorrhagic fever and neurological disease. In some embodiments, the expression of a Murray Valley encephalitis virus gene is reduced. In some embodiments the expression of a human gene that is required for Murray Valley encephalitis virus replication is reduced.

The invention also includes methods for treating patients infected by the dengue flavivirus, or a disease or disorder associated with this virus, e.g., dengue haemorrhagic fever. In some embodiments, the expression of a dengue virus gene is reduced. In some embodiments the expression of a human gene that is required for dengue virus replication is reduced.

Methods of the invention also provide for treating patients infected by the Simian Virus 40 (SV40) or at risk for or afflicted with a disorder mediated by SV40, e.g., tumorigenesis. In some embodiments, the expression of a SV40 gene is reduced. In some embodiments the expression of a human gene that is required for SV40 replication is reduced.

The invention also includes methods for treating patients infected by the Human T Cell Lymphotropic Virus (HTLV), or a disease or disorder associated with this virus, e.g., leukemia and myelopathy. In some embodiments, the expression of a HTLV gene is reduced. In another embodiment the HTLV1 gene is the Tax transcriptional activator. In some embodiments the expression of a human gene that is required for HTLV replication is reduced.

Methods of the invention also provide for treating patients infected by the Moloney-Murine Leukemia Virus (Mo-MuLV) or at risk for or afflicted with a disorder mediated by Mo-MuLV, e.g., T-cell leukemia. In some embodiments, the expression of a Mo-MuLV gene is reduced. In some embodiments the expression of a human gene that is required for Mo-MuLV replication is reduced.

Methods of the invention also provide for treating patients infected by the encephalomyocarditis virus (EMCV) or at risk for or afflicted with a disorder mediated by EMCV, e.g., myocarditis. EMCV leads to myocarditis in mice and pigs and is capable of infecting human myocardial cells. This virus is therefore a concern for patients undergoing xenotransplantation. In some embodiments, the expression of a EMCV gene is reduced. In some embodiments the expression of a human gene that is required for EMCV replication is reduced.

The invention also includes a method for treating patients infected by the measles virus (MV) or at risk for or afflicted with a disorder mediated by MV, e.g., measles. In some embodiments, the expression of a MV gene is reduced. In some embodiments the expression of a human gene that is required for MV replication is reduced.

The invention also includes a method for treating patients infected by the Vericella zoster virus (VZV) or at risk for or afflicted with a disorder mediated by VZV, e.g., chicken pox or shingles (also called zoster). In some embodiments, the expression of a VZV gene is reduced. In some embodiments the expression of a human gene that is required for VZV replication is reduced.

The invention also includes a method for treating patients infected by an adenovirus or at risk for or afflicted with a disorder mediated by an adenovirus, e.g., respiratory tract infection. In some embodiments, the expression of an adenovirus gene is reduced. In some embodiments the expression of a human gene that is required for adenovirus replication is reduced.

The invention includes a method for treating patients infected by a yellow fever virus (YFV) or at risk for or afflicted with a disorder mediated by a YFV, e.g., respiratory tract infection. In some embodiments, the expression of a YFV gene is reduced. In another embodiment, the gene may be one of a group that includes the E, NS2A, or NS3 genes. In some embodiments the expression of a human gene that is required for YFV replication is reduced.

Methods of the invention also provide for treating patients infected by the poliovirus or at risk for or afflicted with a disorder mediated by poliovirus, e.g., polio. In some embodiments, the expression of a poliovirus gene is reduced. In some embodiments the expression of a human gene that is required for poliovirus replication is reduced.

Methods of the invention also provide for treating patients infected by a poxvirus or at risk for or afflicted with a disorder mediated by a poxvirus, e.g., smallpox. In some embodiments, the expression of a poxvirus gene is reduced. In some embodiments the expression of a human gene that is required for poxvirus replication is reduced.

### Other Pathogens

In another, aspect the invention features methods of treating a subject infected with a pathogen, e.g., a bacterial, amoebic, parasitic, or fungal pathogen. The method comprises administering the siRNA of the invention to a subject in need thereof, thereby treating the subject. The nucleic acid that is administered will depend on the type of pathogen being treated. In some embodiments, the nucleic acid may target a pathogen gene. In other embodiments, the nucleic acid may target a host gene.

The target gene can be one involved in growth, cell wall synthesis, protein synthesis, transcription, energy metabolism, e.g., the Krebs cycle, or toxin production.

Thus, the present invention provides for a method of treating patients infected by a plasmodium that causes malaria. In some embodiments, the expression of a plasmodium gene is reduced. In another embodiment, the gene is apical membrane antigen 1 (AMA1). In some embodiments the expression of a human gene that is required for plasmodium replication is reduced.

The invention also includes methods for treating patients infected by the Mycobacterium ulcerans, or a disease or disorder associated with this pathogen, e.g., Buruli ulcers. In some embodiments, the expression of a Mycobacterium ulcerans gene is reduced. In some embodiments the expression of a human gene that is required for Mycobacterium ulcerans replication is reduced.

The invention also includes methods for treating patients infected by the Mycobacterium tuberculosis, or a disease or disorder associated with this pathogen, e.g., tuberculosis. In some embodiments, the expression of a Mycobacterium tuberculosis gene is reduced. In some embodiments the expression of a human gene that is required for Mycobacterium tuberculosis replication is reduced.

The invention also includes methods for treating patients infected by the Mycobacterium leprae, or a disease or disorder associated with this pathogen, e.g., leprosy. In some embodiments, the expression of a Mycobacterium leprae gene is reduced. In some embodiments the expression of a human gene that is required for Mycobacterium leprae replication is reduced.

The invention also includes methods for treating patients infected by the bacteria Staphylococcus aureus, or a disease or disorder associated with this pathogen, e.g., infections of the skin and muscous membranes. In some embodiments, the expression of a Staphylococcus aureus gene is reduced. In some embodiments the expression of a human gene that is required for Staphylococcus aureus replication is reduced.

The invention also includes methods for treating patients infected by the bacteria Streptococcus pneumoniae, or a disease or disorder associated with this pathogen, e.g., pneumonia or childhood lower respiratory tract infection. In some embodiments, the expression of a Streptococcus pneumoniae gene is reduced. In some embodiments the expression of a human gene that is required for Streptococcus pneumoniae replication is reduced.

The invention also includes methods for treating patients infected by the bacteria Streptococcus pyogenes, or a disease or disorder associated with this pathogen, e.g., Strep throat or Scarlet fever. In some embodiments, the expression of a Streptococcus pyogenes gene is reduced. In some embodiments the expression of a human gene that is required for Streptococcus pyogenes replication is reduced.

The invention also includes methods for treating patients infected by the bacteria Chlamydia pneumoniae, or a disease or disorder associated with this pathogen, e.g., pneumonia or childhood lower respiratory tract infection. In some embodiments, the expression of a Chlamydia pneumoniae gene is reduced. In some embodiments the expression of a human gene that is required for Chlamydia pneumoniae replication is reduced.

The invention also includes methods for treating patients infected by the bacteria Mycoplasma pneumoniae, or a disease or disorder associated with this pathogen, e.g., pneumonia or childhood lower respiratory tract infection. In some embodiments, the expression of a Mycoplasma pneumoniae gene is reduced. In some embodiments the expression of a human gene that is required for Mycoplasma pneumoniae replication is reduced.

### Immune Disorders

In one aspect, the invention features, a method of treating a subject, e.g., a human, at risk for or afflicted with a disease or disorder characterized by an unwanted immune response, e.g., an inflammatory disease or disorder, or an autoimmune disease or disorder. The method comprises administering the siRNA of the invention to a subject in need thereof, thereby treating the subject. The nucleic acid that is administered will depend on the type of immune disorder being treated.

In some embodiments the disease or disorder is an ischemia or reperfusion injury, e.g., ischemia or reperfusion injury associated with acute myocardial infarction, unstable angina, cardiopulmonary bypass, surgical intervention e.g., angioplasty, e.g., percutaneous transluminal coronary angioplasty, the response to a transplanted organ or tissue, e.g., transplanted cardiac or vascular tissue; or thrombolysis.

In some embodiments the disease or disorder is restenosis, e.g., restenosis associated with surgical intervention e.g., angioplasty, e.g., percutaneous transluminal coronary angioplasty.

In certain embodiments the disease or disorder is Inflammatory Bowel Disease, e.g., Crohn Disease or Ulcerative Colitis.

In certain embodiments the disease or disorder is inflammation associated with an infection or injury.

In certain embodiments the disease or disorder is asthma, lupus, multiple sclerosis, diabetes, e.g., type II diabetes, arthritis, e.g., rheumatoid or psoriatic.

In certain other embodiments the siRNA compound (e.g., the siRNA in a composition described herein) silences an integrin or co-ligand thereof, e.g., VLA4, VCAM, ICAM.

In certain other embodiments the siRNA compound (e.g., the siRNA in a composition described herein) silences a selectin or co-ligand thereof, e.g., P-selectin, E-selectin (ELAM), I-selectin, P-selectin glycoprotein-1 (PSGL-1).

In certain other embodiments the siRNA compound (e.g., the siRNA in a composition described herein) silences a component of the complement system, e.g., C3, C5, C3aR, C5aR, C3 convertase, C5 convertase.

In certain other embodiments the siRNA compound (e.g., the siRNA in a composition described herein) silences a chemokine or receptor thereof, e.g., TNFI, TNFJ, IL-1I, IL-1J, 1L-2, IL-2R, IL-4, IL-4R, IL-5, IL-6, IL-8, TNFRI, TNFRII, IgE, SCYA11, CCR3.

In other embodiments the siRNA compound (e.g., the siRNA in a composition described herein) silences GCSF, Gro1, Gro2, Gro3, PF4, MIG, Pro-Platelet Basic Protein (PPBP), MIP-1I, MIP-1J, RANTES, MCP-1, MCP-2, MCP-3, CMBKR1, CMBKR2, CMBKR3, CMBKR5, AIF-1, I-309.

### Pain

In one aspect, the invention provides a method of treating a subject, e.g., a human, at risk for or afflicted with acute pain or chronic pain. The method comprises administering the siRNA of the invention to a subject in need thereof, thereby treating the subject. The nucleic acid that is administered will depend on the type of pain being treated.

In certain other embodiments the siRNA compound (e.g., the siRNA in a composition described herein) silences a component of an ion channel.

In certain other embodiments the siRNA compound (e.g., the siRNA in a composition described herein) silences a neurotransmitter receptor or ligand.

In one aspect, the invention provides a method of treating a subject, e.g., a human, at risk for or afflicted with a neurological disease or disorder. The method includes: providing an siRNA compound (e.g., the siRNA in a composition described herein) homologous to and can silence, e.g., by cleavage, a gene which mediates a neurological disease or disorder, and administering the siRNA compound to a subject, thereby treating the subject.

### Neurological Disorders

In certain embodiments the disease or disorder is a neurological disorder, including Alzheimer's Disease or Parkinson Disease. The method comprises administering the siRNA of the invention to a subject in need thereof, thereby treating the subject. The nucleic acid that is administered will depend on the type of neurological disorder being treated.

In certain other embodiments the siRNA compound (e.g., the siRNA in a composition described herein) silences an amyloid-family gene, e.g., APP; a presenilin gene, e.g., PSEN1 and PSEN2, or I-synuclein.

In some embodiments the disease or disorder is a neurodegenerative trinucleotide repeat disorder, e.g., Huntington disease, dentatorubral pallidoluysian atrophy or a spinocerebellar ataxia, e.g., SCA1, SCA2, SCA3 (Machado-Joseph disease), SCA7 or SCA8.

In certain other embodimentsthe siRNA compound (e.g., the siRNA in a composition described herein) silences HD, DRPLA, SCA1, SCA2, MJD1, CACNL1A4, SCA7, SCA8.

### Loss of Heterozygosity

The loss of heterozygosity (LOH) can result in hemizygosity for sequence, e.g., genes, in the area of LOH. This can result in a significant genetic difference between normal and disease-state cells, e.g., cancer cells, and provides a useful difference between normal and disease-state cells, e.g., cancer cells. This difference can arise because a gene or other sequence is heterozygous in euploid cells but is hemizygous in cells having LOH. The regions of LOH will often include a gene, the loss of which promotes unwanted proliferation, e.g., a tumor suppressor gene, and other sequences including, e.g., other genes, in some cases a gene which is essential for normal function, e.g., growth. Methods of the invention rely, in part, on the specific cleavage or silencing of one allele of an essential gene with an siRNA compound (e.g., the siRNA in a composition described herein) of the invention. The siRNA compound (e.g., the siRNA in a composition described herein) is selected such that it targets the single allele of the essential gene found in the cells having LOH but does not silence the other allele, which is present in cells which do not show LOH. In essence, it discriminates between the two alleles, preferentially silencing the selected allele. In essence polymorphisms, e.g., SNPs of essential genes that are affected by LOH, are used as a target for a disorder characterized by cells having LOH, e.g., cancer cells having LOH.

One of ordinary skill in the art can identify essential genes which are in proximity to tumor suppressor genes, and which are within a LOH region which includes the tumor suppressor gene. The gene encoding the large subunit of human RNA polymerase II, POLR2A, a gene located in close proximity to the tumor suppressor gene p53, is such a gene. It frequently occurs within a region of LOH in cancer cells. Other genes that occur within LOH regions and are lost in many cancer cell types include the group comprising replication protein A 70-kDa subunit, replication protein A 32-kD, ribonucleotide reductase, thymidilate synthase, TATA associated factor 2H, ribosomal protein S14, eukaryotic initiation factor 5A, alanyl tRNA synthetase, cysteinyl tRNA synthetase, NaK ATPase, alpha-1 subunit, and transferrin receptor.

Accordingly, the invention features, a method of treating a disorder characterized by LOH, e.g., cancer. The method comprises optionally, determining the genotype of the allele of a gene in the region of LOH and determining the genotype of both alleles of the gene in a normal cell; providing an siRNA compound (e.g., the siRNA in a composition described herein) which preferentially cleaves or silences the allele found in the LOH cells; and administering the iRNA to the subject, thereby treating the disorder.

The invention also includes a siRNA compound (e.g., the siRNA in a composition described herein) disclosed herein, e.g, an siRNA compound (e.g., the siRNA in a composition described herein) which can preferentially silence, e.g., cleave, one allele of a polymorphic gene.

In another aspect, the invention provides a method of cleaving or silencing more than one gene with an siRNA compound (e.g., the siRNA in a composition described herein). In these embodiments the siRNA compound (e.g., the siRNA in a composition described herein) is selected so that it has sufficient homology to a sequence found in more than one gene. For example, the sequence AAGCTGGCCCTGGACATGGAGAT is conserved between mouse lamin B1, lamin B2, keratin complex 2-gene 1 and lamin A/C. Thus an siRNA compound (e.g., the siRNA in a composition described herein) targeted to this sequence would effectively silence the entire collection of genes.

The invention also includes an siRNA compound (e.g., the siRNA in a composition described herein) disclosed herein, which can silence more than one gene.

### Routes of Delivery

For ease of exposition the formulations, compositions and methods in this section are discussed largely with regard to modified siRNA compounds. It may be understood, however, that these formulations, compositions and methods can be practiced with other siRNA compounds, e.g., unmodified siRNA compounds, and such practice is within the invention. A composition that includes a iRNA can be delivered to a subject by a variety of routes. Exemplary routes include: intravenous, topical, rectal, anal, vaginal, nasal, pulmonary, ocular.

The iRNA molecules of the invention can be incorporated into pharmaceutical compositions suitable for administration. Such compositions typically include one or more species of iRNA and a pharmaceutically acceptable carrier. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

The pharmaceutical compositions of the present invention may be administered in a number of ways depending upon whether local or systemic treatment is desired and upon the area to be treated. Administration may be topical (including ophthalmic, vaginal, rectal, intranasal, transdermal), oral or parenteral. Parenteral administration includes intravenous drip, subcutaneous, intraperitoneal or intramuscular injection, or intrathecal or intraventricular administration.

The route and site of administration may be chosen to enhance targeting. For example, to target muscle cells, intramuscular injection into the muscles of interest would be a logical choice. Lung cells might be targeted by administering the iRNA in aerosol form. The vascular endothelial cells could be targeted by coating a balloon catheter with the iRNA and mechanically introducing the DNA.

Formulations for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable. Coated condoms, gloves and the like may also be useful.

Compositions for oral administration include powders or granules, suspensions or solutions in water, syrups, elixirs or non-aqueous media, tablets, capsules, lozenges, or troches. In the case of tablets, carriers that can be used include lactose, sodium citrate and salts of phosphoric acid. Various disintegrants such as starch, and lubricating agents such as magnesium stearate, sodium lauryl sulfate and talc, are commonly used in tablets. For oral administration in capsule form, useful diluents are lactose and high molecular weight polyethylene glycols. When aqueous suspensions are required for oral use, the nucleic acid compositions can be combined with emulsifying and suspending agents. If desired, certain sweetening and/or flavoring agents can be added.

Compositions for intrathecal or intraventricular administration may include sterile aqueous solutions which may also contain buffers, diluents and other suitable additives.

Formulations for parenteral administration may include sterile aqueous solutions which may also contain buffers, diluents and other suitable additives. Intraventricular injection may be facilitated by an intraventricular catheter, for example, attached to a reservoir. For intravenous use, the total concentration of solutes may be controlled to render the preparation isotonic.

For ocular administration, ointments or droppable liquids may be delivered by ocular delivery systems known to the art such as applicators or eye droppers. Such compositions can include mucomimetics such as hyaluronic acid, chondroitin sulfate, hydroxypropyl methylcellulose or poly (vinyl alcohol), preservatives such as sorbic acid, EDTA or benzylchronium chloride, and the usual quantities of diluents and/or carriers.

### Topical Delivery

For ease of exposition the formulations, compositions and methods in this section are discussed largely with regard tonmodified siRNA compounds. It may be understood, however, that these formulations, compositions and methods can be practiced with other siRNA compounds, e.g., unmodified siRNA compounds, and such practice is within the invention. In some embodiments, an siRNA compound, e.g., a double-stranded siRNA compound, or ssiRNA compound, (e.g., a precursor, e.g., a larger siRNA compound which can be processed into a ssiRNA compound, or a DNA which encodes an siRNA compound, e.g., a double-stranded siRNA compound, or ssiRNA compound, or precursor thereof) is delivered to a subject via topical administration. “Topical administration” refers to the delivery to a subject by contacting the formulation directly to a surface of the subject. The most common form of topical delivery is to the skin, but a composition disclosed herein can also be directly applied to other surfaces of the body, e.g., to the eye, a mucous membrane, to surfaces of a body cavity or to an internal surface. As mentioned above, the most common topical delivery is to the skin. The term encompasses several routes of administration including, but not limited to, topical and transdermal. These modes of administration typically include penetration of the skin's permeability barrier and efficient delivery to the target tissue or stratum. Topical administration can be used as a means to penetrate the epidermis and dermis and ultimately achieve systemic delivery of the composition. Topical administration can also be used as a means to selectively deliver oligonucleotides to the epidermis or dermis of a subject, or to specific strata thereof, or to an underlying tissue.

The term “skin,” as used herein, refers to the epidermis and/or dermis of an animal Mammalian skin consists of two major, distinct layers. The outer layer of the skin is called the epidermis. The epidermis is comprised of the stratum corneum, the stratum granulosum, the stratum spinosum, and the stratum basale, with the stratum corneum being at the surface of the skin and the stratum basale being the deepest portion of the epidermis. The epidermis is between 50 μm and 0.2 mm thick, depending on its location on the body.

Beneath the epidermis is the dermis, which is significantly thicker than the epidermis. The dermis is primarily composed of collagen in the form of fibrous bundles. The collagenous bundles provide support for, inter alia, blood vessels, lymph capillaries, glands, nerve endings and immunologically active cells.

One of the major functions of the skin as an organ is to regulate the entry of substances into the body. The principal permeability barrier of the skin is provided by the stratum corneum, which is formed from many layers of cells in various states of differentiation. The spaces between cells in the stratum corneum is filled with different lipids arranged in lattice-like formations that provide seals to further enhance the skins permeability barrier.

The permeability barrier provided by the skin is such that it is largely impermeable to molecules having molecular weight greater than about 750 Da. For larger molecules to cross the skin's permeability barrier, mechanisms other than normal osmosis must be used.

Several factors determine the permeability of the skin to administered agents. These factors include the characteristics of the treated skin, the characteristics of the delivery agent, interactions between both the drug and delivery agent and the drug and skin, the dosage of the drug applied, the form of treatment, and the post treatment regimen. To selectively target the epidermis and dermis, it is sometimes possible to formulate a composition that comprises one or more penetration enhancers that will enable penetration of the drug to a preselected stratum.

Transdermal delivery is a valuable route for the administration of lipid soluble therapeutics. The dermis is more permeable than the epidermis and therefore absorption is much more rapid through abraded, burned or denuded skin. Inflammation and other physiologic conditions that increase blood flow to the skin also enhance transdermal adsorption. Absorption via this route may be enhanced by the use of an oily vehicle (inunction) or through the use of one or more penetration enhancers. Other effective ways to deliver a composition disclosed herein via the transdermal route include hydration of the skin and the use of controlled release topical patches. The transdermal route provides a potentially effective means to deliver a composition disclosed herein for systemic and/or local therapy.

In addition, iontophoresis (transfer of ionic solutes through biological membranes under the influence of an electric field) (Lee et al., Critical Reviews in Therapeutic Drug Carrier Systems, 1991, p. 163), phonophoresis or sonophoresis (use of ultrasound to enhance the absorption of various therapeutic agents across biological membranes, notably the skin and the cornea) (Lee et al., Critical Reviews in Therapeutic Drug Carrier Systems, 1991, p. 166), and optimization of vehicle characteristics relative to dose position and retention at the site of administration (Lee et al., Critical Reviews in Therapeutic Drug Carrier Systems, 1991, p. 168) may be useful methods for enhancing the transport of topically applied compositions across skin and mucosal sites.

The compositions and methods provided may also be used to examine the function of various proteins and genes in vitro in cultured or preserved dermal tissues and in animals. The invention can be thus applied to examine the function of any gene. The methods of the invention can also be used therapeutically or prophylactically. For example, for the treatment of animals that are known or suspected to suffer from diseases such as psoriasis, lichen planus, toxic epidermal necrolysis, ertythema multiforme, basal cell carcinoma, squamous cell carcinoma, malignant melanoma, Paget's disease, Kaposi's sarcoma, pulmonary fibrosis, Lyme disease and viral, fungal and bacterial infections of the skin.

### Pulmonary Delivery

For ease of exposition the formulations, compositions and methods in this section are discussed largely with regard to modified siRNA compounds. It may be understood, however, that these formulations, compositions and methods can be practiced with other siRNA compounds, e.g., unmodified siRNA compounds, and such practice is within the invention. A composition that includes an siRNA compound, e.g., a double-stranded siRNA compound, or ssiRNA compound, (e.g., a precursor, e.g., a larger siRNA compound which can be processed into a ssiRNA compound, or a DNA which encodes an siRNA compound, e.g., a double-stranded siRNA compound, or ssiRNA compound, or precursor thereof) can be administered to a subject by pulmonary delivery. Pulmonary delivery compositions can be delivered by inhalation by the patient of a dispersion so that the composition, for example, iRNA, within the dispersion can reach the lung where it can be readily absorbed through the alveolar region directly into blood circulation. Pulmonary delivery can be effective both for systemic delivery and for localized delivery to treat diseases of the lungs.

Pulmonary delivery can be achieved by different approaches, including the use of nebulized, aerosolized, micellular and dry powder-based formulations. Delivery can be achieved with liquid nebulizers, aerosol-based inhalers, and dry powder dispersion devices. Metered-dose devices are may be used. One of the benefits of using an atomizer or inhaler is that the potential for contamination is minimized because the devices are self contained. Dry powder dispersion devices, for example, deliver drugs that may be readily formulated as dry powders. A iRNA composition may be stably stored as lyophilized or spray-dried powders by itself or in combination with suitable powder carriers. The delivery of a composition for inhalation can be mediated by a dosing timing element which can include a timer, a dose counter, time measuring device, or a time indicator which when incorporated into the device enables dose tracking, compliance monitoring, and/or dose triggering to a patient during administration of the aerosol medicament.

The term “powder” means a composition that consists of finely dispersed solid particles that are free flowing and capable of being readily dispersed in an inhalation device and subsequently inhaled by a subject so that the particles reach the lungs to permit penetration into the alveoli. Thus, the powder is said to be “respirable.” For example, the average particle size is less than about 10 μm in diameter with a relatively uniform spheroidal shape distribution. In some embodiments, the diameter is less than about 7.5 μm and in some embodiments less than about 5.0 μm. Usually the particle size distribution is between about 0.1 μm and about 5 μm in diameter, sometimes about 0.3 μm to about 5 μm.

The term “dry” means that the composition has a moisture content below about 10% by weight (% w) water, usually below about 5% w and in some cases less it than about 3% w. A dry composition can be such that the particles are readily dispersible in an inhalation device to form an aerosol.

The term “therapeutically effective amount” is the amount present in the composition that is needed to provide the desired level of drug in the subject to be treated to give the anticipated physiological response.

The term “physiologically effective amount” is that amount delivered to a subject to give the desired palliative or curative effect.

The term “pharmaceutically acceptable carrier” means that the carrier can be taken into the lungs with no significant adverse toxicological effects on the lungs.

The types of pharmaceutical excipients that are useful as carrier include stabilizers such as human serum albumin (HSA), bulking agents such as carbohydrates, amino acids and polypeptides; pH adjusters or buffers; salts such as sodium chloride; and the like. These carriers may be in a crystalline or amorphous form or may be a mixture of the two.

Bulking agents that are particularly valuable include compatible carbohydrates, polypeptides, amino acids or combinations thereof. Suitable carbohydrates include monosaccharides such as galactose, D-mannose, sorbose, and the like; disaccharides, such as lactose, trehalose, and the like; cyclodextrins, such as 2-hydroxypropyl-.beta.-cyclodextrin; and polysaccharides, such as raffinose, maltodextrins, dextrans, and the like; alditols, such as mannitol, xylitol, and the like. A group of carbohydrates may include lactose, threhalose, raffinose maltodextrins, and mannitol. Suitable polypeptides include aspartame Amino acids include alanine and glycine, with glycine being used in some embodiments.

Additives, which are minor components of the composition of this invention, may be included for conformational stability during spray drying and for improving dispersibility of the powder. These additives include hydrophobic amino acids such as tryptophan, tyrosine, leucine, phenylalanine, and the like.

Suitable pH adjusters or buffers include organic salts prepared from organic acids and bases, such as sodium citrate, sodium ascorbate, and the like; sodium citrate may be used in some embodiments.

Pulmonary administration of a micellar iRNA formulation may be achieved through metered dose spray devices with propellants such as tetrafluoroethane, heptafluoroethane, dimethylfluoropropane, tetrafluoropropane, butane, isobutane, dimethyl ether and other non-CFC and CFC propellants.

### Oral or Nasal Delivery

For ease of exposition the formulations, compositions and methods in this section are discussed largely with regard to modified siRNA compounds. It may be understood, however, that these formulations, compositions and methods can be practiced with other siRNA compounds, e.g., unmodified siRNA compounds, and such practice is within the invention. Both the oral and nasal membranes offer advantages over other routes of administration. For example, drugs administered through these membranes have a rapid onset of action, provide therapeutic plasma levels, avoid first pass effect of hepatic metabolism, and avoid exposure of the drug to the hostile gastrointestinal (GI) environment. Additional advantages include easy access to the membrane sites so that the drug can be applied, localized and removed easily.

In oral delivery, compositions can be targeted to a surface of the oral cavity, e.g., to sublingual mucosa which includes the membrane of ventral surface of the tongue and the floor of the mouth or the buccal mucosa which constitutes the lining of the cheek. The sublingual mucosa is relatively permeable thus giving rapid absorption and acceptable bioavailability of many drugs. Further, the sublingual mucosa is convenient, acceptable and easily accessible.

The ability of molecules to permeate through the oral mucosa appears to be related to molecular size, lipid solubility and peptide protein ionization. Small molecules, less than 1000 daltons appear to cross mucosa rapidly. As molecular size increases, the permeability decreases rapidly. Lipid soluble compounds are more permeable than non-lipid soluble molecules. Maximum absorption occurs when molecules are un-ionized or neutral in electrical charges. Therefore charged molecules present the biggest challenges to absorption through the oral mucosae.

A pharmaceutical composition of iRNA may also be administered to the buccal cavity of a human being by spraying into the cavity, without inhalation, from a metered dose spray dispenser, a mixed micellar pharmaceutical formulation as described above and a propellant. In one embodiment, the dispenser is first shaken prior to spraying the pharmaceutical formulation and propellant into the buccal cavity.

### Devices

For ease of exposition the devices, formulations, compositions and methods in this section are discussed largely with regard to modified siRNA compounds. It may be understood, however, that these devices, formulations, compositions and methods can be practiced with other siRNA compounds, e.g., unmodified siRNA compounds, and such practice is within the invention. An siRNA compound, e.g., a double-stranded siRNA compound, or ssiRNA compound, (e.g., a precursor, e.g., a larger siRNA compound which can be processed into a ssiRNA compound, or a DNA which encodes an siRNA compound, e.g., a double-stranded siRNA compound, or ssiRNA compound, or precursor thereof) can be disposed on or in a device, e.g., a device which implanted or otherwise placed in a subject. Exemplary devices include devices which are introduced into the vasculature, e.g., devices inserted into the lumen of a vascular tissue, or which devices themselves form a part of the vasculature, including stents, catheters, heart valves, and other vascular devices. These devices, e.g., catheters or stents, can be placed in the vasculature of the lung, heart, or leg.

Other devices include non-vascular devices, e.g., devices implanted in the peritoneum, or in organ or glandular tissue, e.g., artificial organs. The device can release a therapeutic substance in addition to an siRNA, e.g., a device can release insulin.

Other devices include artificial joints, e.g., hip joints, and other orthopedic implants.

In one embodiment, unit doses or measured doses of a composition that includes iRNA are dispensed by an implanted device. The device can include a sensor that monitors a parameter within a subject. For example, the device can include pump, e.g., and, optionally, associated electronics.

Tissue, e.g., cells or organs can be treated with an siRNA compound, e.g., a double-stranded siRNA compound, or ssiRNA compound, (e.g., a precursor, e.g., a larger siRNA compound which can be processed into a ssiRNA compound, or a DNA which encodes an siRNA compound, e.g., a double-stranded siRNA compound, or ssiRNA compound, or precursor thereof), ex vivo and then administered or implanted in a subject.

The tissue can be autologous, allogeneic, or xenogeneic tissue. E.g., tissue can be treated to reduce graft versus host disease. In other embodiments, the tissue is allogeneic and the tissue is treated to treat a disorder characterized by unwanted gene expression in that tissue. E.g., tissue, e.g., hematopoietic cells, e.g., bone marrow hematopoietic cells, can be treated to inhibit unwanted cell proliferation.

Introduction of treated tissue, whether autologous or transplant, can be combined with other therapies.

In some implementations, the iRNA treated cells are insulated from other cells, e.g., by a semi-permeable porous barrier that prevents the cells from leaving the implant, but enables molecules from the body to reach the cells and molecules produced by the cells to enter the body.

In one embodiment, the porous barrier is formed from alginate.

In one embodiment, a contraceptive device is coated with or contains an siRNA compound, e.g., a double-stranded siRNA compound, or ssiRNA compound, (e.g., a precursor, e.g., a larger siRNA compound which can be processed into a ssiRNA compound, or a DNA which encodes an siRNA compound, e.g., a double-stranded siRNA compound, or ssiRNA compound, or precursor thereof). Exemplary devices include condoms, diaphragms, IUD (implantable uterine devices, sponges, vaginal sheaths, and birth control devices. In one embodiment, the iRNA is chosen to inactive sperm or egg. In another embodiment, the iRNA is chosen to be complementary to a viral or pathogen RNA, e.g., an RNA of an STD. In some instances, the iRNA composition can include a spermicide.

### Dosage

In one aspect, the invention features a method of administering an siRNA compound, e.g., a double-stranded siRNA compound, or ssiRNA compound, to a subject (e.g., a human subject). The method includes administering a unit dose of the siRNA compound, e.g., a ssiRNA compound, e.g., double stranded ssiRNA compound that (a) the double-stranded part is 19-25 nucleotides (nt) long, for example, 21-23 nt, (b) is complementary to a target RNA (e.g., an endogenous or pathogen target RNA), and, optionally, (c) includes at least one 3′ overhang 1-5 nucleotide long. In one embodiment, the unit dose is less than 1.4 mg per kg of bodyweight, or less than 10, 5, 2, 1, 0.5, 0.1, 0.05, 0.01, 0.005, 0.001, 0.0005, 0.0001, 0.00005 or 0.00001 mg per kg of bodyweight, and less than 200 nmole of RNA agent (e.g., about 4.4×10¹⁶ copies) per kg of bodyweight, or less than 1500, 750, 300, 150, 75, 15, 7.5, 1.5, 0.75, 0.15, 0.075, 0.015, 0.0075, 0.0015, 0.00075, 0.00015 nmole of RNA agent per kg of bodyweight.

The defined amount can be an amount effective to treat or prevent a disease or disorder, e.g., a disease or disorder associated with the target RNA. The unit dose, for example, can be administered by injection (e.g., intravenous or intramuscular), an inhaled dose, or a topical application. In some embodiments dosages may be less than 2, 1, or 0.1 mg/kg of body weight.

In some embodiments, the unit dose is administered less frequently than once a day, e.g., less than every 2, 4, 8 or 30 days. In another embodiment, the unit dose is not administered with a frequency (e.g., not a regular frequency). For example, the unit dose may be administered a single time.

RNAi silencing persists for several days after administering an siRNA composition so, in many instances, it is possible to administer the composition with a frequency of less than once per day, or, for some instances, only once for the entire therapeutic regimen. For example, treatment of some cancer cells may be mediated by a single bolus administration, whereas a chronic viral infection may require regular administration, e.g., once or more per week or once or less per month.

In one embodiment, the effective dose is administered with other traditional therapeutic modalities. In one embodiment, the subject has a viral infection and the modality is an antiviral agent other than an siRNA compound, e.g., other than a double-stranded siRNA compound, or ssiRNA compound. In another embodiment, the subject has atherosclerosis and the effective dose of an siRNA compound, e.g., a double-stranded siRNA compound, or ssiRNA compound, is administered in combination with, e.g., after surgical intervention, e.g., angioplasty.

In one embodiment, a subject is administered an initial dose and one or more maintenance doses of an siRNA compound, e.g., a double-stranded siRNA compound, or ssiRNA compound, (e.g., a precursor, e.g., a larger siRNA compound which can be processed into a ssiRNA compound, or a DNA which encodes an siRNA compound, e.g., a double-stranded siRNA compound, or ssiRNA compound, or precursor thereof). The maintenance dose or doses are generally lower than the initial dose, e.g., one-half less of the initial dose. A maintenance regimen can include treating the subject with a dose or doses ranging from 0.01 μg to 1.4 mg/kg of body weight per day, e.g., 10, 1, 0.1, 0.01, 0.001, or 0.00001 mg per kg of bodyweight per day. The maintenance doses are, for example, administered no more than once every 5, 10, or 30 days. Further, the treatment regimen may last for a period of time which will vary depending upon the nature of the particular disease, its severity and the overall condition of the patient. In certain embodiments the dosage may be delivered no more than once per day, e.g., no more than once per 24, 36, 48, or more hours, e.g., no more than once for every 5 or 8 days. Following treatment, the patient can be monitored for changes in his condition and for alleviation of the symptoms of the disease state. The dosage of the compound may either be increased in the event the patient does not respond significantly to current dosage levels, or the dose may be decreased if an alleviation of the symptoms of the disease state is observed, if the disease state has been ablated, or if undesired side-effects are observed.

The effective dose can be administered in a single dose or in two or more doses, as desired or considered appropriate under the specific circumstances. If desired to facilitate repeated or frequent infusions, implantation of a delivery device, e.g., a pump, semi-permanent stent (e.g., intravenous, intraperitoneal, intracisternal or intracapsular), or reservoir may be advisable.

In one embodiment, the siRNA compound pharmaceutical composition includes a plurality of siRNA compound species. In another embodiment, the siRNA compound species has sequences that are non-overlapping and non-adjacent to another species with respect to a naturally occurring target sequence. In another embodiment, the plurality of siRNA compound species is specific for different naturally occurring target genes. In another embodiment, the siRNA compound is allele specific.

In some cases, a patient is treated with a siRNA compound in conjunction with other therapeutic modalities. For example, a patient being treated for a viral disease, e.g., an HIV associated disease (e.g., AIDS), may be administered a siRNA compound specific for a target gene essential to the virus in conjunction with a known antiviral agent (e.g., a protease inhibitor or reverse transcriptase inhibitor). In another example, a patient being treated for cancer may be administered a siRNA compound specific for a target essential for tumor cell proliferation in conjunction with a chemotherapy.

Following successful treatment, it may be desirable to have the patient undergo maintenance therapy to prevent the recurrence of the disease state, wherein the compound of the invention is administered in maintenance doses, ranging from 0.01 μg to 100 g per kg of body weight (see U.S. Pat. No. 6,107,094).

The concentration of the siRNA compound composition is an amount sufficient to be effective in treating or preventing a disorder or to regulate a physiological condition in humans. The concentration or amount of siRNA compound administered will depend on the parameters determined for the agent and the method of administration, e.g., nasal, buccal, pulmonary. For example, nasal formulations tend to require much lower concentrations of some ingredients in order to avoid irritation or burning of the nasal passages. It is sometimes desirable to dilute an oral formulation up to 10-100 times in order to provide a suitable nasal formulation.

Certain factors may influence the dosage required to effectively treat a subject, including but not limited to the severity of the disease or disorder, previous treatments, the general health and/or age of the subject, and other diseases present. Moreover, treatment of a subject with a therapeutically effective amount of an siRNA compound, e.g., a double-stranded siRNA compound, or ssiRNA compound, (e.g., a precursor, e.g., a larger siRNA compound which can be processed into a ssiRNA compound, or a DNA which encodes an siRNA compound, e.g., a double-stranded siRNA compound, or ssiRNA compound, or precursor thereof) can include a single treatment or, for example, can include a series of treatments. It will also be appreciated that the effective dosage of a siRNA compound such as a ssiRNA compound used for treatment may increase or decrease over the course of a particular treatment. Changes in dosage may result and become apparent from the results of diagnostic assays as described herein. For example, the subject can be monitored after administering a siRNA compound composition. Based on information from the monitoring, an additional amount of the siRNA compound composition can be administered.

Dosing is dependent on severity and responsiveness of the disease condition to be treated, with the course of treatment lasting from several days to several months, or until a cure is effected or a diminution of disease state is achieved. Optimal dosing schedules can be calculated from measurements of drug accumulation in the body of the patient. Persons of ordinary skill can easily determine optimum dosages, dosing methodologies and repetition rates. Optimum dosages may vary depending on the relative potency of individual compounds, and can generally be estimated based on EC50s found to be effective in in vitro and in vivo animal models. In some embodiments, the animal models include transgenic animals that express a human gene, e.g., a gene that produces a target RNA. The transgenic animal can be deficient for the corresponding endogenous RNA. In another embodiment, the composition for testing includes a siRNA compound that is complementary, at least in an internal region, to a sequence that is conserved between the target RNA in the animal model and the target RNA in a human

The inventors have discovered that siRNA compounds described herein can be administered to mammals, particularly large mammals such as nonhuman primates or humans in a number of ways.

In one embodiment, the administration of the siRNA compound, e.g., a double-stranded siRNA compound, or ssiRNA compound, composition is parenteral, e.g., intravenous (e.g., as a bolus or as a diffusible infusion), intradermal, intraperitoneal, intramuscular, intrathecal, intraventricular, intracranial, subcutaneous, transmucosal, buccal, sublingual, endoscopic, rectal, oral, vaginal, topical, pulmonary, intranasal, urethral or ocular. Administration can be provided by the subject or by another person, e.g., a health care provider. The medication can be provided in measured doses or in a dispenser which delivers a metered dose. Selected modes of delivery are discussed in more detail below.

The invention provides methods, compositions, and kits, for rectal administration or delivery of siRNA compounds described herein.

Accordingly, an siRNA compound, e.g., a double-stranded siRNA compound, or ssiRNA compound, (e.g., a precursor, e.g., a larger siRNA compound which can be processed into a ssiRNA compound, or a DNA which encodes a an siRNA compound, e.g., a double-stranded siRNA compound, or ssiRNA compound, or precursor thereof) described herein, e.g., a therapeutically effective amount of a siRNA compound described herein, e.g., a siRNA compound having a double stranded region of less than 40, and, for example, less than 30 nucleotides and having one or two 1-3 nucleotide single strand 3′ overhangs can be administered rectally, e.g., introduced through the rectum into the lower or upper colon. This approach is particularly useful in the treatment of, inflammatory disorders, disorders characterized by unwanted cell proliferation, e.g., polyps, or colon cancer.

The medication can be delivered to a site in the colon by introducing a dispensing device, e.g., a flexible, camera-guided device similar to that used for inspection of the colon or removal of polyps, which includes means for delivery of the medication.

The rectal administration of the siRNA compound is by means of an enema. The siRNA compound of the enema can be dissolved in a saline or buffered solution. The rectal administration can also by means of a suppository, which can include other ingredients, e.g., an excipient, e.g., cocoa butter or hydropropylmethylcellulose.

Any of the siRNA compounds described herein can be administered orally, e.g., in the form of tablets, capsules, gel capsules, lozenges, troches or liquid syrups. Further, the composition can be applied topically to a surface of the oral cavity.

Any of the siRNA compounds described herein can be administered buccally. For example, the medication can be sprayed into the buccal cavity or applied directly, e.g., in a liquid, solid, or gel form to a surface in the buccal cavity. This administration is particularly desirable for the treatment of inflammations of the buccal cavity, e.g., the gums or tongue, e.g., in one embodiment, the buccal administration is by spraying into the cavity, e.g., without inhalation, from a dispenser, e.g., a metered dose spray dispenser that dispenses the pharmaceutical composition and a propellant.

Any of the siRNA compounds described herein can be administered to ocular tissue. For example, the medications can be applied to the surface of the eye or nearby tissue, e.g., the inside of the eyelid. They can be applied topically, e.g., by spraying, in drops, as an eyewash, or an ointment. Administration can be provided by the subject or by another person, e.g., a health care provider. The medication can be provided in measured doses or in a dispenser which delivers a metered dose. The medication can also be administered to the interior of the eye, and can be introduced by a needle or other delivery device which can introduce it to a selected area or structure. Ocular treatment is particularly desirable for treating inflammation of the eye or nearby tissue.

Any of the siRNA compounds described herein can be administered directly to the skin. For example, the medication can be applied topically or delivered in a layer of the skin, e.g., by the use of a microneedle or a battery of microneedles which penetrate into the skin, but, for example, not into the underlying muscle tissue. Administration of the siRNA compound composition can be topical. Topical applications can, for example, deliver the composition to the dermis or epidermis of a subject. Topical administration can be in the form of transdermal patches, ointments, lotions, creams, gels, drops, suppositories, sprays, liquids or powders. A composition for topical administration can be formulated as a liposome, micelle, emulsion, or other lipophilic molecular assembly. The transdermal administration can be applied with at least one penetration enhancer, such as iontophoresis, phonophoresis, and sonophoresis.

Any of the siRNA compounds described herein can be administered to the pulmonary system. Pulmonary administration can be achieved by inhalation or by the introduction of a delivery device into the pulmonary system, e.g., by introducing a delivery device which can dispense the medication. Certain embodiments may use a method of pulmonary delivery by inhalation. The medication can be provided in a dispenser which delivers the medication, e.g., wet or dry, in a form sufficiently small such that it can be inhaled. The device can deliver a metered dose of medication. The subject, or another person, can administer the medication.

Pulmonary delivery is effective not only for disorders which directly affect pulmonary tissue, but also for disorders which affect other tissue.

siRNA compounds can be formulated as a liquid or nonliquid, e.g., a powder, crystal, or aerosol for pulmonary delivery.

Any of the siRNA compounds described herein can be administered nasally. Nasal administration can be achieved by introduction of a delivery device into the nose, e.g., by introducing a delivery device which can dispense the medication. Methods of nasal delivery include spray, aerosol, liquid, e.g., by drops, or by topical administration to a surface of the nasal cavity. The medication can be provided in a dispenser with delivery of the medication, e.g., wet or dry, in a form sufficiently small such that it can be inhaled. The device can deliver a metered dose of medication. The subject, or another person, can administer the medication.

Nasal delivery is effective not only for disorders which directly affect nasal tissue, but also for disorders which affect other tissue siRNA compounds can be formulated as a liquid or nonliquid, e.g., a powder, crystal, or for nasal delivery.

An siRNA compound can be packaged in a viral natural capsid or in a chemically or enzymatically produced artificial capsid or structure derived therefrom.

The dosage of a pharmaceutical composition including a siRNA compound can be administered in order to alleviate the symptoms of a disease state, e.g., cancer or a cardiovascular disease. A subject can be treated with the pharmaceutical composition by any of the methods mentioned above.

Gene expression in a subject can be modulated by administering a pharmaceutical composition including an siRNA compound.

A subject can be treated by administering a defined amount of an siRNA compound, e.g., a double-stranded siRNA compound, or ssiRNA compound, (e.g., a precursor, e.g., a larger siRNA compound which can be processed into a ssiRNA compound) composition that is in a powdered form, e.g., a collection of microparticles, such as crystalline particles. The composition can include a plurality of siRNA compounds, e.g., specific for one or more different endogenous target RNAs. The method can include other features described herein.

A subject can be treated by administering a defined amount of an siRNA compound composition that is prepared by a method that includes spray-drying, i.e., atomizing a liquid solution, emulsion, or suspension, immediately exposing the droplets to a drying gas, and collecting the resulting porous powder particles. The composition can include a plurality of siRNA compounds, e.g., specific for one or more different endogenous target RNAs. The method can include other features described herein.

The siRNA compound, e.g., a double-stranded siRNA compound, or ssiRNA compound, (e.g., a precursor, e.g., a larger siRNA compound which can be processed into a ssiRNA compound, or a DNA which encodes an siRNA compound, e.g., a double-stranded siRNA compound, or ssiRNA compound, or precursor thereof), can be provided in a powdered, crystallized or other finely divided form, with or without a carrier, e.g., a micro- or nano-particle suitable for inhalation or other pulmonary delivery. This can include providing an aerosol preparation, e.g., an aerosolized spray-dried composition. The aerosol composition can be provided in and/or dispensed by a metered dose delivery device.

The subject can be treated for a condition treatable by inhalation, e.g., by aerosolizing a spray-dried siRNA compound, e.g., a double-stranded siRNA compound, or ssiRNA compound, (e.g., a precursor, e.g., a larger siRNA compound which can be processed into a ssiRNA compound, or a DNA which encodes an siRNA compound, e.g., a double-stranded siRNA compound, or ssiRNA compound, or precursor thereof) composition and inhaling the aerosolized composition. The siRNA compound can be an siRNA. The composition can include a plurality of siRNA compounds, e.g., specific for one or more different endogenous target RNAs. The method can include other features described herein.

A subject can be treated by, for example, administering a composition including an effective/defined amount of an siRNA compound, e.g., a double-stranded siRNA compound, or ssiRNA compound, (e.g., a precursor, e.g., a larger siRNA compound which can be processed into a ssiRNA compound, or a DNA which encodes an siRNA compound, e.g., a double-stranded siRNA compound, or ssiRNA compound, or precursor thereof), wherein the composition is prepared by a method that includes spray-drying, lyophilization, vacuum drying, evaporation, fluid bed drying, or a combination of these techniques.

In another aspect, the invention features a method that includes: evaluating a parameter related to the abundance of a transcript in a cell of a subject; comparing the evaluated parameter to a reference value; and if the evaluated parameter has a preselected relationship to the reference value (e.g., it is greater), administering a siRNA compound (or a precursor, e.g., a larger siRNA compound which can be processed into a ssiRNA compound, or a DNA which encodes a siRNA compound or precursor thereof) to the subject. In one embodiment, the siRNA compound includes a sequence that is complementary to the evaluated transcript. For example, the parameter can be a direct measure of transcript levels, a measure of a protein level, a disease or disorder symptom or characterization (e.g., rate of cell proliferation and/or tumor mass, viral load).

In another aspect, the invention features a method that includes: administering a first amount of a composition that comprises an siRNA compound, e.g., a double-stranded siRNA compound, or ssiRNA compound, (e.g., a precursor, e.g., a larger siRNA compound which can be processed into a ssiRNA compound, or a DNA which encodes an siRNA compound, e.g., a double-stranded siRNA compound, or ssiRNA compound, or precursor thereof) to a subject, wherein the siRNA compound includes a strand substantially complementary to a target nucleic acid; evaluating an activity associated with a protein encoded by the target nucleic acid; wherein the evaluation is used to determine if a second amount may be administered. In some embodiments the method includes administering a second amount of the composition, wherein the timing of administration or dosage of the second amount is a function of the evaluating. The method can include other features described herein.

In another aspect, the invention features a method of administering a source of a double-stranded siRNA compound (dssiRNA compound) to a subject. The method includes administering or implanting a source of a dssiRNA compound, e.g., a ssiRNA compound, that (a) includes a double-stranded region that is 19-25 nucleotides long, for example, 21-23 nucleotides, (b) is complementary to a target RNA (e.g., an endogenous RNA or a pathogen RNA), and, optionally, (c) includes at least one 3′ overhang 1-5 nt long. In one embodiment, the source releases dssiRNA compound over time, e.g., the source is a controlled or a slow release source, e.g., a microparticle that gradually releases the dssiRNA compound. In another embodiment, the source is a pump, e.g., a pump that includes a sensor or a pump that can release one or more unit doses.

In one aspect, the invention features a pharmaceutical composition that includes an siRNA compound, e.g., a double-stranded siRNA compound, or ssiRNA compound, (e.g., a precursor, e.g., a larger siRNA compound which can be processed into a ssiRNA compound, or a DNA which encodes an siRNA compound, e.g., a double-stranded siRNA compound, or ssiRNA compound, or precursor thereof) including a nucleotide sequence complementary to a target RNA, e.g., substantially and/or exactly complementary. The target RNA can be a transcript of an endogenous human gene. In one embodiment, the siRNA compound (a) is 19-25 nucleotides long, for example, 21-23 nucleotides, (b) is complementary to an endogenous target RNA, and, optionally, (c) includes at least one 3′ overhang 1-5 nt long. In one embodiment, the pharmaceutical composition can be an emulsion, microemulsion, cream, jelly, or liposome.

In one example the pharmaceutical composition includes an siRNA compound mixed with a topical delivery agent. The topical delivery agent can be a plurality of microscopic vesicles. The microscopic vesicles can be liposomes. In some embodiments the liposomes are cationic liposomes.

In another aspect, the pharmaceutical composition includes an siRNA compound, e.g., a double-stranded siRNA compound, or ssiRNA compound (e.g., a precursor, e.g., a larger siRNA compound which can be processed into a ssiRNA compound, or a DNA which encodes an siRNA compound, e.g., a double-stranded siRNA compound, or ssiRNA compound, or precursor thereof) admixed with a topical penetration enhancer. In one embodiment, the topical penetration enhancer is a fatty acid. The fatty acid can be arachidonic acid, oleic acid, lauric acid, caprylic acid, capric acid, myristic acid, palmitic acid, stearic acid, linoleic acid, linolenic acid, dicaprate, tricaprate, monolein, dilaurin, glyceryl 1-monocaprate, 1-dodecylazacycloheptan-2-one, an acylcarnitine, an acylcholine, or a C₁₋₁₀ alkyl ester, monoglyceride, diglyceride or pharmaceutically acceptable salt thereof.

In another embodiment, the topical penetration enhancer is a bile salt. The bile salt can be cholic acid, dehydrocholic acid, deoxycholic acid, glucholic acid, glycholic acid, glycodeoxycholic acid, taurocholic acid, taurodeoxycholic acid, chenodeoxycholic acid, ursodeoxycholic acid, sodium tauro-24,25-dihydro-fusidate, sodium glycodihydrofusidate, polyoxyethylene-9-lauryl ether or a pharmaceutically acceptable salt thereof.

In another embodiment, the penetration enhancer is a chelating agent. The chelating agent can be EDTA, citric acid, a salicyclate, a N-acyl derivative of collagen, laureth-9, an N-amino acyl derivative of a beta-diketone or a mixture thereof.

In another embodiment, the penetration enhancer is a surfactant, e.g., an ionic or nonionic surfactant. The surfactant can be sodium lauryl sulfate, polyoxyethylene-9-lauryl ether, polyoxyethylene-20-cetyl ether, a perfluorchemical emulsion or mixture thereof.

In another embodiment, the penetration enhancer can be selected from a group consisting of unsaturated cyclic ureas, 1-alkyl-alkones, 1-alkenylazacyclo-alakanones, steroidal anti-inflammatory agents and mixtures thereof. In yet another embodiment the penetration enhancer can be a glycol, a pyrrol, an azone, or a terpenes.

In one aspect, the invention features a pharmaceutical composition including an siRNA compound, e.g., a double-stranded siRNA compound, or ssiRNA compound, (e.g., a precursor, e.g., a larger siRNA compound which can be processed into a ssiRNA compound, or a DNA which encodes an siRNA compound, e.g., a double-stranded siRNA compound, or ssiRNA compound, or precursor thereof) in a form suitable for oral delivery. In one embodiment, oral delivery can be used to deliver an siRNA compound composition to a cell or a region of the gastro-intestinal tract, e.g., small intestine, colon (e.g., to treat a colon cancer), and so forth. The oral delivery form can be tablets, capsules or gel capsules. In one embodiment, the siRNA compound of the pharmaceutical composition modulates expression of a cellular adhesion protein, modulates a rate of cellular proliferation, or has biological activity against eukaryotic pathogens or retroviruses. In another embodiment, the pharmaceutical composition includes an enteric material that substantially prevents dissolution of the tablets, capsules or gel capsules in a mammalian stomach. In some embodiments the enteric material is a coating. The coating can be acetate phthalate, propylene glycol, sorbitan monoleate, cellulose acetate trimellitate, hydroxy propyl methylcellulose phthalate or cellulose acetate phthalate.

In another embodiment, the oral dosage form of the pharmaceutical composition includes a penetration enhancer. The penetration enhancer can be a bile salt or a fatty acid. The bile salt can be ursodeoxycholic acid, chenodeoxycholic acid, and salts thereof. The fatty acid can be capric acid, lauric acid, and salts thereof.

In another embodiment, the oral dosage form of the pharmaceutical composition includes an excipient. In one example the excipient is polyethyleneglycol. In another example the excipient is precirol.

In another embodiment, the oral dosage form of the pharmaceutical composition includes a plasticizer. The plasticizer can be diethyl phthalate, triacetin dibutyl sebacate, dibutyl phthalate or triethyl citrate.

In one aspect, the invention features a pharmaceutical composition including an siRNA compound and a delivery vehicle. In one embodiment, the siRNA compound is (a) is 19-25 nucleotides long, for example, 21-23 nucleotides, (b) is complementary to an endogenous target RNA, and, optionally, (c) includes at least one 3′ overhang 1-5 nucleotides long.

In one embodiment, the delivery vehicle can deliver an siRNA compound, e.g., a double-stranded siRNA compound, or ssiRNA compound, (e.g., a precursor, e.g., a larger siRNA compound which can be processed into a ssiRNA compound, or a DNA which encodes an siRNA compound, e.g., a double-stranded siRNA compound, or ssiRNA compound, or precursor thereof) to a cell by a topical route of administration. The delivery vehicle can be microscopic vesicles. In one example the microscopic vesicles are liposomes. In some embodiments the liposomes are cationic liposomes. In another example the microscopic vesicles are micelles. In one aspect, the invention features a pharmaceutical composition including an siRNA compound, e.g., a double-stranded siRNA compound, or ssiRNA compound, (e.g., a precursor, e.g., a larger siRNA compound which can be processed into a ssiRNA compound, or a DNA which encodes an siRNA compound, e.g., a double-stranded siRNA compound, or ssiRNA compound, or precursor thereof) in an injectable dosage form. In one embodiment, the injectable dosage form of the pharmaceutical composition includes sterile aqueous solutions or dispersions and sterile powders. In some embodiments the sterile solution can include a diluent such as water; saline solution; fixed oils, polyethylene glycols, glycerin, or propylene glycol.

In one aspect, the invention features a pharmaceutical composition including an siRNA compound, e.g., a double-stranded siRNA compound, or ssiRNA compound, (e.g., a precursor, e.g., a larger siRNA compound which can be processed into a ssiRNA compound, or a DNA which encodes an siRNA compound, e.g., a double-stranded siRNA compound, or ssiRNA compound, or precursor thereof) in oral dosage form. In one embodiment, the oral dosage form is selected from the group consisting of tablets, capsules and gel capsules. In another embodiment, the pharmaceutical composition includes an enteric material that substantially prevents dissolution of the tablets, capsules or gel capsules in a mammalian stomach. In some embodiments the enteric material is a coating. The coating can be acetate phthalate, propylene glycol, sorbitan monoleate, cellulose acetate trimellitate, hydroxy propyl methyl cellulose phthalate or cellulose acetate phthalate. In one embodiment, the oral dosage form of the pharmaceutical composition includes a penetration enhancer, e.g., a penetration enhancer described herein.

In another embodiment, the oral dosage form of the pharmaceutical composition includes an excipient. In one example the excipient is polyethyleneglycol. In another example the excipient is precirol.

In another embodiment, the oral dosage form of the pharmaceutical composition includes a plasticizer. The plasticizer can be diethyl phthalate, triacetin dibutyl sebacate, dibutyl phthalate or triethyl citrate.

In one aspect, the invention features a pharmaceutical composition including an siRNA compound, e.g., a double-stranded siRNA compound, or ssiRNA compound, (e.g., a precursor, e.g., a larger siRNA compound which can be processed into a ssiRNA compound, or a DNA which encodes an siRNA compound, e.g., a double-stranded siRNA compound, or ssiRNA compound, or precursor thereof) in a rectal dosage form. In one embodiment, the rectal dosage form is an enema. In another embodiment, the rectal dosage form is a suppository.

In one aspect, the invention features a pharmaceutical composition including an siRNA compound, e.g., a double-stranded siRNA compound, or ssiRNA compound, (e.g., a precursor, e.g., a larger siRNA compound which can be processed into a ssiRNA compound, or a DNA which encodes an siRNA compound, e.g., a double-stranded siRNA compound, or ssiRNA compound, or precursor thereof) in a vaginal dosage form. In one embodiment, the vaginal dosage form is a suppository. In another embodiment, the vaginal dosage form is a foam, cream, or gel.

In one aspect, the invention features a pharmaceutical composition including an siRNA compound, e.g., a double-stranded siRNA compound, or ssiRNA compound, (e.g., a precursor, e.g., a larger siRNA compound which can be processed into a ssiRNA compound, or a DNA which encodes an siRNA compound, e.g., a double-stranded siRNA compound, or ssiRNA compound, or precursor thereof) in a pulmonary or nasal dosage form. In one embodiment, the siRNA compound is incorporated into a particle, e.g., a macroparticle, e.g., a microsphere. The particle can be produced by spray drying, lyophilization, evaporation, fluid bed drying, vacuum drying, or a combination thereof. The microsphere can be formulated as a suspension, a powder, or an implantable solid.

In one aspect, the invention features a spray-dried siRNA compound, e.g., a double-stranded siRNA compound, or ssiRNA compound, (e.g., a precursor, e.g., a larger siRNA compound which can be processed into a ssiRNA compound, or a DNA which encodes an siRNA compound, e.g., a double-stranded siRNA compound, or ssiRNA compound, or precursor thereof) composition suitable for inhalation by a subject, including: (a) a therapeutically effective amount of a siRNA compound suitable for treating a condition in the subject by inhalation; (b) a pharmaceutically acceptable excipient selected from the group consisting of carbohydrates and amino acids; and (c) optionally, a dispersibility-enhancing amount of a physiologically-acceptable, water-soluble polypeptide.

In one embodiment, the excipient is a carbohydrate. The carbohydrate can be selected from the group consisting of monosaccharides, disaccharides, trisaccharides, and polysaccharides. In some embodiments the carbohydrate is a monosaccharide selected from the group consisting of dextrose, galactose, mannitol, D-mannose, sorbitol, and sorbose. In another embodiment the carbohydrate is a disaccharide selected from the group consisting of lactose, maltose, sucrose, and trehalose.

In another embodiment, the excipient is an amino acid. In one embodiment, the amino acid is a hydrophobic amino acid. In some embodiments the hydrophobic amino acid is selected from the group consisting of alanine, isoleucine, leucine, methionine, phenylalanine, proline, tryptophan, and valine. In yet another embodiment the amino acid is a polar amino acid. In some embodiments the amino acid is selected from the group consisting of arginine, histidine, lysine, cysteine, glycine, glutamine, serine, threonine, tyrosine, aspartic acid and glutamic acid.

In one embodiment, the dispersibility-enhancing polypeptide is selected from the group consisting of human serum albumin, α-lactalbumin, trypsinogen, and polyalanine

In one embodiment, the spray-dried siRNA compound composition includes particles having a mass median diameter (MMD) of less than 10 microns. In another embodiment, the spray-dried siRNA compound composition includes particles having a mass median diameter of less than 5 microns. In yet another embodiment the spray-dried siRNA compound composition includes particles having a mass median aerodynamic diameter (MMAD) of less than 5 microns.

In certain other aspects, the invention provides kits that include a suitable container containing a pharmaceutical formulation of an siRNA compound, e.g., a double-stranded siRNA compound, or ssiRNA compound, (e.g., a precursor, e.g., a larger siRNA compound which can be processed into a ssiRNA compound, or a DNA which encodes an siRNA compound, e.g., a double-stranded siRNA compound, or ssiRNA compound, or precursor thereof). In certain embodiments the individual components of the pharmaceutical formulation may be provided in one container. Alternatively, it may be desirable to provide the components of the pharmaceutical formulation separately in two or more containers, e.g., one container for an siRNA compound preparation, and at least another for a carrier compound. The kit may be packaged in a number of different configurations such as one or more containers in a single box. The different components can be combined, e.g., according to instructions provided with the kit. The components can be combined according to a method described herein, e.g., to prepare and administer a pharmaceutical composition. The kit can also include a delivery device.

In another aspect, the invention features a device, e.g., an implantable device, wherein the device can dispense or administer a composition that includes an siRNA compound, e.g., a double-stranded siRNA compound, or ssiRNA compound, (e.g., a precursor, e.g., a larger siRNA compound which can be processed into a ssiRNA compound, or a DNA which encodes an siRNA compound, e.g., a double-stranded siRNA compound, or ssiRNA compound, or precursor thereof), e.g., a siRNA compound that silences an endogenous transcript. In one embodiment, the device is coated with the composition. In another embodiment the siRNA compound is disposed within the device. In another embodiment, the device includes a mechanism to dispense a unit dose of the composition. In other embodiments the device releases the composition continuously, e.g., by diffusion. Exemplary devices include stents, catheters, pumps, artificial organs or organ components (e.g., artificial heart, a heart valve, etc.), and sutures.

As used herein, the term “crystalline” describes a solid having the structure or characteristics of a crystal, i.e., particles of three-dimensional structure in which the plane faces intersect at definite angles and in which there is a regular internal structure. The compositions of the invention may have different crystalline forms. Crystalline forms can be prepared by a variety of methods, including, for example, spray drying.

The invention is further illustrated by the following examples, which should not be construed as further limiting. The contents of all references, pending patent applications and published patents, cited throughout this application are hereby expressly incorporated by reference.

### EXAMPLES

The invention now being generally described, it will be more readily understood by reference to the following examples, which are included merely for purposes of illustration of certain aspects and embodiments of the present invention, and are not intended to limit the invention.

### Example 1

### iRNA Agent Synthesis

### 1. Oigonucleotide Synthesis

Oligonucleotides were synthesized on an AKTAoligopilot synthesizer. Commercially available controlled pore glass solid support (dT-CPG, 500 Å, Prime Synthesis) and RNA phosphoramidites with standard protecting groups, 5′-O-dimethoxytrityl N6-benzoyl-2′-t-butyldimethylsilyl-adenosine-3′-O—N,N′-diisopropyl-2-cyanoethylphosphoramidite, 5′-O-dimethoxytrityl-N4-acetyl-2′-t-butyldimethylsilyl-cytidine-3′-O—N,N′-diisopropyl-2-cyanoethylphosphoramidite, 5′-O-dimethoxytrityl-N2-isobutryl-2′-t-butyldimethylsilyl-guanosine-3′-O—N,N′-diisopropyl-2-cyanoethylphosphoramidite, and 5′-O-dimethoxytrityl-2′-t-butyldimethylsilyl-uridine-3′-O—N,N′-diisopropyl-2-cyanoethylphosphoramidite (Pierce Nucleic Acids Technologies) were used for the oligonucleotide synthesis. The 2′-F phosphoramidites, 5′-O-dimethoxytrityl-N4-acetyl-2′-fluoro-cytidine-3′-O—N,N′-diisopropyl-2-cyanoethyl-phosphoramidite and 5′-O-dimethoxytrityl-2′-fluoro-uridine-3′-O—N,N′-diisopropyl-2-cyanoethyl-phosphoramidite were purchased from (Promega). 2′-F A and G phosphoramidites were custom synthesized as reported in the literature (Kawasaki et al., J. Med. Chem., 1993, 36, 831-841) All phosphoramidites were used at a concentration of 0.2M in acetonitrile (CH₃CN) except for guanosine which was used at 0.2M concentration in 10% THF/ANC (v/v). Coupling/recycling time of 16 minutes was used. The activator was 5-ethyl thiotetrazole (0.75M, American International Chemicals), for the PO-oxidation Iodine/Water/Pyridine was used and the PS-oxidation PADS (2%) in 2,6-lutidine/ACN (1:1 v/v) was used.

The 2′-O-methyl phosphoramidites, 5′-O-dimethoxytrityl-N4-acetyl-2′-O-methyl-cytidine-3′-O—N,N′-diisopropyl-2-cyanoethyl-phosphoramidite and 5′-O-dimethoxytrityl-2′-O-methyl-uridine-3′-O—N,N′-diisopropyl-2-cyanoethyl-phosphoramidite were purchased from ChemGenes.

The 2′-methoxyethyl phosphoramidites, 5′-O-dimethoxytrityl-N4-acetyl-2′-methoxyethyl-5-methyl-cytidine-3′-O—N,N′-diisopropyl-2-cyanoethyl-phosphoramidite and 5′-O-dimethoxytrityl-2′-methoxyethyl-thymidine-3′-O—N,N′-diisopropyl-2-cyanoethyl-phosphoramidite were synthesized in house by methods known in the art, for example see Ross et al. (2005) Nucleosides, Nucleotides, and Nucleic Acids, 24(5-7), 815 and Sivets (2007) Nucleosides, Nucleotides, and Nucleic Acids, 36, 1237. Other prior art references describing the synthesis of 2′-methoxyethyl modified phosphoramidites include U.S. Pat. No. 7,030,230,U.S. Pat. No. 6,013,787, U.S. Pat. No. 6,166,197,U.S. Pat. No. 6,642,367,U.S. Pat. No. 5,760,202 and U.S. Pat. No. 5,861,493.

The LNA phosphoramidite, 5′-O-dimethoxytrityl-N4-acetyl-2′-O,4′-methylene-thymidine-3′-O—N,N′-diisopropyl-2-cyanoethyl-phosphoramidite was purchased from Proligo Blochemie, GmBh, Hamburg, Germany

All phosphoramidites were used at a concentration of 0.2M in acetonitrile (CH₃CN) except for guanosine which was used at 0.2M concentration in 10% THF/ANC (v/v). Coupling/recycling time of 16 minutes was used. The activator was 5-ethyl thiotetrazole (0.75M, American International Chemicals), for the PO-oxidation Iodine/Water/Pyridine was used and the PS-oxidation PADS (2%) in 2,6-lutidine/ACN (1:1 v/v) was used. The cholesterol phosphoramidite was synthesized in house, and used at a concentration of 0.1 M in dichloromethane. Coupling time for the cholesterol phosphoramidite was 16 minutes.

3′-ligand conjugated strands were synthesized using solid support containing the corresponding ligand. For example, the introduction of cholesterol unit in the sequence was performed from a hydroxyprolinol-cholesterol phosphoramidite. Cholesterol was tethered to trans-4-hydroxyprolinol via a 6-aminohexanoate linkage to obtain a hydroxyprolinol-cholesterol moiety. 5′-end Cy-3 and Cy-5.5 (fluorophore) labeled siRNAs were synthesized from the corresponding Quasar-570 (Cy-3) phosphoramidite were purchased from Biosearch Technologies. Conjugation of ligands to 5′-end and or internal position is achieved by using appropriately protected ligand-phosphoramidite building block An extended 15 min coupling of 0.1M solution of phosphoramidite in anhydrous CH₃C_{N} in the presence of 5-(ethylthio)-1H-tetrazole activator to a solid bound oligonucleotide. Oxidation of the internucleotide phosphite to the phosphate was carried out using standard iodine-water as reported (Maurer, N. et al. Spontaneous entrapment of polynucleotides upon electrostatic interaction with ethanol-destabilized cationic liposomes. Biophys J 80, 2310-2326 (2001).) or by treatment with tert-butyl hydroperoxide/acetonitrile/water (10:87:3) with 10 minute oxidation wait time conjugated oligonucleotide. Phosphorothioate was introduced by the oxidation of phosphite to phosphorothioate by using a sulfur transfer reagent such as DDTT (purchased from AM Chemicals), PADS and or Beaucage reagent. The cholesterol phosphoramidite was synthesized, and used at a concentration of 0.1 M in dichloromethane. Coupling time for the cholesterol phosphoramidite was 16 minutes.

### 2. Deprotection-I (Nucleobase Deprotection)

After completion of synthesis, the support was transferred to a 100 ml glass bottle (VWR). The oligonucleotide was cleaved from the support with simultaneous deprotection of base and phosphate groups with 80 mL of a mixture of ethanolic ammonia [ammonia:ethanol (3:1)] for 6.5 h at 55° C. The bottle was cooled briefly on ice and then the ethanolic ammonia mixture was filtered into a new 250 ml bottle. The CPG was washed with 2×40 mL portions of ethanol/water (1:1 v/v). The volume of the mixture was then reduced to ˜30 ml by roto-vap. The mixture was then frozen on dyince and dried under vacuum on a speed vac.

### 3. Deprotection-II (Removal of 2′ TBDMS Group)

The dried residue was resuspended in pyridine-HF and DMSO (3:4:6) and heated at 60° C. for 90 minutes to remove the tert-butyldimethylsilyl (TBDMS) groups at the 2′ position. The reaction was then quenched with 50 ml of 20 mM sodium acetate and pH adjusted to 6.5, and stored in freezer until purification.

### 4. Deprotection Procedure for 2′-F Modified RNA

A mild deprotection/cleavage procedure used for modified RNAs, containing both 2′-fluoro- and 2′-OH containing nucleotides.

Procedure: The support was treated on-column with 0.5 M piperidine/ACN for 20 minutes. This was done on the synthesizer by placing the reagent bottle on one of the bottle positions. The support was washed with acetonitrile and dried in the column under vacuum or by blowing nitrogen through the column. Subsequently, the support was transferred into a container which can be tightly sealed. NH4OH:Ethanol (3:1) was added and the bottle is sealed tightly and shaken at 28-30° C. for ca. 16 hours. The mixture was cooled at −20° C. for 20 min and filtered. The solid support was washed thoroughly with DMSO and the washing solution was combined with the filtrate. The combined solution was cooled at −20° C. for 10 minutes before HF solution was added. The container is tightly capped and shaken at 40° C. for 1 hour. The cleavage solution was stored at −20° C.

### 5. Analysis

The oligoncuelotides were analyzed by high-performance liquid chromatography (HPLC) prior to purification and selection of buffer and column depends on the nature of the sequence and/or conjugated ligand.

### 6. HPLC Purification

The ligand-conjugated oligonucleotides were purified reverse phase preparative HPLC. The unconjugated oligonucleotides were purified by anion-exchange HPLC on a TSK gel column. The buffers were 20 mM sodium phosphate (pH 8.5) in 10% CH₃CN (buffer A) and 20 mM sodium phosphate (pH 8.5) in 10% CH₃CN, 1M NaBr (buffer B). Fractions containing full-length oligonucleotides were pooled, desalted, and lyophilized. Approximately 0.15 OD of desalted oligonucleotides were diluted in water to 150 μl and then pipetted in special vials for CGE and LC/MS analysis. Compounds were finally analyzed by LC-ESMS and CGE.

HPLC Purification 2. The crude oligomers were first analyzed by HPLC (Dionex PA 100). The buffer system was: A=20 mM phosphate pH 11, B=20 mM phosphate, 1.8 M NaBr, pH 11, flow rate 1.0 mL/min, and wavelength 260-280 nm. Inject 5-15 μA of the each sample. The unconjugated samples were purified by HPLC on a TSK-Gel SuperQ-5PW (20) column packed in house (17.3×5 cm). The buffer system was: A=20 mM phosphate in 10% ACN, pH 8.5 and B=20 mM phosphate, 1.0 M NaBr in 10% ACN, pH 8.5, with a flow rate of 50.0 mL/min, and wavelength 260 and 294. The fractions containing the full length oligonucleotides were then pooled together, evaporated and reconstituted to 100 ml with deionised water.

### 7. Desalting of Purified Oligomer

The purified oligonucleotides were desalted using AKTA Explorer (Amersham Biosciences) using Sephadex G-25 column. First column was washed with water at a flow rate of 25 ml/min for 20-30 min. The sample was then applied in 25 ml fractions. The eluted salt-free fractions were combined together, dried down and reconstituted in 50 ml of RNase free water.

### 8. Capillary Gel Electrophoresis (CGE) and Electrospray LC/Ms

Approximately 0.15 OD of desalted oligonucleotides were diluted in water to 150 and then pipetted in special vials for CGE and LC/MS analysis.

TABLE 5
Oligonucleotides containing 2′-F modification

| Seq. | | | |
|---|---|---|---|
| ID | ^{a}Sequence 5′-3′ | Strand | Target |
| 1 | CUUACGCUGAGUACUUCGAdTdT | Sense | Luc |
| 2 | UCGAAGUACUCAGCGUAAGdTdT | Antisense | Luc |
| 3 | CsUfU*ACGCUGAGfU*ACUUCGAdTsdT | Sense | Luc |
| 4 | UsCGAAGfU*ACUCAGCGfU*AAGdTsdT | Antisense | Luc |
| 5 | all PS (AccGAAAGGucuuAccGGAdTdT) | Sense | Luc |
| 6 | all PS (UCCGGuAAGACCUUUCGGUdTdT) | Antisense | Luc |
| 7 | AccGAAAGGucuuAccGGAdTsdT | Sense | Luc |
| 8 | UCCGGuAAGACCUUUCGGUdTsdT | Antisense | Luc |
| 9 | GGAUCAUCUCAAGUCUUACdTdT | Sense | FVII |
| 10 | GUAAGACUUGAGAUGAUCCdTdT | Antisense | FVII |
| 11 | GuAAGAcuuGAGAuGAUccdTdT | Antisense | FVII |
| 12 | all PS (GGAUCAUCUCAAGUCUUACdTdT) | Sense | FVII |
| 13 | all PS (GGAUfCfAUfCfUfCfAAGUfCfUfUfACfdTdT) | Sense | FVII |
| 14 | all PS (ggAfUfCfAfUfCfUfCfAfAfGUfCfUfUfAfCfuu) | Sense | FVII |
| 15 | all PS (GUAAGACUUGAGAUGAUCCdTdT) | Antisense | FVII |
| 16 | all PS (GUfAAGACfUfUfGAGAUfGAUfCfCfdTdT) | Antisense | FVII |
| 17 | all PS(psGfUfAfAfGfAfCfUfUfGfAfGfAfUfGfAfUfCfCfdTdT) | Antisense | FVII |
| 18 | all PS (psguAfAfGfAfCfUfUfGfAfGfAfUfGfAfUfCfCfug) | Antisense | FVII |
| 19 | Q-GGAfUfCAfUfCfUfCAAGfUfCfUfUAfCdTsdT | Sense | FVII |
| 20 | Q-GGAfCfUAfCfUfCfUAAGfUfUfCfUAfCdTsdT | Sense | FVII |
| 21 | GGAfCfUAfCfUfCfUAAGfUfUfCfUAfCdTsdT | Sense | FVII |
| 22 | GfUAGAAfCfUfUAGAGfUAGfUfCfCdTsdT | Antisense | FVII |
| 23 | GGAfUfCAfUfCfUfCAAGfUfCfUfUAfCdTsdT | Sense | FVII |
| 24 | GfUAAGAfCfUfUGAGAfUGAfUfCfCdT*dT | Antisense | FVII |
| 25 | all PS (GGAAUCuuAuAuuuGAUCcAA) | Sense | apoB |
| 26 | all PS (ggAfAfUfCfUfUfAfUfAfUfUfUfGfAfUfCfcaa) | Sense | apoB |
| 27 | all PS (psuuGGAUcAAAuAuAAGAuUCccU) | Antisense | apoB |
| 28 | all PS (psUfUfGfGfAfUfCfAfAfAfUfAfUfAfAfGfAfUfUfCfCfCfUf) | Antisense | apoB |
| 29 | all PS (psuuGfGfAfUfCfAfAfAfUfAfUfAfAfGfAfUfUfCfccu) | Antisense | apoB |
| 30 | all PS (GccuGGAGuuuAuucGGAAdTdT) | Sense | PCSK9 |
| 31 | all PS (gcCfUfGfGfAfGfUfUfUfAUfUfCfGfGfAfAfga) | Sense | PCSK9 |
| 32 | all PS (UUCCGAAuAAACUCcAGGCdTdT) | Antisense | PCSK9 |
| 33 | all PS (psuuCfCfGfAfAfUfAfAfAfCfUfCfCfAfGfGfCfcu) | Antisense | PCSK9 |
| 34 | all PS (psUfUfCfCfGfAfAfUfAfAfAfCfUfCfCfAfGfGfCfdTdT) | Antisense | PCSK9 |
| 35 | all PS (cuGGcuGAAuuucAGAGcAdTdT) | Sense | CD45 |
| 36 | all PS (UGCUCUGAAAUUcAGCcAGdTdT) | Antisense | CD45 |
| 37 | CsUfU*ACGCUGAGfU*ACUUCGAdTsdT-L | Sense | Luc |
| 38 | UsCGAAGfU*ACUCAGCGfU*AAGdTsdT-sL | Antisense | Luc |
| 39 | all PS (AccGAAAGGucuuAccGGAdTdT-L) | Sense | Luc |
| 40 | all PS (UCCGGuAAGACCUUUCGGUdTdT-L) | Antisense | Luc |
| 41 | AfCfCGAAAGGfUfCfUfUAfCfCGGAdTsdT-L | Sense | Luc |
| 42 | fAfCfCfGfAfAfAfGfGfUfCfUfUfAfCfCfGfGfAdTsdT-L | Sense | Luc |
| 43 | fACCfGfAfAfAfGfGUCUUfACCfGfGfAdTsdT-L | Sense | Luc |
| 44 | fAccfGfAfAfAfGfGucuufAccfGfGfAdTsdT-L | Sense | Luc |
| 45 | AfCfCGAAAGGfUfCfUfUAfCfCGGAdTsdT | Sense | Luc |
| 46 | fAccfGfAfAfAfGfGucuufAccfGfGfAdTsdT | Sense | Luc |
| 47 | fACCfGfAfAfAfGfGUCUUfACCfGfGfAdTsdT | Sense | Luc |
| 48 | GGAfCfUAfCfUfCfUAAGfUfUfCfUAfCdTsdT-L | Sense | FVII |
| 49 | GfUAGAAfCfUfUAGAGfUAGfUfCfCdTsdT-L | Antisense | FVII |
| 50 | fGfGfAfCfUfAfCfUfCfUfAfAfGfUfUfCfUfAfCdTsdT | Sense | FVII |
| 51 | fGfUfAfGfAfAfCfUfUfAfGfAfGfUfAfGfUfCfCdTsdT | Antisense | FVII |
| 52 | GGAfCfUAfCfUfCfUAAGfUfUfCfUAfCdTsdT-sL | Sense | FVII |
| 53 | GfUAGAAfCfUfUAGAGfUAGfUfCfCdTsdT-sL | Antisense | FVII |
| 54 | fGfGfACUfACUCUfAfAfGUUCUfACdTsdT | Sense | FVII |
| 55 | fGUfAfGfAfACUUfAfGfAfGUfAfGUCCdTsdT | Antisense | FVII |
| 56 | GGAcfUAcfUcfUAAGfUfUcfUAcdTsdT-L | Sense | FVII |
| 57 | GuAGAAfCuuAGAGuAGufCfCdTsdT-L | Antisense | FVII |
| 58 | fGfGfAcufAcucufAfAfGuuuufAcdTsdT | Sense | FVII |
| 59 | gfUggaafCfUfUagagfUagfUfCfCdTsdT | Antisense | FVII |
| 60 | ggafCfUafCfUfCfUaaafUfUfCfUafCdTsdT | Sense | FVII |
| 61 | fGfGfAcufAcucufAfAfGuuuufAcdTsdT-L | Sense | FVII |
| 62 | gfUggaafCfUfUagagfUagfUfCfCdTsdT-L | Antisense | FVII |
| 63 | ggafCfUafCfUfCfUaaafUfUfCfUafCdTsdT-L | Sense | FVII |
| 64 | fGfGfAcufAcucufAfAfGuuuufAcdTsdT-sL | Sense | FVII |
| 65 | gfUggaafCfUfUagagfUagfUfCfCdTsdT-sL | Antisense | FVII |
| 66 | ggafCfUafCfUfCfUaaafUfUfCfUafCdTsdT-sL | Sense | FVII |

^{a}“fN” indicates 2′-deoxy-2′-fluoro-N; “fU*” indicates 2′-deoxy-2′-fluoro-5-methyl-U, “s” indicates a phosphorothioate linkage, a lower case letter (e.g., “u”) indicates a 2′-OMe sugar modification, “p” at the 5′-end indicates a phosphate, “ps” indicates a phosphorothioate, “all PS” indicates that all internucleotide linkages contain one or more
phosphorothoiate linkages. “L” indicates a 3′ conjugated ligand such as cholesterol, GalNAc, Mannose, Folate, bile acid, fatty acid, steroids, masked oligonucleotides, a polycation or a polyanion. “Q” inidicates 5′ conjugated ligand selected from cholesterol, GalNAc, Mannose, Folate, bile acid, fatty acid, steroids, masked oligo/poly cations/anions.

TABLE 6
siRNA duplexes for Luc and FVII targeting

| | Sense/ | | |
|---|---|---|---|
| | Antisense ID | | |
| Duplex | from Table 5 | Sequence 5′-3′ | Target |
| 1000/2434 | 1/4 | CUU ACG CUG AGU ACU UCG AdTdT | Luc |
| | | U*CG AAG fUAC UCA GCG fUAA GdT*dT | |
| 2433/1001 | 3/2 | C*UfU ACG CUG AGfU ACU UCG AdT*dT | Luc |
| | | UCG AAG UAC UCA GCG UAA GdTdT | |
| 2433/2434 | 3/4 | C*UfU ACG CUG AGfU ACU UCG AdT*dT | Luc |
| | | U*CG AAG fUAC UCA GCG fUAA GdT*dT | |
| 1000/1001 | 1/4 | CUU ACG CUG AGU ACU UCG AdTdT | Luc |
| | | UCG AAG UAC UCA GCG UAA GdTdT | |
| AD-1596 | 9/10 | GGAUCAUCUCAAGUCUUACdTdT | FVII |
| | | GUAAGACUUGAGAUGAUCCdTdT | |
| AD-1661 | 23/24 | GGAfUfCAfUfCfUfCAAGfUfCfUfUAfCdTsdT | |
| | | GfUAAGAfCfUfUGAGAfUGAfUfCfCdT*dT | |

TABLE 7
microRNA and antimicroRNA (Antagomirs)

| Seq. | | | |
|---|---|---|---|
| ID | ^{a}Sequence 5′-3′ | Strand | Target |
| 67 | UGGAGUGUGACAAUGGUGUUUGU | miR-122 | miRNA mimic |
| 68 | UAGCAGCACGUAAAUAUUGGCG | miR-16 | miRNA mimic |
| 69 | CUGACCUAUGAAUUGACAGCC | miR-192 | miRNA mimic |
| 70 | UGUAACAGCAACUCCAUGUGGA | miR-194 | miRNA mimic |
| 71 | UGUUUGUGGUAACAGUGUGAGGU | miR-122 | miRNA mimic |
| 72 | UfsGfsGfsAfsGfsUfsGfsUfsGfsAfsCfsAfsAfsUfsGfsGfsUfs | miR-122 | miRNA mimic |
| | GfsUfsUfsUfsGfsUfs-L | | |
| 73 | UfsGfsGfAfGfUfGfUfGfAfCfAfAfUfGfGfUfGfUfUfsUfsGf | miR-122 | miRNA mimic |
| | sUfs-L | | |
| 74 | UfsGfsGfsAfsAfsUfsGfsUfsGfsAfsCfsAfsGfsUfsGfsUfsUfs | miR-122 | miRNA mimic |
| | GfsUfsGfsUfsGfsUfs-L | | |
| 75 | UfsGfsGfAfAfUfGfUfGfAfCfAfGfUfGfUfUfGfUfGfsUfsGf | miR-122 | miRNA mimic |
| | sUfs-L | | |
| 76 | ascsaaacaccauugucacacuscscsas-L | Antagomir | miR-122 |
| 77 | cscsaucuuuaccagacagugsususas-L | Antagomir | miR-141 |
| 78 | usgsagcuacagugcuucauasuscsas-L | Antagomir | miR-143 |
| 79 | asascucaccgacagcguugaausgsusus-L- | Antagomir | miR-181 |
| 80 | gsgsccguccauuaauagauscsasgs-L | Antagomir | miR-192 |
| 81 | uscscccauagagcugcugcusascsas-L | Antagomir | miR-194 |
| 82 | cscsaucauuacccgccaguasususas-L | Antagomir | miR-200c |
| 83 | cscsacacacuuccuuacauuscscsas-L | Antagomir | miR-206 |
| 84 | csasgcuaugccagcaucuugscscsus-L | Antagomir | miR-31 |
| 85 | cscsaacaacaugaaacuacscsusas-L | Antagomir | miR-196 |
| 86 | csuscugucaaaucauagguscsasus-L | Antagomir | miR-215 |
| 87 | csasaugcaacuacaaugscsascs-L | Antagomir | miR-33 |
| 88 | ususggcauucaccgcgugccsususas-L | Antagomir | miR-124 |
| 89 | gsuscugucaaaucauagguscsasus-L | Antagomir | miR-215 |
| 90 | cscsccuaucacaauuagcaususasas-L | Antagomir | miR-155 |
| 91 | uscsaacaucagucuguaagscsusas-L | Antagomir | miR-21 |
| 92 | ascsaguucuucaacuggcagscsusus-L | Antagomir | miR-22 |
| 93 | csgscauuauuacucacgguascsgsas-L | Antagomir | miR-126 |
| 94 | Ccauugucacacucc | Antagomir | mir-122 |

“fN” indicates 2′-deoxy-2′-fluoro-N; “fU*” indicates 2′-deoxy-2′-fluoro-5-methyl-U, “s” indicates a phosphorothioate linkage, a lower case letter (e.g. “u”) indicates a 2′-OMe sugar modification, “p” at the 5′-end indicates a phosphate, “ps” indicates a phosphorothioate, “all PS” indicates that all internucleotide linkages contain one or more phosphorothioate linkages. “L” indicates a 3′ conjugated ligand such as cholesterol, GalNAc, Mannose, Folate, bile acid, fatty acid, steroids, other carbohydrates, masked oligonucleotides, small molecules, a polycation or a polyanion. “Q” indicates 5′ conjugated ligand selected from cholesterol, GalNAc, Mannose, Folate, bile acid, fatty acid, steroids, masked oligo/poly cations/anions

### Example 2

### siRNA Preparation

### 9. Duplex Formation

Equal amounts, by moles, of the two single strands were mixed together. The mixtures were frozen at −80° C. and dried under vacuum on a speed vac. Dried samples were then dissolved in 1×PBS to the desired concentration. The dissolved samples were heated to 95° C. for 5 min and slowly cooled to room temperature.

TABLE 8
Some of the iRNA agents synthesized and tested.

| Duplex | Strand | SEQ | |
|---|---|---|---|
| No. | Type | ID | Sequence* |
| AD-1596 | Sense | 9 | GGAUCAUCUCAAGUCUUACdTdT |
| | Antisense | 10 | GUAAGACUUGAGAUGAUCCdTdT |
| AD-1661 | Sense | 23 | GGAucAucucAAGucuuAcdTsdT |
| | Antisense | 24 | GuAAGAcuuGAGAuGAuccdTsdT |
| AD-19013 | Sense | 95 | GGAucAucucAAGucuuAcdTsdT |
| | Antisense | 96 | GuAAGAcuuGAGAuGAuccdTsdT |
| AD-19014 | Sense | 97 | GGA(Teo)(m5Ceo)A(Teo)(m5Ceo)(Teo)(m5Ceo)AAG |
| | | | (m5Ceo)(m5Ceo)(Teo)(Teo)A(m5Ceo)dTsdT |
| | Antisense | 98 | G(Teo)AAGA(m5Ceo)(Teo)(Teo)GAGA(Teo)GA(Teo) |
| | | | (m5Ceo)(m5Ceo)dTsdT |
| AD-19015 | Sense | 99 | GGA(Tln)CA(Tln)C(Tln)CAAGCC(Tln)UACdTsdT |
| | Antisense | 100 | G(Tln)AAGAC(Tln)(Tln)GAGA(Tln)GA(Tln)CCdTsdT |
| AD-19016 | Sense | 101 | GGAucAucucAAGucuuAcdTsdT |
| | Antisense | 102 | GuAAGAcuuGAGAuGAuccdTsdT |
| AD-19017 | Sense | 103 | GGA(Teo)(m5Ceo)A(Teo)(m5Ceo)(Teo)(m5Ceo)AAG |
| | | | (m5Ceo)(m5Ceo)(Teo)(Teo)A(m5Ceo)dTsdT |
| | Antisense | 104 | GuAAGAcuuGAGAuGAuccdTsdT |
| AD-19018 | Sense | 105 | GGA(Tln)CA(Tln)C(Tln)CAAGCC(Tln)UACdTsdT |
| | Antisense | 106 | GuAAGAcuuGAGAuGAuccdTsdT |

*s is phosphorothioate backbone linkage, lowercase is 2′-O-methyl nucleotide, (Teo) is 2′-methoxyethyl-thymidine, (5mCeo) is 2′-methoxyethyl-cytidine, (Tln) is 2′-O, 4′-methylene-thymidine (LNA), underlined lower case is 2′-deoxy-2′-fluoronucleotide.

### Example 3

### Serum Stability Assay for siRNA

A medium throughput assay for initial sequence-based stability selection was performed by the “stains all” approach. To perform the assay, an siRNA duplex was incubated in 90% human serum at 37° C. Samples of the reaction mix were quenched at various time points (at 0 minutes, 15, 30, 60, 120, and 240 min) and subjected to electrophoretic analysis (FIG. 1). Cleavage of the RNA over the time course provided information regarding the susceptibility of the siRNA duplex to serum nuclease degradation.

A radiolabeled dsRNA and serum stability assay was used to further characterize siRNA cleavage events. First, a siRNA duplex was 5′ end-labeled with ³²P on either the sense or antisense strand. The labeled siRNA duplex was incubated with 90% human serum at 37° C., and a sample of the solution was removed and quenched at increasing time points. The samples were analyzed by electrophoresis.

### Example 4

### Dual Luciferase Gene Silencing Assays

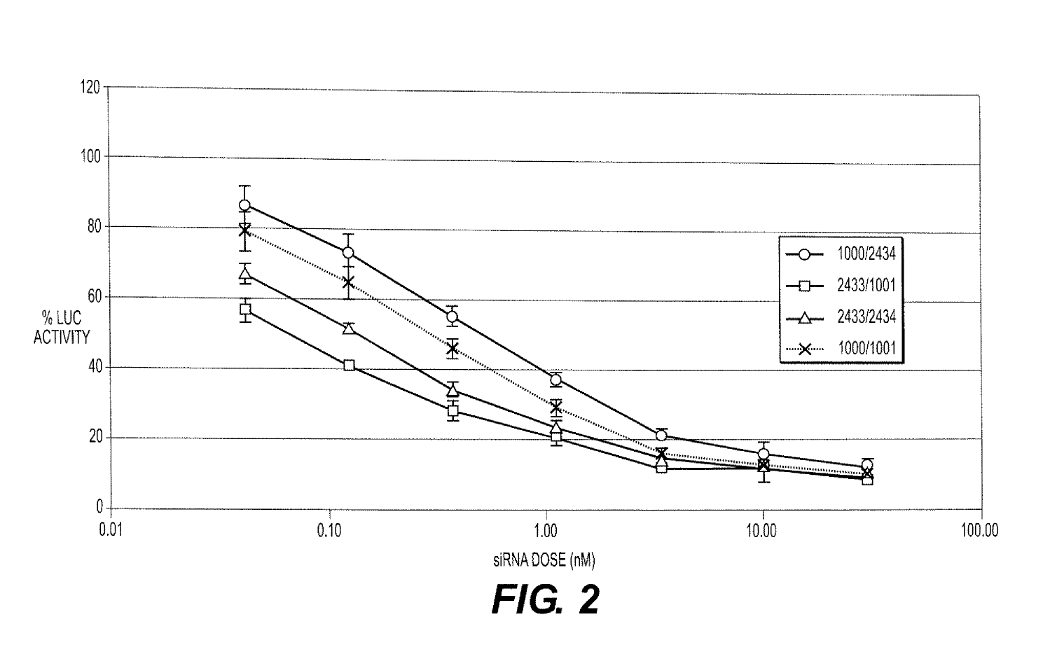

In vitro activity of siRNAs, selected from Example 1 (Table 6), was determined using a high-throughput 96-well plate format luciferase silencing assay. Assays were performed in one of two possible formats. In the first format, HeLa SS6 cells were first transiently transfected with plasmids encoding firefly (target) and renilla (control) luciferase. DNA transfections were performed using Lipofectamine 2000 (Invitrogen) and the plasmids gWiz-Luc (Aldevron, Fargo, N. Dak.) (200 ng/well) and pRL-CMV (Promega, Madison, Wis.) (200 ng/well). After 2 h, the plasmid transfection medium was removed, and the firefly luciferase targeting siRNAs were added to the cells at various concentrations. In the second format, HeLa Dual-luc cells (stably expressing both firefly and renilla luciferase) were directly transfected with firefly luciferase targeting siRNAs. SiRNA transfections were performed using either TransIT-TKO (Mirus, Madison, Wis.) or Lipofectamine 2000 according to manufacturer protocols. After 24 h, cells were analyzed for both firefly and renilla luciferase expression using a plate luminometer (VICTOR², PerkinElmer, Boston, Mass.) and the Dual-Glo Luciferase Assay kit (Promega). Firefly/renilla luciferase expression ratios were used to determine percent gene silencing (FIG. 2) relative to mock-treated (no siRNA) controls.

### Example 5

### Factor VII (FVII) in vitro assay

Cell Seeding for Transfection. Cells are seeded into 96-well plates one day prior to siRNA transfection at a density of 15,000 cells per well in media without antibiotics (150,000 cells/ml media, 100 μl per well).

Standard Transfection Conditions for FVII Stable Cell Lines

Lipofectamine 2000 at a concentration of 0.5 μl/well is used for transfection in a 96 well plate set-up.

Dilute FVII-targeting siRNA or control siRNA to a concentration of 6 nM in OptiMEM.

Mix siRNA and transfection agent (lipofectamine 2000) and allow the complex to form by incubating 20 minutes at room temperature

After 20 minutes, add 50 μl of complexes (total 60 μl volume) to a single well containing cells that were seeded on the previous day (well already contains 100 μl of growth medium). Mix by gently pipetting up and down. Well now contains 150 μl total volume, 1 nM siRNA, 0.5 μl LF 2000 reagent.

Return plate to 37° C. incubator.

Remove media and replace with fresh media (100 μl/well) 24 hours after LF2000 complexes are added to the plate.

24 hours after media exchange, collect media supernatant for FVII activity assay.

Levels of Factor VII protein in the supernatant were determined in samples using a chromogenic assay (Coaset Factor VII, DiaPharma Group, OH or Biophen FVII, Aniara Corporation, OH) according to manufacturer protocols.

In vitro tested siRNAs selected from Example 1 (Table 6) are shown in FIG. 3A.

In vitro tested siRNAs selected from Example 2 are shown in Table 9 and results are shown in FIG. 3B. Presence of the 2′-deoxy-2′-deoxy-2′-fluoronucleotides in the antisense strand enhances the activity of siRNAs relative to the unmodified siRNAs and siRNAs comprising the 2′-O-methyl modification in the antisense strand.

TABLE 9
In vitro tested siRNAs.

| | | Strand | |
|---|---|---|---|
| Duplex No. | Modification | Type | Sequence* |
| AD-1596 | unmod | Sense | GGAUCAUCUCAAGUCUUACdTdT |
| | | Antisense | GUAAGACUUGAGAUGAUCCdTdT |
| AD-1661 | F/F | Sense | GGAucAucucAAGucuuAcdTsdT |
| | | Antisense | GuAAGAcuuGAGAuGAuccdTsdT |
| AD-19013 | OMe/OMe | Sense | GGAucAucucAAGucuuAcdTsdT |
| | | Antisense | GuAAGAcuuGAGAuGAuccdTsdT |
| AD-19016 | OMe/F | Sense | GGAucAucucAAGucuuAcdTsdT |
| | | Antisense | GuAAGAcuuGAGAuGAuccdTsdT |
| | F/OMe | Sense | GGAucAucucAAGucuuAcdTsdT |
| | | Antisense | GuAAGAcuuGAGAuGAuccdTsdT |

*s is phosphorothioate backbone linkage, lowercase is 2′-O-methyl nucleotide, underlined lower case is 2′-decoxy-2′-fluoronucleotide.

### Example 6

### FVII and apoB in vivo Assay

In vivo rodent Factor VII and ApoB silencing experiments. C57BL/6 mice (Charles River Labs, MA) and Sprague-Dawley rats (Charles River Labs, MA) received either saline or siRNA in desired formulations via tail vein injection at a volume of 0.01 mL/g. At various time points post-administration, animals were anesthesized by isofluorane inhalation and blood was collected into serum separator tubes by retroorbital bleed. Serum levels of Factor VII protein were determined in samples using a chromogenic assay (Coaset Factor VII, DiaPharma Group, OH or Biophen FVII, Aniara Corporation, OH) according to manufacturer protocols. A standard curve was generated using serum collected from saline treated animals. In experiments where liver mRNA levels were assessed, at various time points post-administration, animals were sacrificed and livers were harvested and snap frozen in liquid nitrogen. Frozen liver tissue was ground into powder. Tissue lysates were prepared and liver mRNA levels of Factor VII and apoB were determined using a branched DNA assay (QuantiGene Assay, Panomics, Calif.)

Results of the FVII in vivo assay of siRNAs selected from Example 1 (Table 6) are shown in FIG. 4A.

In vivo tested siRNAs selected from Example 2 are shown in Table 10. Results of the FVII in vivo assay are shown in FIG. 4B. siRNAs with 2′-deoxy-2′-deoxy-2′-fluoronucleotides in the antisense and a 2′-modification in the sense strand show reduction in the serum FVII protein levels activity relative to siRNAs with non 2′-F modification in the antisense strand.

TABLE 10
In vivo tested siRNAs.

| | Strand | |
|---|---|---|
| Identifier | Type | Sequence* |
| LNP01_1661 | Sense | GGAucAucucAAGucuuAcdTsdT |
| | Antisense | GuAAGAcuuGAGAuGAuccdTsdT |
| LNP01_19013 | Sense | GGAucAucucAAGucuuAcdTsdT |
| | Antisense | GuAAGAcuuGAGAuGAuccdTsdT |
| LNP01_19014 | Sense | GGA(Teo)(m5Ceo)A(Teo)(m5Ceo)(Teo)(m5Ceo)AAG(m5Ceo) |
| | | (m5Ceo)(Teo)(Teo)A(m5Ceo)dTsdT |
| | Antisense | G(Teo)AAGA(m5Ceo)(Teo)(Teo)GAGA(Teo)GA(Teo)(m5Ceo) |
| | | (m5Ceo)dTsdT |
| LNP01_19015 | Sense | GGA(Tln)CA(Tln)C(Tln)CAAGCC(Tln)UACdTsdT |
| | Antisense | G(Tln)AAGAC(Tln)(Tln)GAGA(Tln)GA(Tln)CCdTsdT |
| LNP01_19016 | Sense | GGAucAucucAAGucuuAcdTsdT |
| | Antisense | GuAAGAcuuGAGAuGAuccdTsdT |
| LNP01_19017 | Sense | GGA(Teo)(m5Ceo)A(Teo)(m5Ceo)(Teo)(m5Ceo)AAG(m5Ceo) |
| | | (m5Ceo)(Teo)(Teo)A(m5Ceo)dTsdT |
| | Antisense | GuAAGAcuuGAGAuGAuccdTsdT |
| LNP01_19018 | Sense | GGA(Tln)CA(Tln)C(Tln)CAAGCC(Tln)UACdTsdT |
| | Antisense | GuAAGAcuuGAGAuGAuccdTsdT |

*s is phosphorothioate backbone linkage, lowercase is 2′-O-methyl nucleotide, (Teo) is 2′-methoxyethyl-thymidine, (5mCeo) is 2′-methoxyethyl-cytidine, (Tln) is 2′-O, 4′-methylene-thymidine (LNA), underlined lower case is 2′-decoxy-2′-fluoronucleotide.

### Example 7

### Cytokine Induction in Human PBMC

Procedure

Isolation of peripheral blood mononuclear cells human blood (PBMC)

Comparison of different FVII siRNA
best positive control for IFN-α direct incubation (DI)
siRNA positive control for direct incubation (DI)
positive control for siRNA transfection
FVII siRNA unmodified AD-1596 and 2′-F modified AD-1661

direct incubation (500 nM)

transfection (130 nM) with Lipofectamine-2000

ELISA with supernatants taken after 24 h; IFN-α.

Results of siRNAs selected from Example 1 are shown in FIGS. 5A and B.

Results of siRNAs selected from Example 2 (FIGS. 5C and D) show that 2′-F modified siRNA B (AD-1661) is non-stimulatory relative to the unmodified siRNA A (AD-1596).

### Example 8

### Binding Affinity and Thermal Stability

Tₘ analysis. Absorbance versus temperature curves were measured at 260 and 280 nm using a DU 800 spectrophotometer (Serial Number 8001373) with software version 2.0, Build 83. Oligonucleotide concentration was 4 μM; and concentration of each strand was determined from the absorbance at 85° C. and extinction coefficients calculated according to Puglisi and Tinoco (Methods Enzymol, 1989, 180, 304-325). Oligonucleotide solutions were heated at a rate of 0.5° C./min in 1 cm path length cells and then cooled to confirm reversibility and lack of evaporation. Tₘ values were obtained from the absorbance versus temperature curves. Standard deviations did not exceed±0.5° C. Each Tm reported was an average of two experiments.

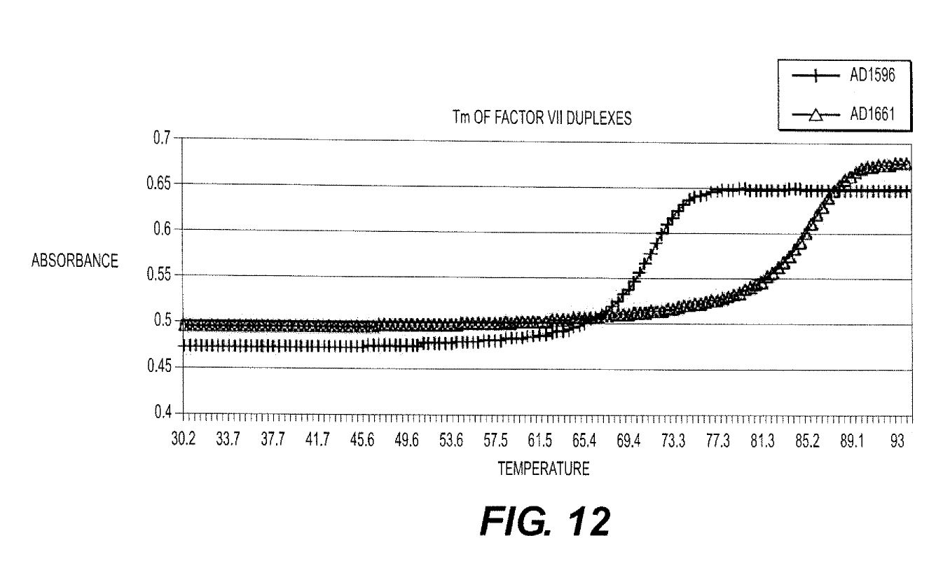

A plot of absorbance vs. temperature yielded thermal denturation of unmodified and 2′-F modified siRNA duplexes, selected from Example 1, are shown in FIG. 12). Thermal stability of 2′-F modified siRNA showed Tm enhancement and hence improved binding affinity.

Absorbance vs. temperature yielded thermal denturation of unmodified and modified siRNA duplexes are also plotted on duplexes selected from Example 2, and results are shown in Table 11.

TABLE 11
Thermal stability of modified siRNAs.

| | Modifications | |
|---|---|---|
| Duplex No. | (Sense strand/antisense strand) | Tm (150 mM NaCl) |
| AD-1596 | Unmodified RNA | 71.8 |
| AD-1661 | 2′-Fluoro/2′-Fluoro | 86.2 |
| AD-19013 | 2′-O-Methyl/2′-O-Methyl | 80.0 |
| AD-19014 | 2′-Methoxyethoxy/2′-Methoxyethoxy | 87.1 |
| AD-19015 | LNA/LNA | >100.0 |
| AD-19016 | 2′-O-Methyl/2′-Fluoro | 83.0 |
| AD-19017 | 2′-Methoxyethoxy/2′-Fluoro | 91.0 |
| AD-19018 | LNA/2′-Fluoro | ~94.0 |

### Example 9

### RP-HPLC Binding Assay of Unmodified and 2′-F Modified siRNA Duplex

Instrument; Agilent 1100-HPLC

Buffer A: 0.1M TEAA

Buffer B: 50% buffer A and 50% accetonitrile

Flow: 0.25 mL/min

Column. XBridge C18 2.5 u, 2.1×50 mm

Temperature 35° C.

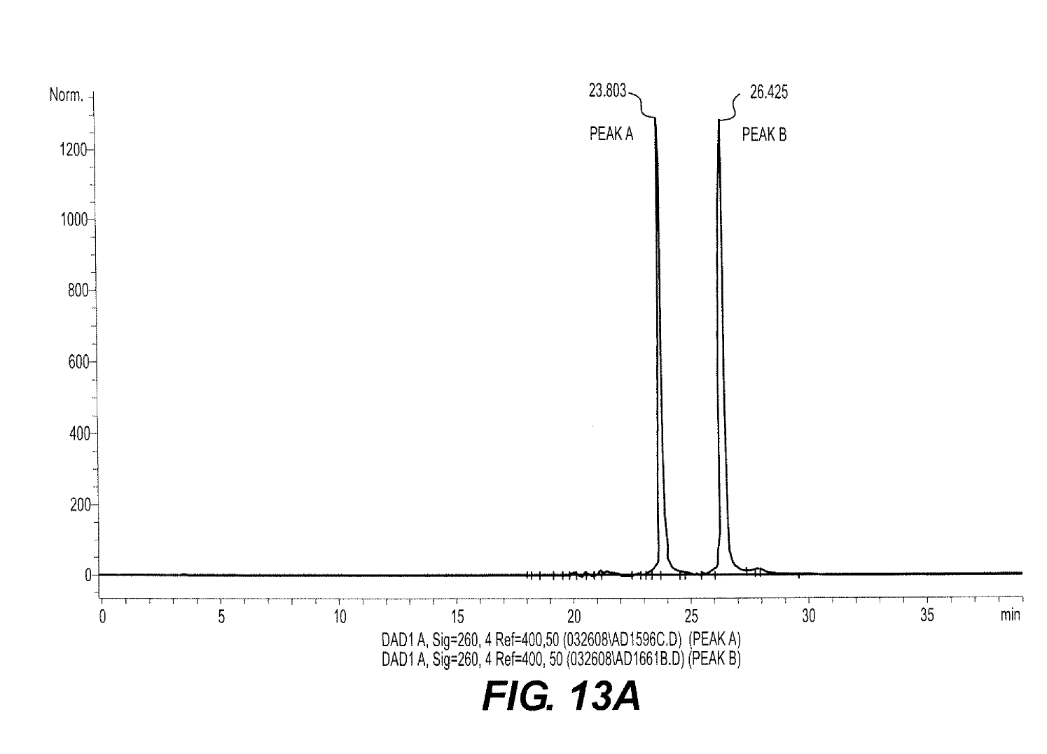

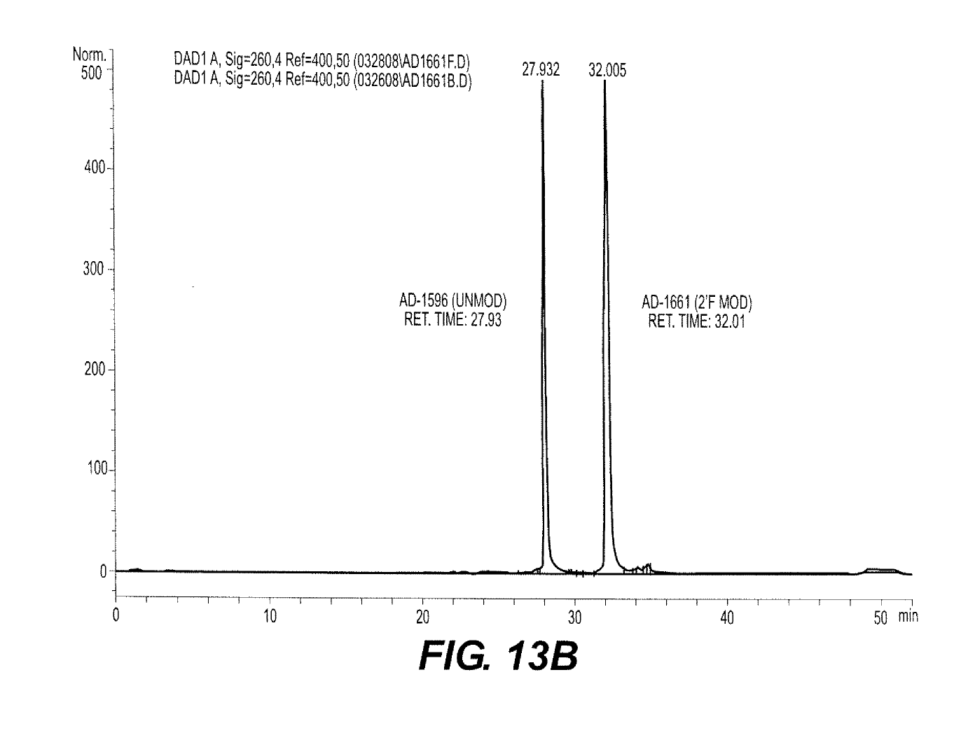

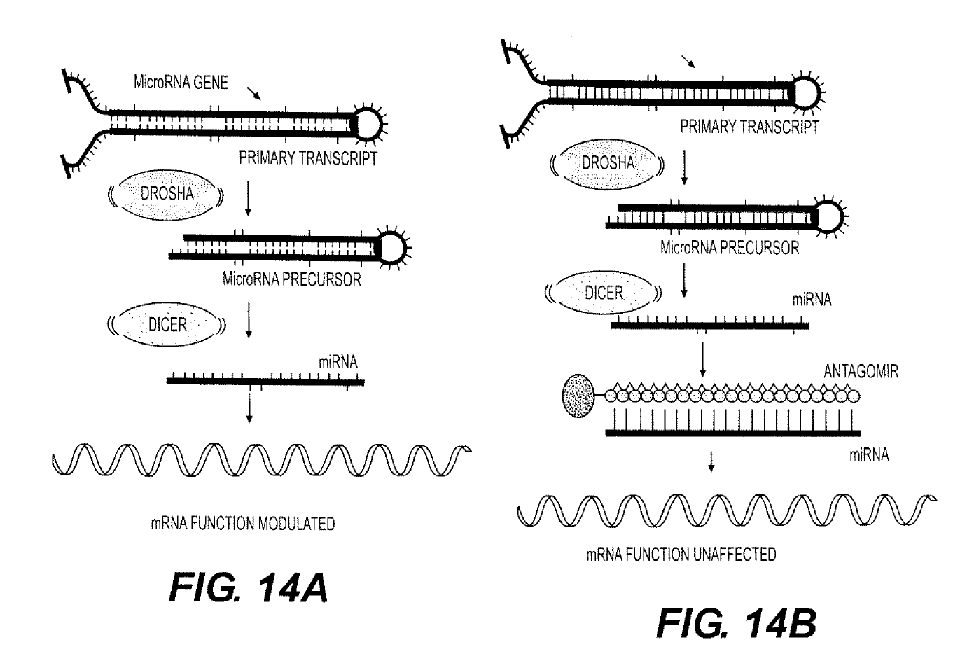

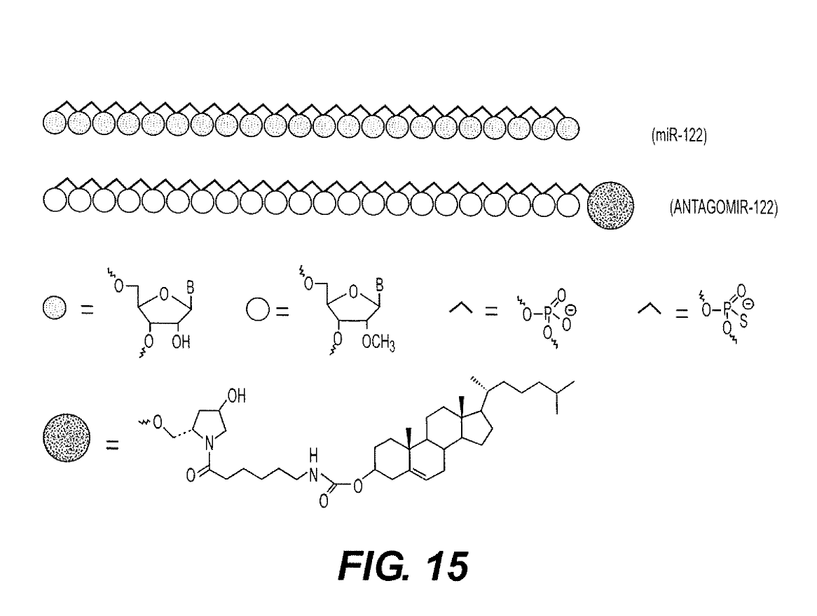

Retention time of both unmodified and 2′-F modified FVII siRNA were compared. The results for siRNAs selected from Example 1 are shown in FIG. 13A, and results for siRNAs selected from Example 2 are shown in FIG. 13B. 2′-F modified siRNA showed longer retention compared to unmodified control which implies improved hydrophobicity with respect control. Increase in hydrophobicity favors serum and plasma protein binding.

### Example 10

### Lipidoid-siRNA Formulation

Lipidoid-based siRNA formulations comprised lipidoid, cholesterol, poly(ethylene glycol)-lipid (PEG-lipid), and siRNA selected from Example 1. Formulations were prepared using a protocol similar to that described by Semple and colleagues (Semple, S. C. et al. Efficient encapsulation of antisense oligonucleotides in lipid vesicles using ionizable aminolipids: formation of novel small multilamellar vesicle structures. Biochim Biophys Acta 1510, 152-166 (2001)). Stock solutions of 98N12-5(1).4HCl MW 1489, mPEG2000-Ceramide C16 (Avanti Polar Lipids) MW 2634 or mPEG₂₀₀₀-DMG MW 2660, and cholesterol MW 387 (Sigma-Aldrich) were prepared in ethanol and mixed to yield a molar ratio of 42:10:48. Mixed lipids were added to 125 mM sodium acetate buffer pH 5.2 to yield a solution containing 35% ethanol, resulting in spontaneous formation of empty lipidoid nanoparticles. Resulting nanoparticles were extruded through a 0.08μ membrane (2 passes). siRNA in 35% ethanol and 50 mM sodium acetate pH 5.2 was added to the nanoparticles at 1:7.5 (wt:wt) siRNA:total lipids and incubated at 37° C. for 30 min Ethanol removal and buffer exchange of siRNA-containing lipidoid nanoparticles was achieved by tangential flow filtration against phosphate buffered saline using a 100,000 MWCO membrane. Finally, the formulation was filtered through a 0.2μ sterile filter. Particle size was determined using a Malvern Zetasizer NanoZS (Malvern, UK). siRNA content was determined by UV absorption at 260 nm and siRNA entrapment efficiency was determined by Ribogreen assay. Resulting particles had a mean particle diameter of approximately 50 nm, with peak width of 20 nm, and siRNA entrapment efficiency of >95%.

### Example 11

### In vivo miRNA Silencing Experiments

C57BL/6NCRL mice (Charles River, Sulzfeld, Germany) received lipidoid formulations of antagomir or anti-miR via tail vein injection at 5 mg/kg (0.5 mg/mL) on three consecutive days. Livers were taken at day 4 and expression levels of miR-122 were determined. Liver tissue was dissolved in Proteinase K-containing cell and tissue lysis buffer (EPICENTRE, Madision, Wis.) and subjected to sonication. Total RNA was extracted with TE-saturated phenol (Roth, Karlsruhe, Germany) and subsequent precipitation in ethanol.

Total liver RNA was simultaneously hybridized in solution to a miR-122-specific probe and the U6 probe. The hybridization conditions allowed detection of U6 RNA and mature miRNA, but not pre-miRNA. Following treatment with S1 nuclease, samples were loaded on denaturing 10% acrylamide gels. Gels were exposed to a phosphoimager screen and analyzed on a Typhoon 9200 instrument (GE Healthcare). Relative signal intensities of miR-122 versus U6 were calculated for each sample. Expression level analysis of miR-122 target genes by branched DNA assay. Assay was performed as described (Krutzfeldt, J. et al. Silencing of microRNAs in vivo with ‘antagomirs’ Nature 438, 685-689 (2005)). Briefly, 30-50 mg of frozen liver tissue was lysed in 1 mL Tissue and Cell Lysis Buffer (EPICENTRE, WI) by sonication. 10-40 μL lysate was used for branched DNA assay, depending on signal strength of target gene. Probe sets were designed using QuantiGene ProbeDesigner software. Target gene expression was assayed according to QuantiGene Detection Assay recommendations and normalized to corresponding GAPDH housekeeper expression from same liver tissue lysate.

The references cited above are all incorporated herein by reference, whether specifically incorporated or not. All publications, patents, patent applications, and GenBank sequences, cited herein are hereby expressly incorporated by reference for all purposes. When definitions of terms in documents that are incorporated by reference herein conflict with those used herein, the definitions used herein govern. Citation of the documents herein is not intended as an admission that any of them is pertinent prior art. All statements as to the date or representation as to the contents of these documents is based on the information available to the applicant and does not constitute any admission as to the correctness of the dates or contents of these documents. Having now fully described this invention, it will be appreciated by those skilled in the art that the same can be performed within a wide range of equivalent parameters, concentrations, and conditions without departing from the spirit and scope of the invention and without undue experimentation.

What is claimed is:

1. A method of modulating the expression of a target gene in an organism comprising administering an iRNA agent, wherein said iRNA comprises at least one 2′-deoxy-2′-fluoro (2′-F) nucleotide in the antisense strand and at least one modified nucleotide in the sense strand, wherein said modified nucleotide is selected from the group consisting of 2′-deoxy-2′-fluoro, 2′-O-methyl (2′-OMe), 2′-methoxyethyl (2′-MOE), and 2′-O,4′-C-methylene (LNA).

2. The method of claim 1, wherein said antisense strand comprises at least one 5′-pyrimidine-purine dinucleotide wherein the pyrimidine is 2′-deoxy-2′-fluoro.

3. The method of claim 1, wherein the 5′-most pyrimidines in all occurrences of sequence motif 5′-pyrimidine-purine-3′ in the antisense strand is a 2′-deoxy-2′-fluoro.

4. The method of claim 1, wherein all pyrimidines are 2′-deoxy-2′-fluoro in the antisense strand.

5. The method of claim 1, wherein said sense strand comprises at least one 5′-pyrimidine-purine-3′ dinucleotide wherein the pyrimidine is modified with modification chosen from a group consisting of 2′-deoxy-2′-fluoro, 2′-O-methyl, 2′-methoxyethyl, and 2′-O,4′-C-methylene.

6. The method of claim 1, wherein the 5′-most pyrimidines in all occurrences of sequence motif 5′-pyrimidine-purine-3′ in the sense strand are modified with modification selected from the group consisting of 2′-deoxy-2′-fluoro, 2′-O-methyl, 2′-methoxyethyl, and 2′-O,4′-C-methylene.

7. The method of claim 1, wherein all pyrimidines in the sense strand are modified with modification selected from the consisting of 2′-deoxy-2′-fluoro, 2′-O-methyl, 2′-methoxyethyl, and 2′-O,4′-C-methylene.

8. The method of claim 1, wherein the 5′-most pyrimidines in all occurrences of sequence motif 5′-pyrimidine-purine-3′ in the antisense strand is a 2′-deoxy-2′-fluoro and said sense strand comprises at least one 5′-pyrimidine-purine-3′ dinucleotide wherein the pyrimidine is modified with modification selected from the group consisting of 2′-deoxy-2′-fluoro, 2′-β-methyl, 2′-methoxyethyl, and 2′-O,4′-C-methylene.

9. The method of claim 1, wherein the 5′-most pyrimidines in all occurrences of sequence motif 5′-pyrimidine-purine-3′ in the antisense strand is a 2′-deoxy-2′-fluoro and the 5′-most pyrimidines in all occurrences of sequence motif 5′-pyrimidine-purine-3′ in the sense strand are modified with modification selected from the group consisting of 2′-deoxy-2′-fluoro, 2′-β-methyl, 2′-methoxyethyl, and 2′-O,4′-C-methylene.

10. The method of claim 1, wherein the 5′-most pyrimidines in all occurrences of sequence motif 5′-pyrimidine-purine-3′ in the antisense strand are 2′-deoxy-2′-fluoro and all pyrimidines in the sense strand are modified with modification selected from the group consisting of 2′-deoxy-2′-fluoro, 2′-O-methyl, 2′-methoxyethyl, and 2′-O,4′-C-methylene.

11. The method of claim 1, wherein all pyrimidines are 2′-deoxy-2′-fluoro in the antisense strand and said sense strand comprises at least one 5′-pyrimidine-purine-3′ dinucleotide wherein the pyrimidine is modified with modification selected from the group consisting of 2′-deoxy-2′-fluoro, 2′-O-methyl, 2′-methoxyethyl, and 2′-O,4′-C-methylene.

12. The method of claim 1, wherein all pyrimidines are 2′-deoxy-2′-fluoro in the antisense strand and the 5′-most pyrimidines in all occurrences of sequence motif 5′-pyrimidine-purine-3′ in the sense strand are modified with modification selected from the group consisting of 2′-deoxy-2′-fluoro, 2′-O-methyl, 2′-methoxyethyl, and 2′-O,4′-C-methylene.

13. The method of claim 1, wherein all pyrimidines are 2′-deoxy-2′-fluoro in the antisense strand and all pyrimidines in the sense strand are modified with modification selected from the group consisting of 2′-deoxy-2′-fluoro, 2′-O-methyl, 2′-methoxyethyl, and 2′-O,4′-C-methylene.

14. A method of decreasing the immunogenicity of an iRNA agent, wherein said iRNA comprises at least one 2′-deoxy-2′-fluoro (2′-F) nucleotide in the antisense strand and at least one modified nucleotide in the sense strand, and wherein said modified nucleotide is selected from the group consisting of 2′-deoxy-2′-fluoro, 2′-O-methyl, 2′-methoxyethyl, and 2′-O,4′-C-methylene, wherein the siRNA molecule has a decreased immunogenicity relative to an siRNA molecule having the identical sequence but comprising fewer or no 2′-F modifications.

15. A composition, comprising a single-stranded oligonucleotide represented by formula I: 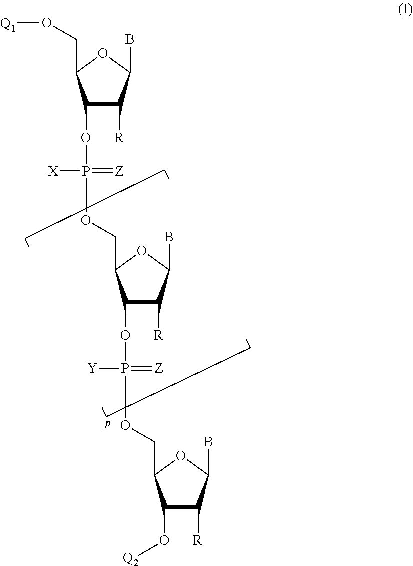
wherein:
X is O or S;
Y is O or S;
Z is O or S;
Q₁ is H, a ligand, PO₃H₂, PO₃HM, PO₃M₂, PO₂SH₂, PO₂SHM, PO₂SM₂, PO₃M, or PO₂SM;
Q₂ is H, a ligand, PO₃H₂, PO₃HM, PO₃M₂, PO₂SH₂, PO₂SHM, PO₂SM₂, PO₃M, or PO₂SM;
M is an alkali cation, alkaline earth dication, or an organic cation or dication;
R is H, OH, OMe, O—CH₂CH₂—OMe, F, O—CH₂C(O)NHMe, OCH₂-(4′-C), or OCH₂CH₂-(4′-C), wherein R represents F at least once;
B is a nucleobase; and
p is an integer ranging from 10 to 98.

16. The composition of claim 15, wherein at least one instance of X or Y is S.

17. The composition of claim 15, wherein a plurality of instances of Y represent S.

18. The composition of claim 15, wherein Y is S; and X is S.

19. The composition of claim 15, wherein p ranges from 14-28.

20. The composition of claim 15, wherein R represents F in a plurality of instances.

21. The composition of claim 15, wherein the phosphorothioate internucleotide linkage is attached to the 5′-hydroxyl of a nucleoside wherein R is F.

22. The composition of claim 15, wherein the phosphorothioate internucleotide linkage is attached to the 3′-hydroxyl of a nucleoside wherein R is F.

23. The composition of claim 15, wherein the at least one nucleotide comprising a 2′-deoxy-2′-fluoro modification is not a 5′ terminal nucleotide or a 3′ terminal nucleotide.

24. The composition of claim 15, wherein Q₁ represents a phosphate or phosphorothioate group; Q₂ represents a phosphate or phosphorothioate group, or both Q₁ and Q₂ represents a phosphate or phosphorothioate group.

25. The composition of claim 15, wherein said single-stranded oligonucleotied comprises a 3′-terminal deoxythymidine.

26. The composition of claim 15, wherein said single-stranded oligonucleotide comprises at least one nucleotide selected from the group consisting of 2′-O-methyl nucleotides, 2′-methoxyethoxy nucleotides, 2′-O—N-methylacetamido nucleotides, LNAs, and ENAs.

27. The composition of claim 15, further comprising at least one ligand covalently attached to the 5′-terminus of the oligonucleotide and/or at least one ligand covalently attached to the 3′-terminus of the oligonucleotide.

28. The composition of claim 27, wherein the ligand comprises a targeting group, a protein-binding agent, or an endosomal release agent.

29. The composition of claim 28, wherein the ligand comprises a targeting group; and said targeting group is selected from the group consisting of folate, cholesterol, bile acids, steroids, β-GalNAc, mannose, an RGD peptide, a peptide, an antibody, and an aptamer.

30. The composition of claim 28, wherein the ligand comprises a protein-binding agent; and said protein-binding agent is selected from the group consisting of cholesterol, lipophiles, ibuprofen, naproxen, ligands capable of binding to albumin, and ligands capable of binding to lipoproteins (LDL or HDL).

31. The composition of claim 28, wherein the attachment of the ligand to the oligonucleotide is biodegradable.

32. The composition of claim 31, wherein the biodegradability is at least partially in response to intracellular pH change, is at least partially in response to intracellular reductive environment, is at least partially in response to peptidase activity, is at least partially in response to esterase activity, or a combination thereof.

33. A pharmaceutical composition, comprising the composition of claim 15, and a pharmaceutically acceptable excipient, carrier or diluent.

34. The composition of claim 15, wherein the siRNA molecule has decreased immunogenicity relative to an siRNA molecule having the identical sequence but comprising fewer or no 2′-deoxy-2′-fluoro modifications.

35. A method of suppressing the endogenous expression of a gene, comprising contacting a cell with an effective amount of the composition of claim 15, wherein the effective amount is an amount that partially or substantially suppresses the endogenous expression of said gene.


SEQUENCE LISTING (147 sequences)

SEQ ID NO: 1 - DNA, 21 bp, Artificial Sequence
  Synthetic Oligonucleotide
cuuacgcugaguacuucgatt

SEQ ID NO: 2 - DNA, 21 bp, Artificial Sequence
  Synthetic Oligonucleotide
ucgaaguacucagcguaagtt

SEQ ID NO: 3 - DNA, 21 bp, Artificial Sequence
  Synthetic Oligonucleotide
cuuacgcugaguacuucgatt

SEQ ID NO: 4 - DNA, 21 bp, Artificial Sequence
  Synthetic Oligonucleotide
ucgaaguacucagcguaagtt

SEQ ID NO: 5 - DNA, 21 bp, Artificial Sequence
  Synthetic Oligonucleotide
accgaaaggucuuaccggatt

SEQ ID NO: 6 - DNA, 21 bp, Artificial Sequence
  Synthetic Oligonucleotide
uccgguaagaccuuucggutt

SEQ ID NO: 7 - DNA, 21 bp, Artificial Sequence
  Synthetic Oligonucleotide
accgaaaggucuuaccggatt

SEQ ID NO: 8 - DNA, 21 bp, Artificial Sequence
  Synthetic Oligonucleotide
uccgguaagaccuuucggutt

SEQ ID NO: 9 - DNA, 21 bp, Artificial Sequence
  Synthetic Oligonucleotide
ggaucaucucaagucuuactt

SEQ ID NO: 10 - DNA, 21 bp, Artificial Sequence
  Synthetic Oligonucleotide
guaagacuugagaugaucctt

SEQ ID NO: 11 - DNA, 21 bp, Artificial Sequence
  Synthetic Oligonucleotide
guaagacuugagaugaucctt

SEQ ID NO: 12 - DNA, 21 bp, Artificial Sequence
  Synthetic Oligonucleotide
ggaucaucucaagucuuactt

SEQ ID NO: 13 - DNA, 21 bp, Artificial Sequence
  Synthetic Oligonucleotide
ggaucaucucaagucuuactt

SEQ ID NO: 14 - DNA, 21 bp, Artificial Sequence
  Synthetic Oligonucleotide
ggaucaucucaagucuuacuu

SEQ ID NO: 15 - DNA, 21 bp, Artificial Sequence
  Synthetic Oligonucleotide
guaagacuugagaugaucctt

SEQ ID NO: 16 - DNA, 21 bp, Artificial Sequence
  Synthetic Oligonucleotide
guaagacuugagaugaucctt

SEQ ID NO: 17 - DNA, 21 bp, Artificial Sequence
  Synthetic Oligonucleotide
guaagacuugagaugaucctt

SEQ ID NO: 18 - DNA, 21 bp, Artificial Sequence
  Synthetic Oligonucleotide
guaagacuugagaugauccug

SEQ ID NO: 19 - DNA, 21 bp, Artificial Sequence
  Synthetic Oligonucleotide
ggaucaucucaagucuuactt

SEQ ID NO: 20 - DNA, 21 bp, Artificial Sequence
  Synthetic Oligonucleotide
ggacuacucuaaguucuactt

SEQ ID NO: 21 - DNA, 21 bp, Artificial Sequence
  Synthetic Oligonucleotide
ggacuacucuaaguucuactt

SEQ ID NO: 22 - DNA, 21 bp, Artificial Sequence
  Synthetic Oligonucleotide
guagaacuuagaguagucctt

SEQ ID NO: 23 - DNA, 21 bp, Artificial Sequence
  Synthetic Oligonucleotide
ggaucaucucaagucuuactt

SEQ ID NO: 24 - DNA, 21 bp, Artificial Sequence
  Synthetic Oligonucleotide
guaagacuugagaugaucctt

SEQ ID NO: 25 - DNA, 21 bp, Artificial Sequence
  Synthetic Oligonucleotide
ggaaucuuauauuugauccaa

SEQ ID NO: 26 - DNA, 21 bp, Artificial Sequence
  Synthetic Oligonucleotide
ggaaucuuauauuugauccaa

SEQ ID NO: 27 - DNA, 23 bp, Artificial Sequence
  Synthetic Oligonucleotide
uuggaucaaauauaagauucccu

SEQ ID NO: 28 - DNA, 23 bp, Artificial Sequence
  Synthetic Oligonucleotide
uuggaucaaauauaagauucccu

SEQ ID NO: 29 - DNA, 23 bp, Artificial Sequence
  Synthetic Oligonucleotide
uuggaucaaauauaagauucccu

SEQ ID NO: 30 - DNA, 21 bp, Artificial Sequence
  Synthetic Oligonucleotide
gccuggaguuuauucggaatt

SEQ ID NO: 31 - DNA, 21 bp, Artificial Sequence
  Synthetic Oligonucleotide
gccuggaguuuauucggaaga

SEQ ID NO: 32 - DNA, 21 bp, Artificial Sequence
  Synthetic Oligonucleotide
uuccgaauaaacuccaggctt

SEQ ID NO: 33 - DNA, 21 bp, Artificial Sequence
  Synthetic Oligonucleotide
uuccgaauaaacuccaggccu

SEQ ID NO: 34 - DNA, 21 bp, Artificial Sequence
  Synthetic Oligonucleotide
uuccgaauaaacuccaggctt

SEQ ID NO: 35 - DNA, 21 bp, Artificial Sequence
  Synthetic Oligonucleotide
cuggcugaauuucagagcatt

SEQ ID NO: 36 - DNA, 21 bp, Artificial Sequence
  Synthetic Oligonucleotide
ugcucugaaauucagccagtt

SEQ ID NO: 37 - DNA, 21 bp, Artificial Sequence
  Synthetic Oligonucleotide
cuuacgcugaguacuucgatt

SEQ ID NO: 38 - DNA, 21 bp, Artificial Sequence
  Synthetic Oligonucleotide
ucgaaguacucagcguaagtt

SEQ ID NO: 39 - DNA, 21 bp, Artificial Sequence
  Synthetic Oligonucleotide
accgaaaggucuuaccggatt

SEQ ID NO: 40 - DNA, 21 bp, Artificial Sequence
  Synthetic Oligonucleotide
uccgguaagaccuuucggutt

SEQ ID NO: 41 - DNA, 21 bp, Artificial Sequence
  Synthetic Oligonucleotide
accgaaaggucuuaccggatt

SEQ ID NO: 42 - DNA, 21 bp, Artificial Sequence
  Synthetic Oligonucleotide
accgaaaggucuuaccggatt

SEQ ID NO: 43 - DNA, 21 bp, Artificial Sequence
  Synthetic Oligonucleotide
accgaaaggucuuaccggatt

SEQ ID NO: 44 - DNA, 21 bp, Artificial Sequence
  Synthetic Oligonucleotide
accgaaaggucuuaccggatt

SEQ ID NO: 45 - DNA, 21 bp, Artificial Sequence
  Synthetic Oligonucleotide
accgaaaggucuuaccggatt

SEQ ID NO: 46 - DNA, 21 bp, Artificial Sequence
  Synthetic Oligonucleotide
accgaaaggucuuaccggatt

SEQ ID NO: 47 - DNA, 21 bp, Artificial Sequence
  Synthetic Oligonucleotide
accgaaaggucuuaccggatt

SEQ ID NO: 48 - DNA, 21 bp, Artificial Sequence
  Synthetic Oligonucleotide
ggacuacucuaaguucuactt

SEQ ID NO: 49 - DNA, 21 bp, Artificial Sequence
  Synthetic Oligonucleotide
guagaacuuagaguagucctt

SEQ ID NO: 50 - DNA, 21 bp, Artificial Sequence
  Synthetic Oligonucleotide
ggacuacucuaaguucuactt

SEQ ID NO: 51 - DNA, 21 bp, Artificial Sequence
  Synthetic Oligonucleotide
guagaacuuagaguagucctt

SEQ ID NO: 52 - DNA, 21 bp, Artificial Sequence
  Synthetic Oligonucleotide
ggacuacucuaaguucuactt

SEQ ID NO: 53 - DNA, 21 bp, Artificial Sequence
  Synthetic Oligonucleotide
guagaacuuagaguagucctt

SEQ ID NO: 54 - DNA, 21 bp, Artificial Sequence
  Synthetic Oligonucleotide
ggacuacucuaaguucuactt

SEQ ID NO: 55 - DNA, 21 bp, Artificial Sequence
  Synthetic Oligonucleotide
guagaacuuagaguagucctt

SEQ ID NO: 56 - DNA, 21 bp, Artificial Sequence
  Synthetic Oligonucleotide
ggacuacucuaaguucuactt

SEQ ID NO: 57 - DNA, 21 bp, Artificial Sequence
  Synthetic Oligonucleotide
guagaacuuagaguagucctt

SEQ ID NO: 58 - DNA, 21 bp, Artificial Sequence
  Synthetic Oligonucleotide
ggacuacucuaaguuuuactt

SEQ ID NO: 59 - DNA, 21 bp, Artificial Sequence
  Synthetic Oligonucleotide
guggaacuuagaguagucctt

SEQ ID NO: 60 - DNA, 21 bp, Artificial Sequence
  Synthetic Oligonucleotide
ggacuacucuaaauucuactt

SEQ ID NO: 61 - DNA, 21 bp, Artificial Sequence
  Synthetic Oligonucleotide
ggacuacucuaaguuuuactt

SEQ ID NO: 62 - DNA, 21 bp, Artificial Sequence
  Synthetic Oligonucleotide
guggaacuuagaguagucctt

SEQ ID NO: 63 - DNA, 21 bp, Artificial Sequence
  Synthetic Oligonucleotide
ggacuacucuaaauucuactt

SEQ ID NO: 64 - DNA, 21 bp, Artificial Sequence
  Synthetic Oligonucleotide
ggacuacucuaaguuuuactt

SEQ ID NO: 65 - DNA, 21 bp, Artificial Sequence
  Synthetic Oligonucleotide
guggaacuuagaguagucctt

SEQ ID NO: 66 - DNA, 21 bp, Artificial Sequence
  Synthetic Oligonucleotide
ggacuacucuaaauucuactt

SEQ ID NO: 67 - RNA, 23 nt, Artificial Sequence
  Synthetic Oligonucleotide microRNA or antagomir
uggagugugacaaugguguuugu

SEQ ID NO: 68 - RNA, 22 nt, Artificial Sequence
  Synthetic Oligonucleotide microRNA or antagomir
uagcagcacguaaauauuggcg

SEQ ID NO: 69 - RNA, 21 nt, Artificial Sequence
  Synthetic Oligonucleotide microRNA or antagomir
cugaccuaugaauugacagcc

SEQ ID NO: 70 - RNA, 22 nt, Artificial Sequence
  Synthetic Oligonucleotide microRNA or antagomir
uguaacagcaacuccaugugga

SEQ ID NO: 71 - RNA, 23 nt, Artificial Sequence
  Synthetic Oligonucleotide microRNA or antagomir
uguuugugguaacagugugaggu

SEQ ID NO: 72 - RNA, 23 nt, Artificial Sequence
  Synthetic Oligonucleotide microRNA or antagomir
uggagugugacaaugguguuugu

SEQ ID NO: 73 - RNA, 23 nt, Artificial Sequence
  Synthetic Oligonucleotide microRNA or antagomir
uggagugugacaaugguguuugu

SEQ ID NO: 74 - RNA, 23 nt, Artificial Sequence
  Synthetic Oligonucleotide microRNA or antagomir
uggaaugugacaguguugugugu

SEQ ID NO: 75 - RNA, 23 nt, Artificial Sequence
  Synthetic Oligonucleotide microRNA or antagomir
uggaaugugacaguguugugugu

SEQ ID NO: 76 - RNA, 23 nt, Artificial Sequence
  Synthetic Oligonucleotide microRNA or antagomir
acaaacaccauugucacacucca

SEQ ID NO: 77 - RNA, 22 nt, Artificial Sequence
  Synthetic Oligonucleotide microRNA or antagomir
ccaucuuuaccagacaguguua

SEQ ID NO: 78 - RNA, 22 nt, Artificial Sequence
  Synthetic Oligonucleotide microRNA or antagomir
ugagcuacagugcuucauauca

SEQ ID NO: 79 - RNA, 24 nt, Artificial Sequence
  Synthetic Oligonucleotide microRNA or antagomir
aacucaccgacagcguugaauguu

SEQ ID NO: 80 - RNA, 21 nt, Artificial Sequence
  Synthetic Oligonucleotide microRNA or antagomir
ggccguccauuaauagaucag

SEQ ID NO: 81 - RNA, 22 nt, Artificial Sequence
  Synthetic Oligonucleotide microRNA or antagomir
uccccauagagcugcugcuaca

SEQ ID NO: 82 - RNA, 22 nt, Artificial Sequence
  Synthetic Oligonucleotide microRNA or antagomir
ccaucauuacccgccaguauua

SEQ ID NO: 83 - RNA, 22 nt, Artificial Sequence
  Synthetic Oligonucleotide microRNA or antagomir
ccacacacuuccuuacauucca

SEQ ID NO: 84 - RNA, 22 nt, Artificial Sequence
  Synthetic Oligonucleotide microRNA or antagomir
cagcuaugccagcaucuugccu

SEQ ID NO: 85 - RNA, 21 nt, Artificial Sequence
  Synthetic Oligonucleotide microRNA or antagomir
ccaacaacaugaaacuaccua

SEQ ID NO: 86 - RNA, 21 nt, Artificial Sequence
  Synthetic Oligonucleotide microRNA or antagomir
cucugucaaaucauaggucau

SEQ ID NO: 87 - RNA, 19 nt, Artificial Sequence
  Synthetic Oligonucleotide microRNA or antagomir
caaugcaacuacaaugcac

SEQ ID NO: 88 - RNA, 22 nt, Artificial Sequence
  Synthetic Oligonucleotide microRNA or antagomir
uuggcauucaccgcgugccuua

SEQ ID NO: 89 - RNA, 21 nt, Artificial Sequence
  Synthetic Oligonucleotide microRNA or antagomir
gucugucaaaucauaggucau

SEQ ID NO: 90 - RNA, 22 nt, Artificial Sequence
  Synthetic Oligonucleotide microRNA or antagomir
ccccuaucacaauuagcauuaa

SEQ ID NO: 91 - RNA, 21 nt, Artificial Sequence
  Synthetic Oligonucleotide microRNA or antagomir
ucaacaucagucuguaagcua

SEQ ID NO: 92 - RNA, 22 nt, Artificial Sequence
  Synthetic Oligonucleotide microRNA or antagomir
acaguucuucaacuggcagcuu

SEQ ID NO: 93 - RNA, 22 nt, Artificial Sequence
  Synthetic Oligonucleotide microRNA or antagomir
cgcauuauuacucacgguacga

SEQ ID NO: 94 - RNA, 15 nt, Artificial Sequence
  Synthetic Oligonucleotide microRNA or antagomir
ccauugucacacucc

SEQ ID NO: 95 - DNA, 21 bp, Artificial Sequence
  Synthetic Oligonucleotide
ggaucaucucaagucuuactt

SEQ ID NO: 96 - DNA, 21 bp, Artificial Sequence
  Synthetic Oligonucleotide
guaagacuugagaugaucctt

SEQ ID NO: 97 - DNA, 21 bp, Artificial Sequence
  Synthetic Oligonucleotide
ggatcatctcaagccttactt

SEQ ID NO: 98 - DNA, 21 bp, Artificial Sequence
  Synthetic Oligonucleotide
gtaagacttgagatgatcctt

SEQ ID NO: 99 - DNA, 21 bp, Artificial Sequence
  Synthetic Oligonucleotide
ggatcatctcaagcctuactt

SEQ ID NO: 100 - DNA, 21 bp, Artificial Sequence
  Synthetic Oligonucleotide
gtaagacttgagatgatcctt

SEQ ID NO: 101 - DNA, 21 bp, Artificial Sequence
  Synthetic Oligonucleotide
ggaucaucucaagucuuactt

SEQ ID NO: 102 - DNA, 21 bp, Artificial Sequence
  Synthetic Oligonucleotide
guaagacuugagaugaucctt

SEQ ID NO: 103 - DNA, 21 bp, Artificial Sequence
  Synthetic Oligonucleotide
ggatcatctcaagccttactt

SEQ ID NO: 104 - DNA, 21 bp, Artificial Sequence
  Synthetic Oligonucleotide
guaagacuugagaugaucctt

SEQ ID NO: 105 - DNA, 21 bp, Artificial Sequence
  Synthetic Oligonucleotide
ggatcatctcaagcctuactt

SEQ ID NO: 106 - DNA, 21 bp, Artificial Sequence
  Synthetic Oligonucleotide
guaagacuugagaugaucctt

SEQ ID NO: 107 - protein, 29 aa, Artificial Sequence
  Endosomolytic Component GALA
AALEALAEALEALAEALEALAEAAAAGGC

SEQ ID NO: 108 - protein, 30 aa, Artificial Sequence
  Endosomolytic Component EALA
AALAEALAEALAEALAEALAEALAAAAGGC

SEQ ID NO: 109 - protein, 15 aa, Artificial Sequence
  Endosomolytic Component.
ALEALAEALEALAEA

SEQ ID NO: 110 - protein, 22 aa, Artificial Sequence
  Endosomolytic Component INF-7
GLFEAIEGFIENGWEGMIWDYG

SEQ ID NO: 111 - protein, 23 aa, Artificial Sequence
  Endosomolytic Component INF HA-2
GLFGAIAGFIENGWEGMIDGWYG

SEQ ID NO: 112 - protein, 48 aa, Artificial Sequence
  Endosomolytic Component diINF-7
GLFEAIEGFIENGWEGMIDGWYGCGLFEAIEGFIENGWEGMIDGWYGC

SEQ ID NO: 113 - protein, 44 aa, Artificial Sequence
  Endosomolytic Component diINF3
GLFEAIEGFIENGWEGMIDGGCGLFEAIEGFIENGWEGMIDGGC

SEQ ID NO: 114 - protein, 35 aa, Artificial Sequence
  Endosomolytic Component GLF
GLFGALAEALAEALAEHLAEALAEALEALAAGGSC

SEQ ID NO: 115 - protein, 34 aa, Artificial Sequence
  Endosomolytic Component GALA-INF3
GLFEAIEGFIENGWEGLAEALAEALEALAAGGSC

SEQ ID NO: 116 - protein, 41 aa, Artificial Sequence
  Endosomolytic Component INF-5
GLFEAIEGFIENGWEGLIDGKGLFEAIEGFIENGWEGLIDG

SEQ ID NO: 117 - protein, 16 aa, Artificial Sequence
  Cell Permeation Peptide Penetratin
RQIKIWFQNRRMKWKK

SEQ ID NO: 118 - protein, 14 aa, Artificial Sequence
  Cell Permeation Peptide Tat fragment (48-60)
GRKKRRQRRRPPQC

SEQ ID NO: 119 - protein, 27 aa, Artificial Sequence
  Cell Permeation Peptide Signal Sequence-based peptide
GALFLGWLGAAGSTMGAWSQPKKKRKV

SEQ ID NO: 120 - protein, 18 aa, Artificial Sequence
  Cell Permeation Peptide PVEC
LLIILRRRIRKQAHAHSK

SEQ ID NO: 121 - protein, 26 aa, Artificial Sequence
  Cell Permeation Peptide Transportan
GWTLNSAGYLLKINLKALAALAKKIL

SEQ ID NO: 122 - protein, 18 aa, Artificial Sequence
  Cell Permeation Peptide Amphiphilic model peptide
KLALKLALKALKAALKLA

SEQ ID NO: 123 - protein, 9 aa, Artificial Sequence
  Cell Permeation Peptide Arg9
RRRRRRRRR

SEQ ID NO: 124 - protein, 10 aa, Artificial Sequence
  Cell Permeation Peptide Bacterial cell wall permeating
KFFKFFKFFK

SEQ ID NO: 125 - protein, 37 aa, Artificial Sequence
  Cell Permeation Peptide LL-37
LLGDFFRKSKEKIGKEFKRIVQRIKDFLRNLVPRTES

SEQ ID NO: 126 - protein, 31 aa, Artificial Sequence
  Cell Permeation Peptide Cecropin P1
SWLSKTAKKLENSAKKRISEGIAIAIQGGPR

SEQ ID NO: 127 - protein, 30 aa, Artificial Sequence
  Cell Permeation Peptide Alpha-defensin
ACYCRIPACIAGERRYGTCIYQGRLWAFCC

SEQ ID NO: 128 - protein, 36 aa, Artificial Sequence
  Cell Permeation Peptide b-defensin
DHYNCVSSGGQCLYSACPIFTKIQGTCYRGKAKCCK

SEQ ID NO: 129 - protein, 12 aa, Artificial Sequence
  Cell Permeation Peptide Bactenecin
RKCRIVVIRVCR

SEQ ID NO: 130 - protein, 42 aa, Artificial Sequence
  Cell Permeation Peptide PR-3
RRRPRPPYLPRPRPPPFFPPRLPPRIPPGFPPRFPPRFPGKR

SEQ ID NO: 131 - protein, 13 aa, Artificial Sequence
  Cell Permeation Peptide Indolicidin
ILPWKWPWWPWRR

SEQ ID NO: 132 - protein, 16 aa, Artificial Sequence
  Hydrophobic MTS-containing peptide RFGF
AAVALLPAVLLALLAP

SEQ ID NO: 133 - protein, 11 aa, Artificial Sequence
  RFGF analogue
AALLPVLLAAP

SEQ ID NO: 134 - protein, 13 aa, Artificial Sequence
  Sequence from HIV Tat protein
GRKKRRQRRRPPQ

SEQ ID NO: 135 - DNA, 20 bp, Artificial Sequence
  Antisense Oligonucleotide
gtgcagtattgtagccaggc

SEQ ID NO: 136 - DNA, 20 bp, Artificial Sequence
  Antisense Oligonucleotide
cctcatggtcacatggatga

SEQ ID NO: 137 - DNA, 20 bp, Artificial Sequence
  Antisense Oligonucleotide
ttggcttctcaagatacctg

SEQ ID NO: 138 - DNA, 20 bp, Artificial Sequence
  Antisense Oligonucleotide
gactcttgcaggaagcggct

SEQ ID NO: 139 - DNA, 21 bp, Artificial Sequence
  Antisense Oligonucleotide for inhibiting the expression of a human Huntingtin gene
cugcacgguucuuugugactt

SEQ ID NO: 140 - DNA, 20 bp, Artificial Sequence
  Antisense Oligonucleotide for inhibiting the expression of a human kinesin-1 gene
acgtggaattataccagcca

SEQ ID NO: 141 - DNA, 21 bp, Artificial Sequence
  Antisense Oligonucleotide for inhibiting the expression of a human VEGF gene
gugcuggccuuggugaggutt

SEQ ID NO: 142 - DNA, 21 bp, Artificial Sequence
  Synthetic siRNA
gucuguguaucacgugacgnn

SEQ ID NO: 143 - DNA, 20 bp, Artificial Sequence
  Synthetic siRNA targeted to a nucleic acid molecule encoding acyl CoA cholesterol acyltransferase-2
gcacgaaggatcccaggcac

SEQ ID NO: 144 - DNA, 20 bp, Artificial Sequence
  Synthetic siRNA targeted to a nucleic acid molecule encoding acyl CoA cholesterol acyltransferase-2
ggatcccctcacctcgtctg

SEQ ID NO: 145 - DNA, 20 bp, Artificial Sequence
  Synthetic siRNA targeted to a nucleic acid molecule encoding acyl CoA cholesterol acyltransferase-2
gttcttggccacataattcc

SEQ ID NO: 146 - DNA, 20 bp, Artificial Sequence
  Synthetic siRNA for inhibiting the expression of apolipoprotein(a)
acctgacaccgggatccctc

SEQ ID NO: 147 - DNA, 23 bp, Artificial Sequence
  Synthetic siRNA sequence conserved between mouse lamin B1, lamin B2, keratin complex 2-gene 1 and lamin A/C
aagctggccctggacatggagat